(19) United States
(12) Patent Application Publication
(10) Pub. No.: US 2026/0097111 A1
(43) Pub. Date: Apr. 9, 2026

(54) ETHER LIPIDS FOR HYPERACTIVATION OF MAMMALIAN DENDRITIC CELLS

(71) Applicant: Corner Therapeutics, Inc., Watertown, MA (US)

(72) Inventors: Jonathan CHOW, Newton, MA (US); Emily A. GOSSELIN, West Roxbury, MA (US); Dania ZHIVAKI, West Roxbury, MA (US); Kelsey FINN, Cambridge, MA (US); Kallanthottathil RAJEEV, Wayland, MA (US)

(73) Assignee: Corner Therapeutics, Inc., Watertown, MA (US)

(21) Appl. No.: 19/120,751

(22) PCT Filed: Oct. 18, 2023
(86) PCT No.: PCT/US2023/077220

§ 371 Date: Apr. 11, 2025

Related U.S. Application Data
(60) Provisional application No. 63/417,667, filed on Oct. 19, 2022.
(60) Provisional application No. 63/441,697, filed on Jan. 27, 2023.
(60) Provisional application No. 63/451,885, filed on Mar. 13, 2023.

(51) Int. Cl. A61K 39/145; A61K 31/661; A61K 35/15; A61K 39/00; A61K 45/06; A61P 31/16; C07F 9/10; C12N 5/0784

(52) U.S. Cl. CPC A61K 39/145; A61K 31/661; A61K 35/15; A61K 45/06; A61P 31/16; C07F 9/10; C12N 5/0639; A61K 2039/5154; A61K 2039/5252; A61K 2039/54; A61K 2039/552; A61K 2039/585

(57) ABSTRACT

The present disclosure relates to ether lipid (ETL) compounds, such as ether phospholipid (ETPL) compounds, and uses thereof in hyperactivating mammalian dendritic cells, such as human dendritic cells or canine dendritic cells. The present disclosure also relates to compositions comprising an ETL, such as an ETPL, and one or more of a pathogen recognition receptor agonist, an antigen, and mammalian dendritic cells, as well as methods for production and use of the compositions.

### CROSS-REFERENCE TO RELATED APPLICATIONS

This application claims priority benefit of U.S. Provisional Patent Application No. 63/417,667, filed Oct. 19, 2022; U.S. Provisional Patent Application No. 63/441,697, filed Jan. 27, 2023; and U.S. Provisional Patent Application No. 63/451,885, filed Mar. 13, 2023. The entire contents of those applications are hereby incorporated by reference herein.

### FIELD

The present disclosure relates to ether lipid compounds, including ether phospholipid compounds, and uses thereof in hyperactivating mammalian dendritic cells, such as human dendritic cells or canine dendritic cells. The present disclosure also relates to compositions comprising an ether lipid compound, such as an ether phospholipid compound, and one or more of a pathogen recognition receptor agonist, an antigen, and human or canine dendritic cells, as well as methods for production and use of the compositions.

### BACKGROUND

Typically, dendritic cell (DC) maturation by vaccine adjuvants such as Toll-like receptor agonists does not lead to IL-1beta secretion. In circumstances such as inflammasome activation, IL-1beta secretion does occur but at the cost of DC death by a lytic process of cell death termed pyroptosis (Evavold et al., J Mol Biol, 430(2):217-237, 2018). However, when DCs are matured using the pathogen-associated molecular pattern (PAMP)-containing molecule, lipopolysaccharide (LPS) and the damage-associated molecular pattern (DAMP)-containing molecule such as PGPC (1-palmitoyl-2-glutaryl-sn-glycero-3-phosphocholine) they produce and secrete IL-1beta without pyroptosing, characterizing these viable DCs as hyperactive (Zanoni et al., Science, 352(6290):1232-1236, 2016). In fact, in mouse models, hyperactivated DCs have demonstrated an improved ability to induce an immune response compared to cells activated using LPS alone (Zhivaki et al., Cell Rep, 33(7):108381, 2020). However, little is known about stimuli effective for hyperactivation of human DCs.

As such, the identification of PAMPs and DAMPs suitable for hyperactivation of human DCs is needed in the art. Additionally, the identification of alternatives to the use of LPS and PGPC for hyperactivation of mammalian DCs is desirable. In particular, while LPS (endotoxin) is a potent PAMP, it is contraindicated for use in humans as it can lead to septic shock.

### BRIEF SUMMARY

The present disclosure relates to ether lipid (ETL) compounds, such as ether phospholipid (ETPL) compounds, and uses thereof in hyperactivating mammalian dendritic cells, such as human dendritic cells or canine dendritic cells. The present disclosure also relates to compositions comprising an ETL, such as an ETPL, and one or more of a pathogen recognition receptor agonist, an antigen, and human or canine dendritic cells, as well as methods for production and use of the compositions.

The present disclosure provides compounds of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (V-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof. In some embodiments, the ETL or ETPL is isolated.

The present disclosure also provides compositions comprising an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; wherein the composition further comprises one or more of a TLR agonist, an antigen, and/or dendritic cells. In some embodiments, the TLR agonist comprises a TLR7/8 agonist. In some embodiments, the ETL or ETPL is isolated.

The present disclosure also provides compositions comprising an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; wherein the composition further comprises a TLR agonist. In some embodiments, the TLR agonist comprises a TLR7/8 agonist. In some embodiments, the composition further comprises an antigen. In some embodiments, the composition further comprises dendritic cells. In some embodiments, the composition further comprises an antigen and dendritic cells. In some embodiments, the ETL or ETPL is isolated.

The present disclosure also provides compositions comprising an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; wherein the composition further comprises an antigen. In some embodiments, the composition further comprises a TLR agonist. In some embodiments, the composition further comprises dendritic cells. In some embodiments, the composition further comprises a TLR agonist and dendritic cells. In some embodiments, the TLR agonist comprises a TLR7/8 agonist. In some embodiments, the ETL or ETPL is isolated.

The present disclosure also provides compositions comprising an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; wherein the composition further comprises dendritic cells. In some embodiments, the composition further comprises an antigen. In some embodiments, the composition further comprises a TLR agonist. In some embodiments, the composition further comprises an antigen and a TLR agonist. In some embodiments, the TLR agonist comprises a TLR7/8 agonist. In some embodiments, the ETL or ETPL is isolated.

The present disclosure provides compounds of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof. In some embodiments, the ETL or ETPL is isolated.

The present disclosure also provides compositions comprising an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; wherein the composition further comprises one or more of a TLR agonist, an antigen, and/or dendritic cells. In some embodiments, the TLR agonist comprises a TLR7/8 agonist. In some embodiments, the ETL or ETPL is isolated.

The present disclosure also provides compositions comprising an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; wherein the composition further comprises a TLR agonist. In some embodiments, the TLR agonist comprises a TLR7/8 agonist. In some embodiments, the composition further comprises an antigen. In some embodiments, the composition further comprises dendritic cells. In some embodiments, the composition further comprises an antigen and dendritic cells. In some embodiments, the ETL or ETPL is isolated.

The present disclosure also provides compositions comprising an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; wherein the composition further comprises an antigen. In some embodiments, the composition further comprises a TLR agonist. In some embodiments, the composition further comprises dendritic cells. In some embodiments, the composition further comprises a TLR agonist and dendritic cells. In some embodiments, the TLR agonist comprises a TLR7/8 agonist. In some embodiments, the ETL or ETPL is isolated.

The present disclosure also provides compositions comprising an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; wherein the composition further comprises dendritic cells. In some embodiments, the composition further comprises an antigen. In some embodiments, the composition further comprises a TLR agonist. In some embodiments, the composition further comprises an antigen and a TLR agonist. In some embodiments, the TLR agonist comprises a TLR7/8 agonist. In some embodiments, the ETL or ETPL is isolated.

The present disclosure provides ether lipid (ETL) compounds, wherein the lipid alkyl chain is a C13-C24 n-alkyl chain or a C13-C22 n-alkyl chain. In some embodiments, the n-alkyl chain is a C18-C22 n-alkyl chain, a C21-C24 n-alkyl chain, or a C22 n-alkyl chain. In some embodiments, the present disclosure provides a composition comprising an ether lipid compound, wherein the lipid alkyl chain is a C13-C24 n-alkyl chain, a C13-C22 n-alkyl chain, a C18-C22 n-alkyl chain, a C21-C24 n-alkyl chain, or a C22 n-alkyl chain, wherein the composition further comprises one or more of a TLR agonist, an antigen, and/or dendritic cells. In some embodiments, the TLR agonist comprises a TLR7/8 agonist.

The present disclosure provides a composition comprising an isolated ether lipid (ETL), and a TLR7/8 agonist, wherein the lipid alkyl chain is a C13-C24 n-alkyl chain or a C13-C22 n-alkyl chain. In some embodiments, the n-alkyl chain is a C18-C22 n-alkyl chain, a C21-C24 n-alkyl chain, or a C22 n-alkyl chain. In some embodiments, the composition further comprises an antigen and/or dendritic cells. In some embodiments, the TLR agonist comprises a TLR7/8 agonist.

The present disclosure provides ether phospholipid (ETPL) compounds, wherein the lipid alkyl chain is a C13-C24 n-alkyl chain or a C13-C22 n-alkyl chain. In some embodiments, the n-alkyl chain is a C18-C22 n-alkyl chain, a C21-C24 n-alkyl chain, or a C22 n-alkyl chain. In some embodiments, the present disclosure provides a composition comprising an ether phospholipid compound, wherein the lipid alkyl chain is a C13-C24 n-alkyl chain, a C13-C22 n-alkyl chain, a C18-C22 n-alkyl chain, a C21-C24 n-alkyl chain, or a C22 n-alkyl chain, wherein the composition further comprises one or more of a TLR agonist, an antigen, and/or dendritic cells. In some embodiments, the TLR agonist comprises a TLR7/8 agonist.

The present disclosure provides a composition comprising an isolated ether phospholipid (ETPL), and a TLR agonist, wherein the lipid alkyl chain is a C13-C24 n-alkyl chain or a C13-C22 n-alkyl chain. In some embodiments, the n-alkyl chain is a C18-C22 n-alkyl chain, a C21-C24 n-alkyl chain, or a C22 n-alkyl chain. In some embodiments, the composition further comprises an antigen and/or dendritic cells. In some embodiments, the TLR agonist comprises a TLR7/8 agonist.

In some aspects, the present disclosure provides ether lipid (ETL) compounds with an n-alkyl chain, wherein the n-alkyl chain is a C21-C24 n-alkyl chain. In some embodiments, the present disclosure provides a composition comprising an ether lipid (ETL) compound with an n-alkyl chain, wherein the n-alkyl chain is a C21-C24 n-alkyl chain, and an antigen. In some embodiments, the composition further comprises dendritic cells and/or a TLR agonist. In some embodiments, the composition further comprises dendritic cells and/or a TLR7/8 agonist.

In some aspects, the present disclosure provides a composition comprising an isolated ether lipid (ETL) with an n-alkyl chain, and an antigen, wherein the n-alkyl chain is a C21-C24 n-alkyl chain. In some embodiments, the composition further comprises dendritic cells and/or a TLR agonist. In some embodiments, the composition further comprises dendritic cells and/or a TLR7/8 agonist.

In some aspects, the present disclosure provides ether phospholipid (ETPL) compounds with an n-alkyl chain, wherein the n-alkyl chain is a C21-C24 n-alkyl chain. In some embodiments, the present disclosure provides a composition comprising an ether phospholipid (ETPL) compound with an n-alkyl chain, wherein the n-alkyl chain is a C21-C24 n-alkyl chain, and an antigen. In some embodiments, the composition further comprises dendritic cells and/or a TLR agonist. In some embodiments, the composition further comprises dendritic cells and/or a TLR7/8 agonist.

In some aspects, the present disclosure provides a composition comprising an isolated ether phospholipid (ETPL) with an n-alkyl chain, and an antigen, wherein the n-alkyl chain is a C21-C24 n-alkyl chain. In some embodiments, the composition further comprises dendritic cells and/or a TLR agonist. In some embodiments, the composition further comprises dendritic cells and/or a TLR7/8 agonist.

In some aspects, the present disclosure provides a composition comprising an ether lipid (ETL) with an n-alkyl chain, and dendritic cells, wherein the n-alkyl chain is a C21-C24 n-alkyl chain. In some embodiments, the composition further comprises a TLR agonist and/or an antigen. In some embodiments, the composition further comprises a TLR7/8 agonist and/or an antigen.

In some aspects, the present disclosure provides a composition comprising an isolated ether lipid (ETL) with an n-alkyl chain, and dendritic cells, wherein the n-alkyl chain is a C21-C24 n-alkyl chain. In some embodiments, the composition further comprises a TLR agonist and/or an antigen. In some embodiments, the composition further comprises a TLR7/8 agonist and/or an antigen.

In some aspects, the present disclosure provides a composition comprising an ether phospholipid (ETPL) with an n-alkyl chain, and dendritic cells, wherein the n-alkyl chain is a C21-C24 n-alkyl chain. In some embodiments, the composition further comprises a TLR agonist and/or an antigen. In some embodiments, the composition further comprises a TLR7/8 agonist and/or an antigen.

In some aspects, the present disclosure provides a composition comprising an isolated ether phospholipid (ETPL) with an n-alkyl chain, and dendritic cells, wherein the n-alkyl chain is a C21-C24 n-alkyl chain. In some embodiments, the composition further comprises a TLR agonist and/or an antigen. In some embodiments, the composition further comprises a TLR7/8 agonist and/or an antigen.

In some aspects, the present disclosure provides ether lipid (ETL) compounds with an n-alkyl chain, wherein the n-alkyl chain is a C16-C20 n-alkyl chain. In some embodiments, the present disclosure provides a composition comprising an ether lipid (ETL) compound with an n-alkyl chain, wherein the n-alkyl chain is a C16-C20 n-alkyl chain, and an antigen. In some embodiments, the composition further comprises dendritic cells and/or a TLR agonist. In some embodiments, the composition further comprises dendritic cells and/or a TLR7/8 agonist.

In some aspects, the present disclosure provides a composition comprising an isolated ether lipid (ETL) with an n-alkyl chain, and an antigen, wherein the n-alkyl chain is a C16-C20 n-alkyl chain. In some embodiments, the composition further comprises dendritic cells and/or a TLR agonist. In some embodiments, the composition further comprises dendritic cells and/or a TLR7/8 agonist.

In some aspects, the present disclosure provides ether phospholipid (ETPL) compounds with an n-alkyl chain, wherein the n-alkyl chain is a C16-C20 n-alkyl chain. In some embodiments, the present disclosure provides a composition comprising an ether phospholipid (ETPL) compound with an n-alkyl chain, wherein the n-alkyl chain is a C16-C20 n-alkyl chain, and an antigen. In some embodiments, the composition further comprises dendritic cells and/or a TLR agonist. In some embodiments, the composition further comprises dendritic cells and/or a TLR7/8 agonist.

In some aspects, the present disclosure provides a composition comprising an isolated ether phospholipid (ETPL) with an n-alkyl chain, and an antigen, wherein the n-alkyl chain is a C16-C20 n-alkyl chain. In some embodiments, the composition further comprises dendritic cells and/or a TLR agonist. In some embodiments, the composition further comprises dendritic cells and/or a TLR7/8 agonist.

In some aspects, the present disclosure provides a composition comprising an ether lipid (ETL) with an n-alkyl chain, and dendritic cells, wherein the n-alkyl chain is a C16-C20 n-alkyl chain. In some embodiments, the composition further comprises a TLR agonist and/or an antigen. In some embodiments, the composition further comprises a TLR7/8 agonist and/or an antigen.

In some aspects, the present disclosure provides a composition comprising an isolated ether lipid (ETL) with an n-alkyl chain, and dendritic cells, wherein the n-alkyl chain is a C16-C20 n-alkyl chain. In some embodiments, the composition further comprises a TLR agonist and/or an antigen. In some embodiments, the composition further comprises a TLR7/8 agonist and/or an antigen.

In some aspects, the present disclosure provides a composition comprising an ether phospholipid (ETPL) with an n-alkyl chain, and dendritic cells, wherein the n-alkyl chain is a C16-C20 n-alkyl chain. In some embodiments, the composition further comprises a TLR agonist and/or an antigen. In some embodiments, the composition further comprises a TLR7/8 agonist and/or an antigen.

In some aspects, the present disclosure provides a composition comprising an isolated ether phospholipid (ETPL) with an n-alkyl chain, and dendritic cells, wherein the n-alkyl chain is a C16-C20 n-alkyl chain. In some embodiments, the composition further comprises a TLR agonist and/or an antigen. In some embodiments, the composition further comprises a TLR7/8 agonist and/or an antigen.

In some embodiments of the preceding aspects, the antigen is present in a biological sample obtained from an individual. In some embodiments, the biological sample comprises biopsy tissue. In some embodiments, the biological sample comprises cells. In other embodiments, the biological sample does not comprise cells. In some embodiments, the biological sample comprises pus from an abscess. In some embodiments, the antigen comprises a proteinaceous antigen. In some embodiments, the antigen comprises a tumor antigen. In some embodiments, the tumor antigen comprises a synthetic or recombinant neoantigen. In some embodiments, the tumor antigen comprises a tumor cell lysate. In some embodiments, the antigen comprises a microbial antigen and the microbial antigen comprises one or more of a viral antigen, a bacterial antigen, a protozoan antigen, and a fungal antigen. In some embodiments, the microbial antigen comprises a purified or recombinant surface protein. In some embodiments, the microbial antigen comprises an inactivated, whole virus.

In some embodiments, the composition does not comprise liposomes. In some embodiments, the composition does not comprise LPS or MPLA. In some embodiments, the composition does not comprise oxPAPC or a species of oxPAPC. In some embodiments, the composition does not comprise HOdiA-PC, KOdiA-PC, HOOA-PC, KOOA-PC, and/or PGPC. In some embodiments, the composition does not comprise lysophosphatidylcholine (LPC). In some embodiments, the composition does not comprise 1-behenoyl-2-hydroxy-sn-glycero-3-phosphocholine [LPC(22:0)].

In some embodiments, the composition further comprises an adjuvant, wherein the adjuvant comprises an aluminum salt adjuvant, a squalene-in-water emulsion, a saponin, or combinations thereof.

In some embodiments, the present disclosure provides a pharmaceutical formulation comprising the composition of any of the preceding aspects and a pharmaceutically acceptable excipient.

In additional aspects, the present disclosure provides a method for production of hyperactivated dendritic cells, the method comprising contacting the dendritic cells with a composition comprising effective amounts of an isolated ether lipid (ETL) with a C13-C22 n-alkyl chain or a C13-C24 n-alkyl chain, and a TLR agonist for production of hyperactivated dendritic cells, wherein the hyperactivated dendritic cells secrete IL-1beta without undergoing pyroptosis. In some embodiments, the dendritic cells are contacted ex vivo with the composition or pharmaceutical formulation of any one of the preceding embodiments. In other embodiments, the dendritic cells are contacted in vivo with the pharmaceutical formulation comprising the composition of any one of the preceding embodiments. In some aspects, the present disclosure provides a pharmaceutical formulation comprising a plurality of the hyperactivated dendritic cells produced by the preceding embodiments, and a pharmaceutically acceptable excipient. In some embodiments, the plurality comprises at least 10³, 10⁴, 10⁵, 10⁶, 10⁷ or 10⁸ hyperactivated DCs. In some embodiments, the TLR agonist comprises a TLR7/8 agonist.

In additional aspects, the present disclosure provides a method for production of hyperactivated dendritic cells, the method comprising contacting the dendritic cells with a composition comprising effective amounts of an isolated ether phospholipid (ETPL) with a C13-C22 n-alkyl chain or a C13-C24 n-alkyl chain, and a TLR agonist for production of hyperactivated dendritic cells, wherein the hyperactivated dendritic cells secrete IL-1beta without undergoing pyroptosis. In some embodiments, the dendritic cells are contacted ex vivo with the composition or pharmaceutical formulation of any one of the preceding embodiments. In other embodiments, the dendritic cells are contacted in vivo with the pharmaceutical formulation comprising the composition of any one of the preceding embodiments. In some aspects, the present disclosure provides a pharmaceutical formulation comprising a plurality of the hyperactivated dendritic cells produced by the preceding embodiments, and a pharmaceutically acceptable excipient. In some embodiments, the plurality comprises at least 10³, 10⁴, 10⁵, 10⁶, 10⁷ or 10⁸ hyperactivated DCs. In some embodiments, the TLR agonist comprises a TLR7/8 agonist.

In additional aspects, the present disclosure provides a composition comprising an isolated ether lipid (ETL) with an n-alkyl chain, and a pathogen recognition receptor (PRR) agonist, wherein the n-alkyl chain is a C13-C22 n-alkyl chain or a C13-C24 n-alkyl chain. In some embodiments, the PRR agonist is an agonist of a toll-like receptor (TLR), a NOD-like receptor (NLR), a RIG-I-like receptor (RLR), or a C-type lectin receptor (CLR). In some embodiments, the PRR agonist is an agonist of a cytosolic DNA sensor (CDS) or a stimulator of IFN genes (STING). In some embodiments, the PRR agonist comprises a TLR7/8 agonist. In some embodiments, the composition further comprises an antigen and/or dendritic cells.

In additional aspects, the present disclosure provides a composition comprising an isolated ether phospholipid (ETPL) with an n-alkyl chain, and a pathogen recognition receptor (PRR) agonist, wherein the n-alkyl chain is a C13-C22 n-alkyl chain or a C13-C24 n-alkyl chain. In some embodiments, the PRR agonist is an agonist of a toll-like receptor (TLR), a NOD-like receptor (NLR), a RIG-I-like receptor (RLR), or a C-type lectin receptor (CLR). In some embodiments, the PRR agonist is an agonist of a cytosolic DNA sensor (CDS) or a stimulator of IFN genes (STING). In some embodiments, the PRR agonist comprises a TLR7/8 agonist. In some embodiments, the composition further comprises an antigen and/or dendritic cells.

In some embodiments of the preceding aspects, the n-alkyl chain of the ether lipid (ETL) is a C21-C24 n-alkyl chain. In some embodiments, the n-alkyl chain of the ETL is a C22 n-alkyl chain.

In some embodiments of the preceding aspects, the n-alkyl chain of the ether phospholipid (ETPL) is a C21-C24 n-alkyl chain. In some embodiments, the n-alkyl chain of the ETPL is a C22 n-alkyl chain.

In some embodiments of the preceding aspects, the ETPL comprises 1-docosyl-sn-glycerol-3-phosphocholine (DGPC). In some embodiments of the preceding aspects, the ETPL comprises 1-docosyl-sn-glycerol-3-phosphate (DGP).

In some embodiments of the preceding aspects, the TLR agonist is a small molecule with a molecule weight of 900 daltons or less. In some embodiments of the preceding aspects, the TLR7/8 agonist is a small molecule with a molecule weight of 900 daltons or less. In some embodiments, the TLR7/8 agonist comprises an imidazoquinoline compound. In some embodiments, the TLR7/8 agonist comprises resiquimod (R848). In some embodiments, the ETPL comprises DGPC, and the TLR7/8 agonist comprises resiquimod (R848). In some embodiments, the ETPL comprises DGP, and the TLR7/8 agonist comprises resiquimod (R848).

The present disclosure further provides compositions for hyperactivation of human dendritic cells, comprising an ether lipid (ETL) compound with an n-alkyl chain, and a pathogen recognition receptor (PRR) agonist, wherein the n-alkyl chain is a C22 n-alkyl chain, and wherein the composition is effective for achieving a higher level of dendritic cell hyperactivation than a comparator composition comprising a comparator compound in place of the ETL. The present disclosure further provides compositions for hyperactivation of human dendritic cells, comprising an isolated ether lipid (ETL) compound with an n-alkyl chain, and a pathogen recognition receptor (PRR) agonist, wherein the n-alkyl chain is a C22 n-alkyl chain, and wherein the composition is effective for achieving a higher level of dendritic cell hyperactivation than a comparator composition comprising a comparator compound in place of the ETL. In some embodiments, the hyperactivation occurs in vitro or ex vivo. In other embodiments, the hyperactivation occurs in vivo. In some embodiments, the higher level of dendritic cell hyperactivation comprises induction of IL-1beta secretion from the human dendritic cells in vitro at a level that is at least 2, 3 or 4 fold higher when contacted with the composition comprising the ETL and the PRR agonist than when contacted with the comparator composition comprising the comparator compound and the PRR agonist, wherein the PRR agonist is LPS. In some embodiments, the concentration of the ETL and the concentration of the comparator compound are the same concentration, optionally in a range of from about 10 μM to about 80 μM, and the LPS is present at a concentration of 1 μg/ml in both the composition and the comparator composition. In some embodiments, the higher level of dendritic cell hyperactivation comprises a lipid activity index for IL-1beta secretion from the human dendritic cells for the composition comprising the ETL and the PRR agonist that is at least 4, 5 or 6 fold higher in activity units than that of the comparator composition comprising the comparator compound and the PRR agonist. In some embodiments, the comparator compound is PGPC. In some embodiments, the comparator compound is 1-behenoyl-2-hydroxy-sn-glycero-3-phosphocholine [LPC(22:0)].

The present disclosure further provides compositions for hyperactivation of human dendritic cells, comprising an ether phospholipid (ETPL) compound with an n-alkyl chain, and a pathogen recognition receptor (PRR) agonist, wherein the n-alkyl chain is a C22 n-alkyl chain, and wherein the composition is effective for achieving a higher level of dendritic cell hyperactivation than a comparator composition comprising a comparator compound in place of the ETPL. The present disclosure further provides compositions for hyperactivation of human dendritic cells, comprising an isolated ether phospholipid (ETPL) compound with an n-alkyl chain, and a pathogen recognition receptor (PRR) agonist, wherein the n-alkyl chain is a C22 n-alkyl chain, and wherein the composition is effective for achieving a higher level of dendritic cell hyperactivation than a comparator composition comprising a comparator compound in place of the ETPL. In some embodiments, the hyperactivation occurs in vitro or ex vivo. In other embodiments, the hyperactivation occurs in vivo. In some embodiments, the higher level of dendritic cell hyperactivation comprises induction of IL-1beta secretion from the human dendritic cells in vitro at a level that is at least 2, 3 or 4 fold higher when contacted with the composition comprising the ETPL and the PRR agonist than when contacted with the comparator composition comprising the comparator compound and the PRR agonist, wherein the PRR agonist is LPS. In some embodiments, the concentration of the ETPL and the concentration of the comparator compound are the same concentration, optionally in a range of from about 10 μM to about 80 μM, and the LPS is present at a concentration of 1 μg/ml in both the composition and the comparator composition. In some embodiments, the higher level of dendritic cell hyperactivation comprises a lipid activity index for IL-1beta secretion from the human dendritic cells for the composition comprising the ETPL and the PRR agonist that is at least 4, 5 or 6 fold higher in activity units than that of the comparator composition comprising the comparator compound and the PRR agonist. In some embodiments, the comparator compound is PGPC. In some embodiments, the comparator compound is 1-behenoyl-2-hydroxy-sn-glycero-3-phosphocholine [LPC(22:0)].

The ether lipid (ETL) compounds, such as isolated ether lipid compounds, and ether phospholipid (ETPL) compounds, such as isolated ether phospholipid compounds, can be administered in the form of micelles.

The ether lipid (ETL) compounds, such as isolated ether lipid compounds, and ether phospholipid (ETPL) compounds, such as isolated ether phospholipid compounds, can be administered in the form of lipid nanoparticles (LNPs).

In some embodiments of the present disclosure, the LNPs of the compositions are enriched in particles with lipid bilayers (liposomes) relative to particles with a single lipid layer (micelle). Specifically, in some embodiments, the LNPs comprise liposomes, and little to substantially no micelles. In some embodiments, the LNPs comprise liposomes, and less than about 10% of the lipid particles present are micelles. In some embodiments, the LNPs comprise liposomes, and less than about 5% of the lipid particles present are micelles. In some embodiments, the LNPs comprise liposomes, and less than about 1% of the lipid particles present are micelles.

In some embodiments, the present disclosure provides lipid nanoparticles comprising an ETL or ETPL compound and at least one further lipid, and uses thereof in hyperactivating mammalian dendritic cells. The present disclosure also relates to compositions comprising an ETL or ETPL compound and at least one further lipid, wherein the compositions further comprise one or more of a pathogen recognition receptor agonist, an antigen, and mammalian dendritic cells, as well as methods for production and use of the compositions.

In some aspects, the present disclosure provides a composition comprising an ETL or ETPL compound and a TLR agonist, such as a TLR7/8 agonist, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-1-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; and the ETL or ETPL and at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the composition further comprises an antigen and/or dendritic cells.

In some aspects, the present disclosure provides a composition comprising an ETL or ETPL compound and an antigen, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; and the ETL or ETPL and at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the composition further comprises a TLR agonist, such as a TLR7/8 agonist, and/or dendritic cells.

In some aspects, the present disclosure provides a composition comprising an ETL or ETPL compound and dendritic cells, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; and the ETL or ETPL and at least one further lipid are part of a lipid nanoparticle (LNP). In additional embodiments, the ETL or ETPL compound is isolated. In some embodiments, the at least one further lipid is selected from the group consisting of an ionizable lipid, a cationic lipid, a further phospholipid, a pegylated lipid, a structural lipid, and mixtures thereof. In some embodiments, the composition further comprises a TLR agonist, such as a TLR7/8 agonist, and/or an antigen.

In some aspects, the present disclosure provides a composition comprising an ETL or ETPL compound and a TLR agonist, such as a TLR7/8 agonist, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; and the ETL or ETPL and at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the composition further comprises an antigen and/or dendritic cells.

In some aspects, the present disclosure provides a composition comprising an ETL or ETPL compound and an antigen, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; and the ETL or ETPL and at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the composition further comprises a TLR agonist, such as a TLR7/8 agonist, and/or dendritic cells.

In some aspects, the present disclosure provides a composition comprising an ETL or ETPL compound and dendritic cells, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; and the ETL or ETPL and at least one further lipid are part of a lipid nanoparticle (LNP). In additional embodiments, the ETL or ETPL compound is isolated. In some embodiments, the at least one further lipid is selected from the group consisting of an ionizable lipid, a cationic lipid, a further phospholipid, a pegylated lipid, a structural lipid, and mixtures thereof. In some embodiments, the composition further comprises a TLR agonist, such as a TLR7/8 agonist, and/or an antigen.

In some embodiments of the preceding aspects, the antigen is present in a biological sample obtained from an individual. In some embodiments, the biological sample comprises biopsy tissue. In some embodiments, the biological sample comprises cells. In other embodiments, the biological sample does not comprise cells. In some embodiments, the biological sample comprises pus from an abscess. In some embodiments, the antigen comprises a proteinaceous antigen. In some embodiments, the antigen comprises a tumor antigen. In some embodiments, the tumor antigen comprises a synthetic or recombinant neoantigen. In some embodiments, the tumor antigen comprises a tumor cell lysate. In some embodiments, the antigen comprises a microbial antigen and the microbial antigen comprises one or more of a viral antigen, a bacterial antigen, a protozoan antigen, and a fungal antigen. In some embodiments, the microbial antigen comprises a purified or recombinant surface protein. In some embodiments, the microbial antigen comprises an inactivated, whole virus.

In some embodiments, the composition does not comprise LPS or MPLA. In some embodiments, the composition does not comprise oxPAPC or a species of oxPAPC. In some embodiments, the composition does not comprise HOdiA-PC, KOdiA-PC, HOOA-PC, KOOA-PC, and/or PGPC. In some embodiments, the composition does not comprise isolated mRNA. In some embodiments, the composition does not comprise a surfactant (e.g., a poloxamer). In some embodiments, the composition does not comprise Poloxamer 407 (KP407), Poloxamer 188 (KP188), and/or Pluronic P123 (P123).

In some embodiments, the composition further comprises an adjuvant, wherein the adjuvant comprises an aluminum salt adjuvant, a squalene-in-water emulsion, a saponin, or combinations thereof.

In some embodiments, the present disclosure provides a pharmaceutical formulation comprising the composition of any of the preceding aspects and a pharmaceutically acceptable excipient. In some embodiments, the formulation does not comprise a surfactant (e.g., a poloxamer). In some embodiments, the formulation does not comprise Poloxamer 407 (KP407), Poloxamer 188 (KP188), and/or Pluronic P123 (P123).

In other aspects, the present disclosure provides a method for production of hyperactivated dendritic cells, the method comprising contacting the dendritic cells with an effective amount of the composition or pharmaceutical formulation of any of the preceding embodiments for production of hyperactivated dendritic cells, wherein the hyperactivated dendritic cells secrete IL-1beta without undergoing pyroptosis, and the ETL or ETPL and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the dendritic cells are contacted ex vivo with the composition or pharmaceutical formulation of any one of the preceding embodiments. In other embodiments, the dendritic cells are contacted in vivo with the pharmaceutical formulation comprising the composition of any one of the preceding embodiments. In some aspects, the present disclosure provides a pharmaceutical formulation comprising a plurality of the hyperactivated dendritic cells produced by the preceding embodiments, and a pharmaceutically acceptable excipient. In some embodiments, the plurality comprises at least 10³, 10⁴, 10⁵, 10⁶, 10⁷ or 10⁸ hyperactivated DCs.

In other aspects, the present disclosure provides a composition comprising an ETL or an ETPL, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; at least one further lipid, and a pathogen recognition receptor (PRR) agonist, and the ETL or ETPL and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the PRR agonist is an agonist of a toll-like receptor (TLR), a NOD-like receptor (NLR), a RIG-I-like receptor (RLR), or a C-type lectin receptor (CLR). In some embodiments, the PRR agonist is an agonist of a cytosolic DNA sensor (CDS) or a stimulator of IFN genes (STING). In some embodiments, the PRR agonist comprises a TLR7/8 agonist. In some embodiments, the composition further comprises an antigen and/or dendritic cells.

In some embodiments of the preceding aspects, the TLR7/8 agonist is a small molecule with a molecule weight of 900 daltons or less. In some embodiments, the TLR7/8 agonist comprises an imidazoquinoline compound. In some embodiments, the TLR7/8 agonist comprises resiquimod (R848).

The present disclosure further provides compositions for hyperactivation of human dendritic cells, comprising an ETL or an ETPL, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof, at least one further lipid, and a pathogen recognition receptor (PRR) agonist, wherein the composition is effective for achieving a higher level of dendritic cell hyperactivation than a comparator composition comprising a comparator compound in place of the ETL or ETPL. In some embodiments, the at least one further lipid is selected from the group consisting of an ionizable lipid, a cationic lipid, a further phospholipid, a pegylated lipid, a structural lipid, and mixtures thereof. In some embodiments, the hyperactivation occurs in vitro or ex vivo. In other embodiments, the hyperactivation occurs in vivo. In some embodiments, the higher level of dendritic cell hyperactivation comprises induction of IL-1beta secretion from the human dendritic cells in vitro at a level that is at least 2, 3 or 4 fold higher when contacted with the composition comprising the ETL or ETPL and the PRR agonist than when contacted with the comparator composition comprising the comparator compound and the PRR agonist, wherein the PRR agonist is LPS. In some embodiments, the concentration of the ETL or ETPL and the concentration of the comparator compound are the same concentration, optionally in a range of from about 10 μM to about 80 μM, and the LPS is present at a concentration of 1 μg/ml in both the composition and the comparator composition. In some embodiments, the higher level of dendritic cell hyperactivation comprises a lipid activity index for IL-1beta secretion from the human dendritic cells for the composition comprising the ETL or ETPL and the PRR agonist that is at least 4, 5 or 6 fold higher in activity units than that of the comparator composition comprising the comparator compound and the PRR agonist. In some embodiments, the comparator compound is PGPC.

In other aspects, the present disclosure provides a composition comprising an ETL or an ETPL, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; at least one further lipid, and a pathogen recognition receptor (PRR) agonist, and the ETL or ETPL and the at least one further lipid are part of a lipid nanoparticle (LNP). In some embodiments, the at least one further lipid is selected from the group consisting of an ionizable lipid, a cationic lipid, a further phospholipid, a pegylated lipid, a structural lipid, and mixtures thereof. In some embodiments, the PRR agonist is an agonist of a toll-like receptor (TLR), a NOD-like receptor (NLR), a RIG-I-like receptor (RLR), or a C-type lectin receptor (CLR). In some embodiments, the PRR agonist is an agonist of a cytosolic DNA sensor (CDS) or a stimulator of IFN genes (STING). In some embodiments, the PRR agonist comprises a TLR7/8 agonist. In some embodiments, the composition further comprises an antigen and/or dendritic cells.

In some embodiments of the preceding aspects, the TLR7/8 agonist is a small molecule with a molecule weight of 900 daltons or less. In some embodiments, the TLR7/8 agonist comprises an imidazoquinoline compound. In some embodiments, the TLR7/8 agonist comprises resiquimod (R848).

The present disclosure further provides compositions for hyperactivation of human dendritic cells, comprising an ETL or an ETPL, wherein the ETL or ETPL compound is a compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof, at least one further lipid, and a pathogen recognition receptor (PRR) agonist, wherein the composition is effective for achieving a higher level of dendritic cell hyperactivation than a comparator composition comprising a comparator compound in place of the ETL or ETPL. In some embodiments, the at least one further lipid is selected from the group consisting of an ionizable lipid, a cationic lipid, a further phospholipid, a pegylated lipid, a structural lipid, and mixtures thereof. In some embodiments, the hyperactivation occurs in vitro or ex vivo. In other embodiments, the hyperactivation occurs in vivo. In some embodiments, the higher level of dendritic cell hyperactivation comprises induction of IL-1beta secretion from the human dendritic cells in vitro at a level that is at least 2, 3 or 4 fold higher when contacted with the composition comprising the ETL or ETPL and the PRR agonist than when contacted with the comparator composition comprising the comparator compound and the PRR agonist, wherein the PRR agonist is LPS. In some embodiments, the concentration of the ETL or ETPL and the concentration of the comparator compound are the same concentration, optionally in a range of from about 10 μM to about 80 μM, and the LPS is present at a concentration of 1 μg/ml in both the composition and the comparator composition. In some embodiments, the higher level of dendritic cell hyperactivation comprises a lipid activity index for IL-1beta secretion from the human dendritic cells for the composition comprising the ETL or ETPL and the PRR agonist that is at least 4, 5 or 6 fold higher in activity units than that of the comparator composition comprising the comparator compound and the PRR agonist. In some embodiments, the comparator compound is PGPC.

In any of the embodiments disclosed herein, the ether lipid can be in the form of a pharmaceutically acceptable salt.

In any of the embodiments disclosed herein, the ether phospholipid can be in the form of a pharmaceutically acceptable salt.

In any of the embodiments disclosed herein, wherever a compound of Formula (I), Formula (II), Formula (III), Formula (III-A), Formula (III-A-1), Formula (III-A-2), Formula (II-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 is disclosed in an embodiment, the disclosure also encompasses the use of a Compound of any other Formula or other specific Compound instead in that embodiment.

The disclosure of methods comprising administering the compounds and compositions of the present disclosure to a subject (e.g., subject in need thereof), are also relevant to uses of the compounds and compositions for treating or preventing a disease or disorder or a treating a subject having a disease or disorder, and uses of the compounds and compositions in the manufacture of a medicament for treating or preventing a disease or disorder or treating a subject having a disease or disorder.

In any of the embodiments disclosed herein that comprise an antigen, the antigen may comprise one or more viral antigens. In some embodiments, the one or more viral antigens comprise one or both of influenza A and influenza B antigens. In some embodiments, the one or both of influenza A and influenza B antigens comprise one or both of hemagglutinin and nucleoprotein. In some embodiments, the viral antigens comprise inactivated virions, optionally wherein the inactivated virions comprise inactivated, split virions. In some embodiments comprising both influenza A and influenza B antigens, the antigens are of an H1N1 influenza A virus, an H3N2 influenza A virus, a Victoria lineage influenza B virus, and a Yamagata lineage influenza B virus.

### BRIEF DESCRIPTION OF THE DRAWINGS

Data presented in bar graphs of the following figures are shown as means with error bars representing standard deviation (SD). *p<0.05, **p<0.01, ***p<0.001, ****p<0.0001, and ns=not significant.

FIG. 1A shows cell viability and FIG. 1B shows IL-1β secretion by human monocyte-derived dendritic cells (moDCs) under the indicated test conditions.

FIG. 2A shows cell viability and FIG. 2B shows IL-1β secretion by human moDCs under the indicated test conditions.

FIG. 3A shows cell viability and FIG. 3B shows IL-1β secretion by human moDCs under the indicated test conditions.

FIG. 4A shows IL-1β secretion, FIG. 4B shows cell viability, and FIG. 4C shows TNFα secretion by human moDCs under the indicated test conditions.

FIG. 5 shows dendritic cell migration from the skin to the draining lymph nodes under the indicated test conditions.

FIG. 6 shows survival rates of mice bearing LLC1 tumors that were immunized with PBS or a whole tumor lysate in the presence of a PAMP and a DAMP.

FIG. 7 shows IFNγ-secreting cells in draining lymph nodes of immunized mice.

FIG. 8 shows IL-1β secretion by human moDCs treated with 22:0 Lyso PC, DPD (Compound 9), Compound 10, or vehicle, with and without R848.

FIG. 9 shows viability of cells treated with 22:0 Lyso PC, DPD (Compound 9), Compound 10, or vehicle, with and without R848.

FIG. 10 shows IL-6 secretion by human moDCs treated with 22:0 Lyso PC, Compound 9 (DPD), Compound 2 (DGP), Compound 7, Compound 8, or vehicle, without R848, with R848, and with R848 and MCC950.

FIG. 11 shows IL-1β secretion by human moDCs treated with 22:0 Lyso PC, Compound 9 (DPD), Compound 2 (DGP), Compound 7, Compound 8, or vehicle, without R848, with R848, and with R848 and MCC950.

FIG. 12 shows viability of cells treated with 22:0 Lyso PC, Compound 9 (DPD), Compound 2 (DGP), Compound 7, Compound 8, or vehicle, without R848, with R848, and with R848 and MCC950.

FIG. 13 shows IL-6 secretion by human moDCs treated with Compound 11, Compound 12, or vehicle, without R848, with R848, and with R848 and MCC950.

FIG. 14 shows IL-1β secretion by human moDCs treated with Compound 11, Compound 12, or vehicle, without R848, with R848, and with R848 and MCC950.

FIG. 15 shows viability of cells treated with Compound 11, Compound 12, or vehicle, without R848, with R848, and with R848 and MCC950.

FIG. 16 shows IL-6 secretion by human moDCs treated with Compound 1, 4, 6, 11, 12, 13, 14, 15, 16, 2, 22:0 LPC, or vehicle, without R848, with R848, and with R848 and MCC950. Compound concentration tested was 41.25 micromolar.

FIG. 17 shows IL-1β secretion by human moDCs treated with Compound 1, 4, 6, 11, 12, 13, 14, 15, 16, 2, 22:0 LPC, or vehicle, without R848, with R848, and with R848 and MCC950. Compound concentration tested was 41.25 micromolar.

FIG. 18 shows cell viability of cells treated with Compound 1, 4, 6, 11, 12, 13, 14, 15, 16, 2, 22:0 LPC, or vehicle, without R848, with R848, and with R848 and MCC950.

FIG. 19 shows cell viability of cells treated with Compound 1, 2, 22:0 LPC, or vehicle, without R848, with R848, and with R848 and MCC950. Compound concentration tested was 20.6 micromolar.

FIG. 20 shows IL-1β secretion by human moDCs treated with Compound 1, 2, 22:0 LPC, or vehicle, without R848, with R848, and with R848 and MCC950. Compound concentration tested was 20.6 micromolar.

FIG. 21 shows IL-1β secretion by human moDCs under the indicated test conditions. The moDCs in each plot were derived from a distinct healthy donor (HD) and symbols represent values obtained from biological replicates. Ordinary two-way ANOVA was conducted, followed by Tukey's multiple comparisons with a single pool variance.

FIG. 22 shows cell viability as determined by measuring lactate dehydrogenase (LDF) release after treatment of human moDCs under the indicated test conditions. Symbols represent the mean value of biological triplicates from moDCs derived from a given healthy donor (HD93, HD94, HD95, and HD96). Dashed lines indicate an acceptable range in cell viability.

FIG. 23 shows the number of live CD11c+CD209+ cells as determined by flow cytometry that were present in a fixed volume acquired from every sample. Symbol shapes are unique to each healthy donor. Statistical testing was completed using repeated measures one-way ANOVA followed by Tukey's comparisons with individual variances.

FIG. 24A shows the percentage of live CD11c+CD209+ cells expressing CD83, and FIG. 24B shows the mean fluorescence intensity (MFI) of CD83 staining of live CD11c+CD209+ cells. Symbol shapes are unique to each donor. Statistical testing was completed using repeated measures one-way ANOVA followed by Tukey's comparisons with individual variances.

FIG. 25A shows the percentage of live CD11c+CD209+ cells expressing CD86, and FIG. 25B shows the MFI of CD86 staining of live CD11c+CD209+ cells. Symbol shapes are unique to each donor. Statistical testing was completed using repeated measures one-way ANOVA followed by Tukey's comparisons with individual variances.

FIG. 26A shows the percentage of live CD11c+CD209+ cells expressing CD40, and FIG. 26B shows the MFI of CD40 staining of live CD11c+CD209+ cells. Symbol shapes are unique to each donor. Statistical testing was completed using repeated measures one-way ANOVA followed by Tukey's comparisons with individual variances.

FIG. 27A shows the percentage of live CD11c+CD209+ cells expressing MHC class I (HLA-ABC), and FIG. 27B shows the MFI of MHC class I staining of live CD11c+CD209+ cells. Symbol shapes are unique to each donor. Statistical testing was completed using repeated measures one-way ANOVA followed by Tukey's comparisons with individual variances.

FIG. 28A shows the percentage of live CD11c+CD209+ cells expressing MHC class II (HLA-DR), and FIG. 28B shows the MFI of MHC class II staining of live CD11c+CD209+ cells. Symbol shapes are unique to each donor. Statistical testing was completed using repeated measures one-way ANOVA followed by Tukey's comparisons with individual variances.

FIG. 29A shows the percentage of live CD11c+CD209+ cells expressing CCR7, and FIG. 29B shows the MFI of CCR7 staining of live CD11c+CD209+ cells. Symbol shapes are unique to each donor. Statistical testing was completed using repeated measures one-way ANOVA followed by Tukey's comparisons with individual variances.

FIG. 30 shows the concentration of IL-1β present in cell culture supernatant after treatment of moDCs for 24 hours under the indicated conditions. Graph shows data from individual human donor samples, and symbols represent values obtained from biological replicates. For statistical comparisons, ordinary two-way ANOVA was conducted, followed by Tukey's multiple comparisons test with a single pooled variance.

FIG. 31A shows cell viability as determined by measuring LDH activity of cell culture supernatant of moDCs treated for 24 hours under the indicated conditions. FIG. 31B shows cell viability as determined by measurement of luminescent signal induced by ATP by using CellTiter-Glo 2.0 reagent after lysis of moDCs for 24 hours under the indicated conditions. The x-axis labeling applies to both panels. Symbols in graphs represent biological replicates from a donor. Dashed lines indicate acceptable ranges in cell viability.

FIG. 32A-C shows NF-kB-dependent gene expression by human moDCs after treatment under the indicated conditions. FIG. 32A shows the concentration of IL-6, FIG. 32B shows the concentration of IL-10, and FIG. 32C shows the concentration of IL12p70 present in cell culture supernatant after treatment of moDCs for 24 hours. Symbols represent biological replicates. For statistical comparisons, ordinary two-way ANOVA was conducted, followed by Tukey's multiple comparisons test with a single pooled variance.

FIG. 33A-B shows IRF-dependent gene expression by human moDCs after treatment under the indicated conditions. FIG. 33A shows the concentration of IP-10, and FIG. 33B shows the concentration of IFNα2 present in cell culture supernatant after treatment of moDCs for 24 hours. Symbols represent biological replicates. For statistical comparisons, ordinary two-way ANOVA was conducted, followed by Tukey's multiple comparisons test with a single pooled variance.

FIG. 34A-C shows migration of human moDCs derived from three different donors (HD87, HD92 and HD93), after treatment with the indicated stimuli. In brief, cells were plated in the apical chamber of 5 μm pore transwells. Media containing indicated concentrations of CCL19 were added to the basal chambers, and cells were incubated overnight. Migration of moDC was quantified by enumerating cells in the basal chamber. Symbols represent biological replicates. For statistical comparisons, ordinary two-way ANOVA was conducted, followed by Tukey's multiple comparisons test with a single pooled variance.

FIG. 35A-B shows the effects of hyperactivation of human moDCs on T-cells. FIG. 35A shows the concentration of IL-6 present in cell culture supernatant after treatment of moDC and memory CD4+ T cell cocultures with the indicated stimuli for 2 days. FIG. 35B shows the concentration of IL-6 present in cell culture supernatant after treatment of CD4+ T cells with the indicated stimuli for 2 days. IL-6 was measured from cell culture supernatants using a Lumit immunoassay. Columns represent mean values, and data points represent values of biological replicates. Ordinary two-way ANOVA was conducted, followed by Tukey's multiple comparisons with a single pool variance.

FIG. 36A-B shows that stimulation of human moDCs with R848 and DGP mediates hyperactivation in cocultures. FIG. 36A shows the concentration of IL-1β present in cell culture supernatant after treatment of moDC and memory CD4+ T cell cocultures with the indicated stimuli for 2 days. IL-1β was measured from cell culture supernatants using a Lumit immunoassay. FIG. 36B shows cell viability of after treatment of moDC and memory CD4+ T cell cocultures with the indicated stimuli for 2 days. Columns represent mean values, and data points represent values of biological replicates. Ordinary two-way ANOVA was conducted, followed by Tukey's multiple comparisons with a single pool variance.

FIG. 37A-C shows that Th1 responses are induced by human moDCs stimulated with R848 and DGP. FIG. 37A shows the concentration of IFNγ present in cell culture supernatants after treatment of moDC and memory CD4+ T cell cocultures with the indicated stimuli (with anti-CD3) for 2 days. FIG. 37B shows IFNγ present in cell culture supernatant after treatment of moDCs alone, moDCs and CD4+ T cells, and CD4+ T cells alone with 2.85 μM R848, 82.5 μM DGP, and 0.1 ng/mL anti-CD3 for 2 days. FIG. 37C shows the concentration of IFNγ present in cell culture supernatant after treatment of moDC and memory CD4+ T cell cocultures with the indicated stimuli (without anti-CD3) for 2 days. IFNγ was measured using a Lumit immunoassay. Columns represent mean values, and data points represent values of biological replicates. Ordinary two-way ANOVA was conducted, followed by Tukey's multiple comparisons with a single pool variance.

FIG. 38A-C shows that minimal amounts of Th2 cytokines are induced by human moDCs stimulated with R848 and DGP. FIG. 38A shows the concentration of IL-4, FIG. 38B shows the concentration of IL-5, and FIG. 38C shows the concentration of IL-13 present in cell culture supernatants after treatment of moDC and memory CD4+ T cell cocultures with the indicated stimuli for 2 days. Cytokines were measured using Lumit immunoassays. Columns represent mean values, and data points represent values of biological replicates. Ordinary two-way ANOVA was conducted, followed by Tukey's multiple comparisons with a single pool variance.

FIG. 39A-F shows that Th17 responses are induced by human moDCs stimulated with R848 and DGP. FIG. 39A shows the concentration of IL-17A, FIG. 39B shows the concentration of IL-17F, and FIG. 39C shows the concentration of IL-IL-22 present in cell culture supernatants after treatment of moDC and memory CD4+ T cell cocultures with the indicated stimuli for 2 days. FIG. 39D shows the concentration of IL-17A, FIG. 39E shows the concentration of IL-17F, and FIG. 39F shows the concentration of IL-22 present in cell culture supernatants after treatment of moDCs alone, moDCs and CD4+ T cells, and CD4+ T cells alone with 2.85 μM R848, 41.3 μM DGP, and 0.1 ng/mL anti-CD3 for 2 days. Cytokines were measured using Lumit immunoassays. Columns represent mean values, and data points represent values of biological replicates. Ordinary two-way ANOVA was conducted, followed by Tukey's multiple comparisons with a single pool variance.

FIG. 40 shows that R848 in combination with DGP (Compound 2) enhances antigen-specific reactivation of CD8+ T-cells. In brief, the concentration of IFNγ present in cell culture supernatants of CD8+ T cells co-cultured with pre-treated Flt3L-DCs for 96 hours was quantified as a measure T-cell activation.

FIG. 41 shows a process for reducing DGP (Compound 2) DP (drug product) size, which increases DC hyperactivation in vitro and in vivo, by using jet milling micronization of DGP DS (drug substance) and homogenization of DGP DP. Sonication was used in place of homogenization for initial size reduction studies.

FIG. 42 shows that micronization, sonication, and the combination of the two decreases DGP DP size.

FIG. 43 shows that micronization and/or sonication of the DGP DP increases IL-1β secretion by human moDCs when treated with R848 and DGP DP compared to unmodified DGP DP.

FIG. 44 shows that micronization and/or sonication of the DGP DP increases CCR7 expression on DCs migrating to draining lymph nodes 4 hours post-administration of R848 and DGP DP compared to unmodified DGP DP.

FIG. 45A shows the frequency and FIG. 45B shows the absolute number of SIINFEKL⁺ CD8⁺ T cells in the blood of immunized mice. Data from groups of 5 mice are shown with each symbol representing one mouse.

FIG. 46A shows the frequency and FIG. 46B shows the absolute number of SIINFEKL⁺ CD8⁺ T cells in the draining lymph nodes of immunized mice. Data from groups of 4-5 mice are shown with each symbol representing one mouse.

FIG. 47 shows the frequency of OVA-specific, IFNγ-secreting T cells in draining lymph nodes of immunized mice. IFNγ-secreting cells were measured by ELISPOT assay after cells were cultured in the presence or absence of an OVA peptivator for 18 hours. Data from groups of 4-5 mice are shown with each symbol representing one mouse.

FIG. 48A shows structures of cationic and ionizable lipids suitable for use in the lipid nanoparticles (LNPs) of the present disclosure. FIG. 48B shows structures of other types of lipids suitable for use in LNPs of the present disclosure. See also, Hou et al., Nature Review Materials, 6:1078-1094, 2021, which is incorporated herein by reference.

FIG. 49 shows a heat map representing the normalized concentration of cytokines and chemokines that were detected at 2 hours, 24 hours, and 48 hours post injection. Abbreviations: MCP1=monocyte chemoattractant protein 1, MIP-1α=macrophage inflammatory protein-1 alpha, MIP-1β=macrophage inflammatory protein-1 beta, Rantes=Regulated on Activation, Normal T Expressed and Secreted, Eotaxin=eosinophil chemotaxin, MDC=macrophage derived chemokine, KC=keratinocyte-derived chemokine, IP-10=interferon-inducible protein 10, IFNα=interferon alpha, IFNβ=interferon beta, TNFα=tumor necrosis factor alpha, IL-6=interleukin 6, IL-10=interleukin 10, IL-12p40=interleukin 12 subunit P40, IL-12p70=interleukin 12 subunit P70, IL-23=interleukin 23, IL-27=interleukin 27, TSLP=thymic stromal lymphopoietin, MIG=monokine induced by gamma.

FIG. 50A-D are graphs representing the absolute number of monocytes (FIG. 50A), moDCs (FIG. 50B), macrophages (FIG. 50C), and cDCs (FIG. 50D) in dLN at 4 hours, and 48 hours post injection. There were five mice/group with each symbol representing one mouse.

FIG. 51A-D are graphs representing the absolute number of monocytes (FIG. 51A), moDCs (FIG. 51B), macrophages (FIG. 51C) and cDCs (FIG. 51D) in spleen at 4 hours, and 48 hours post injection. There were five mice/group with each symbol representing one mouse. Some Samples were excluded due to low cell viability post-dissociation.

FIG. 52A-D are graphs representing the MFI of CD69 expression on the surface of monocytes (FIG. 52A), moDCs (FIG. 52B), macrophages (FIG. 52C) and cDCs (FIG. 52D) in dLN at 4 hours, or 48 hours post injection.

FIG. 53A-D are graphs representing the MFI of CD69 expression on the surface of (FIG. 53A), moDCs (FIG. 53B), macrophages (FIG. 53C) and cDCs (FIG. 53D) in spleen at 4 hours, and 48 hours post injection.

FIG. 54A-B are graphs representing the MFI of CCR7 expression on the surface of DCs in the dLN (FIG. 54A) and the spleen (FIG. 54B) at 4 hours, and 48 hours post injection.

FIG. 55 shows spleen weights of immunized mice at endpoint. Each symbol represents one mouse, with 7 mice/group.

FIG. 56A shows the frequency of IFNγ SFCs for each sample and restimulation condition. FIG. 56B shows the frequency of Afluria-specific SFCs for each mouse after subtraction of background from unstimulated condition. Statistical significance was determined by Student's t-test. Each symbol represents one mouse, with 4-7 mice/group.

FIG. 57A shows the concentration of IFNγ determined for each sample and restimulation condition. FIG. 57B shows Afluria-specific IFNγ secretion for each mouse after subtraction of background from unstimulated condition. Statistical significance was determined by Student's t-test. Each symbol represents one mouse, with 4-7 mice/group.

FIG. 58A shows the frequency of IL-5 SFCs for each sample and restimulation condition. FIG. 58B shows the frequency of Afluria-specific SFCs for each mouse after subtraction of background from unstimulated condition. Statistical significance was determined by Student's t-test. Each symbol represents one mouse, with n=4-7 mice/group.

FIG. 59A shows the concentration of IL-5 determined for each sample and restimulation condition. FIG. 59B shows Afluria-specific IL-5 secretion for each mouse after subtraction of background from unstimulated condition. Statistical significance was determined by Student's t-test. Each symbol represents one mouse, with n=4-7 mice/group.

FIG. 60A shows the ratio of IFNγ SFCs to IL-5 SFCs for each sample. FIG. 60B shows the ratio of IFNγ to IL-5 concentration for each sample. Each symbol represents one mouse, with 6-7 mice/group.

FIG. 61 shows geometric mean titers (GMT) of antigen-specific antibodies in a hemagglutinin inhibition (HAI) assay performed with the Afluria vaccine as viral antigen.

FIG. 62A shows antigen-specific IgG in serum of immunized mice detected by ELISA with Afluria vaccine as coating antigen. FIG. 62B shows the affinity of antigen-specific IgG in serum of immunized mice detected by ELISA with Afluria vaccine as coating antigen. Statistical significance was determined by Student's t-test. Each symbol represents one mouse, with 4-7 mice/group.

FIG. 63A-G shows the frequencies of DCs (FIG. 63A), CD8+ TCMs (FIG. 63B), CD4+ TCMs (FIG. 63C), CD8+ TEMs (FIG. 63D), CD4+ TEMs (FIG. 63E), GC B (FIG. 63F), and TFH cells (FIG. 63G). Each symbol represents one mouse, with 4-7 mice/group.

FIG. 64A-D show hyperactivation of canine PBCMs. FIG. 64A shows relative viability as measured by ATP content in each condition compared to R848 alone. FIG. 64B shows IL-1β, FIG. 64C shows IL-6, and FIG. 64D shows IFNγ secretion in cell culture supernatants after 48-hour stimulation with the indicated treatments. Each symbol represents one canine donor, with n=4 donors. Statistical significance was determined by One-Way ANOVA followed by a Dunnet's multiple comparison test.

FIG. 65A shows clinical scores, FIG. 65B shows changes in weight, and FIG. 65C shows survival of immunized mice after live influenza (PR8) virus challenge.

FIG. 66A shows influenza (PR8) virus load and FIG. 66B shows concentration of influenza virus hemagglutinin (HA) antigen in bronchoalveolar lavage (BAL) fluid of immunized mice on day 5 post-challenge.

FIG. 67A shows titers of anti-hemagglutinin (HA) IgG, and FIG. 67B shows anti-nucleoprotein (NP) IgG antibodies in serum of immunized mice prior to influenza virus challenge.

FIG. 68A shows percentages of and FIG. 68B shows absolute numbers of influenza nucleoprotein-specific CD8+ T cells in blood of immunized mice prior to influenza virus challenge. Statistical significance was determined by one-way ANOVA with Tukey post-hoc analysis

### DETAILED DESCRIPTION

The present disclosure relates to ether lipid (ETL) compounds, such as ether phospholipid (ETPL) compounds, and uses thereof in hyperactivating human dendritic cells. The present disclosure also relates to compositions comprising an ETL, such as an ETPL, and one or more of a pathogen recognition receptor agonist, an antigen, and human dendritic cells, as well as methods for production and use of the compositions. In further embodiments, the dendritic cells are non-human dendritic cells, with the proviso that the dendritic cells are not rodent dendritic cells.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., a molecular weight of about 900 daltons, refers to a molecular weight of from 810 daltons to 990 daltons).

An “effective amount” or a “sufficient amount” of a substance is that amount sufficient to effect beneficial or desired results, including clinical results, and, as such, an “effective amount” depends upon the context in which it is being applied. For instance, in the context of administering an immunogenic composition, an effective amount contains sufficient antigen, and one or both of an ether lipid (ETL) compound such as an ether phospholipid (ETPL) compound, and a PRR agonist, to stimulate an immune response against the antigen (e.g., antigen-reactive antibody and/or cellular immune response).

The terms “individual” and “subject” refer to mammals. “Mammals” include, but are not limited to, humans, non-human primates (e.g., monkeys), farm animals, sport animals, rodents (e.g., mice and rats), and pets (e.g., dogs and cats). In some embodiments, the subject is a human patient, such as a human patient suffering from cancer and/or an infectious disease.

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is otherwise associated during production of the material (e.g., removed from its original environment). As an example, when used in reference to an ETL, such as an ETPL, an isolated ETL or ETPL is at least 90%, 95%, 96%, 97%, 98% or 99% pure as determined by thin layer chromatography (TLC), high pressure liquid chromatography (HPLC), or gas chromatography (GC). As a further example, when used in reference to a recombinant protein, an isolated protein refers to a protein that has been removed from the culture medium of the host cell that produced the protein. As a further example, when used in reference to a synthesized compound, an isolated compound or a purified compound has been removed from the reaction mixture in which it was synthesized.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions are intended to be sterile.

“Excipients” as used herein include pharmaceutically acceptable excipients, carriers, vehicles or stabilizers that are nontoxic to the cell or mammal being exposed thereto at the dosages and concentrations employed. Often the physiologically acceptable excipient is an aqueous pH buffered solution.

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. Antigens when present in the compositions of the present disclosure can be synthetic or isolated from nature. Antigens suitable for administration in the methods of the present disclosure include any molecule capable of eliciting an antigen-specific B cell or T cell response. Haptens are included within the scope of “antigen.” A “hapten” is a low molecular weight compound that is not immunogenic by itself but is rendered immunogenic when conjugated with a generally larger immunogenic molecule (carrier).

“Polypeptide antigens” can include purified native peptides, synthetic peptides, recombinant peptides, crude peptide extracts, or peptides in a partially purified or unpurified active state (such as peptides that are part of attenuated or inactivated viruses, microorganisms or cells), or fragments of such peptides. Polypeptide antigens are preferably at least eight amino acid residues in length.

The term “agonist” is used in the broadest sense and includes any molecule that activates signaling through a receptor. In some embodiments, the agonist binds to the receptor. For instance, a TLR8 agonist binds to a TLR8 receptor and activates a TLR8-signaling pathway.

“Alkyl” refers to monovalent saturated aliphatic hydrocarbyl groups. Cₓ alkyl refers to an alkyl group having x number of carbon atoms. Cₓ-C_{y} alkyl or C_{x-y} alkyl refers to an alkyl group having between x number and y number of carbon atoms, inclusive. An “n-alkyl” group refers to a straight-chain, i.e. linear, alkyl group.

“Alkylene” refers to divalent saturated aliphatic hydrocarbyl groups.

“Alkenyl” refers to monovalent hydrocarbyl groups having at least one double bond (>C═C<). Cₓ alkenyl refers to an alkenyl group having x number of carbon atoms. Cₓ-C_{y} alkenyl or C_{x-y} alkenyl refers to an alkenyl group having between x number and y number of carbon atoms, inclusive.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 2-fold to 2,000-fold, or from 5-fold to 500-fold or over, or from 2, 5, 10, 50, or 100-fold to 500, 1,000, 2,000, 5,000, or 10,000-fold.

Conversely, “inhibition” of a response or parameter includes reducing and/or repressing that response or parameter when compared to otherwise same conditions except for a parameter of interest, or alternatively, as compared to another condition (e.g., decrease in abnormal cell proliferation after administration of a composition comprising an ETL compound such as an ETPL compound, and one or more of a pathogen recognition receptor agonist, an antigen, and human dendritic cells, as compared to the administration of a placebo composition or no treatment). For example, “inhibition” of an immune response means a decrease in the response. Depending upon the parameter measured, the decrease may be from 2-fold to 2,000-fold, or from 5-fold to 500-fold or over, or from 2, 5, 10, 50, or 100-fold to 500, 1,000, 2,000, 5,000, or 10,000-fold.

The relative terms “higher” and “lower” refer to a measurable increase or decrease, respectively, in a response or parameter when compared to otherwise same conditions except for a parameter of interest, or alternatively, as compared to another condition. For instance, a “higher level of DC hyperactivation” refers to a level of DC hyperactivation as a consequence of a treatment condition (comprising an ETL compounds, such as an ETPL compound, of the present disclosure) that is at least 2, 3, 4, 5, 6, 7, 8, 9, or 10-fold above a level of DC hyperactivation as a consequence of a control condition (e.g., no ETL or ETPL, PGPC, oxPAPC, etc.). Likewise, a “lower level of DC hyperactivation” refers to a level of DC hyperactivation as a consequence of a treatment condition (comprising an ETL compound, such as an ETPL compound, of the present disclosure) that is at least 2, 3, 4, 5, 6, 7, 8, 9, or 10-fold below a level of DC hyperactivation as a consequence of a control condition (e.g., no ETL or ETPL, PGPC, oxPAPC, etc.). In some embodiments, the control condition comprises a comparator compound in the place of the ETL compound, which may be an ETPL compound, of the treatment condition.

As used herein the term “immunization” refers to a process that increases a mammalian subject's reaction to antigen and therefore improves its ability to resist or overcome infection and/or resist disease.

The term “vaccination” as used herein refers to the introduction of vaccine into a body of a mammalian subject.

“Adjuvant” refers to a substance which, when added to a composition comprising an antigen, enhances or potentiates an immune response to the antigen in the mammalian recipient upon exposure.

The terms “treating” or “treatment” of a disease refer to executing a protocol, which may include administering one or more therapeutic agents to an individual (human or otherwise), in an effort to obtain beneficial or desired results in the individual, including clinical results. Beneficial or desired clinical results include, but are not limited to, alleviation or amelioration of one or more signs or symptoms of a disease, diminishment of extent of disease, stabilized (i.e., not worsening) state of disease, preventing spread of disease, delay or slowing of disease progression, amelioration or palliation of the disease state, and remission (whether partial or total). “Treatment” also can mean prolonging survival as compared to expected survival of an individual not receiving treatment. Further, “treating” and “treatment” may occur by administration of one dose of a therapeutic agent or therapeutic agents, or may occur upon administration of a series of doses of a therapeutic agent or therapeutic agents. “Treating” or “treatment” does not require complete alleviation of signs or symptoms, and does not require a cure, and specifically includes protocols that have only a palliative effect on the individual. “Palliating” a disease or disorder means that the extent and/or undesirable clinical manifestations of the disease or disorder are lessened and/or time course of progression of the disease or disorder is slowed, as compared to the expected untreated outcome.

The compounds described herein can be administered in any pharmaceutically acceptable form, such as in the form of a pharmaceutically acceptable salt, or in free base or free acid form if said form is pharmaceutically acceptable. The compounds described herein, or pharmaceutically acceptable salts thereof, can be administered in pharmaceutically acceptable carriers or excipients. As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration. “Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases which can be used to prepared salts include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

### I. Ether Lipid (ETL) and Ether Phospholipid (ETPL) Compounds

An “ether lipid” (ETL) or “ether lipid molecule” refers to a glycerol molecule bearing a hydrocarbyl group on one of the hydroxyl groups of the glycerol. The remaining hydroxyl groups can be unsubstituted (free hydroxyl groups) or can be substituted. The hydrocarbyl group can be an aliphatic hydrocarbyl group, such as an alkyl group, such as an n-alkyl group. The alkyl group or n-alkyl group in any of the compounds as disclosed herein is preferably unsubstituted, i.e., it consists of only carbon and hydrogen atoms.

An “ether phospholipid” (ETPL) or “ether phospholipid molecule” is a particular type of ether lipid, and refers to a glycerol molecule bearing a phosphate group on a hydroxyl of the glycerol and bearing one hydrocarbyl group on one of the other two hydroxyl groups of the glycerol. The remaining hydroxyl group can be unsubstituted (a free hydroxyl group) or can be substituted. The hydrocarbyl group can be an aliphatic hydrocarbyl group, such as an alkyl group, such as an n-alkyl group. The alkyl group or n-alkyl group in any of the compounds as disclosed herein is preferably unsubstituted, i.e., it consists of only carbon and hydrogen atoms.

The current disclosure provides ether lipids, such as ether phospholipids. The current disclosure provides isolated ether lipids, such as isolated ether phospholipids.

In some embodiments, provided herein are ether lipid compounds of Formula (I), such as an isolated ether lipid (ETL) with an alkyl chain of Formula (I):

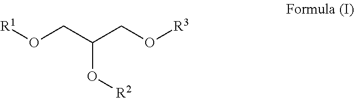

where R¹ is H or

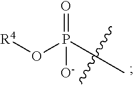

R² is H, C₁-C₄ alkyl, —(C═O)—NH₂, —(C═O)—NH(R⁵), —(C═O)—N(R⁵)₂, or —CH₂—C₆H₅;

R³ is C₁₃-C₂₄ n-alkyl;

where R⁴ is H or (CH₃)₃N⁺—(CH₂)₂—; and

each R⁵ is independently C₁-C₄ alkyl;

or a protonated or deprotonated form thereof; or a salt thereof, such as a pharmaceutically acceptable salt thereof; and all stereoisomers thereof.

In some embodiments, provided herein are ether lipid compounds of Formula (II), such as an isolated ether lipid (ETL) with an alkyl chain of Formula (II):

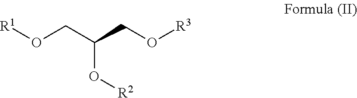

where R¹ is H or

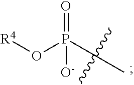

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a salt thereof, such as a pharmaceutically acceptable salt thereof.

In some embodiments, provided herein are ether lipid compounds of Formula (III), such as an isolated ether lipid (ETL) with an alkyl chain of Formula (III):

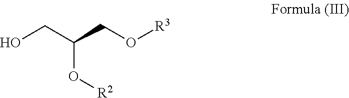

where R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl; and
each R⁵ is independently C₁-C₄ alkyl;
or a salt thereof, such as a pharmaceutically acceptable salt thereof.

In some embodiments, provided herein are ether lipid compounds of Formula (III-A), such as an isolated ether lipid (ETL) with an alkyl chain of Formula (III-A):

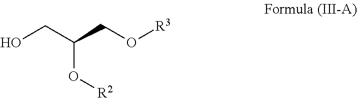

where R² is —(C═O)—NH₂, —(C═O)—NH(R⁵), or —(C═O)—N(R⁵)₂;
R³ is C₁₃-C₂₄ n-alkyl; and
each R⁵ is independently C₁-C₄ alkyl;
or a salt thereof, such as a pharmaceutically acceptable salt thereof. In some embodiments, R² is —(C═O)—NH₂. In some embodiments, R² is —(C═O)—NH—CH₃. In some embodiments, R² is —(C═O)—N(CH₃)₂. In some embodiments, R³ is C₂₁-C₂₄ n-alkyl. In some embodiments, R² is —(C═O)—NH₂ and R³ is C₂₁-C₂₄ n-alkyl. In some embodiments, R² is —(C═O)—NH—CH₃ and R³ is C₂₁-C₂₄ n-alkyl. In some embodiments, R² is —(C═O)—N(CH₃)₂ and R³ is C₂₁-C₂₄ n-alkyl. In some embodiments, R² is —(C═O)—NH₂ and R³ is C₂₂ n-alkyl. In some embodiments, R² is —(C═O)—NH—CH₃ and R³ is C₂₂ n-alkyl. In some embodiments, R² is —(C═O)—N(CH₃)₂ and R³ is C₂₂ n-alkyl.

In some embodiments, provided herein are ether lipid compounds of Formula (III-A-1), such as an isolated ether lipid (ETL) with an alkyl chain of Formula (III-A-1):

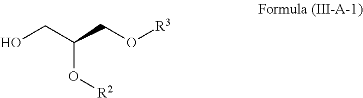

where R² is —(C═O)—NH₂, —(C═O)—NH(R⁵), or —(C═O)—N(R⁵)₂;
R³ is C₂₁-C₂₄ n-alkyl; and
each R⁵ is independently C₁-C₄ alkyl;
or a salt thereof, such as a pharmaceutically acceptable salt thereof. In some embodiments, R² is —(C═O)—NH₂. In some embodiments, R² is —(C═O)—NH—CH₃. In some embodiments, R² is —(C═O)—N(CH₃)₂. In some embodiments, R³ is C₂₂ n-alkyl. In some embodiments, R² is —(C═O)—NH₂ and R³ is C₂₂ n-alkyl. In some embodiments, R² is —(C═O)—NH—CH₃ and R³ is C₂₂ n-alkyl. In some embodiments, R² is —(C═O)—N(CH₃)₂ and R³ is C₂₂ n-alkyl.

In some embodiments, provided herein are ether lipid compounds of Formula (III-A-2), such as an isolated ether lipid (ETL) with an alkyl chain of Formula (III-A-2):

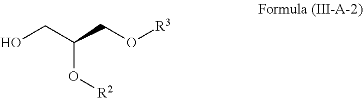

where R² is —(C═O)—NH₂, —(C═O)—NH(R⁵), or —(C═O)—N(R⁵)₂;
R³ is C₁₆-C₂₀ n-alkyl; and
each R⁵ is independently C₁-C₄ alkyl;
or a salt thereof, such as a pharmaceutically acceptable salt thereof. In some embodiments, R² is —(C═O)—NH₂. In some embodiments, R² is —(C═O)—NH—CH₃. In some embodiments, R² is —(C═O)—N(CH₃)₂. In some embodiments, R³ is C₁₈ n-alkyl. In some embodiments, R² is —(C═O)—NH₂ and R³ is C₁₈ n-alkyl. In some embodiments, R² is —(C═O)—NH—CH₃ and R³ is C₁₈ n-alkyl. In some embodiments, R² is —(C═O)—N(CH₃)₂ and R³ is C₁₈ n-alkyl.

In some embodiments, provided herein are ether lipid compounds of Formula (III-B), such as an isolated ether lipid (ETL) with an alkyl chain of Formula (III-B):

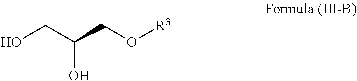

where R³ is C₁₃-C₂₄ n-alkyl; or a salt thereof, such as a pharmaceutically acceptable salt thereof. In some embodiments, R³ is C₂₁-C₂₄ n-alkyl. In some embodiments, R³ is C₂₂ n-alkyl.

In some embodiments, provided herein are ether lipid compounds of Formula (III-B-1), such as an isolated ether lipid (ETL) with an alkyl chain of Formula (III-B-1):

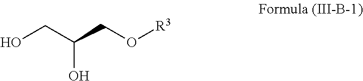

where R³ is C₂₁-C₂₄ n-alkyl; or a salt thereof, such as a pharmaceutically acceptable salt thereof. In some embodiments, R³ is C₂₂ n-alkyl.

In some embodiments, provided herein are ether lipid compounds of Formula (III-B-2), such as an isolated ether lipid (ETL) with an alkyl chain of Formula (III-B-2):

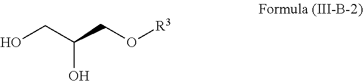

where R³ is C₁₃-C₂₀ n-alkyl; or a salt thereof, such as a pharmaceutically acceptable salt thereof. In some embodiments, R³ is C₁₈ n-alkyl.

In some embodiments, provided herein are ether lipid compounds of Formula (IV), such as an isolated ether phospholipid (ETPL) with an alkyl chain of Formula (IV):

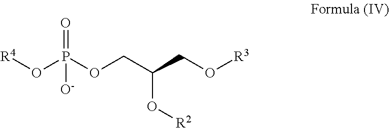

where R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
R⁴ is H or (CH₃)₃N⁺—(CH₂)₂—; and
each R⁵ is independently C₁-C₄ alkyl;
or a protonated or deprotonated form thereof; or a salt thereof, such as a pharmaceutically acceptable salt thereof.

In some embodiments, provided herein are ether lipid compounds of Formula (IV-A), such as an isolated ether phospholipid (ETPL) with an alkyl chain of Formula (IV-A):

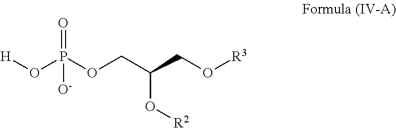

where R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl; and
each R⁵ is independently C₁-C₄ alkyl;
or a protonated or deprotonated form thereof; or a salt thereof, such as a pharmaceutically acceptable salt thereof. In some embodiments, R² is H. In some embodiments, R² is —(C═O)—NH₂. In some embodiments, R² is —(C═O)—NH—CH₃. In some embodiments, R² is —(C═O)—N(CH₃)₂. In some embodiments, R³ is C₂₁-C_{2A} n-alkyl. In some embodiments, R² is H and R³ is C₂₁-C_{2A} n-alkyl. In some embodiments, R² is —(C═O)—NH₂ and R³ is C₂₁-C₂₄ n-alkyl. In some embodiments, R² is —(C═O)—NH—CH₃ and R³ is C₂₁-C₂₄ n-alkyl. In some embodiments, R² is —(C═O)—N(CH₃)₂ and R³ is C₂₁-C₂₄ n-alkyl. In some embodiments, R² is H and R³ is C₂₂ n-alkyl. In some embodiments, R² is —(C═O)—NH₂ and R³ is C₂₂ n-alkyl. In some embodiments, R² is —(C═O)—NH—CH₃ and R³ is C₂₂ n-alkyl. In some embodiments, R² is —(C═O)—N(CH₃)₂ and R³ is C₂₂ n-alkyl.

In some embodiments, provided herein are ether lipid compounds of Formula (IV-A-1), such as an isolated ether phospholipid (ETPL) with an alkyl chain of Formula (IV-A-1):

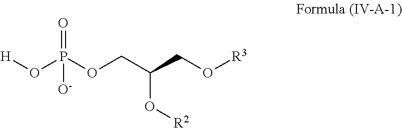

where R² is H, C₁-C₄ alkyl, —(C═O)—NH₂, —(C═O)—NH(R⁵), —(C═O)—N(R⁵)₂, or —CH₂—C₆H₅;
R³ is C₂₁-C₂₄ n-alkyl; and
each R⁵ is independently C₁-C₄ alkyl;
or a protonated or deprotonated form thereof; or a salt thereof, such as a pharmaceutically acceptable salt thereof. In some embodiments, R² is H. In some embodiments, R² is —(C═O)—NH₂. In some embodiments, R² is —(C═O)—NH—CH₃. In some embodiments, R² is —(C═O)—N(CH₃)₂. In some embodiments, R³ is C₂₂ n-alkyl. In some embodiments, R² is H and R³ is C₂₂ n-alkyl. In some embodiments, R² is —(C═O)—NH₂ and R³ is C₂₂ n-alkyl. In some embodiments, R² is —(C═O)—NH—CH₃ and R³ is C₂₂ n-alkyl. In some embodiments, R² is —(C═O)—N(CH₃)₂ and R³ is C₂₂ n-alkyl.

In some embodiments, provided herein are ether lipid compounds of Formula (IV-A-2), such as an isolated ether phospholipid (ETPL) with an alkyl chain of Formula (IV-A-2):

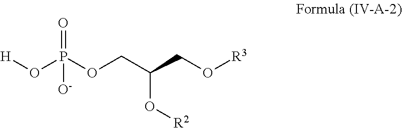

where R² is H, C₁-C₄ alkyl, —(C═O)—NH₂, —(C═O)—NH(R⁵), —(C═O)—N(R⁵)₂, or —CH₂—C₆H₅;
R³ is C₁₆-C₂₀ n-alkyl; and
each R⁵ is independently C₁-C₄ alkyl;
or a protonated or deprotonated form thereof; or a salt thereof, such as a pharmaceutically acceptable salt thereof. In some embodiments, R² is H. In some embodiments, R² is —(C═O)—NH₂. In some embodiments, R² is —(C═O)—NH—CH₃. In some embodiments, R² is —(C═O)—N(CH₃)₂. In some embodiments, R³ is C₁₈ n-alkyl. In some embodiments, R² is H and R³ is C₁₈ n-alkyl. In some embodiments, R² is —(C═O)—NH₂ and R³ is C₁₈ n-alkyl. In some embodiments, R² is —(C═O)—NH—CH₃ and R³ is C₁₈ n-alkyl. In some embodiments, R² is —(C═O)—N(CH₃)₂ and R³ is C₁₈ n-alkyl.

In some embodiments, provided herein are ether lipid compounds of Formula (IV-B), such as an isolated ether phospholipid (ETPL) with an alkyl chain of Formula (IV-B):

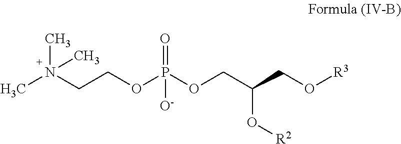

where R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl; and
each R⁵ is independently C₁-C₄ alkyl;
or a protonated form thereof; or a salt thereof, such as a pharmaceutically acceptable salt thereof.

In some embodiments, provided herein are ether lipid compounds of Formula (IV-B-1), such as an isolated ether phospholipid (ETPL) with an alkyl chain of Formula (IV-B-1):

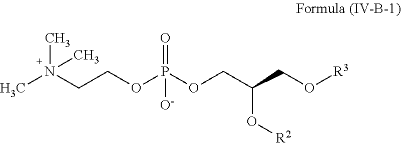

where R² is H, C₁-C₄ alkyl, —(C═O)—NH₂, —(C═O)—NH(R⁵), —(C═O)—N(R⁵)₂, or —CH₂—C₆H₅;
R³ is C₂₁-C₂₄ n-alkyl; and
each R⁵ is independently C₁-C₄ alkyl;
or a protonated form thereof; or a salt thereof, such as a pharmaceutically acceptable salt thereof. In some embodiments, R² is H. In some embodiments, R² is —(C═O)—NH₂. In some embodiments, R² is —(C═O)—NH—CH₃. In some embodiments, R² is —(C═O)—N(CH₃)₂. In some embodiments, R³ is C₂₂ n-alkyl. In some embodiments, R² is H and R³ is C₂₂ n-alkyl. In some embodiments, R² is —(C═O)—NH₂ and R³ is C₂₂ n-alkyl. In some embodiments, R² is —(C═O)—NH—CH₃ and R³ is C₂₂ n-alkyl. In some embodiments, R² is —(C═O)—N(CH₃)₂ and R³ is C₂₂ n-alkyl.

In some embodiments, provided herein are ether lipid compounds of Formula (IV-B-2), such as an isolated ether phospholipid (ETPL) with an alkyl chain of Formula (IV-B-2):

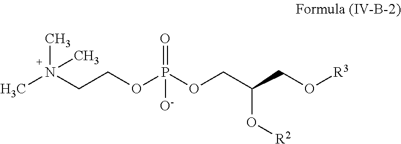

where R² is H, C₁-C₄ alkyl, —(C═O)—NH₂, —(C═O)—NH(R⁵), —(C═O)—N(R⁵) or —CH₂—C₆H₅;
R³ is C₁₆-C₂₀ n-alkyl; and
each R⁵ is independently C₁-C₄ alkyl;
or a protonated form thereof; or a salt thereof, such as a pharmaceutically acceptable salt thereof. In some embodiments, R² is H. In some embodiments, R² is —(C═O)—NH₂. In some embodiments, R² is —(C═O)—NH—CH₃. In some embodiments, R² is —(C═O)—N(CH₃)₂. In some embodiments, R³ is C₁₈ n-alkyl. In some embodiments, R² is H and R³ is C₁₈ n-alkyl. In some embodiments, R² is —(C═O)—NH₂ and R³ is C₁₈ n-alkyl. In some embodiments, R² is —(C═O)—NH—CH₃ and R³ is C₁₈ n-alkyl. In some embodiments, R² is —(C═O)—N(CH₃)₂ and R³ is C₁₈ n-alkyl.

In some embodiments, provided herein are ether lipid compounds of Formula (IV-C), such as an isolated ether phospholipid (ETPL) with an alkyl chain of Formula (IV-C):

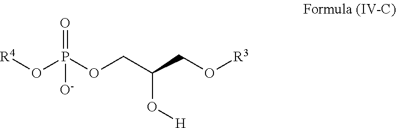

where R³ is C₁₃-C₂₄ n-alkyl; and
R⁴ is H or (CH₃)₃N⁺—(CH₂)₂—;
or a protonated or deprotonated form thereof; or a salt thereof, such as a pharmaceutically acceptable salt thereof.

In some embodiments, provided herein are ether lipid compounds of Formula (IV-D), such as an isolated ether phospholipid (ETPL) with an alkyl chain of Formula (IV-D):

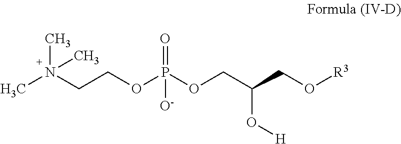

where R³ is C₁₃-C₂₄ n-alkyl;
or a protonated form thereof; or a salt thereof, such as a pharmaceutically acceptable salt thereof. In some embodiments, R³ is C₁₆-C₂₀ n-alkyl. In some embodiments, R³ is C₂₁-C₂₄ n-alkyl. In some embodiments, R³ is C₂₂ n-alkyl.

In some embodiments, provided herein are ether lipid compounds of Formula (IV-E), such as an isolated ether phospholipid (ETPL) with an alkyl chain of Formula (IV-E):

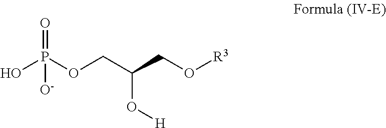

where R³ is C₁₃-C₂₄ n-alkyl;
or a protonated or deprotonated form thereof; or a salt thereof, such as a pharmaceutically acceptable salt thereof. In some embodiments, R³ is C₂₁-C₂₄ n-alkyl.

In some embodiments, provided herein are ether lipid compounds of Formula (IV-F), such as an isolated ether phospholipid (ETPL) with an alkyl chain of Formula (IV-F):

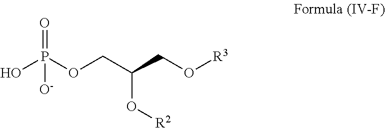

wherein R² is H, —(C═O)—NH₂, —(C═O)—NH(R⁵), —(C═O)—N(R⁵)₂, or —CH₂—C₆H₅; R³ is C₂₁-C₂₄ n-alkyl; and each R⁵ is independently C₁-C₄ alkyl; or a protonated or deprotonated form thereof; or a salt thereof. In some embodiments, R² is H. In some embodiments, R² is —(C═O)—NH₂. In some embodiments, R² is —(C═O)—NH(R⁵). In some embodiments, R² is —(C═O)—N(R⁵)₂. In some embodiments, R³ is C₂₁ n-alkyl. In some embodiments, R³ is unsubstituted. In some embodiments, R⁵ is —CH₃. Formula (IV-F) is a combination of certain compounds of Formula (IV-A) and Formula (IV-E).

In some embodiments, provided herein are ether lipid compounds of Formula (A), such as an isolated ether lipid (ETL) with an alkyl chain of Formula (I):

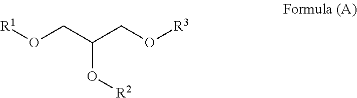

where R¹ is H or

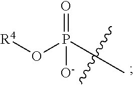

R² is H, C₁-C₄ alkyl, —(C═O)—NH₂, —(C═O)—NH(R⁵), —(C═O)—N(R⁵)₂, or —CH₂—C₆H₅;
R³ is C₁₀-C₃₀ n-alkyl;
where R⁴ is H or (CH₃)₃N⁺—(CH₂)₂—; and
each R⁵ is independently C₁-C₄ alkyl;
or a protonated or deprotonated form thereof; or a salt thereof, such as a pharmaceutically acceptable salt thereof; and all stereoisomers thereof.

In some embodiments, the ether phospholipid (ETPL) with an n-alkyl chain is a compound of Formula (IV), where R⁴ is (CH₃)₃N⁺—(CH₂)₂—; R² is H; R³ is C₂₂ n-alkyl, and the compound is 1-docosyl-sn-glycerol-3-phosphocholine (DGPC):

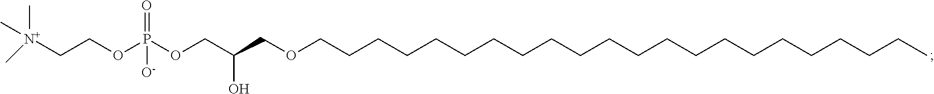 [SMILES: CCCCCCCCCCCCCCCCCCCCCCOC[C@@H](O)COP(=O)([O-])OCC[N+](C)(C)C]

or a protonated form thereof; or a salt thereof, such as a pharmaceutically acceptable salt thereof.

In some embodiments, the isolated ether phospholipid (ETPL) with an n-alkyl chain is a compound of Formula (IV), where R⁴ is (CH₃)₃N⁺—(CH₂)₂—; R² is H; R³ is C₂₂ n-alkyl, and the compound is 1-docosyl-sn-glycerol-3-phosphocholine (DGPC):

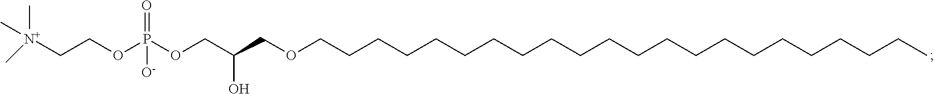 [SMILES: CCCCCCCCCCCCCCCCCCCCCCOC[C@@H](O)COP(=O)([O-])OCC[N+](C)(C)C]

or a protonated form thereof; or a salt thereof, such as a pharmaceutically acceptable salt thereof.

In some embodiments, the ether phospholipid (ETPL) with an n-alkyl chain is a compound of Formula (IV), where R⁴ is H; R² is H; R³ is C₂₂ n-alkyl, and the compound is 1-docosyl-sn-glycerol-3-phosphate (DGP):

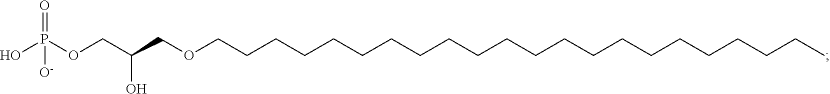 [SMILES: CCCCCCCCCCCCCCCCCCCCCCOC[C@@H](O)COP(=O)([O-])O]

or a protonated or deprotonated form thereof; or a salt thereof, such as a pharmaceutically acceptable salt thereof.

In some embodiments, the isolated ether phospholipid (ETPL) with an n-alkyl chain is a compound of Formula (IV), where R⁴ is H; R² is H; R³ is C₂₂ n-alkyl, and the compound is 1-docosyl-sn-glycerol-3-phosphate (DGP):

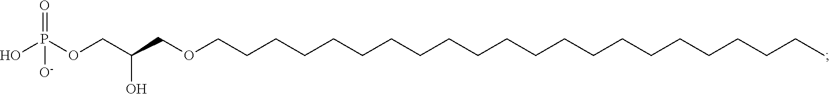 [SMILES: CCCCCCCCCCCCCCCCCCCCCCOC[C@@H](O)COP(=O)([O-])O]

or a protonated or deprotonated form thereof; or a salt thereof, such as a pharmaceutically acceptable salt thereof.

Ether lipid (ETL) and ether phospholipid (ETPL) compounds of the present disclosure have an alkyl chain in which the n-alkyl chain is a C13-C22 n-alkyl chain or a C13-C24 n-alkyl chain. In some embodiments, the n-alkyl chain is a C18-C22 n-alkyl chain or a C21-C24 n-alkyl chain. In some embodiments, the n-alkyl chain is a C16-C20 n-alkyl chain. In some embodiments, the n-alkyl chain is a C21-C24 n-alkyl chain. In some preferred embodiments, the n-alkyl chain is a C22 n-alkyl chain. Structures of exemplary ETPL and ETL compounds of the present disclosure are shown in Table I and Table II below, respectively. The structures shown in Table I and Table II can alternatively be protonated or deprotonated forms of the structures shown in Table I and Table II, that is, where protonated indicates a proton on any or all phosphate oxygens depicted as O⁻ below, and where deprotonated indicates removal of a proton from any or all phosphate OH group; and/or can be a salt of the structures shown in Table I and Table II, such as a pharmaceutically acceptable salt thereof.

TABLE I
Ether Phospholipids

| Compound | Structure |
|---|---|
| 1 DGPC | 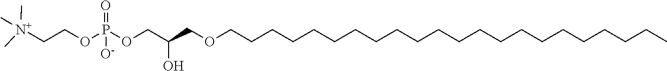 [SMILES: CCCCCCCCCCCCCCCCCCCCCCOC[C@@H](O)COP(=O)([O-])OCC[N+](C)(C)C] |
| 2 DGP | 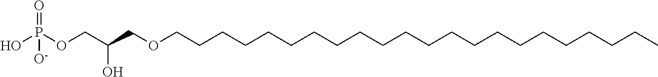 [SMILES: CCCCCCCCCCCCCCCCCCCCCCOC[C@@H](O)COP(=O)([O-])O] |
| 3 | 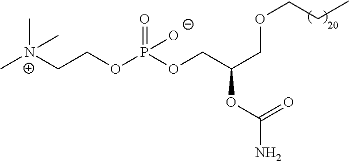 |
| 4 | 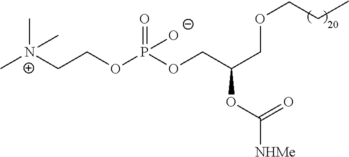 |
| 5 | 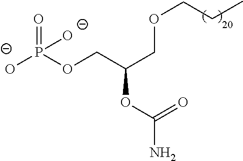 |
| 6 | 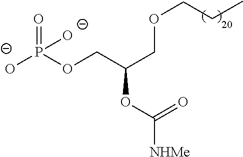 |
| 16 | 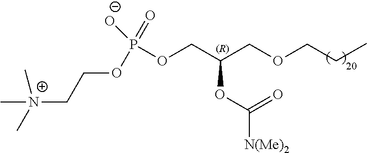 |

TABLE II
Ether Lipids

| Compound | Structure |
|---|---|
| 7 DHC | 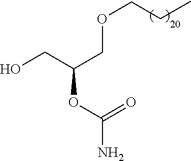 |
| 8 DHMC | 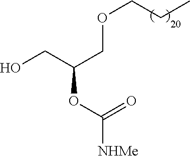 |
| 9 DPD | 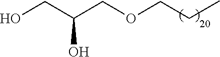 |
| 10 | 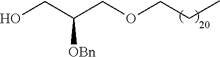 |
| 11 | 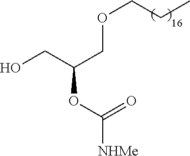 |
| 12 | 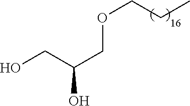 |
| 14 | 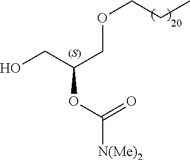 |

### II. Pathogen Recognition Receptor Agonists

Compositions and methods of the present disclosure may further comprise a pathogen recognition receptor (PRR) agonist. In some embodiments, the PRR agonist comprises an agonist of a toll-like receptor (TLR), a NOD-like receptor (NLR), a RIG-I-like receptor (RLR), or a C-type lectin receptor (CLR). In other embodiments, the PRR agonist comprises a cytosolic DNA sensor (CDS) or a stimulator of IFN genes (STING). In some embodiments, the PRR agonist comprises a TLR7/8 agonist.

### A. TLR Agonists and TLR7/8 Agonists

The term “TLR agonist” as used herein refers to an agonist of at least one TLR. The term “TLR7/8 agonist” as used herein refers to an agonist of TLR7 and/or TLR8. In one aspect, the TLR7/8 agonist is a TLR7 agonist. In a further aspect, the TLR7/8 agonist is a TLR8 agonist. In a further aspect, the TLR7/8 agonist is an agonist of both TLR7 and TLR8. TLR7/8 agonists of the present disclosure are suitable for hyperactivating human dendritic cells in the presence of LPC.

In some aspects, the TLR agonist is a small molecule. In some aspects, the TLR7/8 agonist is a small molecule. In some embodiments, the TLR7/8 agonist is a small molecule with a molecule weight of 900 daltons or less, or a salt thereof. That is, the small molecule TLR7/8 agonist is not a large molecule like a recombinant protein or a synthetic oligonucleotide, which is regulatable by the U.S. FDA's Center for Biologics Evaluation and Research. Rather the small molecule TLR7/8 agonist is regulatable by the FDA's Center for Drug Evaluation and Research. In some embodiments, the small molecule has a molecule weight of from about 90 to about 900 daltons. In some embodiments, the TLR7/8 agonist comprises an imidazoquinoline compound. In some preferred embodiments, the TLR7/8 agonist comprises resiquimod (R848).

### B. Other PRR Agonists

In some aspects, the pathogen recognition receptor (PRR) agonist comprises a toll-like receptor (TLR) agonist with the proviso that the TLR agonist does not comprise a TLR7/8 agonist. In some embodiments, the TLR agonist comprises an agonist of one or more of TLR2, TLR3, TLR4, TLR5, TLR9 and TLR13. In some embodiments, the PRR agonist is a TLR2/6 agonist, such as Pam2CSK4. In other embodiments, the TLR agonist is a TLR4 agonist such as monophosphoryl lipid A (MPLA). However, in preferred embodiments, the TLR agonist is not an agonist of TLR2, TLR4 and/or TLR9. For instance, in preferred embodiments, the TLR9 agonist is not a TLR4 ligand such as LPS (endotoxin).

In additional aspects, the PRR agonist comprises a NOD-like receptor (NLR) agonist. In further aspects, the PRR agonist comprises a RIG-I-like receptor (RLR) agonist. In additional aspects, the PRR agonist comprises a C-type lectin receptor (CLR) agonist. In still further aspects, the PRR agonist comprises a CDS agonist or a STING agonist.

### III. Antigens

Compositions and methods of the present disclosure may further comprise an antigen. In some embodiments, the antigen comprises a proteinaceous antigen. The terms “polypeptide” and “protein” are used interchangeably herein to refer to proteinaceous antigens that comprise peptide chains that are at least 8 amino acids in length. In some embodiments, the proteinaceous antigen is from 8 to 1800 amino acids, 9 to 1000 amino acids, or 10 to 100 amino acids in length. In some embodiments, the antigen comprises a synthetic protein or a recombinant protein. In other embodiments, the antigen comprises a protein purified from a biological sample. The polypeptide may be post-translationally modified such as by phosphorylation, hydroxylation, sulfonation, palmitoylation, and/or glycosylation.

In some embodiments, the antigen is a tumor antigen that comprises the amino acid sequence of at least one full length protein or fragment thereof. In some embodiments, the tumor antigen comprises an amino acid sequence or fragment thereof from an oncoprotein. In some embodiments, the mammalian antigen is a neoantigen or encoded by a gene comprising a mutation relative to the gene present in normal cells from a mammalian subject. Neoantigens are thought to be particularly useful in enabling T cells to distinguish between cancer cells and non-cancer cells (see, e.g., Schumacher and Schreiber, Science, 348:69-74, 2015). In other embodiments, the tumor antigen comprises a viral antigen, such as an antigen of a cancer-causing virus.

In some embodiments, the tumor antigen is a fusion protein comprising two or more polypeptides, wherein each polypeptide comprises an amino acid sequence from a different tumor antigen or non-contiguous amino acid sequences from the same tumor antigen. In some of these embodiments, the fusion protein comprises a first polypeptide and a second polypeptide, wherein each polypeptide comprises non-contiguous amino acid sequences from the same tumor antigen.

In some embodiments, the antigen is a microbial antigen. In some embodiments, the microbial antigen comprises a viral antigen, a bacterial antigen, a protozoan antigen, a fungal antigen, or combinations thereof. In some embodiments, the microbial antigen comprises a surface protein or other antigenic subunit of a microbe. In other embodiments, the microbial antigen comprises an inactivated or attenuated microbe. For instance, the microbial antigen may comprise an inactivated virus, such as a chemically or genetically-inactivated virus. Alternatively, the microbial antigen may comprise a virus-like particle.

In some embodiments, the antigen may be present in a biological sample obtained from an individual, such as a human patient. For instance, the antigen may comprise cancer cells. In a further aspect, the antigen may comprise microbially-infected cells, such as virally-infected cells.

### IV. Dendritic Cells

Compositions and methods of the present disclosure may further comprise dendritic cells (DCs), which are antigen presenting cells that are thought to bridge the innate and adaptive immune systems of mammals. In preferred embodiments, the DCs are subset-1 conventional DCs (cDC1s, previously referred to as myeloid DC1s), as opposed to plasmacytoid DCs (pDCs).

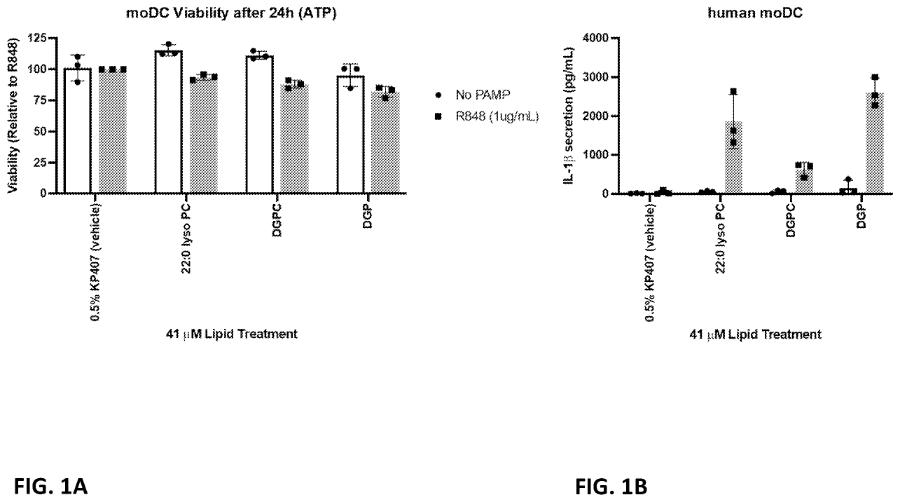

In some embodiments, the DCs are hyperactive DCs that express high levels of CD40 and IL-12p70. As used herein, the term “hyperactive dendritic cells” refer to a cell state in which DCs are able to secrete IL-1β while maintaining cellular viability (e.g., without undergoing pyroptosis). In this way, hyperactivated dendritic cells are able to stimulate robust T cell immunity (FIG. 1), which apparently combines the benefits of activated and pyroptotic dendritic cells (Zhivaki et al., Cell Reports, 33 (7), 2020, 108381).

### V. Pharmaceutical Formulations

Some compositions of the present disclosure are pharmaceutical formulations comprising a pharmaceutically acceptable excipient, and an ETL compound, such as an ETPL compound. Some compositions of the present disclosure are pharmaceutical formulations comprising a pharmaceutically acceptable excipient, and a lipid nanoparticle (LNP) comprising an ETL or ETPL compound and at least one further lipid. In some embodiments, the pharmaceutical formulations further comprise a PRR agonist, a dendritic cell, an antigen, an adjuvant, or any combination thereof. Pharmaceutical formulations of the present disclosure may be in the form of a solution or a suspension. Alternatively, the pharmaceutical formulations may be a dehydrated solid (e.g., freeze dried or spray dried solid). The pharmaceutical formulations of the present disclosure are preferably sterile, and preferably essentially endotoxin-free. The term “pharmaceutical formulations” is used interchangeably herein with the terms “medicinal product” and “medicament”. In some embodiments, the pharmaceutical formation comprises specific ratios of the various components based on the intended purpose of the formulation. In some embodiments, the pharmaceutical formulations comprise an ETL compound, such as an ETPL compound, and non-ionic surfactant. In some embodiments, the non-ionic surfactant comprises an ethylene oxide-propylene oxide copolymer (that is, a poloxamer), such as Poloxamer-407 (CAS Registry No. 977057-91-2).

### A. Excipients

Pharmaceutically acceptable excipients of the present disclosure include for instance, solvents, buffering agents, tonicity adjusting agents, bulking agents, and preservatives (See, e.g., Pramanick et al., Pharma Times, 45:65-77, 2013). In some embodiments, the pharmaceutical formulations may comprise an excipient that functions as one or more of a solvent, a buffering agent, a tonicity adjusting agent, and a bulking agent (e.g., sodium chloride in saline may serve as both an aqueous vehicle and a tonicity adjusting agent). Pharmaceutically acceptable excipients of the present disclosure also include detergents, wetting agents, thickening agents, emulsifiers, foaming agents, and dispersants, as well as surfactants.

Many of the lipids disclosed herein are sparingly soluble in water. Surfactants can be used to solubilize the lipids in aqueous formulations. A wide variety of surfactants are available, which can be classified as anionic surfactants, non-ionic surfactants, cationic surfactants, and zwitterionic surfactants.

Some examples of non-ionic surfactants include poloxamers, which are triblock copolymers of ethylene oxide and propylene oxide of the general formula: HO—[CH₂CH₂—O—][CH₂CH(CH₃)—O—]_{b}—[CH₂—CH₂—O—]ₐ—H, where a is typically about 2 to 130 and b is typically about 15 to 67. Some poloxamers are sold under the trade name Pluronic® (PLURONIC is a registered trademark of BASF SE, Ludwigshafen, Germany). Examples of poloxamers are Poloxamer 407 (KP407; a=101, b=56); Poloxamer 188 (KP188; a=80, b=27); Pluronic® P84 (P-84; a=19, b=39); and Pluronic® P123 (P-123; a=20, b=70) (the foregoing values for a and b can be subject to slight variation).

Other non-ionic surfactants include the Cremophor® series (CREMAPHOR is a registered trademark of BASF SE, Ludwigshafen, Germany). Cremophor® surfactants include Cremophor® EL (K EL), a mixture of polyoxyethylated triglycerides produced by reacting castor oil with ethylene oxide in a molar ratio of approximately 1:35, and Cremophor® RH40 (also known as Kolliphor® RH40; KOLLIPHOR is a registered trademark of BASF SE), obtained by reacting 40 moles of ethylene oxide with 1 mole of hydrogenated castor oil.

In some embodiments, the pharmaceutical formulations comprise an aqueous vehicle as a solvent. Suitable vehicles include for instance sterile water, saline solution, phosphate buffered saline, and Ringer's solution. In some embodiments, the composition is isotonic.

The pharmaceutical formulations may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical formulations may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical formulations may comprise a preservative. Suitable preservatives include for instance antioxidants and antimicrobial agents. However, in preferred embodiments, the pharmaceutical formulation is prepared under sterile conditions and is in a single use container, and thus does not necessitate inclusion of a preservative. Methods for preparing sterile, pharmaceutically acceptable compositions include steam sterilization, dry-heat sterilization, gas sterilization, ionizing radiation, or sterile filtration. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

In some embodiments, the pharmaceutical formulation is a homogenous solution. In some embodiments, the homogenous solution is supplied in a pre-filled syringe. In some embodiments, the pharmaceutical formulation is supplied as a suspension. In some embodiments, the suspension is refrigerated. In some embodiments, the suspension is frozen. In some embodiments, methods provided herein further comprise the step of warming the refrigerated suspension to room temperature and/or agitating the suspension to ensure that the active ingredient(s) are dissolved and/or evenly distributed in solution prior to administration. In some embodiments, methods provided herein further comprise the step of thawing the frozen suspension and warming to room temperature and/or agitating the suspension to ensure that the active ingredient(s) are dissolved and/or evenly distributed in solution prior to administration. In some embodiments, the suspension is diluted prior to administration. In some embodiments, the suspension is supplied as a pre-filled syringe. In some embodiments, the suspension comprises a pharmaceutically acceptable excipient, e.g., surfactant, glycerol, non-ionic surfactant, buffer, glycol, salt, or any combination thereof.

The pharmaceutical formulations of the present disclosure are suitable for parenteral administration. That is, the pharmaceutical formulations of the present disclosure are not intended for enteral administration (e.g., not by oral, gastric, or rectal administration).

### B. Adjuvants

Pharmaceutically acceptable adjuvants of the present disclosure include for instance, an aluminum salt adjuvant, a squalene-in-water emulsion, a saponin, or combinations thereof. In some embodiments, the adjuvant is an aluminum salt adjuvant selected from the group consisting of amorphous aluminum hydroxyphosphate sulfate, aluminum hydroxide, aluminum phosphate, potassium aluminum sulfate, and combinations thereof. In other embodiments, the adjuvant is a squalene-in-water emulsion such as MF59 or AS03. In other embodiments, the adjuvant is a saponin, such as Quil A or QS-21, as in AS01 or AS02.

### C. Kits

Also provided herein are kits comprising at least one pharmaceutical formulation described herein. In some embodiments, the kit comprises a lyophilized or freeze-dried pharmaceutical formulation (e.g., one unit dose in a vial) disclosed herein and a solution for dissolving, diluting, and/or reconstituting the lyophilized pharmaceutical composition. In some embodiments, the solution for reconstituting or dilution is supplied as a pre-filled syringe. In some embodiments, the kit comprises a frozen suspension of a pharmaceutical formulation (e.g., one unit dose in a vial). In some embodiments, the kit includes a buffer that helps to prevent aggregation upon reconstituting the pharmaceutical composition disclosed herein. In some embodiments, the pharmaceutical composition is provided in a pre-filled syringe. In some embodiments, a kit comprises a dual-chamber syringe or container wherein one of the chambers contains a buffer for dissolving or diluting the pharmaceutical composition. In some embodiments, the kit comprises a syringe for injection. In some embodiments, the reconstituted solution is filtered before administration. In some embodiments, the kit comprises a filter or a filter syringe for filtering the reconstituted pharmaceutical composition before administration. In some embodiments, the kit further comprises instructions for use, e.g., instructions for hyperactivating cells.

### D. Particle Size of Drug Product

The particle size of the drug particles can affect the uptake of drug by cells. Particle size can be controlled by milling of the drug substance, such as DGP (Compound 2) by techniques well-known in the pharmaceutical arts. Dry milling techniques that can be used include, but are not limited to, jet milling, hammer milling, and pin milling. Wet milling techniques that can be used include, but are not limited to, rotor-stator milling, colloid milling, and media milling. Milling of the drug substance can be performed before further steps in the method, such as combining the drug substance with solutions, buffers, and/or other components to form a suspension.

The drug substance can be combined with solutions or buffers, such as phosphate-buffered saline and a poloxamer (for example, poloxamer 407 or poloxamer 188), to give a drug product. Further procedures can be used to reduce particle size in the drug product, including sonication and homogenization.

In embodiments, about 50% of the particles in the drug product have a diameter less than about 40 microns (D₅₀<40 microns). In embodiments, about 50% of the particles in the drug product have a diameter less than about 30 microns (D₅₀<30 microns). In embodiments, about 50% of the particles in the drug product have a diameter less than between about 20 microns and about 40 microns (D₅₀<20 microns to 40 microns). In embodiments, about 50% of the particles in the drug product have a diameter less than between about 20 microns and about 30 microns (D₅₀<20 microns to 30 microns). In embodiments, about 50% of the particles in the drug product have a diameter less than about 20 microns (D₅₀<20 microns). In embodiments, about 50% of the particles in the drug product have a diameter less than between about 10 microns and about 30 microns (D₅₀<10 microns to 30 microns). In embodiments, about 50% of the particles in the drug product have a diameter less than between about 10 microns (D₅₀<10 microns). In embodiments, about 50% of the particles in the drug product have a diameter less than between about 10 microns and about 20 microns (D₅₀<10 microns to 20 microns). In embodiments, about 50% of the particles in the drug product have a diameter less than between about 5 microns and about 20 microns (D₅₀<5 microns to 20 microns). In embodiments, about 50% of the particles in the drug product have a diameter less than between about 5 microns (D₅₀<5 microns).

In embodiments, about 50% of the particles in the drug product have a diameter less than about 40 microns (D₉₀<40 microns). In embodiments, about 50% of the particles in the drug product have a diameter less than about 30 microns (D₉₀<30 microns). In embodiments, about 50% of the particles in the drug product have a diameter less than between about 20 microns and about 40 microns (D₉₀<20 microns to 40 microns). In embodiments, about 50% of the particles in the drug product have a diameter less than between about 20 microns and about 30 microns (D₉₀<20 microns to 30 microns). In embodiments, about 50% of the particles in the drug product have a diameter less than about 20 microns (D₉₀<20 microns). In embodiments, about 50% of the particles in the drug product have a diameter less than between about 10 microns and about 30 microns (D₉₀<10 microns to 30 microns). In embodiments, about 50% of the particles in the drug product have a diameter less than between about 10 microns (D₉₀<10 microns). In embodiments, about 50% of the particles in the drug product have a diameter less than between about 10 microns and about 20 microns (D₉₀<10 microns to 20 microns). In embodiments, about 50% of the particles in the drug product have a diameter less than between about 5 microns and about 20 microns (D₉₀<5 microns to 20 microns). In embodiments, about 50% of the particles in the drug product have a diameter less than between about 5 microns (D₉₀<5 microns).

The particles of the drug product as described herein can comprise i) one or more of a surfactant, such as a non-ionic surfactant, such as a poloxamer or a Pluronic, such as Poloxamer 407, Poloxamer 188, Pluronic 84, or Pluronic 123; a wetting agent, such as P407, P188, or polysorbate 80; or a thickening agent, such as carboxymethyl cellulose; and ii) an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof. The particles can have a size or size range as indicated above.

### VI. Methods for Production

The present disclosure relates, in some aspects, to methods for preparing hyperactivated dendritic cells, and methods for preparing immunogenic compositions. The immunogenic compositions are suitable for hyperactivation of dendritic cells in vitro, ex vivo, or in vivo.

In one aspect, the present disclosure provides a method for production of hyperactivated dendritic cells (DCs), the method comprising contacting dendritic cells with effective amounts of an isolated ether lipid (ETL) (such as an isolated ether phospholipid (ETPL)) with an n-alkyl chain, and a PRR agonist for production of hyperactivated dendritic cells, wherein the hyperactivated dendritic cells secrete IL-1beta without undergoing pyroptosis. In some embodiments, the DCs are isolated, while in other embodiments, the DCs are present within a biological sample obtained from a mammalian subject, such as a human patient. In some embodiments, the DCs are monocyte-derived DCs, preferably cDC1s.

In a further aspect, the present disclosure provides a method for production of an immunogenic composition, the method comprising combining an antigen with effective amounts of an isolated ether lipid (ETL) (such as an isolated ether phospholipid (ETPL)) with an n-alkyl chain, and a PRR agonist for production of an immunogenic composition. In some embodiments, the antigen comprises a proteinaceous antigen that is present in or purified from a biological sample obtained from a mammalian subject. In some embodiments, the proteinaceous antigen is a synthetic or recombinant protein. In some preferred embodiments, the antigen is a tumor antigen. In some preferred embodiments, the antigen is a microbial antigen.

In specific embodiments, the present disclosure provide a method for production of an immunogenic composition, the method comprising:
a) depleting leukocytes from a suspension of cells prepared from a tumor to obtain a tumor cell-enriched suspension;
b) lysing cells from the tumor cell-enriched suspension to obtain a tumor cell lysate; and
c) contacting the tumor cell lysate with an isolated ether lipid (ETL) (such as an isolated ether phospholipid (ETPL)) with an n-alkyl chain and a PRR agonist to obtain the immunogenic composition. In some embodiments, the leukocytes are depleted from the tumor cell-enriched cell suspension by contacting the tumor cell-enriched suspension with an antibody specific to leukocytes. In some embodiments, the leukocytes are depleted by contacting the tumor cell-enriched suspension with an anti-CD45 antibody. In some embodiments, the cells are lysed by a physical disruption-based cell lysis method, such as, but not limited to, mechanical lysis, liquid homogenization, sonication, freeze-thaw, or manual grinding. In some preferred embodiments, the cells are lysed by one or more freeze-thaw cycles.

In some embodiments of the afore-mentioned methods, the alkyl chain of the ETL (such as an ETPL) is a C13-C22 n-alkyl chain or a C13-C24 n-alkyl chain. In some embodiments, the alkyl chain of the ETL (such as an ETPL) is a C18-C22 n-alkyl chain or a C18-C24 n-alkyl chain. In some preferred embodiments, the alkyl chain of the ETL (such as an ETPL) is a C22 n-alkyl chain. In some preferred embodiments, the ETPL is DGPC. In some preferred embodiments, the ETPL is DGP. In some embodiments, the PRR agonist is a TLR agonist. In some embodiments, the PRR agonist is a TLR7/8 agonist. In some preferred embodiments, the TLR7/8 agonist is an imidazoquinoline compound, which in particularly preferred embodiments is resiquimod (R848).

### VII. Further Lipids

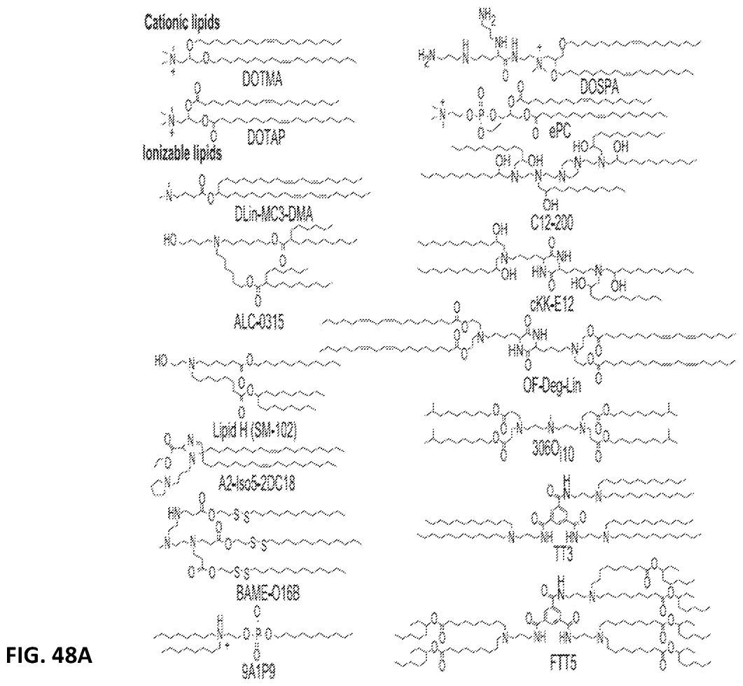

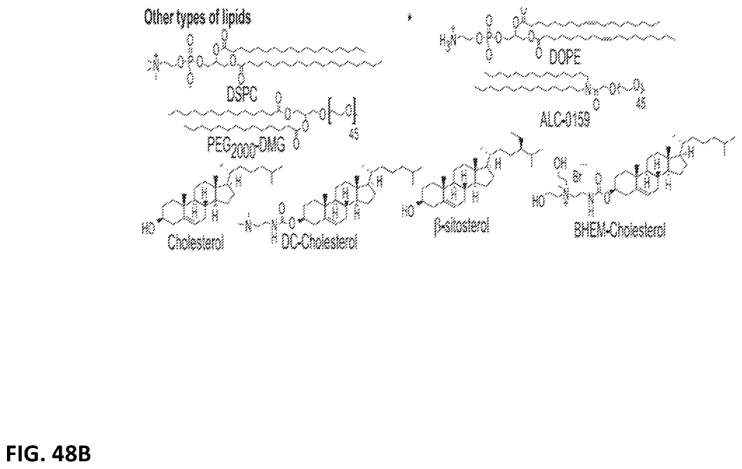

Compositions and methods of the present disclosure comprise at least one further lipid, wherein the LPC and the at least one further lipid are part of a lipid nanoparticle (LNP). In some embodiments, the at least one further lipid comprises an ionizable lipid, a cationic lipid, a further phospholipid, a pegylated lipid, a structural lipid, or a mixture thereof. In some embodiments, the LNP comprises a first phospholipid (lysophosphatidylcholine with a single C13-C24 acyl chain [LPC:C13-C24]), an ionizable lipid, a second phospholipid, a pegylated lipid, and a structural lipid. Structures of further lipids suitable for use in the compositions and methods of the present disclosure are shown in FIG. 48A and FIG. 48B (reproduced from Hou et al., Nature Review Materials, 6:1078-1094, 2021).

In some embodiments, the at least one further lipid comprises one or both of a further phospholipid and a structural lipid, optionally wherein the further phospholipid comprises 1,2-distearoyl-sn-glycero-3-phosphocholine (DSPC), and the structural lipid comprises cholesterol. In some embodiments, the at least one further lipid comprises or further comprises a pegylated lipid, optionally wherein the pegylated lipid comprises polyethylene glycol [PEG]2000 dimyristoyl glycerol [DMG]. In some embodiments, at least one further lipid comprises or further comprises an ionizable lipid, optionally wherein the ionizable lipid comprises (6Z,9Z,28Z,31Z)-heptatriaconta-6,9,28,31-tetraen-19-yl 4-(dimethylamino)butanoate (DLin-MC3-DMA) or analogs or derivatives thereof. In some embodiments, the at least one further lipid comprises at least one lipid from the following list (disclosed in Hou et al., Nature Review Materials 6, 1078-1094 (2021)); these lipids include 306Oi10, tetrakis(8-methylnonyl) 3,3′,3″,3′″-(((methylazanediyl) bis(propane-3,1 diyl))bis(azanetriyl))tetrapropionate; 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; Q-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B,bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1Hcyclopenta[a]phenanthren-3-yl)oxy)carbonyl)amino)-N,N-bis(2-hydroxyethyl)-Nmethylethan-1-aminium bromide; C₁₂-200, 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; DC-Cholesterol, 3|-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino)butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminiumtrifluoroacetate; DOTAP, 1,2-dioleoyl-3-trimethylammonium-propane; DOTMA,1,2-di-O-octadecenyl-3-trimethylammonium-propane; DSPC, 1,2-distearoyl-snglycero-3-phosphocholine; ePC, ethylphosphatidylcholine; FTT5, hexa(octan-3-yl)9,9′,9″,9″,9″″,9″″-((((benzene-1,3,5-tricarbonyl)yris(azanediyl)) tris (propane-3,1-diyl))tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,127,12″Z,12″Z)-tetrakis (octadeca-9,12-dienoate); PEG2000-DMG, 1,2-dimyristoylrac-glycero-3-methoxypolyethylene glycol-2000; TT3, N1,N3,N5-tris(3-(didodecylamino)propyl)benzene-1,3,5-tricarboxamide, and are illustrated in FIG. 48A and FIG. 48B.

VIII. mRNA Encoding an Antigen

Compositions and methods of the present disclosure comprise an mRNA encoding an antigen or are otherwise suitable for use with a formulation comprising an mRNA encoding an antigen. In some embodiments, the antigen is a proteinaceous antigen. The terms “polypeptide” and “protein” are used interchangeably herein in reference to antigens that comprise peptide chains that are at least 8 amino acids in length. In some embodiments, the antigen is from 8 to 1800 amino acids, 9 to 1000 amino acids, or 10 to 100 amino acids in length. The polypeptide may be post-translationally modified such as by phosphorylation, hydroxylation, sulfonation, palmitoylation, and/or glycosylation.

In some embodiments, the antigen is a tumor antigen that comprises the amino acid sequence of at least one full length protein or fragment thereof. In some embodiments, the tumor antigen comprises an amino acid sequence or fragment thereof from an oncoprotein. In some embodiments, the mammalian antigen is a neoantigen or encoded by a gene comprising a mutation relative to the gene present in normal cells from a mammalian subject. Neoantigens are thought to be particularly useful in enabling T cells to distinguish between cancer cells and non-cancer cells (see, e.g., Schumacher and Schreiber, Science, 348:69-74, 2015). In other embodiments, the tumor antigen comprises a viral antigen, such as an antigen of a cancer-causing virus.

In some embodiments, the tumor antigen is a fusion protein comprising two or more polypeptides, wherein each polypeptide comprises an amino acid sequence from a different tumor antigen or non-contiguous amino acid sequences from the same tumor antigen. In some of these embodiments, the fusion protein comprises a first polypeptide and a second polypeptide, wherein each polypeptide comprises non-contiguous amino acid sequences from the same tumor antigen.

In some embodiments, the antigen is a microbial antigen. In some embodiments, the microbial antigen comprises a viral antigen, a bacterial antigen, a protozoan antigen, a fungal antigen, or combinations thereof. In some embodiments, the microbial antigen comprises a surface protein or other antigenic subunit of a microbe.

In some preferred embodiments, the mRNA comprises a 5′ untranslated region (5′UTR) at the 5′ end of the coding region and a 3′ untranslated region (3′UTR) at the 3′ end of the coding region. In some preferred embodiments, the mRNA comprises one or both of a 5′ cap structure and a polyA tail.

### IX. Lipid-Based Delivery Vehicles

Compositions and methods of the present disclosure comprise a lipid-based delivery vehicle for the mRNA encoding an antigen. In some embodiments, the vehicle is a lipid nanoparticle (LNP). In other embodiments, the vehicle is a lipid that forms a complex with the mRNA (RNA-Lipoplex).

In some embodiments, the LNP comprises an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; and at least one lipid selected from the group consisting of an ionizable lipid, a cationic lipid, a second phospholipid, a pegylated lipid, a structural lipid, and mixtures thereof. In some embodiments, the ether lipid (ETL) or ether phospholipid (ETPL) is isolated. In some embodiments, the at least one lipid comprises an ionizable lipid. In some embodiments, the at least one lipid comprises a cationic lipid. In some embodiments, the at least one lipid comprises a second phospholipid. In some embodiments, the at least one lipid comprises a pegylated lipid. In some embodiments, the at least one lipid comprises a structural lipid. In some embodiments, the at least one lipid comprise an ionizable lipid, a second phospholipid, a pegylated lipid, and a structural lipid.

In some embodiments, the LNP comprises an ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; and at least one lipid selected from the group consisting of an ionizable lipid, a cationic lipid, a second phospholipid, a pegylated lipid, a structural lipid, and mixtures thereof. In some embodiments, the ether lipid (ETL) or ether phospholipid (ETPL) is isolated. In some embodiments, the at least one lipid comprises an ionizable lipid. In some embodiments, the at least one lipid comprises a cationic lipid. In some embodiments, the at least one lipid comprises a second phospholipid. In some embodiments, the at least one lipid comprises a pegylated lipid. In some embodiments, the at least one lipid comprises a structural lipid. In some embodiments, the at least one lipid comprise an ionizable lipid, a second phospholipid, a pegylated lipid, and a structural lipid.

In some embodiments, the lipid component of RNA-Lipoplex comprises one or more lipids. In some preferred embodiments, the one or more lipids comprise a first lipid and a second lipid, wherein the first lipid is distinct from the second lipid. In some embodiments, the first lipid is a cationic lipid and the second lipid is a neutral or anionic lipid.

Structure of lipids suitable for use in the lipid-based mRNA delivery vehicles of the present disclosure are shown in FIG. 48A and FIG. 48B (reproduced from Hou et al., Nature Review Materials, 6:1078-1094, 2021).

### X. Methods of Use

In some aspects, the present disclosure relates to methods of use of any one of the compositions or formulations described herein, which comprise an ETL compound, such as an ETPL compound. In some embodiments, the compositions or formulations further comprise a PRR agonist, a dendritic cell, an antigen, an adjuvant, or any combination thereof. The methods of use are suitable for a plurality of uses involving stimulating an immune response. In some embodiments, the methods of use comprise methods of treating cancer. In some embodiments, the methods of use comprise methods of inhibiting abnormal cell proliferation. In some embodiments, the methods of use comprise methods of treating an infectious disease. The methods comprise administering an effective amount of a formulation or a composition described herein to an individual in need thereof to achieve a specific outcome. The individual is a mammalian subject, such as a human patient. In other embodiments, the individual is a non-human patient. In some embodiments, the individual is a canine patient. That is in some embodiments, the methods of use involve clinical uses, while in other embodiments the methods of use involve pre-clinical and/or veterinary uses. For preclinical uses, the mammalian subject may be a non-human primate (e.g., monkey or ape) or a rodent (e.g., mouse or rat). For veterinary uses the mammalian subject may be a farm animal (e.g., cow), a sport animal (e.g., horse), or a pet (e.g., companion animal such as a dog or cat).

### A. Stimulation of an Immune Response

In brief, the present disclosure provides methods of stimulating an immune response in an individual, comprising administering to the individual a composition or formulation described herein in an amount sufficient to stimulate an immune response in the individual. “Stimulating” an immune response (used interchangeably with “eliciting” an immune response), means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response comprises one or more of the group consisting of: stimulating cytokine production; stimulating B lymphocyte proliferation; stimulating interferon pathway-associated gene expression; stimulating chemoattractant-associated gene expression; and stimulating dendritic cell DC maturation. Methods for measuring stimulation of an immune response are known in the art.

For instance, the present disclosure provides methods of inducing an antigen-specific immune response in an individual by administering to the individual a composition or formulation described herein in an amount sufficient to induce an antigen-specific immune response in the individual. In preferred embodiments, the composition or formulation comprises the antigen. In some embodiments, the composition or formulation is administered to a tissue of the individual comprising the antigen. The immune response may comprise one or both of an antigen-specific antibody response and an antigen-specific cytotoxic T lymphocyte (CTL) response. “Inducing” an antigen-specific antibody response means increasing titer of the antigen-specific antibodies above a threshold level such as a pre-administration baseline titer or a seroprotective level. “Inducing” an antigen-specific CTL response means increasing frequency of antigen-specific CTL found in peripheral blood above a pre-administration baseline frequency.

Analysis (both qualitative and quantitative) of the immune response can be by any method known in the art, including, but not limited to, measuring antigen-specific antibody production (including measuring specific antibody subclasses), activation of specific populations of lymphocytes such as B cells and helper T cells, production of cytokines such as IFN-alpha, IFN-gamma, IL-6, IL-12 and/or release of histamine. Methods for measuring antigen-specific antibody responses include enzyme-linked immunosorbent assay (ELISA). Activation of specific populations of lymphocytes can be measured by proliferation assays, and with fluorescence-activated cell sorting (FACS). Production of cytokines can also be measured by ELISA. In some embodiments, methods of stimulating an immune response comprise stimulation of interleukin-1beta (IL-1β) secretion, interferon-gamma (IFN-γ) secretion, and/or tumor necrosis factor-alpha (TNF-α) secretion by monocyte-derived dendritic cells or peripheral blood mononuclear cells. In some embodiments, methods of stimulating an immune response comprise stimulation of secretion of one or more of IFN-γ, IL-17a, IL-17f, and IL-22 by memory CD4+ T cells. In some embodiments, methods of stimulating an immune response comprise increasing Th1 differentiation of naïve CD4+ T cells. In some preferred embodiments, at least 50%, 55%, 60%, 65%, 70% or 75% of the cells contacted with a composition of the present disclosure remain viable at 40-56 hours (or about 48 hours) post-contact.

In some embodiments, the methods are suitable for stimulating an anti-tumor immune response. In other embodiments, the methods are suitable for stimulating an anti-microbe immune response. In some embodiments, the anti-microbe response is an anti-bacterial immune response. In some embodiments, the anti-microbe response is an anti-fungal immune response. In some embodiments, the anti-microbe response is, an anti-viral immune response. In some embodiments, the anti-microbe response is an anti-protozoan immune response.

### B. Treating or Preventing Disease

The present disclosure further provides methods of treating or preventing a disease in an individual, comprising administering to the individual a composition or formulation described herein in an amount sufficient to treat or prevent a disease in the individual. In some embodiments, the disease is cancer. In some embodiments, the disease is abnormal cell proliferation. In other embodiments, the disease is an infectious disease.

In one aspect, the methods may comprise administering a composition comprising an ETL compound, such as an ETPL compound, to a subject in need thereof. In some embodiments, the compositions further comprise a PRR agonist, an antigen, an adjuvant, or any combination thereof. In a further aspect, the methods involve adoptive cell therapy, and comprise administering a composition comprising a dendritic cell, such as a hyperactivated dendritic cell, and an ETL compound (such as an ETPL compound) to a subject in need thereof. In some embodiments, the compositions further comprise a PRR agonist, an antigen, an adjuvant, or any combination thereof.

In some embodiments, the methods involve treating cancer in an individual or otherwise treating a mammalian subject with cancer. In some embodiments, the methods comprise: a) preparing an immunogenic composition comprising a tumor cell lysate, an isolated ether lipid (ETL) (such as an isolated ether phospholipid (ETPL)) having an n-alkyl chain, and a toll-like receptor (TLR) agonist, such as a toll-like receptor 7/8 (TLR7/8) agonist, wherein the tumor cell lysate is or has been prepared from a sample of a tumor obtained from the subject with cancer, and the alkyl chain is a C13-C22 n-alkyl chain or a C13-C24 n-alkyl chain; and b) administering to the subject an effective amount of the immunogenic composition. In some embodiments, the cancer is a hematologic cancer, such as a lymphoma, a leukemia, or a myeloma. In other embodiments, the cancer is a non-hematologic cancer, such as a sarcoma, a carcinoma, or a melanoma. In some embodiments, the cancer is malignant.

In some embodiments, the methods involve inhibiting abnormal cell proliferation in an individual. “Abnormal cell proliferation” refers to proliferation of a benign tumor or a malignant tumor. The malignant tumor may be a metastatic tumor.

In some embodiments, the methods involve treating or preventing an infectious disease in an individual. In some embodiments, the infectious disease is caused by a viral infection. In other embodiments, the infectious disease is caused by a bacterial infection. In further embodiments, the infectious disease is caused by a fungal infection. In still further embodiments, the infectious disease is caused by a protozoal infection. Of particular importance are infectious diseases caused by zoonotic pathogens that infect humans as well as other animals such as mammals or birds. In some embodiments, the zoonotic pathogen is transmitted to humans via an intermediate species (vector).

### ENUMERATED EMBODIMENTS

The following enumerated embodiments are representative of aspects of the disclosure. The features of each of the embodiments are combinable with any of the other embodiments where appropriate and practical.

Embodiment 1. A composition comprising an isolated ether lipid (ETL) of Formula (I):

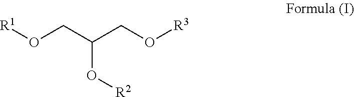

wherein:
R¹ is H or

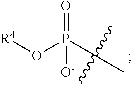

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated form thereof; or a pharmaceutically acceptable salt thereof; and
a TLR7/8 agonist.

Embodiment 2. The composition of embodiment 1, wherein R³ is C₁₈-C₂₂ n-alkyl or C₂₁-C₂₄ n-alkyl.

Embodiment 3. The composition of embodiment 1 or embodiment 2, further comprising an antigen.

Embodiment 4. The composition of any one of embodiments 1-3, further comprising dendritic cells.

Embodiment 5. A composition comprising an isolated ether lipid (ETL) of Formula (I):

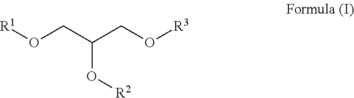

wherein:
R¹ is H or

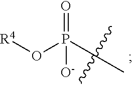

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated form thereof; or a pharmaceutically acceptable salt thereof; and
an antigen.

Embodiment 6. The composition of embodiment 5, further comprising dendritic cells.

Embodiment 7. The composition of embodiment 5 or embodiment 6, further comprising a TLR7/8 agonist.

Embodiment 8. A composition comprising an isolated ether lipid (ETL) of Formula (I):

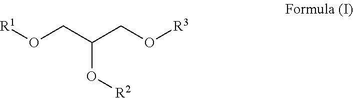

wherein:
R¹ is H or

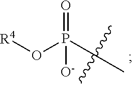

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated form thereof; or a pharmaceutically acceptable salt thereof; and
dendritic cells.

Embodiment 9. The composition of embodiment 8, further comprising a TLR7/8 agonist.

Embodiment 10. The composition of embodiment 8 or embodiment 9, further comprising an antigen.

Embodiment 11. A composition of any one of embodiments 1-10, wherein R³ is C₂₂ n-alkyl.

Embodiment 12. The composition of any one of embodiments 1-11, wherein the ETL is an ether phospholipid (ETPL) which comprises 1-docosyl-sn-glycerol-3-phosphocholine (DGPC), or a pharmaceutically acceptable salt thereof.

Embodiment 13. The composition of any one of embodiments 1-11, wherein the ETL is an ETPL which comprises 1-docosyl-sn-glycerol-3-phosphate (DGP), or a pharmaceutically acceptable salt thereof.

Embodiment 14. The composition of any one of embodiments 1-13, wherein the TLR7/8 agonist is a small molecule with a molecule weight of 900 daltons or less.

Embodiment 15. The composition of embodiment 14, wherein the TLR7/8 agonist comprises an imidazoquinoline compound.

Embodiment 16. The composition of embodiment 15, wherein the TLR7/8 agonist comprises resiquimod (R848).

Embodiment 17. The composition of embodiment 14 or embodiment 15, wherein the TLR7/8 agonist does not inhibit NLR family pyrin domain containing 3 (NLRP3).

Embodiment 18. The composition of embodiment 13, wherein the ETPL comprises one or both of DGPC and DGP, and the TLR7/8 agonist comprises resiquimod (R848).

Embodiment 19. The composition of any one of embodiments 1-18, wherein the antigen is present in a biological sample obtained from an individual.

Embodiment 20. The composition of embodiment 19, wherein the biological sample comprises biopsy tissue.

Embodiment 21. The composition of embodiment 19, wherein the biological sample comprises cells.

Embodiment 22. The composition of embodiment 19, wherein the biological sample does not comprise cells.

Embodiment 23. The composition of embodiment 19, wherein the biological sample comprises pus from an abscess.

Embodiment 24. The composition of any one of embodiments 1-23, wherein the antigen comprises a proteinaceous antigen.

Embodiment 25. The composition of embodiment 24, wherein the antigen comprises a tumor antigen.

Embodiment 26. The composition of embodiment 25, wherein the tumor antigen comprises a synthetic or recombinant neoantigen.

Embodiment 27. The composition of embodiment 26, wherein the tumor antigen comprises a tumor cell lysate.

Embodiment 28. The composition of embodiment 24, wherein the antigen comprises a microbial antigen and the microbial antigen comprises one or more of a viral antigen, a bacterial antigen, a protozoan antigen, and a fungal antigen.

Embodiment 29. The composition of embodiment 28, wherein the microbial antigen comprises a purified or recombinant surface protein.

Embodiment 30. The composition of embodiment 28, wherein the microbial antigen comprises an inactivated, whole virus.

Embodiment 31. The composition of any one of embodiments 1-30, wherein the composition does not comprise liposomes.

Embodiment 32. The composition of any one of embodiments 1-31, wherein the composition does not comprise LPS or MPLA.

Embodiment 33. The composition of any one of embodiments 1-32, wherein the composition does not comprise oxPAPC or a species of oxPAPC, optionally wherein the composition does not comprise HOdiA-PC, KOdiA-PC, HOOA-PC, KOOA-PC, and/or PGPC.

Embodiment 34. The composition of embodiment 33, wherein the composition does not comprise lysophosphatidylcholine (LPC), optionally wherein the composition does not comprise 1-behenoyl-2-hydroxy-sn-glycero-3-phosphocholine [LPC(22:0)].

Embodiment 35. The composition of any one of embodiments 1-34, further comprising an adjuvant, wherein the adjuvant comprises an aluminum salt adjuvant, a squalene-in-water emulsion, a saponin, or combinations thereof.

Embodiment 36. A pharmaceutical formulation comprising the composition of any one of embodiments 1-35 and a pharmaceutically acceptable excipient.

Embodiment 37. A method for production of hyperactivated dendritic cells, the method comprising contacting the dendritic cells with a composition comprising effective amounts of an isolated ether lipid (ETL) of Formula (I):

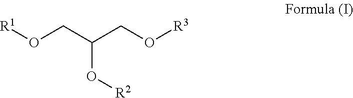

wherein:
R¹ is H or

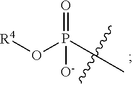

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated form thereof; or a pharmaceutically acceptable salt thereof; and
a TLR7/8 agonist for production of hyperactivated dendritic cells, wherein the hyperactivated dendritic cells secrete IL-1beta without undergoing pyroptosis.

Embodiment 38. The method of embodiment 37, wherein the dendritic cells are contacted ex vivo with the composition of any one of embodiments 1-35 or the formulation of embodiment 36.

Embodiment 39. The method of embodiment 37, wherein the dendritic cells are contacted in vivo with the formulation of embodiment 36.

Embodiment 40. A pharmaceutical formulation comprising at least 10³, 10⁴, 10⁵ or 10⁶ of the hyperactivated dendritic cells produced by the method of embodiment 38, and a pharmaceutically acceptable excipient.

Embodiment 41. A method of stimulating an immune response against an antigen, comprising administering an effective amount of the formulation of embodiment 36 to an individual in need thereof to stimulate the immune response against the antigen.

Embodiment 42. A method of treating cancer, comprising administering an effective amount of the formulation of embodiment 36 to an individual in need thereof to treat the cancer.

Embodiment 43. A method of inhibiting abnormal cell proliferation, comprising administering an effective amount of the formulation of embodiment 36 to an individual in need thereof to inhibit abnormal cell proliferation.

Embodiment 44. A method of treating an infectious disease, comprising administering an effective amount of the formulation of embodiment 36 to an individual in need thereof to treat the infectious disease.

Embodiment 45. Use of the formulation of embodiment 36 for inducing an immune response against the antigen in an individual in need thereof.

Embodiment 46. Use of the formulation of embodiment 36 for inducing an anti-tumor immune response in an individual in need thereof, wherein the individual is or was tumor-bearing.

Embodiment 47. Use of the formulation of embodiment 36 for inducing an anti-microbe immune response in an individual in need thereof, wherein the individual is infected with the microbe or has not been exposed to the microbe.

Embodiment 48. The composition, formulation, method or use of any one of embodiments 19-47, wherein the individual is a mammalian subject.

Embodiment 49. The composition, formulation, method or use of any one of embodiments 19-47, wherein the individual is a human subject.

Embodiment 50. A method of preparing an immunogenic composition, the method comprising:
a) depleting leukocytes from a suspension of cells prepared from a tumor to obtain a tumor cell-enriched suspension;
b) lysing cells from the tumor cell-enriched suspension to obtain a tumor cell lysate; and
c) contacting the tumor cell lysate with an isolated ether lipid (ETL) of Formula (I):

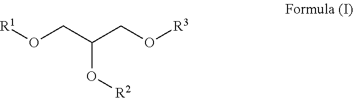

wherein:
R¹ is H or

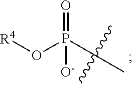

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated form thereof; or a pharmaceutically acceptable salt thereof; and
a toll-like receptor 7/8 (TLR7/8) agonist to obtain the immunogenic composition.

Embodiment 51. The method of embodiment 50, wherein the leukocytes are depleted in step a) by negative selection using an anti-CD45 antibody.

Embodiment 52. The method of embodiment 50 or embodiment 51, wherein the cells are lysed in step b) by one or more freeze-thaw cycles.

Embodiment 53. The method of any one of embodiments 50-52, wherein R³ is C₁₈-C₂₂ alkyl or C₁₈-C₂₄ alkyl.

Embodiment 54. The method of embodiment 53, wherein the ETL comprises one or both of DGPC and DGP, or a pharmaceutically acceptable salt thereof.

Embodiment 55. The method of any one of embodiments 50-54, wherein the TLR7/8 agonist is a small molecule with a molecule weight of 900 daltons or less.

Embodiment 56. The method of embodiment 55, wherein the TLR7/8 agonist comprises an imidazoquinoline compound.

Embodiment 57. The method of embodiments 56, wherein the TLR7/8 agonist comprises resiquimod (R848).

Embodiment 58. The method of embodiment 55 or embodiment 56, wherein the TLR7/8 agonist does not inhibit NLR family pyrin domain containing 3 (NLRP3).

Embodiment 59. The method of embodiment 54, wherein the ETL comprises one or both of DGPC and DGP or a pharmaceutically acceptable salt thereof, and the TLR7/8 agonist comprises resiquimod (R848).

Embodiment 60. The method of any one of embodiments 50-59, further comprising before step a) obtaining a sample from the tumor from a mammalian subject with cancer and preparing the suspension of cells from the sample.

Embodiment 61. An immunogenic composition prepared by the method of any one of embodiments 50-60.

Embodiment 62. A method of eliciting an anti-cancer immune response, the method comprising administering to a mammalian subject with cancer an effective amount of the immunogenic composition of embodiment 61.

Embodiment 63. The method of embodiment 62, wherein the anti-cancer immune response comprises cellular immune response.

Embodiment 64. The method of embodiment 63, wherein the anti-cancer immune response comprises cancer antigen-induced IL-1beta secretion and/or activation of CD8+ T lymphocytes.

Embodiment 65. The method of any one of embodiments 62-64, wherein the cancer is a non-hematologic cancer.

Embodiment 66. The method of embodiment 65, wherein the non-hematologic cancer is a carcinoma, a sarcoma, or a melanoma.

Embodiment 67. The method of any one of embodiments 62-64, wherein the cancer is a lymphoma.

Embodiment 68. A method of treating cancer, the method comprising:
a) preparing an immunogenic composition comprising a tumor cell lysate, an isolated ether lipid (ETL) of Formula (I):

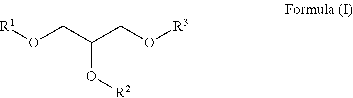

wherein:
R is H or

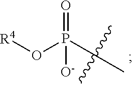

R² is H, C₁-C₄ alkyl, —(C═O)—NH₂, —(C═O)—NH(R⁵), —(C═O)—N(R⁵)₂, or —CH₂—C₆H₅;
R; is C₁₃-C₂₄ n-alkyl;
where R⁴ is H or (CH₃)₃N⁺—(CH₂)₂—; and
each R⁵ is independently C₁-C₄ alkyl;
or a protonated form thereof; or a pharmaceutically acceptable salt thereof; and
a toll-like receptor 7/8 (TLR7/8) agonist, wherein the tumor cell lysate is or has been prepared from a sample of a tumor obtained from the mammalian subject with cancer; and
b) administering to the subject an effective amount of the immunogenic composition.

Embodiment 69. The method of any one of embodiments 62-68, wherein R³ is a C₁₈-C₂₂ alkyl chain or a C₁₈-C₂₄ alkyl chain.

Embodiment 70. The method of embodiment 68, wherein the ETL comprises one or both of DGPC and DGP, or a pharmaceutically acceptable salt thereof.

Embodiment 71. The method of any one of embodiments 62-70, wherein the TLR7/8 agonist is a small molecule with a molecule weight of 900 daltons or less.

Embodiment 72. The method of embodiment 71, wherein the TLR7/8 agonist comprises an imidazoquinoline compound.

Embodiment 73. The method of embodiment 72, wherein the TLR7/8 agonist comprises resiquimod (R848).

Embodiment 74. The method of embodiment 70, wherein the ETPL comprises DGPC or a pharmaceutically acceptable salt thereof, and the TLR7/8 agonist comprises resiquimod (R848).

Embodiment 75. The method of embodiment 70, wherein the ETPL comprises DGP or a pharmaceutically acceptable salt thereof, and the TLR7/8 agonist comprises resiquimod (R848).

Embodiment 76. The method of any one of claims 68-75, further comprising administering to the subject an effective amount of an additional therapeutic agent.

Embodiment 77. The method of embodiment 76, wherein the additional therapeutic agent comprises one or more of the group consisting of an immune checkpoint inhibitor, an antineoplastic agent, and radiation therapy.

Embodiment 78. A composition comprising an isolated ether lipid (ETL) of Formula (I):

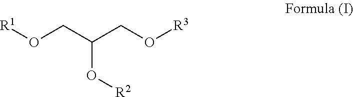

wherein:
R¹ is H or

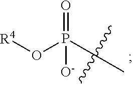

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated form thereof; or a pharmaceutically acceptable salt thereof; and
a pathogen recognition receptor (PRR) agonist.

Embodiment 79. The composition of embodiment 78, wherein the PRR agonist is an agonist of a toll-like receptor (TLR), a NOD-like receptor (NLR), a RIG-I-like receptor (RLR), or a C-type lectin receptor (CLR).

Embodiment 80. The composition of embodiment 78, wherein the PRR agonist is an agonist of a cytosolic DNA sensor (CDS) or a stimulator of IFN genes (STING).

Embodiment 81. The composition of embodiment 78, wherein the PRR agonist comprises one or more of R848, TL8-506, LPS, Pam2CSK4, and ODN 2336.

Embodiment 82. The composition of any one of embodiments 78-81, further comprising an antigen.

Embodiment 83. The composition of any one of embodiments 78-82, further comprising dendritic cells.

Embodiment 84. A pharmaceutical formulation comprising the composition of any one of embodiments 78-83 and a pharmaceutically acceptable excipient.

Embodiment 85. A pharmaceutical formulation comprising an isolated ether lipid (ETL) of Formula (I):

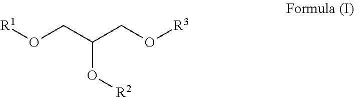

wherein:
R¹ is H or

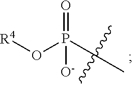

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated form thereof; or a pharmaceutically acceptable salt thereof; and
a pharmaceutically acceptable excipient.

Embodiment 86. The pharmaceutical formulation of embodiment 84 or embodiment 85, wherein the alkyl chain is a C₂₂ n-alkyl chain.

Embodiment 87. The pharmaceutical formulation of embodiment 86, wherein the ETL comprises one or both of DGPC and DGP, or a pharmaceutically acceptable salt thereof.

Embodiment 88. A composition for hyperactivation of human dendritic cells, comprising an isolated ether lipid (ETL) of Formula (I):

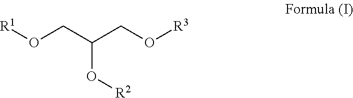

wherein:
R¹ is H or

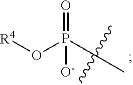

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated form thereof; or a pharmaceutically acceptable salt thereof; and
a pathogen recognition receptor (PRR) agonist, wherein the alkyl chain is a C₂₂ n-alkyl chain, and wherein the composition is effective for achieving a higher level of dendritic cell hyperactivation than a comparator composition comprising PGPC in place of the ETL.

Embodiment 89. The composition of embodiment 88, wherein R³ is C₂₂ n-alkyl.

Embodiment 90. The composition of embodiment 88 or embodiment 89, wherein the higher level of dendritic cell hyperactivation comprises induction of IL-1beta secretion from the human dendritic cells in vitro at a level that is at least 2, 3 or 4 fold higher when contacted with the composition comprising the ETL and the PRR agonist than when contacted with the comparator composition comprising the PGPC and the PRR agonist, wherein the PRR agonist is LPS.

Embodiment 91. The composition of embodiment 90, wherein the concentration of the ETL and the concentration of the PGPC are the same concentration in a range of from about 10 μM to about 80 μM, and the LPS is present at a concentration of 1 μg/ml in both the composition and the comparator composition.

Embodiment 92. The composition of embodiment 90, wherein the higher level of dendritic cell hyperactivation comprises a lipid activity index for IL-1beta secretion from the human dendritic cells for the composition comprising the ETL and the PRR agonist that is at least 4, 5 or 6 fold higher in activity units than that of the comparator composition comprising the PGPC and the PRR agonist.

Embodiment 93. The composition, formulation, method or use of any one of embodiments 19-47, wherein the individual is a human subject.

Embodiment 94. The composition, formulation, method or use of any one of embodiments 19-47, wherein the individual is a canine subject.

Embodiment 95. The composition, formulation, method or use of any one of embodiments 60-92, wherein the mammalian subject is a human patient.

Embodiment 96. The composition, formulation, method or use of any one of embodiments 60-92, wherein the mammalian subject is a non-human patient.

Embodiment 97. The composition, formulation, method or use of any one of embodiments 60-92, wherein the mammalian subject is a canine patient.

Embodiment 98. The composition, formulation, method or use of any one of embodiment 1-93 or 95, wherein the dendritic cells are human dendritic cells.

Embodiment 99. The composition, formulation, method or use of any one of embodiment 1-48, 50-87 or 97, wherein the dendritic cells are canine dendritic cells.

Embodiment 100. The composition, method or use of embodiment 98 or embodiment 99, wherein the dendritic cells are present in a composition comprising peripheral blood mononuclear cells (PBMCs).

Embodiment 101. The composition, method or use of any one of embodiments 37-49 or embodiments 98-99, wherein the hyperactivated dendritic cells secrete one or both of IFNγ and TNFα.

Embodiment 102. The composition, formulation, method or use of any one of embodiments 1-101, comprising a surfactant.

Embodiment 103. The composition, formulation, method or use of embodiment 102, wherein the surfactant comprises a non-ionic surfactant.

Embodiment 104. The composition, formulation, method or use of embodiment 103, wherein the non-ionic surfactant comprises an ethylene oxide-propylene oxide copolymer.

Embodiment 105. The composition, formulation, method or use of embodiment 103, wherein the non-ionic surfactant comprises one or more of Poloxamer 407, Poloxamer 188, and P123.

Embodiment 106. The composition, formulation, method or use of embodiment 103, wherein the non-ionic surfactant comprises Poloxamer 407.

Embodiment 107. The composition, formulation, method or use of any one of embodiments 103-106, wherein i) the ETL is dissolved in an alcohol to form an ETL alcohol solution; ii) the ETL alcohol solution is mixed with the non-ionic surfactant to form a mixture; and iii) the alcohol is evaporated from the mixture to form particles comprising the ETL and the non-ionic surfactant.

Embodiment 108. The composition, formulation, method or use of any one of embodiments 103-107, wherein the non-ionic surfactant is present in an amount of about 2.5% to 25% (w/w), optionally about 5% to 20% (w/w), optionally about 15% (w/w).

Embodiment 109. The composition, formulation, method or use of any one of embodiments 103-108, wherein the ETL and non-ionic surfactant are present in particles with a diameter of about 1000 to 15,000 nanometers, optionally with a diameter of about 5000 nanometers.

Embodiment A1. A composition comprising an isolated ether lipid (ETL) of Formula (I):

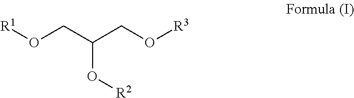

wherein:
R¹ is H or

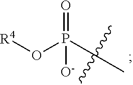

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
a TLR agonist.

Embodiment A2. The composition of embodiment A1, wherein the TLR agonist comprises a TLR7/8 agonist.

Embodiment A3. The composition of embodiment A1 or embodiment A2, wherein R³ is C₁₈-C₂₂ n-alkyl or C₂₁-C₂₄ n-alkyl.

Embodiment A4. The composition of any one of embodiments A1-A3, wherein R³ is C₁₆-C₂₀ n-alkyl.

Embodiment A5. The composition of any one of embodiments A1-A4, further comprising an antigen.

Embodiment A6. The composition of any one of embodiments A1-A5, further comprising dendritic cells.

Embodiment A7. A composition comprising an isolated ether lipid (ETL) of Formula (I):

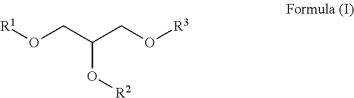

wherein:
R¹ is H or

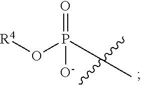

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
an antigen.

Embodiment A8. The composition of embodiment A7, further comprising dendritic cells.

Embodiment A9. The composition of embodiment A7 or embodiment A8, further comprising a TLR agonist.

Embodiment A10. The composition of embodiment A9, wherein the TLR agonist comprises a TLR7/8 agonist.

Embodiment A11. A composition comprising an isolated ether lipid (ETL) of Formula (I):

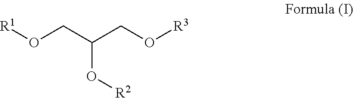

wherein:

R¹ is H or

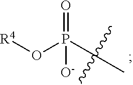

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
dendritic cells.

Embodiment A12. The composition of embodiment A11, further comprising a TLR agonist.

Embodiment A13. The composition of embodiment A12, wherein the TLR agonist comprises a TLR7/8 agonist.

Embodiment A14. The composition of any one of embodiments A11-A13, further comprising an antigen.

Embodiment A15. A composition of any one of embodiments A1-A14, wherein R³ is C₂₂ n-alkyl.

Embodiment A16. The composition of any one of embodiments A1-A15, wherein the ETL is an ether phospholipid (ETPL) which comprises 1-docosyl-sn-glycerol-3-phosphocholine (DGPC), or a pharmaceutically acceptable salt thereof.

Embodiment A17. The composition of any one of embodiments A1-A15, wherein the ETL is an ETPL which comprises 1-docosyl-sn-glycerol-3-phosphate (DGP), or a pharmaceutically acceptable salt thereof.

Embodiment A18. The composition of any one of embodiments A1-A17, wherein the TLR agonist is a small molecule with a molecule weight of 900 daltons or less.

Embodiment A19. The composition of any one of embodiments A1-A18, wherein the TLR agonist comprises a TLR7/8 agonist.

Embodiment A20. The composition of embodiment A19, wherein the TLR7/8 agonist comprises an imidazoquinoline compound.

Embodiment A21. The composition of embodiment A19, wherein the TLR7/8 agonist comprises resiquimod (R848).

Embodiment A22. The composition of any one of embodiments A18-A20, wherein the TLR7/8 agonist does not inhibit NLR family pyrin domain containing 3 (NLRP3).

Embodiment A23. The composition of any one of embodiments A1-A14, wherein the ETPL comprises one or both of DGPC and DGP, and the TLR7/8 agonist comprises resiquimod (R848).

Embodiment A24. The composition of any one of embodiments A1-A23, wherein the antigen is present in a biological sample obtained from an individual.

Embodiment A25. The composition of embodiment A24, wherein the biological sample comprises biopsy tissue.

Embodiment A26. The composition of embodiment A24, wherein the biological sample comprises cells.

Embodiment A27. The composition of embodiment A24, wherein the biological sample does not comprise cells.

Embodiment A28. The composition of embodiment A24, wherein the biological sample comprises pus from an abscess.

Embodiment A29. The composition of any one of embodiments A1-A28, wherein the antigen comprises a proteinaceous antigen.

Embodiment A30. The composition of embodiment A29, wherein the antigen comprises a tumor antigen.

Embodiment A31. The composition of embodiment A30, wherein the tumor antigen comprises a synthetic or recombinant neoantigen.

Embodiment A32. The composition of embodiment A30, wherein the tumor antigen comprises a tumor cell lysate.

Embodiment A33. The composition of embodiment A29, wherein the antigen comprises a microbial antigen and the microbial antigen comprises one or more of a viral antigen, a bacterial antigen, a protozoan antigen, and a fungal antigen.

Embodiment A34. The composition of embodiment A33, wherein the microbial antigen comprises a purified or recombinant surface protein.

Embodiment A35. The composition of embodiment A33, wherein the microbial antigen comprises an inactivated, whole virus.

Embodiment A36. The composition of any one of embodiments A1-A35, wherein the composition does not comprise liposomes.

Embodiment A37. The composition of any one of embodiments A1-A36, wherein the composition does not comprise LPS or MPLA.

Embodiment A38. The composition of any one of embodiments A1-A37, wherein the composition does not comprise oxPAPC or a species of oxPAPC, optionally wherein the composition does not comprise HOdiA-PC, KOdiA-PC, HOOA-PC, KOOA-PC, and/or PGPC.

Embodiment A39. The composition of any one of embodiments A1-A38, wherein the composition does not comprise lysophosphatidylcholine (LPC), optionally wherein the composition does not comprise 1-behenoyl-2-hydroxy-sn-glycero-3-phosphocholine [LPC(22:0)].

Embodiment A40. The composition of any one of embodiments A1-A39, further comprising an adjuvant, wherein the adjuvant comprises an aluminum salt adjuvant, a squalene-in-water emulsion, a saponin, or combinations thereof.

Embodiment A41. A pharmaceutical formulation comprising the composition of any one of embodiments A1-A40 and a pharmaceutically acceptable excipient.

Embodiment A42. A method for production of hyperactivated dendritic cells, the method comprising contacting the dendritic cells with a composition comprising effective amounts of an isolated ether lipid (ETL) of Formula (I):

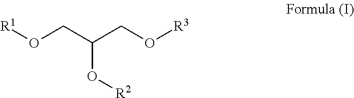

wherein:
R¹ is H or

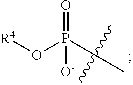

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
a TLR7/8 agonist for production of hyperactivated dendritic cells, wherein the hyperactivated dendritic cells secrete IL-1beta without undergoing pyroptosis.

Embodiment A43. The method of embodiment A42, wherein the dendritic cells are contacted ex vivo with the composition of any one of embodiments A1-A40 or the formulation of embodiment A41.

Embodiment A44. The method of embodiment A42, wherein the dendritic cells are contacted in vivo with the formulation of embodiment A41.

Embodiment A45. A pharmaceutical formulation comprising at least 10³, 10⁴, 10⁵ or 10⁶ of the hyperactivated dendritic cells produced by the method of embodiment A43, and a pharmaceutically acceptable excipient.

Embodiment A46. A method of stimulating an immune response against an antigen, comprising administering an effective amount of the formulation of embodiment A41 to an individual in need thereof to stimulate the immune response against the antigen.

Embodiment A47. A method of treating cancer, comprising administering an effective amount of the formulation of embodiment A41 to an individual in need thereof to treat the cancer.

Embodiment A48. A method of inhibiting abnormal cell proliferation, comprising administering an effective amount of the formulation of embodiment A41 to an individual in need thereof to inhibit abnormal cell proliferation.

Embodiment A49. A method of treating an infectious disease, comprising administering an effective amount of the formulation of embodiment A41 to an individual in need thereof to treat the infectious disease.

Embodiment A50. Use of the formulation of embodiment A41 for inducing an immune response against the antigen in an individual in need thereof.

Embodiment A51. Use of the formulation of embodiment A41 for inducing an anti-tumor immune response in an individual in need thereof, wherein the individual is or was tumor-bearing.

Embodiment A52. Use of the formulation of embodiment A41 for inducing an anti-microbe immune response in an individual in need thereof, wherein the individual is infected with the microbe or has not been exposed to the microbe.

Embodiment A53. The composition, formulation, method or use of any one of embodiments A24-A52, wherein the individual is a mammalian subject.

Embodiment A54. The composition, formulation, method or use of any one of embodiments A24-A52, wherein the individual is a human subject.

Embodiment A55. A method of preparing an immunogenic composition, the method comprising:
a) depleting leukocytes from a suspension of cells prepared from a tumor to obtain a tumor cell-enriched suspension;
b) lysing cells from the tumor cell-enriched suspension to obtain a tumor cell lysate; and
c) contacting the tumor cell lysate with an isolated ether lipid (ETL) of Formula (I):

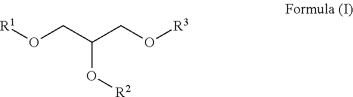

wherein:
R¹ is H or

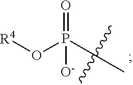

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
a toll-like receptor (TLR) agonist to obtain the immunogenic composition.

Embodiment A56. The method of embodiment A55, wherein the TLR agonist comprises a TLR7/8 agonist.

Embodiment A57. The method of embodiment A55 or embodiment A56, wherein the leukocytes are depleted in step a) by negative selection using an anti-CD45 antibody.

Embodiment A58. The method of any one of embodiments A55-A57, wherein the cells are lysed in step b) by one or more freeze-thaw cycles.

Embodiment A59. The method of any one of embodiments A55-A58, wherein R³ is C₁₈-C₂₂ alkyl or C₁₈-C₂₄ alkyl.

Embodiment A60. The method of any one of embodiments A55-A58, wherein R³ is C₁₆-C₂₀ alkyl.

Embodiment A61. The method of any one of embodiments A55-A58, wherein R³ is C₂₁-C₂₄ alkyl.

Embodiment A62. The method of embodiment A59 or embodiment A61, wherein the ETL comprises one or both of DGPC and DGP, or a pharmaceutically acceptable salt thereof.

Embodiment A63. The method of any one of embodiments A55-A62, wherein the TLR7/8 agonist is a small molecule with a molecule weight of 900 daltons or less.

Embodiment A64. The method of embodiment A63, wherein the TLR7/8 agonist comprises an imidazoquinoline compound.

Embodiment A65. The method of embodiment A64, wherein the TLR7/8 agonist comprises resiquimod (R848).

Embodiment A66. The method of any one of embodiments A63-A65, wherein the TLR7/8 agonist does not inhibit NLR family pyrin domain containing 3 (NLRP3).

Embodiment A67. The method of embodiment A62, wherein the ETL comprises one or both of DGPC and DGP or a pharmaceutically acceptable salt thereof, and the TLR7/8 agonist comprises resiquimod (R848).

Embodiment A68. The method of any one of embodiments A55-A67, further comprising before step a) obtaining a sample from the tumor from a mammalian subject with cancer and preparing the suspension of cells from the sample.

Embodiment A69. An immunogenic composition prepared by the method of any one of embodiments A55-A68.

Embodiment A70. A method of eliciting an anti-cancer immune response, the method comprising:
administering to a mammalian subject with cancer an effective amount of the immunogenic composition of embodiment A69.

Embodiment A71. The method of embodiment A70, wherein the anti-cancer immune response comprises cellular immune response.

Embodiment A72. The method of embodiment A63, wherein the anti-cancer immune response comprises cancer antigen-induced IL-1beta secretion and/or activation of CD8+ T lymphocytes.

Embodiment A73. The method of any one of embodiments A62-A64, wherein the cancer is a non-hematologic cancer.

Embodiment A74. The method of embodiment A65, wherein the non-hematologic cancer is a carcinoma, a sarcoma, or a melanoma.

Embodiment A75. The method of any one of embodiments A70-A74, wherein the cancer is a lymphoma.

Embodiment A76. A method of treating cancer, the method comprising:
a) preparing an immunogenic composition comprising a tumor cell lysate, an isolated ether lipid (ETL) of Formula (I):

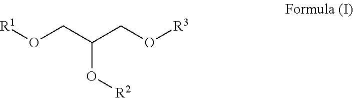

wherein:
R¹ is H or

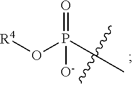

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
a toll-like receptor (TLR) agonist, wherein the tumor cell lysate is or has been prepared from a sample of a tumor obtained from the mammalian subject with cancer; and
b) administering to the subject an effective amount of the immunogenic composition.

Embodiment A77. The method of embodiment A76, wherein the TLR agonist comprises a TLR7/8 agonist.

Embodiment A78. The method of any one of embodiments A70-A77, wherein R³ is a C₁₈-C₂₂ alkyl chain or a C₁₈-C₂₂ alkyl chain.

Embodiment A79. The method of any one of embodiments A70-A77, wherein R³ is C₁₆-C₂₀ alkyl.

Embodiment A80. The method of any one of embodiments A70-A77, wherein R³ is C₂₁-C₂₄ alkyl.

Embodiment A81. The method of embodiment A76 or embodiment A77, wherein the ETL comprises one or both of DGPC and DGP, or a pharmaceutically acceptable salt thereof.

Embodiment A82. The method of any one of embodiments A70-A81, wherein the TLR7/8 agonist is a small molecule with a molecule weight of 900 daltons or less.

Embodiment A83. The method of embodiment A82, wherein the TLR7/8 agonist comprises an imidazoquinoline compound.

Embodiment A84. The method of embodiment A83, wherein the TLR7/8 agonist comprises resiquimod (R848).

Embodiment A85. The method of embodiment A81, wherein the ETPL comprises DGPC or a pharmaceutically acceptable salt thereof, and the TLR7/8 agonist comprises resiquimod (R848).

Embodiment A86. The method of embodiment A81, wherein the ETPL comprises DGP or a pharmaceutically acceptable salt thereof, and the TLR7/8 agonist comprises resiquimod (R848).

Embodiment A87. The method of any one of claims 68-75, further comprising administering to the subject an effective amount of an additional therapeutic agent.

Embodiment A88. The method of embodiment A76, wherein the additional therapeutic agent comprises one or more of the group consisting of an immune checkpoint inhibitor, an antineoplastic agent, and radiation therapy.

Embodiment A89. A composition comprising an isolated ether lipid (ETL) of Formula (I):

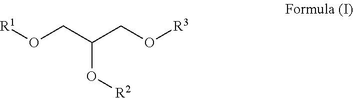

wherein:
R¹ is H or

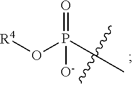

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
a pathogen recognition receptor (PRR) agonist.

Embodiment A90. The composition of embodiment A89, wherein the PRR agonist is an agonist of a toll-like receptor (TLR), a NOD-like receptor (NLR), a RIG-I-like receptor (RLR), or a C-type lectin receptor (CLR).

Embodiment A91. The composition of embodiment A89, wherein the PRR agonist is an agonist of a cytosolic DNA sensor (CDS) or a stimulator of IFN genes (STING).

Embodiment A92. The composition of embodiment A89, wherein the PRR agonist comprises one or more of R848, TL8-506, LPS, Pam2CSK4, and ODN 2336.

Embodiment A93. The composition of any one of embodiments A89-A92, further comprising an antigen.

Embodiment A94. The composition of any one of embodiments A89-A93, further comprising dendritic cells.

Embodiment A95. A pharmaceutical formulation comprising the composition of any one of embodiments A89-A94 and a pharmaceutically acceptable excipient.

Embodiment A96. A pharmaceutical formulation comprising an isolated ether lipid (ETL) of Formula (I):

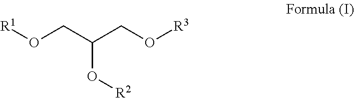

wherein:
R¹ is H or

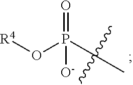

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
a pharmaceutically acceptable excipient.

Embodiment A97. The pharmaceutical formulation of embodiment A95 or embodiment A96, wherein the alkyl chain is a C₂₂ n-alkyl chain.

Embodiment A98. The pharmaceutical formulation of embodiment A97, wherein the ETL comprises one or both of DGPC and DGP, or a pharmaceutically acceptable salt thereof.

Embodiment A99. A composition for hyperactivation of human dendritic cells, comprising an isolated ether lipid (ETL) of Formula (I):

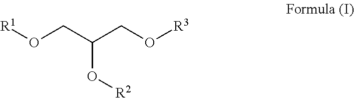

wherein:
R¹ is H or

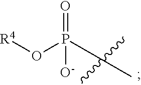

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
a pathogen recognition receptor (PRR) agonist, wherein the alkyl chain is a C₂₂ n-alkyl chain, and wherein the composition is effective for achieving a higher level of dendritic cell hyperactivation than a comparator composition comprising PGPC in place of the ETL.

Embodiment A100. The composition of embodiment A99, wherein R³ is C₂₂ n-alkyl.

Embodiment A101. The composition of embodiment A99 or embodiment A100, wherein the higher level of dendritic cell hyperactivation comprises induction of IL-1beta secretion from the human dendritic cells in vitro at a level that is at least 2, 3 or 4 fold higher when contacted with the composition comprising the ETL and the PRR agonist than when contacted with the comparator composition comprising the PGPC and the PRR agonist, wherein the PRR agonist is LPS.

Embodiment A102. The composition of embodiment A101, wherein the concentration of the ETL and the concentration of the PGPC are the same concentration in a range of from about 10 μM to about 80 μM, and the LPS is present at a concentration of 1 μg/ml in both the composition and the comparator composition.

Embodiment A103. The composition of embodiment A101, wherein the higher level of dendritic cell hyperactivation comprises a lipid activity index for IL-1beta secretion from the human dendritic cells for the composition comprising the ETL and the PRR agonist that is at least 4, 5 or 6 fold higher in activity units than that of the comparator composition comprising the PGPC and the PRR agonist.

Embodiment A104. The composition, formulation, method or use of any one of embodiments A24-A52, wherein the individual is a human subject.

Embodiment A105. The composition, formulation, method or use of any one of embodiments A24-A52, wherein the individual is a canine subject.

Embodiment A106. The composition, formulation, method or use of any one of embodiments A68-A103, wherein the mammalian subject is a human patient.

Embodiment A107. The composition, formulation, method or use of any one of embodiments A68-A103, wherein the mammalian subject is a non-human patient.

Embodiment A108. The composition, formulation, method or use of any one of embodiments A68-A103, wherein the mammalian subject is a canine patient.

Embodiment A109. The composition, formulation, method or use of any one of embodiments A1-A104 or A106, wherein the dendritic cells are human dendritic cells.

Embodiment A110. The composition, formulation, method or use of any one of embodiments A1-A53, A55-A98 or A108, wherein the dendritic cells are canine dendritic cells.

Embodiment A111. The composition, method or use of embodiment A109 or embodiment A110, wherein the dendritic cells are present in a composition comprising peripheral blood mononuclear cells (PBMCs).

Embodiment A112. The composition, method or use of any one of embodiments A42-A54 or embodiments A109-A110, wherein the hyperactivated dendritic cells secrete one or both of IFNγ and TNFα.

Embodiment A113. The composition, formulation, method or use of any one of embodiments A1-A112, comprising a surfactant.

Embodiment A114. The composition, formulation, method or use of embodiment A113, wherein the surfactant comprises a non-ionic surfactant.

Embodiment A115. The composition, formulation, method or use of embodiment A114, wherein the non-ionic surfactant comprises an ethylene oxide-propylene oxide copolymer.

Embodiment A116. The composition, formulation, method or use of embodiment A114, wherein the non-ionic surfactant comprises one or more of Poloxamer 407, Poloxamer 188, and P123.

Embodiment A117. The composition, formulation, method or use of embodiment A114, wherein the non-ionic surfactant comprises Poloxamer 407.

Embodiment A118. The composition, formulation, method or use of any one of embodiments A114-A117, wherein i) the ETL is dissolved in an alcohol to form an ETL alcohol solution; ii) the ETL alcohol solution is mixed with the non-ionic surfactant to form a mixture; and iii) the alcohol is evaporated from the mixture to form particles comprising the ETL and the non-ionic surfactant.

Embodiment A119. The composition, formulation, method or use of any one of embodiments A104-A118, wherein the non-ionic surfactant is present in an amount of about 2.5% to 25% (w/w), optionally about 5% to 20% (w/w), optionally about 15% (w/w).

Embodiment A120. The composition, formulation, method or use of any one of embodiments A104-A119, wherein the ETL and non-ionic surfactant are present in particles with a diameter of about 1000 to 15,000 nanometers, optionally with a diameter of about 5000 nanometers.

Embodiment A121. An isolated ether lipid (ETL) of Formula (I):

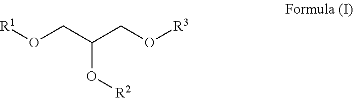

wherein R is H or

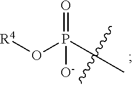

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
wherein R is H or (CH₃)₃N⁺—(CH₂)₂—; and
each R⁵ is independently C₁-C₄ alkyl;
or a protonated or deprotonated form thereof; or a salt thereof.

Embodiment A122. The isolated ether lipid of embodiment A121, wherein the isolated ether lipid is a compound of Formula (II):

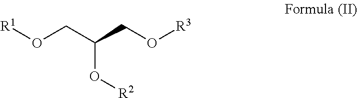

wherein R¹ is H or

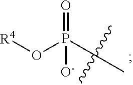

R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
wherein R⁴ is H or (CH₃)₃N⁻—(CH₂)₂—; and
each R⁵ is independently C₁-C₄ alkyl;
or a protonated or deprotonated form thereof; or a salt thereof.

Embodiment A123. The isolated ether lipid of embodiment A121, wherein the isolated ether lipid is a compound of Formula (III):

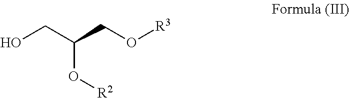

wherein R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl; and
each R⁵ is independently C₁-C₄ alkyl;
or a salt thereof.

Embodiment A124. The isolated ether lipid of embodiment A121, wherein the isolated ether lipid is an isolated ether phospholipid (ETPL) compound of Formula (IV):

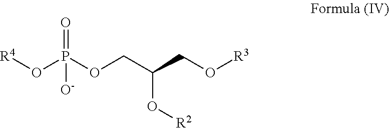

wherein R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
R⁴ is H or (CH₃)₃N⁺—(CH₂)₂—; and
each R⁵ is independently C₁-C₄ alkyl;
or a protonated or deprotonated form thereof; or a salt thereof.

Embodiment A125. The isolated ether lipid of embodiment A121, wherein the isolated ether lipid is an isolated ether phospholipid (ETPL) compound of Formula (IV-A):

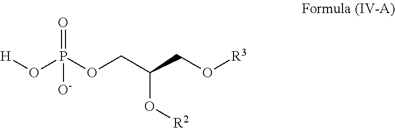

wherein R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl; and
each R⁵ is independently C₁-C₄ alkyl;
or a protonated or deprotonated form thereof; or a salt thereof.

Embodiment A126. The isolated ether lipid of embodiment A121, wherein the isolated ether lipid is an isolated ether phospholipid (ETPL) compound of Formula (IV-B):

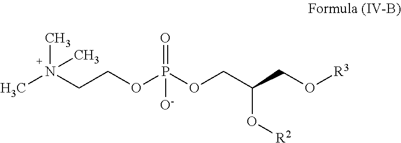

wherein R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl; and
each R⁵ is independently C₁-C₄ alkyl;
or a protonated form thereof; or a salt thereof.

Embodiment A127. The isolated ether lipid of embodiment A121, wherein the isolated ether lipid is an isolated ether phospholipid (ETPL) compound of Formula (IV-C):

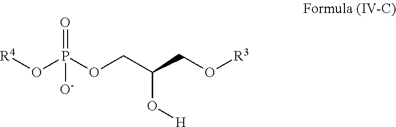

wherein R³ is C₁₃-C₂₄ n-alkyl; and
R⁴ is H or (CH₃)₃N⁺—(CH₂)₂—;
or a protonated or deprotonated form thereof; or a salt thereof

Embodiment A128. A compound of formula 2:

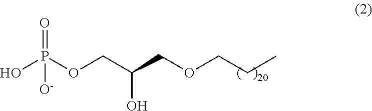

or a protonated form thereof; or a pharmaceutically acceptable salt thereof.

Embodiment A129. The compound of embodiment A128, wherein said compound is isolated.

Embodiment A130. An isolated compound 1 of formula 1:

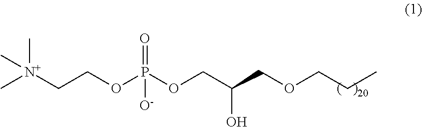

or a protonated form thereof; or a pharmaceutically acceptable salt thereof.

Embodiment A131. A compound of Formula (III-A-1):

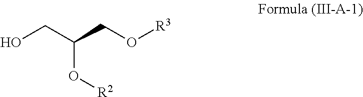

wherein:
R² is —(C═O)—NH₂, —(C═O)—NH(R⁵), or —(C═O)—N(R⁵)₂;
R³ is C₂₁-C₂₄ n-alkyl; and
each R⁵ is independently C₁-C₄ alkyl;
or a pharmaceutically acceptable salt thereof.

Embodiment A132. The compound of embodiment A131, wherein R² is —(C═O)—NH₂.

Embodiment A133. The compound of embodiment A131, wherein R² is —(C═O)—NH—CH.

Embodiment A134. The compound of embodiment A131, wherein R² is —(C═O)—N(CH₃)₂.

Embodiment A135. The compound of any one of embodiments A131-A134, wherein R³ is C₂₂ n-alkyl.

Embodiment A136. A compound 7 of formula 7:

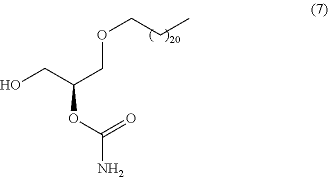

or a pharmaceutically acceptable salt thereof.

Embodiment A137. The compound of embodiment A136, wherein said compound is isolated.

Embodiment A138. A compound 8 of formula 8:

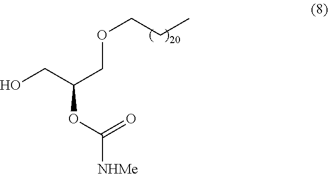

or a pharmaceutically acceptable salt thereof.

Embodiment A139. The compound of embodiment A138, wherein said compound is isolated.

Embodiment A140. A composition comprising the compound of any one of embodiments A121-A139 and a pharmaceutically acceptable excipient.

Embodiment A141. The composition of embodiment A140, wherein the pharmaceutically acceptable excipient comprises phosphate-buffered saline.

Embodiment A142. The composition of embodiment A140, wherein the pharmaceutically acceptable excipient comprises an aqueous solution of poloxamer 407.

Embodiment A143. The composition of embodiment A140, wherein the pharmaceutically acceptable excipient comprises phosphate-buffered saline and poloxamer 407.

Embodiment A144. The composition of any one of embodiments A140-A143, wherein said composition is sterile.

Embodiment A145. An article of manufacture comprising a container enclosing a liquid formulation of the compound of any one of embodiments A121-A139 and a pharmaceutically acceptable excipient.

Embodiment A146. The article of manufacture of embodiment A145, wherein the container is a syringe.

Embodiment A147. The article of manufacture of embodiment A146, wherein the syringe is further contained within an injection device.

Embodiment A148. The article of manufacture of embodiment A147, wherein the injection device is an auto-injector.

Embodiment A149. A composition comprising an isolated ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Compound 1, Compound 2, Compound 3, Compound 4, Compound 5, Compound 6, Compound 7, Compound 8, Compound 9, Compound 10, Compound 11, Compound 12, or Compound 13 as disclosed herein; or a protonated or deprotonated form thereof where possible, or a pharmaceutically acceptable salt thereof; and at least one further lipid, wherein the at least one further lipid is selected from the group consisting of an ionizable lipid, a cationic lipid, a further phospholipid, a pegylated lipid, a structural lipid, and mixtures thereof.

Embodiment A150. The composition of embodiment A149, wherein the ETL or ETPL and the at least one further lipid are part of a lipid nanoparticle (LNP).

Embodiment A151. The composition of embodiment A149 or embodiment A150, further comprising an antigen.

Embodiment A152. The composition of any one of embodiments A149-A151, further comprising dendritic cells.

Embodiment A153. The composition of any one of embodiments A149-A152, further comprising a TLR agonist.

Embodiment A154. The composition of any one of embodiments A149-A152, further comprising a TLR7/8 agonist.

Embodiment A155. The composition of any one of embodiments A149-A154, wherein the ether lipid (ETL) or ether phospholipid (ETPL) is a compound of Formula (II), or a protonated or deprotonated form thereof where possible, or a pharmaceutically acceptable salt thereof.

Embodiment A156. The composition of any one of embodiments A149-A154, wherein the ether lipid (ETL) or ether phospholipid (ETPL) is a compound of Formula (III), or a protonated or deprotonated form thereof where possible, or a pharmaceutically acceptable salt thereof.

Embodiment A157. The composition of any one of embodiments A149-A154, wherein the ether lipid (ETL) or ether phospholipid (ETPL) is a compound of Formula (III-A), or a protonated or deprotonated form thereof where possible, or a pharmaceutically acceptable salt thereof.

Embodiment A158. The composition of any one of embodiments A149-A154, wherein the ether lipid (ETL) or ether phospholipid (ETPL) is a compound of Formula (III-A-1), or a protonated or deprotonated form thereof where possible, or a pharmaceutically acceptable salt thereof.

Embodiment A159. The composition of any one of embodiments A149-A154, wherein the ether lipid (ETL) or ether phospholipid (ETPL) is a compound of Formula (III-A-2), or a protonated or deprotonated form thereof where possible, or a pharmaceutically acceptable salt thereof.

Embodiment A160. The composition of any one of embodiments A149-A154, wherein the ether lipid (ETL) or ether phospholipid (ETPL) is a compound of Formula (III-B), or a protonated or deprotonated form thereof where possible, or a pharmaceutically acceptable salt thereof.

Embodiment A161. The composition of any one of embodiments A149-A154, wherein the ether lipid (ETL) or ether phospholipid (ETPL) is a compound of Formula (III-B-1), or a protonated or deprotonated form thereof where possible, or a pharmaceutically acceptable salt thereof.

Embodiment A162. The composition of any one of embodiments A149-A154, wherein the ether lipid (ETL) or ether phospholipid (ETPL) is a compound of Formula (III-B-2), or a protonated or deprotonated form thereof where possible, or a pharmaceutically acceptable salt thereof.

Embodiment A163. The composition of any one of embodiments A149-A154, wherein the ether lipid (ETL) or ether phospholipid (ETPL) is a compound of Formula (IV), or a protonated or deprotonated form thereof where possible, or a pharmaceutically acceptable salt thereof.

Embodiment A164. The composition of any one of embodiments A149-A154, wherein the ether lipid (ETL) or ether phospholipid (ETPL) is a compound of Formula (IV-A), or a protonated or deprotonated form thereof where possible, or a pharmaceutically acceptable salt thereof.

Embodiment A165. The composition of any one of embodiments A149-A154, wherein the ether lipid (ETL) or ether phospholipid (ETPL) is a compound of Formula (IV-A-1), or a protonated or deprotonated form thereof where possible, or a pharmaceutically acceptable salt thereof.

Embodiment A166. The composition of any one of embodiments A149-A154, wherein the ether lipid (ETL) or ether phospholipid (ETPL) is a compound of Formula (IV-A-2), or a protonated or deprotonated form thereof where possible, or a pharmaceutically acceptable salt thereof.

Embodiment A167. The composition of any one of embodiments A149-A154, wherein the ether lipid (ETL) or ether phospholipid (ETPL) is a compound of Formula (IV-B), or a protonated or deprotonated form thereof where possible, or a pharmaceutically acceptable salt thereof.

Embodiment A168. The composition of any one of embodiments A149-A154, wherein the ether lipid (ETL) or ether phospholipid (ETPL) is a compound of Formula (IV-B-1), or a protonated or deprotonated form thereof where possible, or a pharmaceutically acceptable salt thereof.

Embodiment A169. The composition of any one of embodiments A149-A154, wherein the ether lipid (ETL) or ether phospholipid (ETPL) is a compound of Formula (IV-B-2), or a protonated or deprotonated form thereof where possible, or a pharmaceutically acceptable salt thereof.

Embodiment A170. The composition of any one of embodiments A149-A154, wherein the ether lipid (ETL) or ether phospholipid (ETPL) is a compound of Formula (IV-C), or a protonated or deprotonated form thereof where possible, or a pharmaceutically acceptable salt thereof.

Embodiment A171. The composition of any one of embodiments A149-A154, wherein the ether lipid (ETL) or ether phospholipid (ETPL) is a compound of Formula (IV-D), or a protonated or deprotonated form thereof where possible, or a pharmaceutically acceptable salt thereof.

Embodiment A172. The composition of any one of embodiments A149-A154, wherein the ether lipid (ETL) or ether phospholipid (ETPL) is a compound of Formula (IV-E), or a protonated or deprotonated form thereof where possible, or a pharmaceutically acceptable salt thereof.

Embodiment A173. The composition of any one of embodiments A149-A154, wherein the ether lipid (ETL) or ether phospholipid (ETPL) is Compound 1, or a protonated or deprotonated form thereof where possible, or a pharmaceutically acceptable salt thereof.

Embodiment A174. The composition of any one of embodiments A149-A154, wherein the ether lipid (ETL) or ether phospholipid (ETPL) is Compound 2, or a protonated or deprotonated form thereof where possible, or a pharmaceutically acceptable salt thereof.

Embodiment A175. The composition of any one of embodiments A149-A154, wherein the ether lipid (ETL) or ether phospholipid (ETPL) is Compound 3, or a protonated or deprotonated form thereof where possible, or a pharmaceutically acceptable salt thereof.

Embodiment A176. The composition of any one of embodiments A149-A154, wherein the ether lipid (ETL) or ether phospholipid (ETPL) is Compound 4, or a protonated or deprotonated form thereof where possible, or a pharmaceutically acceptable salt thereof.

Embodiment A177. The composition of any one of embodiments A149-A154, wherein the ether lipid (ETL) or ether phospholipid (ETPL) is Compound 5, or a protonated or deprotonated form thereof where possible, or a pharmaceutically acceptable salt thereof.

Embodiment A178. The composition of any one of embodiments A149-A154, wherein the ether lipid (ETL) or ether phospholipid (ETPL) is Compound 6, or a protonated or deprotonated form thereof where possible, or a pharmaceutically acceptable salt thereof.

Embodiment A179. The composition of any one of embodiments A149-A154, wherein the ether lipid (ETL) or ether phospholipid (ETPL) is Compound 7, or a protonated or deprotonated form thereof where possible, or a pharmaceutically acceptable salt thereof.

Embodiment A180. The composition of any one of embodiments A149-A154, wherein the ether lipid (ETL) or ether phospholipid (ETPL) is Compound 8, or a protonated or deprotonated form thereof where possible, or a pharmaceutically acceptable salt thereof.

Embodiment A181. The composition of any one of embodiments A149-A154, wherein the ether lipid (ETL) or ether phospholipid (ETPL) is Compound 9, or a protonated or deprotonated form thereof where possible, or a pharmaceutically acceptable salt thereof.

Embodiment A182. The composition of any one of embodiments A149-A154, wherein the ether lipid (ETL) or ether phospholipid (ETPL) is Compound 10, or a protonated or deprotonated form thereof where possible, or a pharmaceutically acceptable salt thereof.

Embodiment A183. The composition of any one of embodiments A149-A154, wherein the ether lipid (ETL) or ether phospholipid (ETPL) is Compound 11, or a protonated or deprotonated form thereof where possible, or a pharmaceutically acceptable salt thereof.

Embodiment A184. The composition of any one of embodiments A149-A154, wherein the ether lipid (ETL) or ether phospholipid (ETPL) is Compound 12, or a protonated or deprotonated form thereof where possible, or a pharmaceutically acceptable salt thereof.

Embodiment A185. The composition of any one of embodiments A149-A154, wherein the ether lipid (ETL) or ether phospholipid (ETPL) is Compound 13, or a protonated or deprotonated form thereof where possible, or a pharmaceutically acceptable salt thereof.

### Synthetic Schemes

The following synthetic schemes describe general synthetic procedures, which can be used as described or further modified or combined to prepare the compounds disclosed herein. Additionally, the chemical reactions in the Synthetic Examples provided herein can be readily adapted to prepare the compounds disclosed herein. For example, the synthesis of non-exemplified compounds disclosed herein can be successfully performed by modifications known to the skilled artisan, such as by using alternative protecting groups, by utilizing other suitable reagents known in the art other than those described, or by making routine modifications of reaction conditions. Other reactions disclosed herein or known in the art will be recognized as being applicable for preparing other compounds disclosed herein.

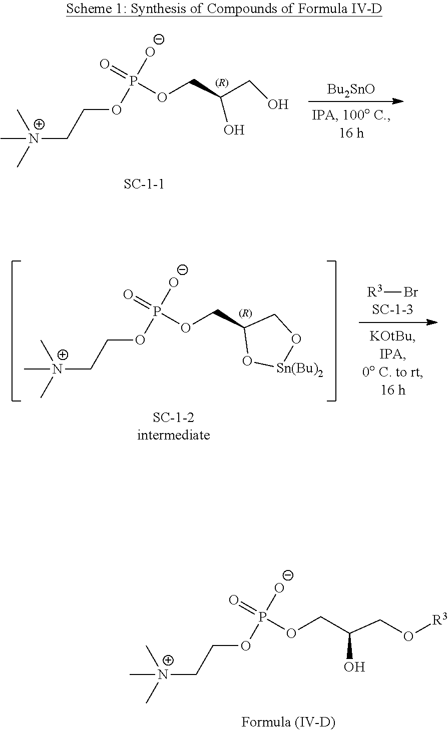

Compounds of Formula (IV-D) can be readily prepared according to Scheme 1. Starting material SC-1-1, (R)-2,3-dihydroxypropyl (2-(trimethylammonio)ethyl) phosphate, is commercially available (CAS No. 28319-77-9; suppliers include Ambeed, Arlington Heights, Illinois, United States). Bu₂SnO (2.89 g, 0.0116 mol) can be used to form intermediate SC-1-2, followed by reaction with R³—Br, where R³ is C₁₃-C₂₄ n-alkyl, to yield compounds of Formula (IV-D).

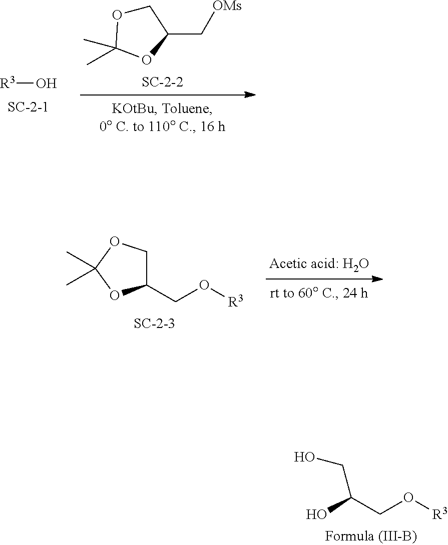

Compounds of Formula (III-B) can be prepared according to Scheme 2. Alcohol SC-2-1, where R³ is C₁₃-C₂₄ n-alkyl, is reacted with (S)-(2,2-dimethyl-1,3-dioxolan-4-yl)methyl 4-methylbenzenesulfonate SC-2-2 (commercially available from Sigma-Aldrich, Saint Louis, Missouri, United States) to form intermediate SC-2-3. Opening the dioxolane ring with acetic acid yields compounds of Formula (III-B).

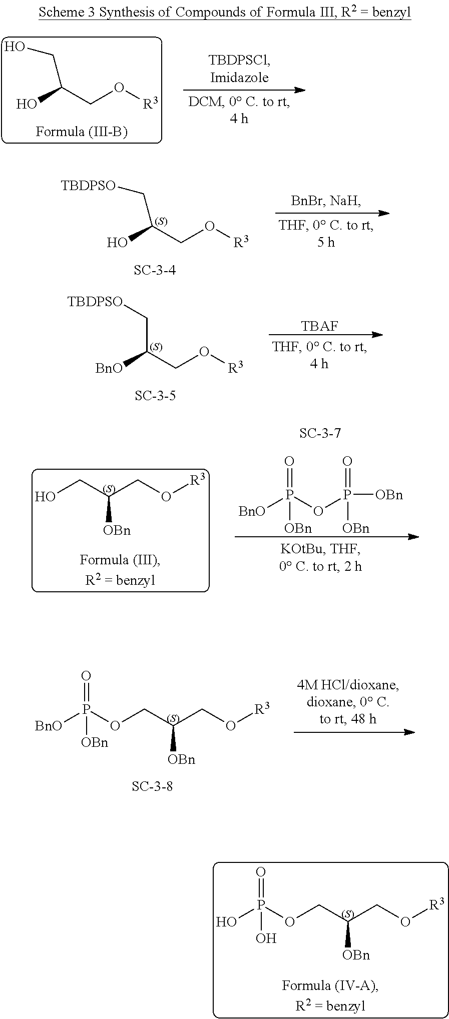

Scheme 3 outlines synthesis of compounds of Formula (III), where R² is benzyl, and of Formula (IV-A), where R²=benzyl. Starting from the compounds of Formula (III-B) prepared in Scheme 2, the terminal hydroxy group is protected, for example with a TBDPSCl group. Benzyl bromide is then added to the unprotected 2-hydroxy group. The terminal hydroxy group is deprotected, yielding compounds of Formula (III), where R² is benzyl and R³ is C₁₃-C₂₄ n-alkyl.

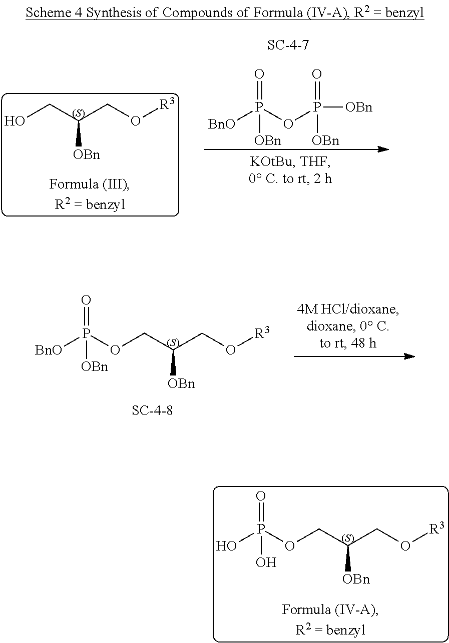

As shown in Scheme 4, a phosphate group can be added to compounds of Formula (III), where R² is benzyl, in order to prepare compounds of Formula (IV-A), where R² is benzyl, by reacting the compounds of Formula (III) where R² is benzyl with tetrabenzyl pyrophosphate (tetrabenzyl diphosphate), and then removing the benzyl groups from the phosphate to provide the compounds of Formula (IV-A), where R² is benzyl and R³ is C₁₃-C₂₄ n-alkyl.

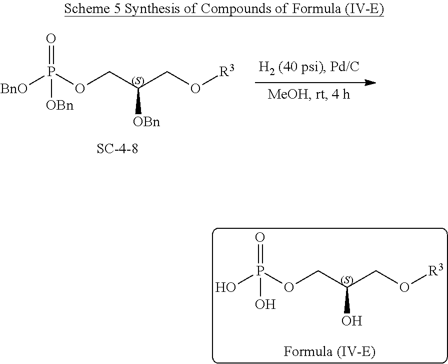

Compounds of Formula (IV-E) can be prepared starting from the SC-4-8 intermediate in Scheme 4, and removing all of the benzyl groups, for example, using catalytic hydrogenation as shown in Scheme 5, where R³ is C₁₃-C₂₄ n-alkyl.

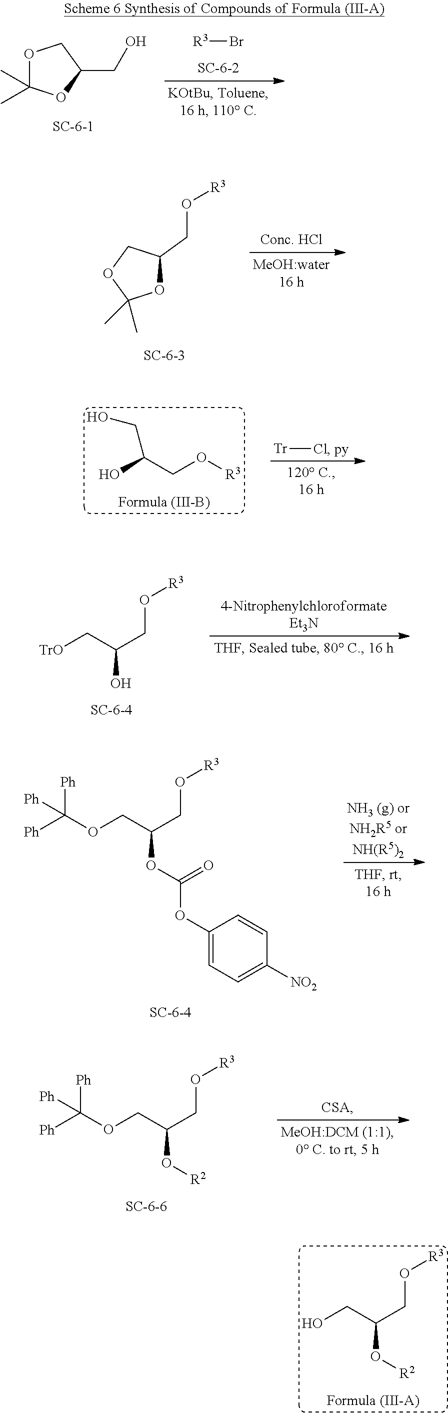

Compounds of Formula (III-A) can be prepared as shown in Scheme 6, proceeding through compounds of Formula (III-B) as intermediates. In Scheme 6, R² is —(C═O)—NH₂, —(C═O)—NH(R⁵), or —(C═O)—N(R⁵)₂; each R⁵ is independently C₁-C₄ alkyl; and R³ is C₁₃-C₂₄ n-alkyl.

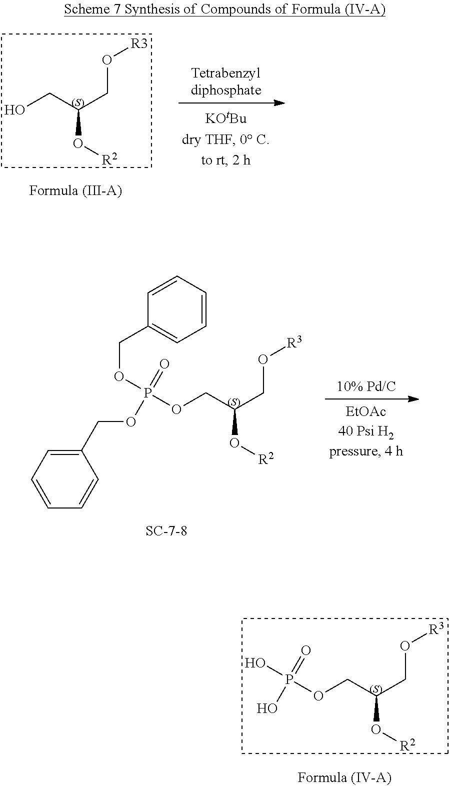

Compounds of Formula (IV-A) can be prepared as shown in Scheme 7, starting from compounds of Formula (III-A), where R² is —(C═O)—NH₂, —(C═O)—NH(R⁵), or —(C═O)—N(R⁵)₂; each R⁵ is independently C₁-C₄ alkyl; and R³ is C₁₃-C₂₄ n-alkyl.

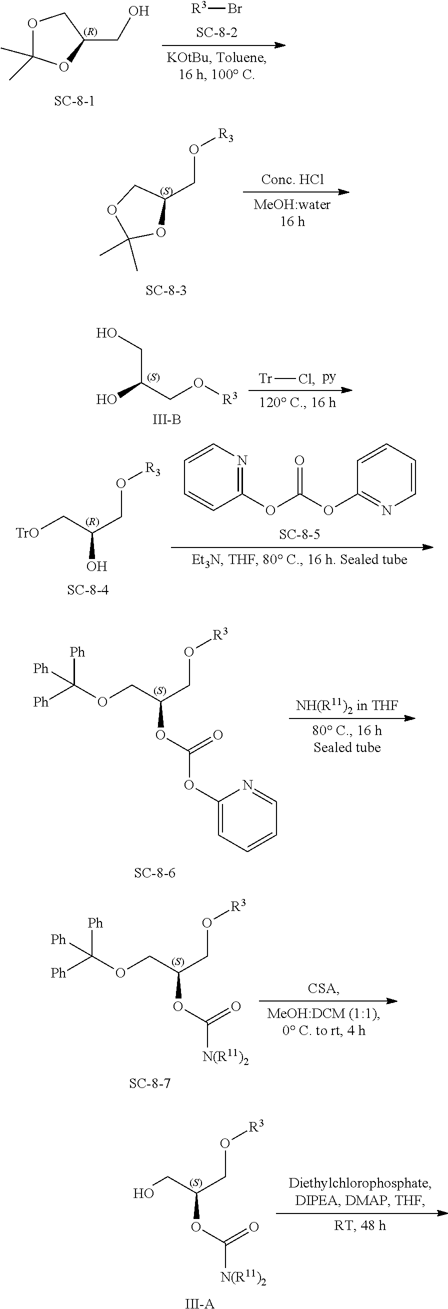

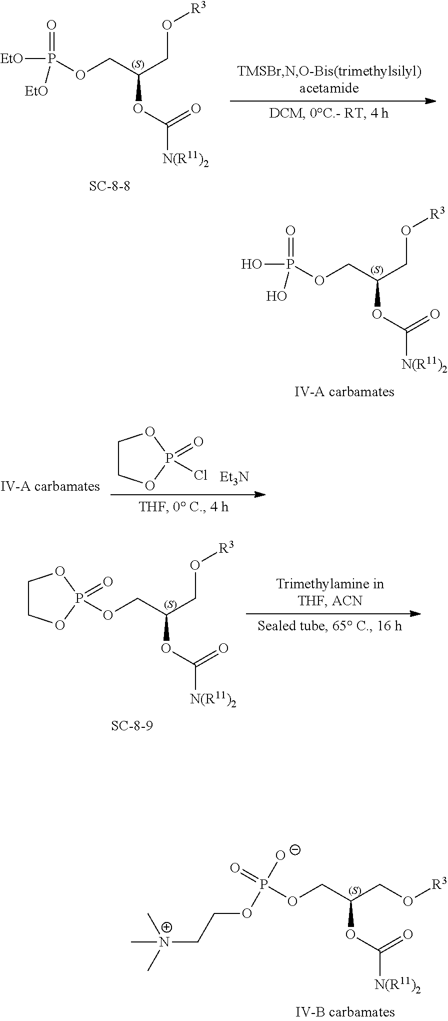

Compounds of Formula (III-A), Formula (IV-A), and Formula (IV B) can be prepared as shown in Scheme 8, where R³ is C₁₃-C₂₄ n-alkyl and each R¹¹ is independently selected from H or methyl. Synthesis of carbamates is shown. To synthesize compounds of Formula (IV-B) with an alkyl or benzyl group at the 2-hydroxy position of the glycerol moiety, an alkylating or benzylating agent such as an alkyl bromide or benzyl bromide is used in place of reagent SC-8-5, di(pyridin-2-yl) carbonate. To synthesize compounds of Formula (IV-C) with a free hydroxy group at the 2-hydroxy position of the glycerol moiety, a protecting group can be placed on that hydroxy group and removed at the end of the synthesis (such as a benzyl group, added with benzyl bromide in place of reagent SC-8-5, and removed with catalytic hydrogenation).

### EXAMPLES

Abbreviations: BM (bone marrow); BMDC (bone marrow-derived dendritic cell); CDS (cytosolic DNA sensor); CLR (C-type lectin receptor); DAMP (damage-associated molecular pattern); DC (dendritic cell); DGPC (1-docosyl-sn-glycerol-3-phosphocholine); DGP (1-docosyl-sn-glycerol-3-phosphate); dLN (draining lymph node); HOdiA-PC (1-Palmitoyl-2-(5-hydroxy-8-oxo-6-octenedioyl)-sn-glycero-3-phosphatidylcholine); HOOA-PC (1-palmitoyl-2-(5-hydroxy-8-oxooct-6-enoyl)-sn-glycero-3-phosphocholine); IFNγ (interferon-gamma); IL-1b/IL1-beta/IL-1β (Interleukin-1beta); KOdiA-PC (1-(Palmitoyl)-2-(5-keto-6-octene-dioyl) phosphatidylcholine); KOOA-PC (1-palmitoyl-(5-keto-8-oxo-6-octenoyl)-sn-glycero-3-phosphocholine); KP407 (poloxamer 407); LPC/Lyso PC (lysophosphatidylcholine); Lyso PC(22:0) (1-behenoyl-2-hydroxy-sn-glycero-3-phosphocholine); LPS (lipopolysaccharide); MFI (mean fluorescence intensity); moDC (monocyte-derived dendritic cell); MPLA (monophosphoryl lipid A); NLR (NOD-like receptor); oxPAPC (oxidized I-palmitoyl-2-arachidonyl-sn-glycero-3-phosphorylcholine); PAMP (pathogen-associated molecular pattern); PBMCs (peripheral blood mononuclear cells); PGPC (1-palmitoyl-2-glutaryl-sn-glycero-3-phosphocholine); POVPC (1-palmitoyl-2-(5′-oxo-valeroyl)-sn-glycero-3-phosphocholine); PRR (pathogen recognition receptor); RLR (RIG-I-like receptor); R848 (resiquimod); STING (stimulator of IFN genes); TNFα (tumor necrosis factor-alpha); TLR (toll-like receptor); and WTL (whole tumor lysate).

### SYNTHETIC EXAMPLES

### Example S-1: Synthesis of Compound 1

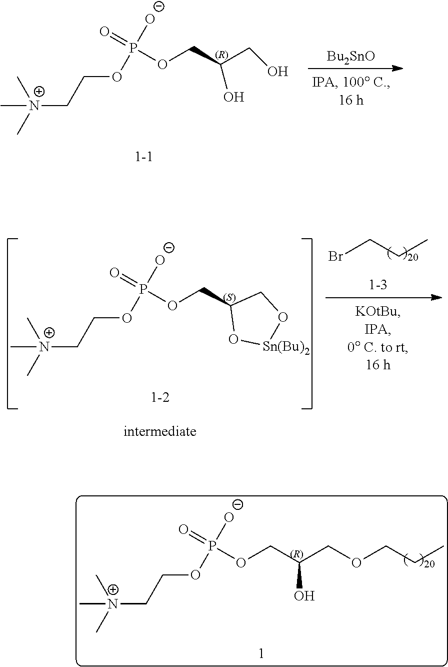

To a stirred solution of (R)-2,3-dihydroxypropyl (2-(trimethylammonio)ethyl) phosphate 1-1 (2 g, 0.0077 mol) in IPA (150 mL), was added Bu₂SnO (2.89 g, 0.0116 mol) and reaction mixture was heated at 100° C. for 16 h. Progress of reaction was monitored by TLC. The crude reaction mixture was evaporated on rota vapour to obtain crude material. The obtained crude material was used for next step without analysis.

To a stirred solution of crude intermediate 1-2 (2 g, 0.0040 mol) in IPA (150 mL), was added KOtBu (0.672 g, 0.006 mol) and 1-bromo docosane (1-3) (1.86 g, 0.0048 mol) at 0° C. and reaction mixture was stirred at RT for 16 h. Progress of reaction was monitored by TLC. The crude reaction mixture was evaporated on rota vapour to obtain crude material. The obtained crude material was purified by column chromatography (silica gel basified with NH₄OH) using 30% MeOH in DCM and 10% NH₄OH as an eluent. The impure material was re-purified by Combiflash chromatography (ELSD); 12 g column, using 40% MeOH in DCM and 10% NH₄OH as an eluent to afford Compound 1 (75 mg, 0.13 mmol, 3.3%) as a white solid. HRMS: 566.3606; HPLC-ELSD: 97.13%; ¹H NMR (CDCl₃) 400 MHz) δ ppm 4.28-4.27 (m, 1H), 3.92-3.85 (m, 3H), 3.64-3.61 (m, 3H), 3.46-3.43 (m, 3H), 3.24 (bs, 9H), 1.59-1.52 (m, 2H), 1.42-1.24, (m, 40H), 0.89 (t, J=6.4 Hz, 3H).

### Example S-2: Synthesis of Compound 9, Compound 2, and Compound 10

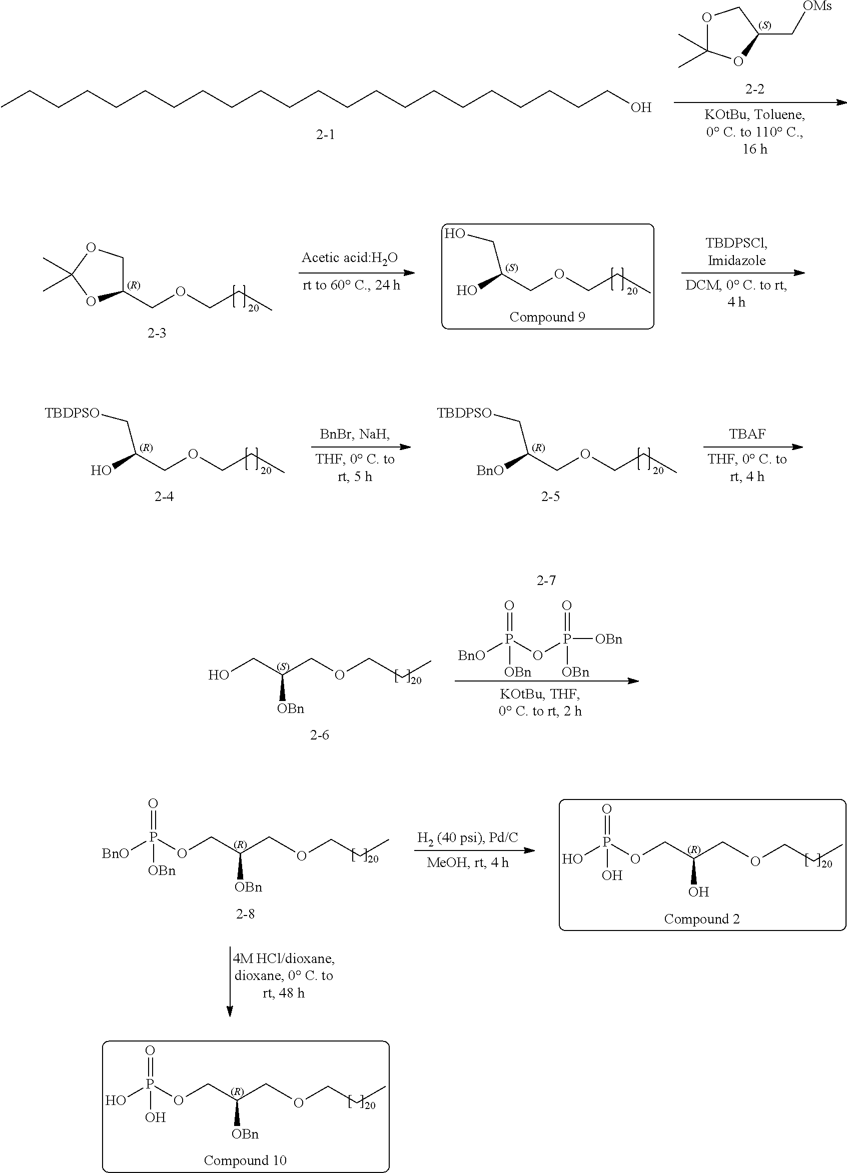

### Synthesis of (R)-4-((docosyloxy)methyl)-2,2-dimethyl-1,3-dioxolane (2-3)

To a stirred solution of 1-docosanol 2-1 (10 g, 0.0306 mol) and (S)-(2,2-dimethyl-1,3-dioxolan-4-yl)methyl 4-methylbenzenesulfonate 2-2 (7.77 g, 0.0367 mol) in toluene (150 mL), was added KOtBu (6.86 g, 0.0612 mol) at 0° C. and heated to 110° C. for 16 h. Progress of the reaction was monitored by TLC. After completion of the reaction, the reaction mixture was concentrated under vacuum to obtain crude material. The obtained crude material was purified by MPLC Flash column chromatography using 10% EtOAc in hexane as eluent to afford (R)-4-((docosyloxy)methyl)-2,2-dimethyl-1,3-dioxolane (2-3, 15 g, 0.0340 mmol, 38%) as an off white solid. HPLC (ELSD): 97.39%, ¹H NMR (CDCl₃, 400 MHz): δ ppm 4.29-4.23 (m, 1H), 4.07-4.04 (m, 1H), 3.74-3.71 (m, 1H), 3.53-3.39 (m, 3H), 1.57-1.51 (m, 1H), 1.42 (s, 3H), 1.37 (m, 3H), 1.31-1.14 (m, 40H), 0.89-0.82 (m, 3H).

### Synthesis of Compound 9

A solution of (R)-4-((docosyloxy)methyl)-2,2-dimethyl-1,3-dioxolan 2-3 (15 g, 0.034 mol) in acetic acid:H₂O (10:1 by volume) was heated to 60° C. and the reaction mixture was stirred for 16 h. Progress of the reaction was monitored by TLC analysis. After completion of the reaction, the reaction mixture was concentrated under vacuum to obtain crude material. The obtained crude material was washed with n-hexane and dried under vacuum to afford (S)-3-(docosyloxy)propane-1,2-diol (9.00 g, 66%) as an off-white solid. HPLC (ELSD): 86.61%, ¹H NMR (CDCl3, 400 MHz): δ ppm 3.86 (brs, 1H), 3.71-3.67 (m, 2H), 3.53-3.44 (m, 4H), 2.62 (brs, 1H), 2.19 (brs, 1H), 1.61-1.54 (m, 2H), 1.31-1.14 (m, 38H), 0.89-0.82 (m, 3H).

The crude material (1.00 g, 2.49 mmol) was purified by dissolving in 20% EtOAc; Hexane (200 mL), stirred for 30 min. After 30 min, solid was filtered and dried under vacuum to afford Compound 9 (600 mg, 1.49 mmol, 60%) as an off-white solid. HPLC (ELSD): 98.33%, ¹H NMR (CDCl₃. 400 MHz): δ ppm 3.86-3.85 (m, 1H), 3.70-3.66 (m, 2H), 3.53-3.44 (m, 4H), 2.63-2.62 (m, 1H), 2.19 (brs, 1H), 1.62-1.53 (m, 2H), 1.31-1.14 (m, 38H), 0.89-0.82 (m, 3H).

### Synthesis of (R)-1-((tert-butyldiphenylsilyl)oxy)-3-(docosyloxy)propan-2-ol (2-4)

To a stirred solution of crude (S)-3-(docosyloxy)propane-1,2-diol (Compound 9) (6 g, 0.0149 mol) in DCM (150 mL), was added imidazole (2.54 g 0.0374 mol), TBDPSCl (4.7 mL, 0.0179 mol) at 0° C. and the reaction mixture was stirred at RT for 4 h. Progress of the reaction was monitored by TLC analysis. After completion of the reaction, the reaction mixture was diluted with water and extracted the product into with DCM (2×200 mL). The combined organic layers were dried over Na₂SO₄ and concentrated under vacuum to obtain crude material. The obtained crude material was purified by MPLC Flash column chromatography; 12 g Clariscep C-series, using 10% EtOAc in hexane as eluent to afford (R)-1-((tert-butyldiphenylsilyl)oxy)-3-(docosyloxy)propan-2-ol (2-4) (5.00 g, 52%) as an off-white solid. ¹H NMR (CDCl₃, 400 MHz): δ ppm 7.67-7.65 (m, 5H), 7.42-7.36 (m, 5H), 3.89-3.71 (m, 1H), 3.70-3.53 (m, 2H), 3.52-3.41 (m, 4H), 1.61 (brs, 1H), 1.56-1.53 (m, 2H), 1.31-1.14 (m, 38H), 1.22 (s, 9H), 0.89-0.86 (m, 3H).

### Synthesis of (R)-(2-(benzyloxy)-3-(docosyloxy)propoxy)(tert-butyl)diphenylsilane (2-5)

To a stirred solution of (R)-1-((tert-butyldiphenylsilyl)oxy)-3-(docosyloxy)propan-2-ol (2-4) (3 g, 0.0046 mol) in THF (50 mL), was added NaH (0.45 g, 0.0938 mol) at 0° C. and stirred for 20 min. After 20 min, BnBr (0.7 mL, 0.0056 mol) was added at RT and the reaction mixture was stirred at RT for 4 h. Progress of the reaction was monitored by TLC analysis. After completion of the reaction, the reaction mixture was quenched with ice and the product was extracted into EtOAc (100 mL). The combined organic layers were dried over Na₂SO₄ and concentrated under vacuum to obtain crude material. The obtained crude material was purified by MPLC Flash column chromatography using 10% EtOAc in hexane as eluent to afford (R)-(2-(benzyloxy)-3-(docosyloxy)propoxy)(tert-butyl)diphenylsilane (2-5) (2.2 g, 65%) as a pale yellow gummy. ¹H NMR (CDCl₃, 400 MHz): δ ppm 7.61-7.58 (m, 5H), 7.34-7.26 (m, 10H), 4.60 (s, 2H), 3.69-3.68 (m, 2H) 3.63-3.33 (m, 5H), 1.51-1.45 (m, 2H), 1.31-1.14 (m, 38H), 1.01 (s, 9H), 0.82-0.77 (m, 3H).

### Synthesis of (S)-2-(benzyloxy)-3-(docosyloxy)propan-1-ol (2-6)

To a stirred solution of (R)-(2-(benzyloxy)-3-(docosyloxy)propoxy)(tert-butyl)diphenylsilane (2-5, 2.9 g, 0.003 mol) in THF (30 mL), was added TBAF (8 mL, 0.007 mol) at 0° C. and the reaction mixture was stirred at RT for 4 h. Progress of the reaction was monitored by TLC analysis. After completion of the reaction, the reaction mixture was quenched with ice and extracted the product into EtOAc (100 mL). The combined organic layers were dried over Na₂SO₄ and concentrated under vacuum to obtain crude material. The obtained crude material was purified by MPLC Flash column chromatography using 10% EtOAc in hexane as eluent to afford (S)-2-(benzyloxy)-3-(docosyloxy)propan-1-ol (2-6, 2.0 g, 98%) as an off-white solid. ¹H NMR (DMSO-d₆, 400 MHz, ¹H NMR (CDCl₃): δ ppm 7.35-7.28 (m, 5H), 4.73-4.61 (m, 2H), 3.68-3.65 (m, 1H) 3.61-3.42 (m, 7H), 2.19 (brs, 1H), 1.62-1.54 (m, 2H), 1.31-1.14 (m, 38H), 0.92-0.82 (m, 3H).

### Synthesis of (R)-dibenzyl (2-(benzyloxy)-3-(docosyloxy)propyl) phosphate (2-8)

To a stirred solution of (S)-2-(benzyloxy)-3-(docosyloxy)propan-1-ol (2-6, 600 mg, 1.224 mmol) in THF (20 mL), was added KOtBu (205 mg, 1.836 mmol) and tetrabenzyl diphosphate 2-7 (790 mg, 1.469 mmol) at 0° C. and stirred at rt for 2 h. The completion of the reaction was monitored by TLC. Progress of the reaction was monitored by TLC analysis. After completion of the reaction, the reaction mixture was quenched using NH₄Cl with ice and the product was extracted into EtOAc (100 mL). The combined organic layers were dried over Na₂SO₄ and concentrated under vacuum to obtain crude material. The obtained crude material was purified by MPLC Flash column chromatography using 20% EtOAc in hexane as eluent to afford (R)-dibenzyl (2-(benzyloxy)-3-(docosyloxy)propyl) phosphate (2-8, 450 mg, 49%) as an off-white solid. HPLC (ELSD): 90.68%, ¹H NMR (CDCl₃, 400 MHz): δ ppm 7.34-7.26 (m, 15H), 5.04-5.02 (m, 4H) 4.63-4.62 (m, 2H), 4.17-4.07 (m, 2H) 3.72-3.70 (m, 1H), 3.48-3.36 (m, 4H), 1.54-1.51 (m, 2H), 1.31-1.14 (m, 38H), 0.90-0.86 (m, 3H).

### Synthesis of Compound 2:

To a stirred solution of (R)-dibenzyl (2-(benzyloxy)-3-(docosyloxy)propyl) phosphate 2-8 (450 mg, 0.599 mmol) in MeOH (50 mL), was added Pd(OH)₂ (45 mg) and the reaction mixture was stirred at it under H2 balloon pressure (40 psi) for 4 h. Progress of the reaction was monitored by TLC. After completion of the reaction, the reaction mixture was filtered over a celite bed and the filtrate was concentrated under vacuum to obtain crude material. The crude material was washed with diethyl ether, filtered and dried under vacuum to afford Compound 2 (145 mg, 0.27 mmol, 50%) as an off-white solid. HPLC (ELSD): 99.14%, HRMS (M+1): 481.3962; ¹H NMR (CDCl3, 400 MHz): δ ppm 3.80-3.73 (m, 3H), 3.40-3.29 (m, 4H), 1.50-1.40 (m, 2H), 1.31-1.21 (m, 40H), 0.87-0.84 (m, 3H).

### Synthesis of Compound 10

To a stirred solution of (R)-dibenzyl (2-(benzyloxy)-3-(docosyloxy)propyl) phosphate 2-8 (500 mg, 0.666 mmol) in dioxane (5 mL), was added dioxane in HCl (4.0 M) (20 mL) at 0° C. and the reaction mixture stirred at rt for 48 h. Progress of the reaction was monitored by TLC. After completion of the reaction, the reaction mixture was concentrated under vacuum and residue was diluted with water. The obtained solid was filtered and dried under vacuum to obtain impure material. The obtained impure material was further purified by stirring in ACN (100 mL) for 30 min., filtered and solid was dried under vacuum to afford Compound 10 (120 mg, 0.17 mmol, 31%) as an off-white solid. HPLC (ELSD): 99.13%, HRMS (M+1): 571.4449; ¹H NMR (CDCl₃, 400 MHz): δ ppm 7.28-7.13 (m, 5H), 4.64-4.56 (m, 2H), 3.99-3.95 (m, 2H), 3.72 (bs, 1H), 3.51-3.22 (m, 4H), 1.48-1.43 (m, 2H), 1.30-1.21 (m, 40H), 0.80-0.87 (m, 3H).

### Alternative Synthesis of Compound 9 and Compound 2

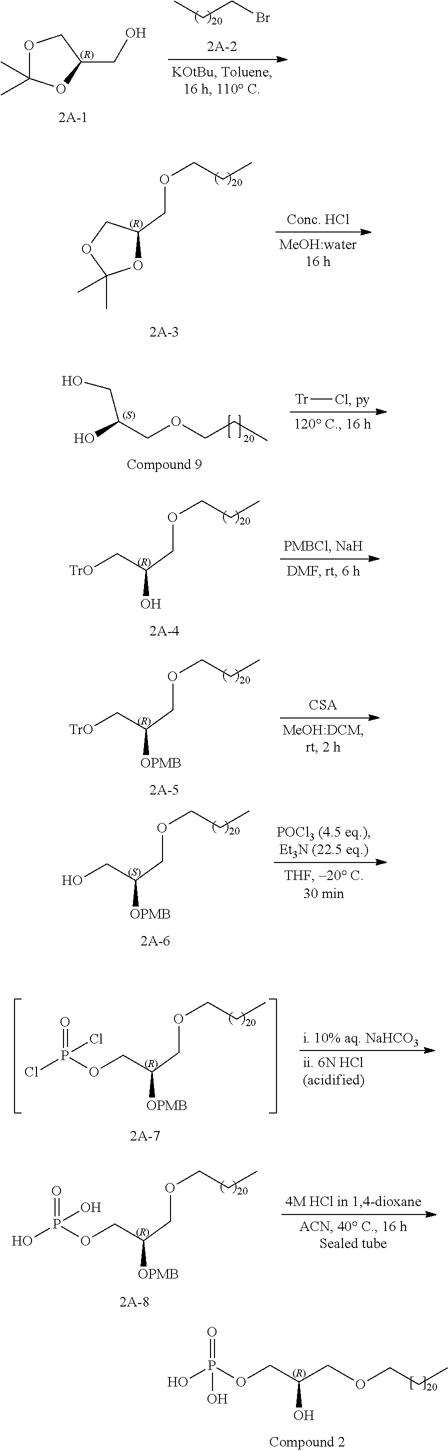

### Synthesis of (R)-4-((docosyloxy)methyl)-2,2-dimethyl-1,3-dioxolane (2A-3)

To the stirred solution of 1-bromodocosane (2A-2) (16.96 g, 128.3664 mmol) in toluene at 0° C., potassium tertiary-butoxide (28.8 g, 256.7328 mmol) and (R)-(2,2-dimethyl-1,3-dioxolan-4-yl)methanol (2A-1) (16.96 g, 128.3664 mmol) was added. The reaction mixture became a thick mass. The reaction mixture was stirred at RT for 1 h and then the reaction mixture was heated to 110° C. for 16 h. The completion of the reaction was monitored by TLC. After completion of the reaction, ether was added to the reaction mixture and stirred for 10 min. Brine solution was added to the reaction mixture and extracted with ether. The organic layer was dried over anhydrous Na₂SO₄ and concentrated under reduced pressure to get crude product (60 g) as brown colored solid. Confirmed by 1H NMR. ¹H NMR (CDCl3) 400 MHz δ ppm 5.01-4.91 (m, 1H), 4.29-4.22 (m, 1H), 4.06-4.04 (m, 1H), 3.74-3.72 (m, 1H), 3.53-3.39 (m, 3H), 1.59-1.53 (m, 2H), 1.45-1.18 (brm, 44H), 0.86 (t, J=13.6 Hz, 3H). ¹H NMR showed the desired product along with impurities; ¹H NMR values were assigned on the basis of product peaks in the next step.

### Synthesis of Compound 9

To a stirred solution of (R)-4-((docosyloxy)methyl)-2,2-dimethyl-1,3-dioxolane (2A-3) (60 g, 136.1315 mmol) in MeOH (500 mL), conc. HCl (125 mL) was added and heated to 70° C. for 16 h. The completion of the reaction was monitored by TLC. The reaction mixture was diluted with water and filtered, the filtered solid was again stirred with water, and filtered to get a solid. The solid was stirred with hexane and filtered to get product, which contained water. Acetonitrile was added to the product and distilled three times to remove moisture to afford Compound 9 (30 g, 55% over two steps) as an off white solid. Confirmed by ¹H NMR (CDCl₃) 400 MHz). ¹H NMR (CDCl3) 400 MHz δ ppm 3.86 (bs, 1H), 3.71-3.66 (m, 2H), 3.53-3.44 (m, 4H), 2.59 (bs, 1H), 2.15 (bs, 1H), 1.58-1.54 (m, 2H), 1.38-1.18 (bs, 38H), 0.88 (t, J=6.4 Hz, 3H).

### Synthesis of (R)-1-(docosyloxy)-3-(trityloxy) propan-2-ol (2A-4)

To a stirred solution of Compound 9 ((S)-3-(docosyloxy)propane-1,2-diol (20.0 g, 49.913 mmol)) in pyridine (100.0 mL) at 0° C., trityl chloride (13.91 g, 49.913 mmol) was added at 0° C. and heated to 120° C. for 16 h in a sealed tube. Starting material, pyridine, and trityl chloride were anhydrous, as moisture hinders the reaction. The completion of the reaction was monitored by TLC. After completion of the reaction, the reaction mixture was evaporated under reduced pressure to get crude. The crude product was purified by combi flash using 5% EtOAc in hexane as eluent. After evaporation of fractions, the product was washed with n-pentane (500.0 mL) stirred for 1 h, filtered, and dried to get the white solid as a desired compound with contamination of trityl impurity. The desired product 2A-4 (19.4 g, 60%) was obtained as white solid and characterized and confirmed by 1H NMR. ¹H NMR (400 MHz, CDCl₃): δ=7.25-7.43 (m, 15H), 3.94 (m, 1H), 3.42-3.52 (m, 4H), 3.18 (m, 2H), 1.22-1.48 (m, 38H), 0.87 (m, 3H).

### Synthesis of (R)-((3-(docosyloxy)-2-((4-methoxybenzyl)oxy)propoxy)methanetriyl) tribenzene (2A-5)

To a stirred solution of (R)-1-(docosyloxy)-3-(trityloxy) propan-2-ol (2A-4) (10.00 g, 15.55 mmol)) in DMF (150 mL) was added NaH (1.55 g, 38.88 mmol) at 0° C. and the reaction mixture was stirred for 20 min. After 20 min, p-methoxybenzyl chloride (3.14 mL, 23.32 mmol) was added dropwise and the reaction mixture was stirred for 16 h. The completion of the reaction was monitored by TLC. The reaction mixture was quenched with ice cold water (100 mL) and extracted with EtOAc (2×150 mL). The organic layer was dried over Na₂SO₄ and evaporated under vacuum to obtain crude material. The obtained crude material was purified by combi-flash chromatography (40 g column) using 0.5% EtOAc and 1% triethylamine in hexane as eluent to afford the title compound 2A-5 as a colorless liquid (12 g, impure; trityl impurity was not separated at this stage). Confirmed by ¹H NMR. ¹H NMR (400 MHz, CDCl₃): δ=7.45-6.84 (m, 19H), 4.59-4.57 (m, 2H), 3.81-3.80 (m, 3H), 3.73-3.70 (m, 1H), 3.55-3.53 (m, 2H), 3.40-3.36 (m, 2H), 3.20-3.19 (m, 2H), 1.52-1.49 (m, 2H), 1.30-1.20 (m, 38H), 0.89-0.84 (m, 3H).

### Synthesis of (S)-3-(docosyloxy)-2-((4-methoxybenzyl)oxy)propan-1-ol (2A-6)

To a stirred solution of (R)-((3-(docosyloxy)-2-((4-methoxybenzyl)oxy)propoxy)methanetriyl) tribenzene (2A-5) (12.00 g, 15.72 mmol)) in DCM:MeOH (120 mL) was added camphorsulfonic acid (3.65 g, 15.72 mmol) at 0° C. and the reaction mixture was stirred at rt for 2 h. The completion of the reaction was monitored by TLC. The reaction mixture was diluted with DCM (200 mL) and washed with water (2×150 mL). The organic layer was dried over Na₂SO₄ and evaporated under vacuum to obtain crude material. The obtained crude material was purified by combi-flash chromatography (40 g column) using 20% EtOAc and 1% triethylamine in hexane as eluent to afford the title compound 2A-6 (4.1 g, 50%) as a white solid, confirmed by ¹H NMR. ¹H NMR (400 MHz, CDCl₃): δ=7.28 (d, J=8.8 Hz, 2H), 6.88 (d, J=8.4 Hz, 2H), 4.66-4.53 (m, 2H), 3.73 (s, 3H), 3.72-3.66 (m, 1H), 3.65-3.62 (m, 2H), 3.57-3.51 (m, 2H), 3.45-3.42 (m, 2H), 2.16-2.13 (m, 1H), 1.57-1.54 (m, 2H), 1.31-1.19 (m, 38H), 0.89-0.86 (m, 3H).

### Synthesis of (R)-3-(docosyloxy)-2-((4-methoxybenzyl) oxy) propyl dihydrogen phosphate (2A-8)

To a stirred solution of distilled POCl₃ (0.8 mL, 8.5567 mmol) in THF (7 mL) at −20° C. (salt ice mixture) a solution of (S)-3-(docosyloxy)-2-((4-methoxybenzyl) oxy) propan-1-ol (2A-6) (1 g, 1.9199 mmol) and dry Et₃N (6 mL, 43.0477 mmol) in dry THF (7 mL) was added dropwise over a period of 10 min and stirred at −20° C. for another 20 min. Initially the reaction mixture was off white for the first 20 min and slowly turned to pale yellow. The completion of the reaction was monitored by TLC. The reaction mixture was slowly quenched with 10% aq NaHCO₃ solution (5 mL) and stirred at the same temperature for 30 min. The temperature of the ice bath reached −10° C., then the reaction mixture was acidified with 6 N HCl, and extracted with DCM (60 mL). The DCM layer was washed with 10% NaHCO₃ solution (2×30 mL), the aqueous layer was separated and acidified with conc. HCl and extracted with ethyl acetate and DCM. The combined organic layers were dried over anhydrous Na₂SO₄ and concentrated under reduced pressure at 40° C. to give the title compound 2A-8 as an off white solid (0.6 g, 52%). Confirmed by 1H NMR, 31P NMR.

¹H NMR (CD3OD) 400 MHz VT at 50° C. δ ppm 7.29 (d, J=8.4 Hz, 2H), 6.87 (d, J=8.4 Hz, 2H), 4.65-4.57 (m, 2H), 4.05-3.98 (m, 1H), 3.79-3.76 (bs, 4H), 3.55-3.48 (m, 2H), 3.43 (t, J=6.8 Hz, 2H), 1.58-1.51 (m, 2H), 1.33-1.21 (bs, 38H), 0.89 (t, J=6.4 Hz, 3H)

31P NMR (CDCl3) 400 MHz) δ ppm 0.719 (t). ¹H NMR and ³¹P NMR were recorded in CD₃OD and CD₃OD+CDCl₃ at 50° C.-60° C. due to the low solubility of the compound.

HPLC: tRet 7.705 min (99.56%) HPLC Method conditions: Column: LUNA HILIC (250*4.6) mm, 5 μm, 200A Mobile phase-A: 10 Mm Ammonium Acetate in (Aq); Mobile phase-B: ACN 100% Method-T/% B: −0/10, 2/10, 6/100, 13/100, 14/10, 15/10 Flow rate: 1.0 ml/min Column temp: 30° C. Diluent: THF

### Synthesis of (R)-3-(docosyloxy)-2-hydroxypropyl dihydrogen phosphate (Compound 2)

To a stirred suspension of (R)-3-(docosyloxy)-2-((4-methoxybenzyl) oxy) propyl dihydrogen phosphate) (2A-8) (200 mg, 0.3328 mmol) in acetonitrile (10 mL), was added 4M HCl in 1,4-dioxane (0.2 mL) at RT and stirred the reaction mixture at 40° C. for 16 h in sealed tube. Acetonitrile was added to the reaction mixture, and solvent removed with a dropper, and this process repeated three times, then the solvent was removed under reduced pressure at 40° C. to afford Compound 2 as off white solid (140 mg, 87.5%). Confirmed by 1H NMR, 31P NMR.

¹H NMR (CD3OD) 400 MHz VT at 50° C. δ ppm 4.02-3.89 (m, 3H), 3.19-3.45 (m, 4H), 1.61-1.54 (m, 2H), 1.37-1.21 (bs, 38H), 0.89 (t, J=6.8 Hz, 3H).

31P NMR (CD3OD) 400 MHz VT at 50° C. δ ppm 0.76 (t) Note: ¹HNMR, ³¹PNMR were recorded in CD3OD and CD3OD+CDCl₃ at 50° C.-60° C. due to the low solubility of compound.

HPLC: tRet 7.746 min (98.43%) HPLC Method conditions: Column: LUNA HILIC (250*4.6) mm, 5 μm, 200A Mobile phase-A: 10 Mm Ammonium Acetate in (Aq); Mobile phase-B: ACN 100% Method-T/% B: −0/10, 2/10, 6/100, 13/100, 14/10, 15/10 Flow rate: 1.0 ml/min Column temp: 30° C. Diluent: ACN+H₂O.

### Example S-3: Synthesis of Compound 7

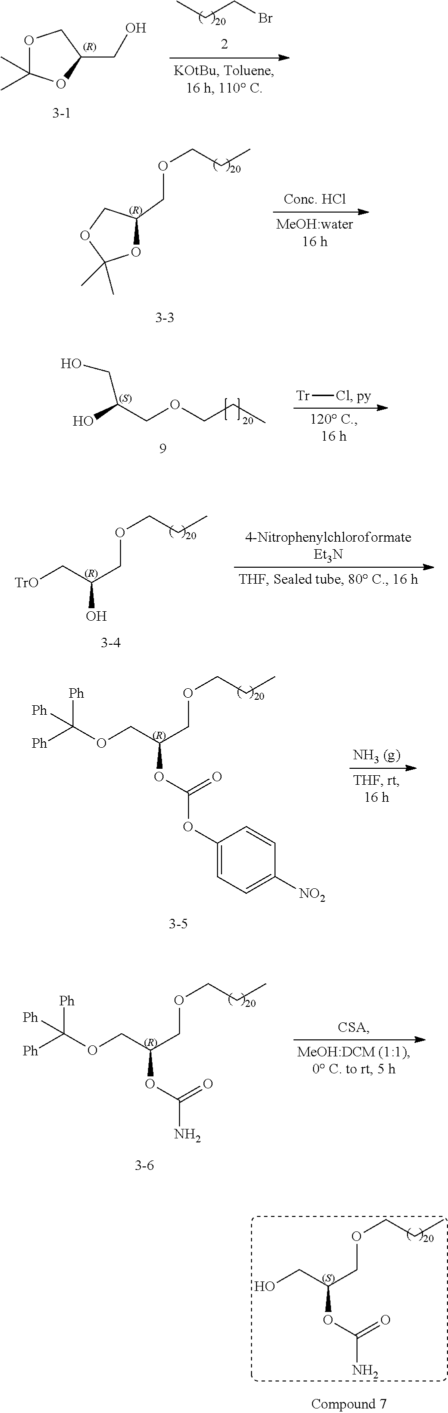

### Synthesis of (R)-4-((docosyloxy)methyl)-2,2-dimethyl-1,3-dioxolane (3-3)

To the stirred solution of 1-Bromodocosane (3-1) (16.96 g, 128.3664 mmol) in Toluene at 0° C., Potassium tertiarybutoxide (28.8 g, 256.7328 mmol) and (R)-(2,2-dimethyl-1,3-dioxolan-4-yl)methanol (3-2) (50 g, 128.3664 mmol) was added, the reaction mixture becomes thick mass and stirring was stopped, The reaction mixture stirred at RT for 1 h and then the reaction mixture was heated to 110° C. for 16 h. The completion of the reaction was monitored by TLC. After completion of the reaction, diethyl ether was added to the reaction mixture and stirred for 10 min, brine solution was added to the reaction mixture and extracted with diethyl ether. The organic layer was dried over anhydrous Na₂SO₄ and concentrated under reduced pressure to afford (R)-4-((docosyloxy)methyl)-2,2-dimethyl-1,3-dioxolane (3-3) as brown colour solid (57 g, crude). HPLC (ELSD): 88.67%, ¹H NMR (CDCl₃, 400 MHz): δ ppm 4.29-4.23 (m, 1H), 4.06-4.04 (m, 1H), 3.74-3.71 (m, 1H), 3.53-3.41 (m, 3H), 1.57-1.51 (m, 2H), 1.45 (s, 3H), 1.38 (m, 3H), 1.31-1.14 (m, 40H), 0.89-0.86 (m, 3H).

### Synthesis of Compound 9:

To the stirred solution of (R)-4-((docosyloxy)methyl)-2,2-dimethyl-1,3-dioxolane (3-3) (57 g, 129.3250 mmol) in MeOH (500 mL), Conc. HCl (125 mL), was added and heated to 70° C. for 16 h. The completion of the reaction was monitored by TLC. The reaction mixture was diluted with water and filtered, the filtered solid was again stirred with water and filtered to get solid. The solid was stirred with hexane and filtered to obtain pure material. The obtained material contains water, acetonitrile was added to the product and co-distilled thrice (to remove traces of water) to afford Compound 9 as an off-white solid (28 g, 54% over two steps). HPLC (ELSD): 99.70%, ¹H NMR (CDCl₃. 400 MHz): δ ppm 3.88-3.83 (m, 1H), 3.71-3.67 (m, 2H), 3.53-3.44 (m, 4H), 2.59-2.58 (m, 1H), 2.15 (t, J=5.6 Hz, 1H), 1.58-1.54 (m, 2H), 1.31-1.28 (m, 40H), 0.88 (t, J=6.8 Hz, 3H).

### Synthesis of (R)-1-(docosyloxy)-3-(trityloxy) propan-2-ol (3-4)

To a stirred solution of (S)-3-propoxypropane-1,2-diol (3-3) (25 g, 0.0625 mol) in pyridine (150 mL), was added trityl chloride (15.6 g, 0.0.0562 mol) at 0° C. and the reaction mixture was stirred at 120° C. for 16 h. The completion of the reaction was monitored by TLC. After completion of the reaction, the reaction mixture was concentrated under vacuum to afford crude material. The obtained crude material was purified by MPLC Flash Column chromatography using 10% EtOAc in hexane as eluent to afford (R)-1-(docosyloxy)-3-(trityloxy) propan-2-ol (3-4) as an off-white solid (25 g, 62%). ¹H NMR (CDCl3, 400 MHz): δ ppm 7.44-7.21 (m, 15H), 3.97-3.93 (m, 1H), 3.52-3.41 (m, 4H), 3.22-3.15 (m, 2H), 2.42 (d, J=3.6 Hz, 1H), 1.57-1.52 (m, 2H), 1.31-1.28 (m, 40H), 0.87 (t, J=6.4 Hz, 3H).

### Synthesis of (R)-4-nitrophenyl (1-propoxy-3-(trityloxy)propan-2-yl) carbonate (3-5)

To the stirred solution (R)-1-(docosyloxy)-3-(trityloxy)propan-2-ol (3-4) (1.2 g, 1.866 mmol) in THF (30 mL) at RT, Et₃N (0.52 mL, 3.732 mmol) was added followed by the addition of 4-nitrophenylchloroformate (0.56 g, 2.7993 mmol). The reaction mixture was heated to 80° C. for 16 h in sealed tube. TLC indicated starting material along with the formation of product further Et₃N (1.3 mL, 9.33 mmol) and 4-nitrophenylchloroformate (1.88 g, 9.33 mmol) was added and heated to 80° C. for 16 h in sealed tube. The completion of the reaction was monitored by TLC. The reaction mixture was concentrated under vacuum. The residue was dissolved in EtOAc and washed with brine solution, combined organic layer was dried over anhydrous Na₂SO₄ and concentrated under vacuum to obtain crude material. The obtained crude material was purified by combi-flash column chromatography using 30% EtOAc in hexane as eluent to afford (R)-4-nitrophenyl (1-propoxy-3-(trityloxy)propan-2-yl) carbonate (5) as off-white solid (1.2 g, impure). HPLC (ELSD): 99.89%, ¹H NMR (CDCl₃, 400 MHz): δ ppm 8.28-8.25 (m, 2H), 7.45-7.23 (m, 17H), 5.17-5.14 (m, 1H), 3.70-3.63 (m, 2H), 3.45-3.34 (m, 4H), 1.56-1.53 (m, 2H), 1.31-1.28 (m, 40H), 0.87 (t, J=6.8 Hz, 3H).

### Synthesis of (R)-1-(docosyloxy)-3-(trityloxy)propan-2-yl carbamate (3-6)

To the stirred solution of (R)-1-(docosyloxy)-3-(trityloxy)propan-2-yl (4-nitrophenyl) carbonate (3-5) (1.2 g, 1.4849 mmol) in THF at 0° C. ammonia gas was purged and stirred at RT for 16 h in sealed tube. The completion of the reaction was monitored by TLC. The reaction mixture was concentrated under vacuum to obtain crude material. The obtained crude material was purified by combi-flash column chromatography using 5% EtOAc in hexane to remove 4-nitrophenol impurity and then eluted with 30% EtOAc in hexane as eluent to afford (R)-1-(docosyloxy)-3-(trityloxy)propan-2-yl carbamate (3-6) as colourless waxy solid (0.75 g, 73.62%). ¹H NMR (CDCl₃, 400 MHz): δ ppm 7.45-7.23 (m, 15H), 5.09-5.04 (m, 1H), 4.66 (brs, 2H), 3.65-3.60 (m, 2H), 3.44-3.37 (m, 2H), 3.35-3.23 (m, 2H) 1.50-1.47 (m, 2H), 1.31-1.28 (m, 40H), 0.87 (t, J=6.4 Hz, 3H).

### Synthesis of (S)-1-(docosyloxy)-3-hydroxypropan-2-yl carbamate (Compound 7)

To the stirred solution of (R)-1-(docosyloxy)-3-(trityloxy) propan-2-yl carbamate (3-6) (0.75 g, 1.0932 mmol) in MeOH and DCM (1:1, 10 mL), was added CSA (0.05 g, 0.21864 mmol) and reaction mixture was stirred at RT for 16 h. The completion of the reaction was monitored by TLC. The reaction mixture was filtered, solid was washed with ether and dried under vacuum to afford (S)-1-(docosyloxy)-3-hydroxypropan-2-yl carbamate (Compound 7) as an off-white solid (0.33 g, 68.03%). HPLC (ELSD): 99.85%, HRMS: 444.4623. ¹H NMR (CDCl₃. 400 MHz): δ ppm 4.90-4.85 (m, 1H), 4.72 (brs, 2H), 3.84 (t, J=5.6 Hz, 3H), 3.66-3.64 (m, 2H), 3.49-3.42 (m, 2H), 2.45 (t, J=6.4 Hz, 1H), 1.55-1.53 (m, 2H), 1.31-1.28 (m, 40H), 0.88 (t, J=6.4 Hz, 3H).

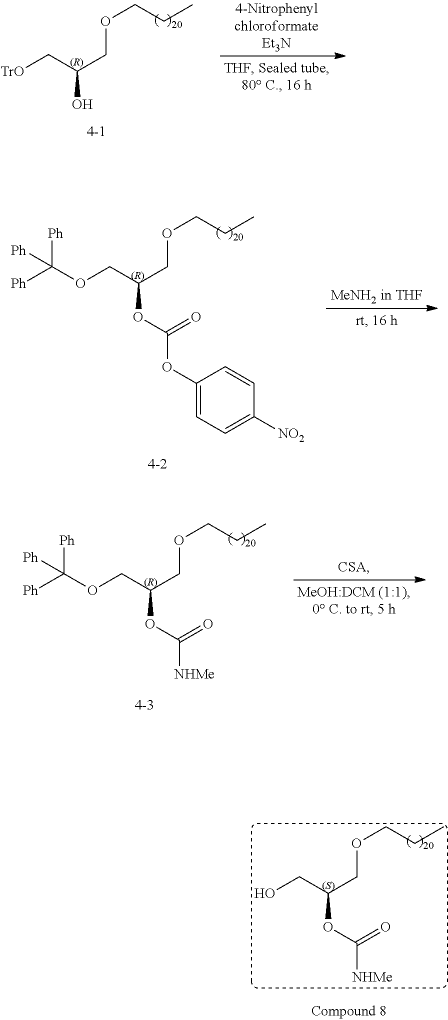

### Synthesis of (R)-4-nitrophenyl (1-propoxy-3-(trityloxy)propan-2-yl) carbonate (4-2)

To the stirred solution of (R)-1-(docosyloxy)-3-(trityloxy)propan-2-ol (4-1) (2.5 g, 0.0038 mol) in THF (30 mL) at RT, Et₃N (1.1 mL, 0.0076 mol) was added followed by the addition of 4-nitrophenylchloroformate (1.17 g, 0.0058 mol) was added and stirred at 80° C. for 16 h. The completion of the reaction was monitored by TLC. The reaction mixture was concentrated under vacuum. The residue was dissolved in EtOAc and washed with brine solution, combined organic layer was dried over anhydrous Na₂SO₄ and concentrated under vacuum to obtain crude material. The obtained crude material was purified by combi-flash column chromatography using 20% EtOAc in hexane as eluent to afford (R)-4-nitrophenyl (1-propoxy-3-(trityloxy)propan-2-yl) carbonate (4-2) as an off-white solid (3 g, impure). ¹H NMR (CDCl₃. 400 MHz): δ ppm 8.27 (d, J=9.2 Hz, 2H), 7.45-7.23 (m, 17H), 5.17-5.14 (m, 1H), 3.70-3.63 (m, 2H), 3.45-3.34 (m, 4H), 1.56-1.53 (m, 2H), 1.31-1.28 (m, 40H), 0.87 (t, J=6.8 Hz, 3H).

### Synthesis of (R)-1-(docosyloxy)-3-(trityloxy)propan-2-yl methylcarbamate (4-3)

To the stirred solution (R)-1-(docosyloxy)-3-(trityloxy) propan-2-yl (4-nitrophenyl) carbonate (4-2) (3.5 g, 0.0043 mmol) in THF (40 mL) at 0° C. was added methyl amine solution in THF (5 mL) and the reaction mixture was stirred at rt for 16 h. The completion of the reaction was monitored by TLC. The reaction mixture was concentrated under vacuum to obtain crude material. The obtained crude material was purified by combi-flash column chromatography using 20% EtOAc in hexane as eluent to afford (R)-1-(docosyloxy)-3-(trityloxy)propan-2-yl methylcarbamate (4-3) as an off-white solid (3 g, impure). ¹H NMR (CDCl₃, 400 MHz): δ ppm 7.43-7.20 (m, 15H), 6.82 (brs, 1H) 5.09-5.04 (m, 1H), 4.72-4.71 (m, 1H), 3.67-3.58 (m, 2H), 3.44-3.37 (m, 2H), 3.27-3.22 (m, 2H), 2.80 (d, J=4.8 Hz, 3H), 1.50-1.47 (m, 2H), 1.31-1.28 (m, 40H), 0.87 (t, J=6.4 Hz, 3H).

### Synthesis of (S)-1-(docosyloxy)-3-hydroxypropan-2-yl methylcarbamate (Compound 8)

To a stirred solution of (R)-1-(docosyloxy)-3-(trityloxy) propan-2-yl methylcarbamate (4-3) (3 g, 0.00429 mmol) in DCM; MeOH (40 mL), was added CSA (0.9 g, 0.00429 mol) at 0° C. and the reaction mixture was stirred at rt for 1 h. The completion of the reaction was monitored by TLC. The residue was dissolved in EtOAc and washed with brine solution, and the combined organic layer was dried over anhydrous Na₂SO₄ and concentrated under vacuum to obtain crude material. The obtained crude material was purified by combi-flash column chromatography using 30% EtOAc in hexane as eluent to afford (S)-1-(docosyloxy)-3-hydroxypropan-2-yl methylcarbamate Compound 8 as an off-white solid. HPLC (ELSD): 99.88%, ¹H NMR (CDCl₃, 400 MHz): δ ppm 4.90 (brs, 1H), 4.77 (brs, 1H), 3.83 (brs, 2H), 3.64-3.63 (m, 2H), 3.49-3.44 (m, 2H), 2.82 (d, J=5.2 Hz, 3H), 2.56 (brs, 1H), 1.59 (brs, 2H), 1.31-1.28 (m, 40H), 0.90 (t, J=6.4 Hz, 3H).

### Example S-5: Synthesis of Compound 12, Compound 11, and Compound 13

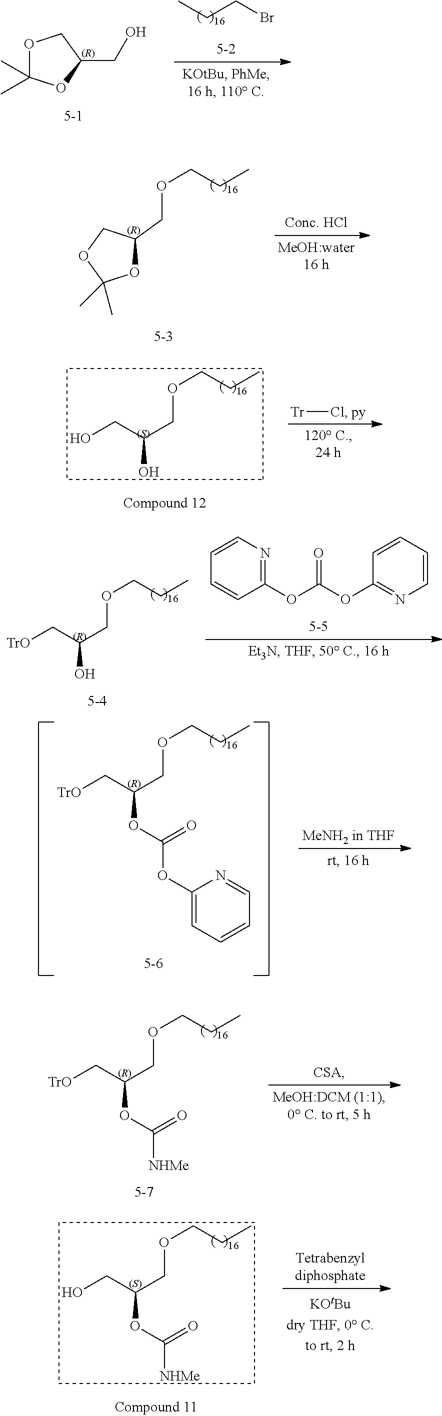

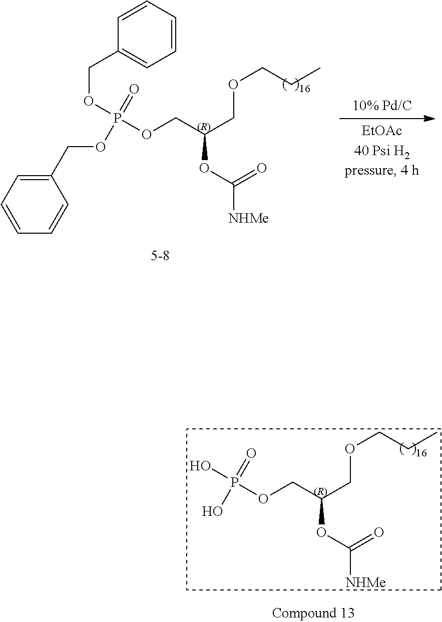

### Synthesis of (R)-2,2-dimethyl-4-((octadecyloxy)methyl)-1,3-dioxolane (5-3)

To the stirred solution of 1-bromooctadecane (5-2, 25.22 g, 75.66 mmol, 1.0 eq) in toluene at 0° C., potassium tertiary butoxide (16.97 g, 151.32 mmol, 2.0 eq) and (R)-(2,2-dimethyl-1,3-dioxolan-4-yl)methanol (5-1, 10.0 g, 75.66 mmol, 1.0 eq) was added. The thick mass of the reaction mixture stirred at RT for 1 h and heated to 110° C. for 16 h. The completion of the reaction was monitored by TLC. After completion of the reaction, diethyl ether (500.0 mL) was added to the reaction mixture and stirred for 10 min, brine solution (500.0 mL) was added to the reaction mixture and extracted with ether. The organic layer was dried over anhydrous sodium sulphate and concentrated under reduced pressure to get crude product 5-3 (29 g) as brown colored semi solid. Confirmed by crude ¹H NMR.

¹H NMR (400 MHz, CDCl₃): δ=4.23-4.29 (m, 1H), 4.23-4.29 (m, 1H), 4.04-4.06 (m, 1H), 3.72-3.74 (m, 1H), 3.41-3.53 (m, 3H), 1.55-1.57 (m, 2H), 1.38 (m, 30H), 0.86-0.89 (m, 3H).

### Synthesis of ((S)-3-(octadecyloxy)propane-1,2-diol) (Compound 12)

To the stirred solution of (R)-2,2-dimethyl-4-((octadecyloxy)methyl)-1,3-dioxolane 5-3 (14.0 g, 37.695 mmol, 1.0 eq) in methanol (140 mL), Conc. HCl (41.5 ML), was added stirred and heated to 70° C. for 16 h. TLC monitored the completion of the reaction. All the solvents were evaporated, then acetonitrile (100.0 mL) was added to crude reaction mixture and stirred for 2 h. Off-white free flow solid was precipitated out which filtered and dried. This crude powder was triturated with n-pentane (50.0 mL) filtered and dried to afford Compound 12 as an off-white solid (7.4 g, 58% over two steps). Confirmed by ¹H NMR (CDCl₃) 400 MHz), CDCl3+D2O and HRMS. ¹H NMR (400 MHz, CDCl₃): δ=3.84-3.88 (m, 1H), 3.71-3.72 (m, 2H), 3.44-3.54 (m, 3H), 2.57-2.58 (d, j=4.8 HZ, 1H), 2.12-2.15 (m, 1H), 1.22-1.4 (m, 32H), 0.86-0.89 (t, 3H). HPLC: 99.79%. HRMS: 344.59 (Complies), Dimer Mass: (689.13) Complies.

### Synthesis of (R)-1-(octadecyloxy)-3-(trityloxy)propan-2-ol (5-4)

To the stirred solution of (S)-3-(octadecyloxy)propane-1,2-diol Compound 12 (1.3 g, 3.772 mmol, 1.0 eq) in pyridine (5.0 mL) at 0° C., trityl chloride (1.05 g, 3.772 mmol) was added and heated to 120° C. for 16 h in sealed tube. Starting material, pyridine, and trityl chloride were anhydrous, as moisture hinders the reaction. Completion of the reaction was monitored by TLC. The reaction mixture was evaporated under reduced pressure and the crude product was purified by combi-flash using 5% ethyl acetate in hexane as eluent. Fractions were collected and concentrated, and finally the compound was triturated with n-pentane (25.0 mL) and filtered and dried to afford title compound 5-4 (1.3 g, 62%) as an off-white solid with traces of deprotected trityl alcohol impurity. Confirmed by ¹H NMR (CDCl₃) 400 MHz

¹H NMR (400 MHz, CDCl₃): δ=7.18-7.50 (m, 15H), 3.93-3.97 (m, 1H), 3.42-3.52 (m, 4H), 3.16-3.22 (m, 2H), 1.50-1.56 (m, 2H), 1.22-1.38 (m, 30H), 0.86-0.93 (t, 3H).

### Synthesis of (R)-1-propoxy-3-(trityloxy)propan-2-yl pyridin-2-yl carbonate (5-6)

To the stirred solution of (R)-1-(octadecyloxy)-3-(trityloxy)propan-2-ol (5-4) (1.0 g, 1.703 mmol, 1.0 eq) in dry THF (10.0 mL) added triethyl amine (1.42 mL, 10.218 mmol, 6.0 eq), followed by di(pyridin-2-yl) carbonate 5-5 (0.736 g, 3.407 mmol, 2.0 eq) at r.t, then the reaction mixture was stirred at 80° C. for 16 h in a sealed tube. TLC monitored the completion of the reaction. As the intermediate 5-6 was unstable, it was directly used for the next step without work-up. Formation of intermediate 5-6 was confirmed by ¹H NMR (CDCl₃) 400 MHz: δ=7.28-7.48 (m, 20H), 3.2-3.8 (m, 4H), 1.28-1.38 (m, 32H).

### Synthesis of (R)-1-(octadecyloxy)-3-(trityloxy)propan-2-yl methylcarbamate (5-7)

To the stirred solution of intermediate (R)-1-propoxy-3-(trityloxy)propan-2-yl pyridin-2-yl carbonate 5-6 (1.0 g, 1.446 mmol, 1.0 eq) in dry THF (20.0 mL) was added 7% methyl amine in THF (10.0 mL) at 0° C. The reaction mixture was stirred at 80° C. for 16 h in a sealed tube. The completion of the reaction was monitored by TLC. The reaction mixture was evaporated under reduced pressure. The crude product dissolved in ethyl acetate (50.0 mL) extracted with water (25.0 mL×2), the organic layer was dried over anhydrous sodium sulphate and concentrated to get the title compound 5-7 (1.2 g, crude) as an amber colored semi solid. The crude product was characterized by ¹H NMR (CDCl₃, 400 MHz) and used further in the next step without purification.

¹H NMR (400 MHz, CDCl₃): δ=7.26-7.43 (m, 15H), 5.079 (m, 1H), 4.66 (m, 1H), 2.81 (m, 3H), 1.25-1.55 (m, 32H), 1.22-1.38 (m, 30H), 0.88 (t, 3H).

### Synthesis of (S)-1-hydroxy-3-(octadecyloxy)propan-2-yl methylcarbamate (Compound 11)

To the stirred solution of (R)-1-(octadecyloxy)-3-(trityloxy)propan-2-yl methylcarbamate 5-7 (1.2 g, 1.863 mmol, 1.0 eq) in a mixture of dry DCM:Dry MeOH (1:1) 10.0 mL:10.0 mL solvents at 0° C. was added DL-10-camphor sulphonic acid (CSA; 0.433 g, 1.863 mmol, 1.1 eq) and the reaction mixture was stirred at room temperature for 5 h. Completion of the reaction was monitored by TLC. The reaction mixture was diluted with DCM (100.0 mL) and extracted with water (50.0 mL×2). The organic layer was dried over anhydrous sodium sulphate and concentrated to get the crude compound. The crude product was triturated with n-pentane (20.0 mL), to afford Compound 11 (420.0 mg, 56.0%) as an off-white solid. The structure of product was confirmed by 1H NMR (400 MHz) in CDCl₃, CDCl₃+D₂O and HRMS.

¹H NMR (400 MHz, CDCl₃): δ=4.487-4.89 (m, 1H), 4.76 (m, 1H), 3.80-3.83 (m, 2H), 3.59-3.66 (m, 2H), 3.42-3.48 (m, 2H), 2.80-2.82 (d, 3H), 2.54-2.57 (m, 1H), 1.52-1.57 (m, 2H), 1.20-1.4 (m, 32H), 0.86-0.89 (t, 3H). HRMS: 401.43 (Complies), HPLC: 99.84%,

### Synthesis of (R)-1-((bis(benzyloxy)phosphoryl)oxy)-3-(octadecyloxy)propan-2-yl methylcarbamate (5-8)

To the stirred solution of (S)-1-hydroxy-3-(octadecyloxy)propan-2-yl methylcarbamate Compound 11 (1.0 g, 2.489 mmol, 1.0 eq) in Dry THF (80.0 mL) solvent at 0° C. was added potassium tert-butoxide (0.279 g, 2.489 mmol, 2.0 eq) stirred for 5 min. Tetrabenzyl diphosphate (1.34 g, 2.489 mmol, 1.0 eq) was added and the reaction mixture was stirred at rt for 3 h. Completion of the reaction was monitored by TLC. The reaction mixture was diluted with ethyl acetate (50.0 mL), and extracted with water (50.0 mL×2). The organic layer was dried over anhydrous sodium sulphate and concentrated to get 2.0 g crude compound. The crude compound was purified by prep-HPLC to afford compound 5-8 (750.0 mg, 45%) as a white solid. Confirmed by ¹H NMR, ³¹P NMR.

¹H NMR (400 MHz, CDCl₃): δ=4.46 (m, 1H), 4.71 (m, 1H), 4.18-4.27 (m, 2H), 2.66-2.70 (m, 2H) 1.48-1.51 (m, 2H), 1.29 (m, 30H), 0.86-0.89 (t, 3H). ³¹P NMR: Complies

### Synthesis of (R)-1-(octadecyloxy)-3-(phosphonooxy)propan-2-yl methylcarbamate (Compound 13)

To the stirred solution of (R)-1-((bis(benzyloxy)phosphoryl)oxy)-3-(octadecyloxy)propan-2-yl methylcarbamate 5-8 (400.0 mg, 0.604 mmol, 1.0 eq) in MeOH, 20% Pd(OH)₂/C (100.0 mg) was added and hydrogenated at 40 PSI for 4 h. The completion of the reaction monitored by TLC. The reaction mixture was filtered through Celite and washed with 250 mL of methanol and the filtrate was passed through a micron filter NYL 0.45 um and concentrated to get crude product. The crude compound was triturated with n-pentane and dried under high vacuum to afford Compound 13 as a white solid (350 mg, 99%). Confirmed by 1H NMR, 31P NMR and HRMS.

¹H NMR (400 MHz, CDOD): δ=4.35-4.40 (m, 1H), 4.14-4.18 (m, 1H), 4.05-4.08 (m, 1H), 3.47-3.55 (m, 2H), 3.37-3.39 (m, 2H), 2.59-2.64 (d, 3H), 1.42-1.47 (m, 2H), 1.20-1.30 (m, 30H), 0.78-0.81 (t, 3H). HRMS: 482.01 (Complies), HPLC: 99.87%.

### Example S-6: Synthesis of Compound 6

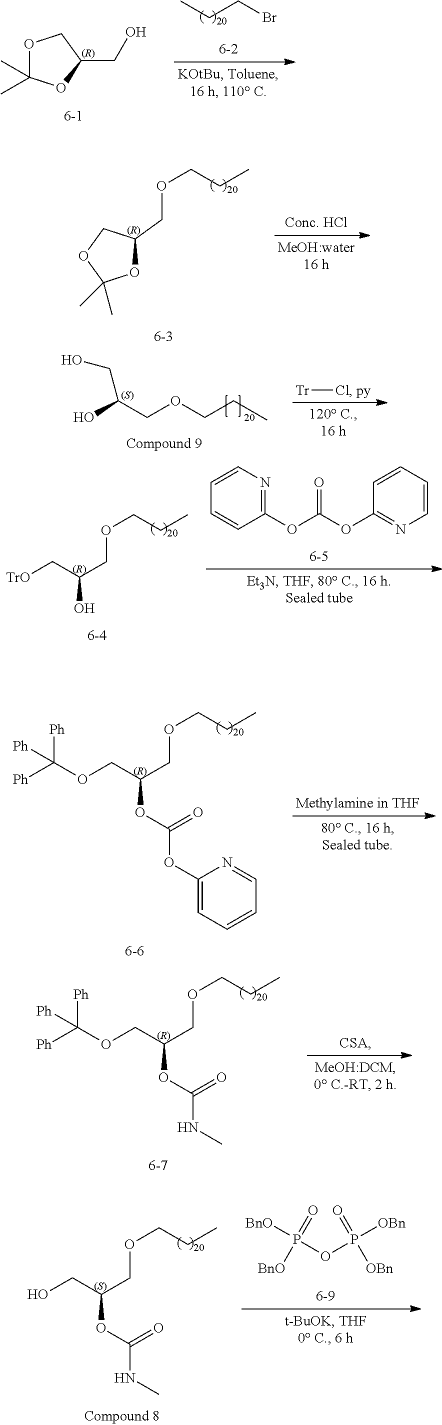

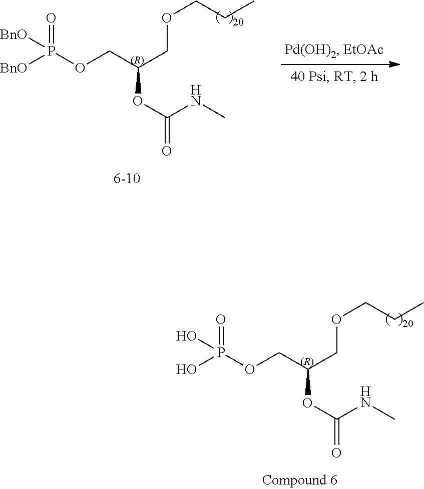

### Synthesis of (R)-4-((docosyloxy)methyl)-2,2-dimethyl-1,3-dioxolane (6-3)

To a stirred solution of 1-bromodocosane (6-2) (16.96 g, 128.3664 mmol) in toluene at 0° C., potassium tertiary-butoxide (28.8 g, 256.7328 mmol) and (R)-(2,2-dimethyl-1,3-dioxolan-4-yl)methanol (6-1) (16.96 g, 128.3664 mmol) was added. The reaction mixture became a thick mass. The reaction mixture stirred at RT for 1 h and then the reaction mixture was heated to 110° C. for 16 h. The completion of the reaction was monitored by TLC. After completion of the reaction, ether was added to the reaction mixture and stirred for 10 min. Brine solution was added to the reaction mixture and extracted with ether. The organic layer was dried over anhydrous Na₂SO₄ and concentrated under reduced pressure to get crude product (60 g) as brown color solid. Confirmed by 1H NMR. ¹H NMR (CDCl3) 400 MHz δ ppm 5.01-4.91 (m, 1H), 4.29-4.22 (m, 1H), 4.06-4.04 (m, 1H), 3.74-3.72 (m, 1H), 3.53-3.39 (m, 3H), 1.59-1.53 (m, 2H), 1.45-1.18 (brm, 44H), 0.86 (t, J=13.6 Hz, 3H). The ¹H NMR showed the desired product along with impurities; the ¹H NMR values were assigned on the basis of product peaks in the next step.

### Synthesis of Compound 9

To the stirred solution of (R)-4-((docosyloxy)methyl)-2,2-dimethyl-1,3-dioxolane (6-3) (60 g, 136.1315 mmol) in MeOH (500 mL), conc. HCl (125 mL) was added and heated to 70° C. for 16 h. The completion of the reaction was monitored by TLC. The reaction mixture was diluted with water and filtered, the filtered solid was again stirred with water and filtered to get the solid. The solid was stirred with hexane and filtered to get product. The product contained water. Acetonitrile was added to the product and distilled three times to remove moisture to afford Compound 9 (30 g, 55% over two steps) as an off white solid. Confirmed by ¹H NMR (CDCl₃) 400 MHz). ¹H NMR (CDCl3) 400 MHz δ ppm 3.86 (bs, 1H), 3.71-3.66 (m, 2H), 3.53-3.44 (m, 4H), 2.59 (bs, 1H), 2.15 (bs, 1H), 1.58-1.54 (m, 2H), 1.38-1.18 (bs, 38H), 0.88 (t, J=6.4 Hz, 3H).

### Synthesis of (R)-1-(docosyloxy)-3-(trityloxy) propan-2-ol (6-4)

To the stirred solution of Compound 9 ((S)-3-(docosyloxy)propane-1,2-diol (20.0 g, 49.913 mmol)) in pyridine (100.0 mL) at 0° C. trityl chloride (13.91 g, 49.913 mmol) was added at 0° C. and heated to 120° C. for 16 h in a sealed tube. The completion of the reaction was monitored by TLC. After completion of the reaction, the reaction mixture was evaporated under reduced pressure to get crude product. The crude product was purified by combi-flash using 5% EtOAc in hexane as eluent. After evaporation of fractions, the product was washed with n-pentane (500.0 mL) stirred for 1 h, filtered and dried to get the white solid as a desired compound with contamination of trityl impurity. The desired product 6-4 (19.4 g, 60%) was obtained as white solid and characterized and confirmed by 1H NMR. ¹H NMR (400 MHz, CDCl₃): δ=7.25-7.43 (m, 15H), 3.94 (m, 1H), 3.42-3.52 (m, 4H), 3.18 (m, 2H), 1.22-1.48 (m, 38H), 0.87 (m, 3H).

### Synthesis of (R)-1-(docosyloxy)-3-(trityloxy)propan-2-yl pyridin-2-yl carbonate (6-6)

To the stirred solution of (R)-1-(docosyloxy)-3-(trityloxy)propan-2-ol (5 g, 7.7759 mmol) in THF at RT, Et₃N (4.3 mL, 31.1036 mmol) was added followed by the addition of di(pyridin-2-yl) carbonate (3.36 g, 15.5518 mmol) and stirred at 80° C. for 16 h in a sealed tube. Starting material, pyridine, and trityl chloride were anhydrous, as moisture hinders the reaction. The completion of the reaction was monitored by TLC. Due to the instability of product reaction mixture, this crude mixture of (R)-1-(docosyloxy)-3-(trityloxy)propan-2-yl pyridin-2-yl carbonate 6-6 (5 g, crude, pale brown reaction mixture) was used for the next step without work-up.

¹H NMR (CDCl3) 400 MHz) δ ppm 7.80-7.76 (m, 1H), 7.47-7.27 (m, 16H), 6.57 (d, J=9.2 Hz, 1H), 6.30-6.27 (m, 1H), 5.14-5.12 (m, 1H), 3.75-3.33 (m, 6H), 1.52-1.49 (m, 2H), 1.4-1.2 (bs, 38H), 0.87 (t, J=6.4 Hz, 3H). The crude ¹H NMR showed the desired product along with impurities.

### Synthesis of (R)-1-(docosyloxy)-3-(trityloxy)propan-2-yl methylcarbamate (6-7)

To a stirred solution of (R)-1-(docosyloxy)-3-(trityloxy)propan-2-yl picolinate (6-6) (5 g, 6.6835 mmol) in THF (50 nL) was added 7% methyl amine in THF (10 mL) at 0° C., stirred and heated at 80° C. for 16 h in sealed tube. The completion of the reaction was monitored by TLC. The reaction mixture was evaporated under reduced pressure to get crude. The crude product was dissolved in ethyl acetate (200 mL), washed with water (250 mL×2), extracted and separated. The organic layer was dried over anhydrous sodium sulphate and concentrated to afford (R)-1-(docosyloxy)-3-(trityloxy)propan-2-yl methylcarbamate 6-7 (6 g, crude) as a pale brown solid, and characterized by 1H NMR. ¹H NMR (CDCl3) 400 MHz) δ ppm 7.43-7.42 (m, 15H), 5.08-5.06 (m, 1H), 3.65-3.21 (m, 6H), 2.8 (d, J=4.4 Hz, 3H), 1.50-1.43 (m, 2H), 1.31-1.27 (brs, 38H), 0.87 (t, J=6.4 Hz, 3H).

### Synthesis of (S)-1-(docosyloxy)-3-hydroxypropan-2-yl methylcarbamate (Compound 8)

To a stirred solution of (R)-1-(docosyloxy)-3-(trityloxy)propan-2-yl methylcarbamate (6-7) (6 g, 8.5773 mmol) in mixture of DCM:MeOH (1:1) 60 mL:60 mL solvents at 0° C. was added DL-10-camphor sulphonic acid (1.99 g, 8.5773 mmol). The whole reaction mixture was stirred at r.t for 2 h. Completion of the reaction was monitored by TLC. The reaction mixture was diluted with DCM (200 mL) and washed with water (50.0 mL×2), extracted, and separated, and the organic layer was dried over anhydrous sodium sulphate and concentrated to get the crude compound. The crude was washed with n-pentane (100 mL), stirred for 15 min, and an off-white solid was precipitated, which was filtered and dried to afford Compound 8 (1.5 g) as an off-white solid. ¹H NMR (CDCl3) 400 MHz) δ ppm 4.90-4.87 (m, 1H), 4.80 (bs, 1H), 3.85-3.81 (m, 2H), 3.64-3.61 (m, 2H), 3.47-3.43 (m, 2H), 2.81 (d, J=5.2 Hz, 3H), 2.62 (t, J=6 Hz, 1H), 1.63-1.52 (m, 2H), 1.4-1.2 (brs, 38H), 0.88 (t, J=6.4 Hz, 3H).

### Synthesis of (R)-1-((bis(benzyloxy)phosphoryl)oxy)-3-(docosyloxy)propan-2-yl methylcarbamate (6-10)

To the stirred solution of Compound 8 ((S)-1-(docosyloxy)-3-hydroxypropan-2-yl methylcarbamate (0.5 g, 1.092 mmol)) in dry THF (100 mL) solvent at 0° C. was added potassium tert-butoxide (0.245 g, 2.1846 mmol). The mixture was stirred for 10 min, tetrabenzyl diphosphate (1.17 g, 2.1846 mmol) was added, and the reaction mixture was stirred at 0° C. for 3 h. TLC indicated starting material along with product formation. Further potassium tertiary-butoxide (0.12 g, 1.0923 mmol) and tetrabenzyldiphosphate (0.6 g, 1.0923 mmol) was added and stirred for another 3 h at 0° C. Completion of the reaction was monitored by TLC. The reaction mixture was quenched with ice water and extracted with ethyl acetate (2×100 mL). The organic layer was dried over anhydrous sodium sulphate and concentrated to get 1.3 g crude compound and purified by Chiral prep-HPLC purification, resulting in separation of Peak-1 and Peak-2. Peak-1 (as a pale pink liquid) was afford as the title compound 6-10 (130 mg). Confirmed by 1H NMR, 31P NMR.

¹H NMR (CDCl3) 400 MHz) δ ppm 7.36-7.3 (bs, 10H), 5.1-5.0 (m, 4H), 4.69 (bs, 1H), 4.30-4.27 (m, 1H), 4.19-4.13 (m, 1H), 355-3.51 (m, 2H), 3.41-3.36 (m, 2H), 2.67 (bs, 3H), 1.52-1.47 (m, 2H), 1.33-1.24 (bs, 38H), 0.88 (t, J=6.4 Hz, 3H.

31P NMR (CDCl3) 400 MHz) δ ppm −1.913 (bs), HRMS: (M+1)=718.4727.

HPLC: t_{Ret} 11.017 min (99.35%) HPLC Method conditions: Column: Kinetex EVO, C₁₈ (150*4.6) mm, 5 μm, 100A Mobile phase-A: 0.1% Formic Acid in (Aq); Mobile phase-B: ACN 100% Method-T/% B: −0/60, 2/60, 6/100, 16/100, 17/60, 18/60 Flow rate: 1.5 ml/min Column temp: 30° C. Diluent: THF

### Synthesis of (R)-1-(docosyloxy)-3-(phosphonooxy)propan-2-yl methylcarbamate: Compound 6

To the stirred solution of (R)-1-((bis(benzyloxy)phosphoryl)oxy)-3-(docosyloxy)propan-2-yl methylcarbamate 6-10 (130 mg, 0.181 mmol) in EtOAc, 20% Pd(OH)₂/C (50 mg) was added and hydrogenated at 40 PSI for 2 h. The completion of the reaction was monitored by TLC. The reaction mixture was diluted with 10% MeOH in DCM and filtered through Celite. The filtrate was concentrated under reduced pressure to get crude product. The crude product was washed with pentane and dried to get Compound 6 as an off-white solid (50 mg). Confirmed by 1H NMR, 31P NMR.

¹H NMR (CD3OD) 400 MHz VT at 50° C. δ ppm 4.38-4.36 (bs, 1H), 4.19-4.16 (m, 1H), 4.08-4.04 (m, 1H), 3.56-3.5 (m, 2H), 3.38 (t, J=6.4 Hz, 2H), 2.60 (s, 3H), 1.49-1.42 (m, 2H), 1.3-1.11 (bs, 38H).

31P NMR (CDCl3) 400 MHz) δ ppm 0.276 (bs)

HPLC: tRet 7.616 min (99.58%) HPLC Method conditions: Column: LUNA HILIC (250*4.6) mm, 5 μm, 200A Mobile phase-A: 10 Mm Ammonium Acetate in (Aq); Mobile phase-B: ACN 100% Method-T/% B: −0/10, 2/10, 6/100, 13/100, 14/10, 15/10 Flow rate: 1.0 ml/min Column temp: 30° C. Diluent: THF.

### Example S-7: Synthesis of Compound 4

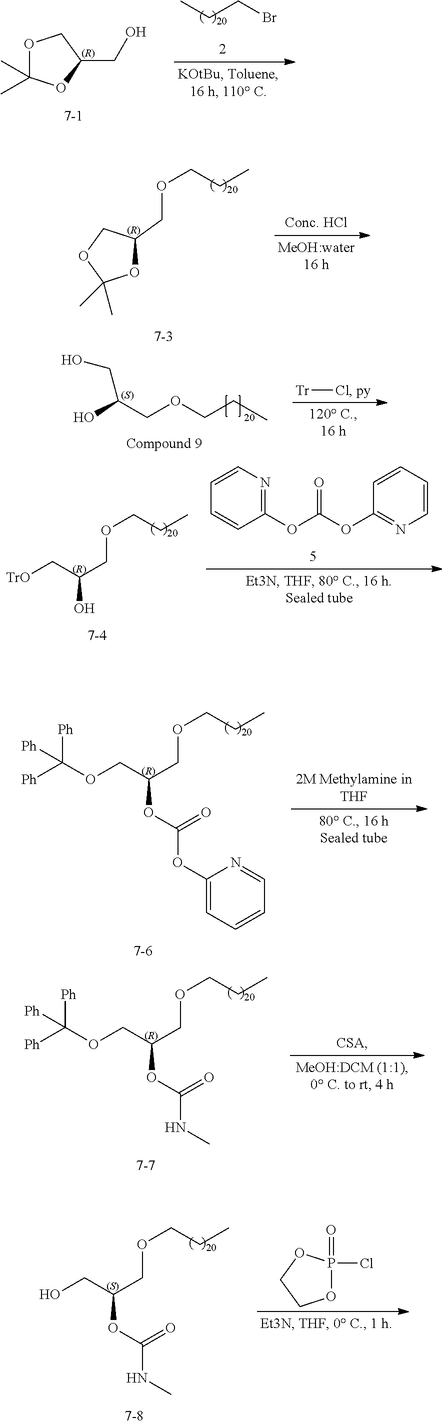

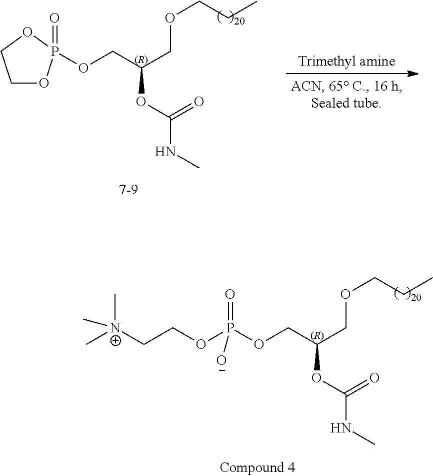

### Synthesis of (R)-4-((docosyloxy)methyl)-2,2-dimethyl-1,3-dioxolane (7-3)

To the stirred solution of 1-bromodocosane (16.96 g, 128.3664 mmol) in toluene at 0° C., potassium tertiary-butoxide (28.8 g, 256.7328 mmol) and (R)-(2,2-dimethyl-1,3-dioxolan-4-yl)methanol (16.96 g, 128.3664 mmol) was added. The reaction mixture became a thick mass and stirring was stopped. The reaction mixture stirred at RT for 1 h and then heated to 110° C. for 16 h. The completion of the reaction was monitored by TLC. After completion of the reaction, ether was added to the reaction mixture and stirred for 10 min, brine solution was added to the reaction mixture and extracted with ether. The organic layer was dried over anhydrous Na₂SO₄ and concentrated under reduced pressure to get crude product 3 (60 g) as brown color solid. Confirmed by 1H NMR. ¹H NMR (CDCl3) 400 MHz δ ppm 5.01-4.91 (m, 1H), 4.29-4.22 (m, 1H), 4.06-4.04 (m, 1H), 3.74-3.72 (m, 1H), 3.53-3.39 (m, 3H), 1.59-1.53 (m, 2H), 1.45-1.18 (brm, 40H), 0.86 (t, J=13.6 Hz, 3H). Crude 1H NMR shows the desired product along with impurities; 1H NMR values were assigned based on product peaks in the next step.

### Synthesis of Compound 9

To the stirred solution of (R)-4-((docosyloxy)methyl)-2,2-dimethyl-1,3-dioxolane (60 g, 136.1315 mmol) in MeOH (500 mL), Conc. HCl (125 mL), was added and heated to 70° C. for 16 h. The completion of the reaction was monitored by TLC. The reaction mixture was diluted with water and filtered, the filtered solid was again stirred with water and filtered to get solid. The solid was stirred with hexane and filtered to get product. The product contains water, Acetonitrile was added to the product and distilled thrice to remove moisture to afford Compound 9 as an off-white solid (30 g, 55% over two steps). Confirmed by ¹H NMR (CDCl₃) 400 MHz): ¹H NMR (CDCl3) 400 MHz δ ppm 3.86 (bs, 1H), 3.71-3.66 (m, 2H), 3.53-3.44 (m, 4H), 2.59 (bs, 1H), 2.15 (bs, 1H), 1.58-1.54 (m, 2H), 1.38-1.18 (bs, 40H), 0.88 (t, J=6.4 Hz, 3H).

### Synthesis of (R)-1-(docosyloxy)-3-(trityloxy) propan-2-ol (7-4)

To the stirred solution of (S)-3-(docosyloxy)propane-1,2-diol (20.0 g, 49.913 mmol) in pyridine (100.0 mL) at 0° C., trityl chloride (13.91 g, 49.913 mmol) was added at 0° C. and heated to 120° C. for 16 h in sealed tube. (Note that the starting material, pyridine, and tritylchloride should be anhydrous; if the reaction mixture contains any moisture, the reaction will not proceed.) The completion of the reaction was monitored by TLC. After completion of the reaction, the reaction mixture was evaporated under reduced pressure to get crude. The crude product was purified by combiflash chromatography using 5% EtOAc in hexane as eluent. After evaporation of fractions, the product was washed with n-pentane (500 mL), stirred for 1 h, filtered and dried to get the white solid as the desired compound with some contamination of trityl chloride. The desired product 7-4 (19.4 g, 60% as a white solid was obtained and characterized and confirmed by 1HNMR: ¹H NMR (400 MHz, CDCl₃): δ=7.25-7.43 (m, 15H), 3.94 (m, 1H), 3.42-3.52 (m, 4H), 3.18 (m, 2H), 1.58-1.54 (m, 2H), 1.22-1.48 (m, 38H), 0.87 (m, 3H). The crude 1H NMR showed the desired product along with impurities. 1H NMR values were assigned on the basis of product peaks in a following step.

### Synthesis of (R)-1-(docosyloxy)-3-(trityloxy)propan-2-yl pyridin-2-yl carbonate (7-6)

To the stirred solution of (R)-1-(docosyloxy)-3-(trityloxy)propan-2-ol (5 g, 7.7759 mmol) in THF at RT, Et₃N (4.3 mL, 31.1036 mmol) was added followed by the addition of di(pyridin-2-yl) carbonate (3.36 g, 15.5518 mmol) and stirred at 80° C. for 16 h in a sealed tube. The completion of the reaction was monitored by TLC. Due to instability of product reaction mixture, this crude mixture was taken into the next step without work-up to afford (R)-1-(docosyloxy)-3-(trityloxy)propan-2-yl pyridin-2-yl carbonate 7-6 (5 g, crude) as pale brown reaction mixture. ¹H NMR (CDCl3) 400 MHz) δ ppm 7.80-7.76 (m, 1H), 7.47-7.27 (m, 16H), 6.57 (d, J=9.2 Hz, 1H), 6.30-6.27 (m, 1H), 5.14-5.12 (m, 1H), 3.75-3.33 (m, 6H), 1.52-1.49 (m, 2H), 1.4-1.2 (bs, 38H), 0.87 (t, J=6.4 Hz, 3H). The crude 1H NMR showed the desired product along with impurities. 1H NMR values were assigned on the basis of product peaks in a following step.

### Synthesis of (R)-1-(docosyloxy)-3-(trityloxy)propan-2-yl methylcarbamate (7-7)

To the crude compound (R)-1-(docosyloxy)-3-(trityloxy)propan-2-yl pyridin-2-yl carbonate in THF (100 ml) was added 2M methylamine in THF (50 mL), and heated to 80° C. for 16 h. The completion of the reaction was monitored by TLC. The reaction mixture was evaporated under reduced pressure to get crude. The crude product was dissolved in EtOAc (300 mL) and washed with water (2×200 mL), the organic layer was dried over anhydrous Na₂SO₄ and concentrated under reduced pressure to get product. The crude compound was proceeded to next step as such. (9 g, Crude) and characterized by 1HNMR: ¹H NMR (CDCl3) 400 MHz) δ ppm 7.43-7.18 (m, 15H), 5.08-5.06 (m, 1H), 4.74-4.72 (m, 1H), 3.65-3.60 (m, 2H), 3.43-3.36 (m, 2H), 3.27-3.21 (m, 2H), 2.80 (d, J=4.8 Hz, 3H), 1.50-1.47 (m, 2H), 1.28 (brs, 40H), 0.87 (t, J=6.4 Hz, 3H).

### Synthesis of (S)-1-(docosyloxy)-3-hydroxypropan-2-yl methylcarbamate (7-8)

To the stirred solution of (R)-1-(docosyloxy)-3-(trityloxy)propan-2-yl methylcarbamate (9.0 g, 12.865 mmol) in MeOH and DCM 100 mL (1:1) was added camphorsulphonic acid (2.98 g, 12.865 mmol) at 0° C. The reaction mixture was stirred at RT for 2 h. The completion of the reaction was monitored by TLC. The reaction mixture was diluted with DCM (300.0 mL) and washed with water (200 mL×2), then the organic layer was dried over anhydrous sodium sulphate and concentrated to get the crude compound. Crude was washed with pentane to get the pure title compound (2.8 g, pure) as off-white solid and characterized by 1HNMR: ¹H NMR (CDCl3) 400 MHz) δ ppm 5.02 (bs, 1H), 4.89-4.87 (m, 1H), 3.80-3.77 (m, 2H), 3.63-3.61 (m, 2H), 3.47-3.43 (m, 2H), 3.01-2.98 (m, 1H), 2.79 (d, J=4.8 Hz, 3H), 1.57-1.52 (m, 2H), 1.28 (s, 40H), 0.88 (t, J=6.4 Hz, 3H).

### Synthesis of (R)-1-(docosyloxy)-3-((2-oxido-1,3,2-dioxaphospholan-2-yl)oxy)propan-2-yl methylcarbamate (7-9)

To the stirred solution of (S)-1-(docosyloxy)-3-hydroxypropan-2-yl methylcarbamate (0.3 g, 0.655 mmol) and Et₃N (0.27 mL, 1.967 mmol) in THF (5 mL) was added 2-chloro-1,3,2-dioxaphospholane-2-oxide (0.18 mL, 1.967 mmol) at 0° C., and the reaction mixture was stirred for 1 h. The completion of the reaction was monitored by TLC. The reaction mixture was filtered, the filtrate was concentrated under vacuum, and the obtained crude compound (0.3 g) was characterized by 1HNMR and used in the next step. ¹H NMR (CDCl3) 400 MHz) δ ppm 4.98-4.96 (m, 1H), 4.46-4.43 (m, 2H), 4.28-4.25 (m, 4H), 3.74-3.71 (m, 2H), 3.55-3.44 (m, 2H), 2.69 (s, 3H), 1.55-1.52 (m, 2H), 1.30 (bs, 40H), 0.90-0.87 (m, 3H).

### Synthesis of (R)-3-(docosyloxy)-2-((methylcarbamoyl)oxy) propyl (2-(trimethylammonio) ethyl) phosphate: Compound 4

To the stirred solution of (R)-1-(docosyloxy)-3-((2-oxido-1,3,2-dioxaphospholan-2-yl) oxy) propan-2-yl methylcarbamate (0.3 g, 0.532 mmol) in ACN (15 mL) was added solution of 2M trimethylamine (3 mL) at 0° C. and reaction mixture was stirred for 16 h at 65° C. The completion of the reaction was monitored by TLC. The reaction mixture was cooled to 0° C. then solid precipitated out which was filtered and dried under vacuum to obtain crude material. Crude compound washed with water (15 mL) and acetonitrile (30 mL) to afford the title compound (0.23 g, pure) as an off white solid and characterized by ¹H NMR: ¹H NMR (CDCl3) 400 MHz) δ ppm 4.97-4.95 (m, 1H), 4.27-4.25 (m, 2H), 4.01-3.96 (m, 2H), 3.63-3.58 (m, 4H), 3.48-3.40 (m, 2H), 3.22 (s, 9H), 2.69 (s, 3H), 1.55-1.53 (m, 2H), 1.28 (bs, 40H), 0.90 (t, J=5.6 Hz, 3H). 31P NMR (CD₃OD, 162 MHz): 0.17 ppm. HRMS=622.6053.

### Example S-8: Synthesis of Compound 14, Compound 15, and Compound 16

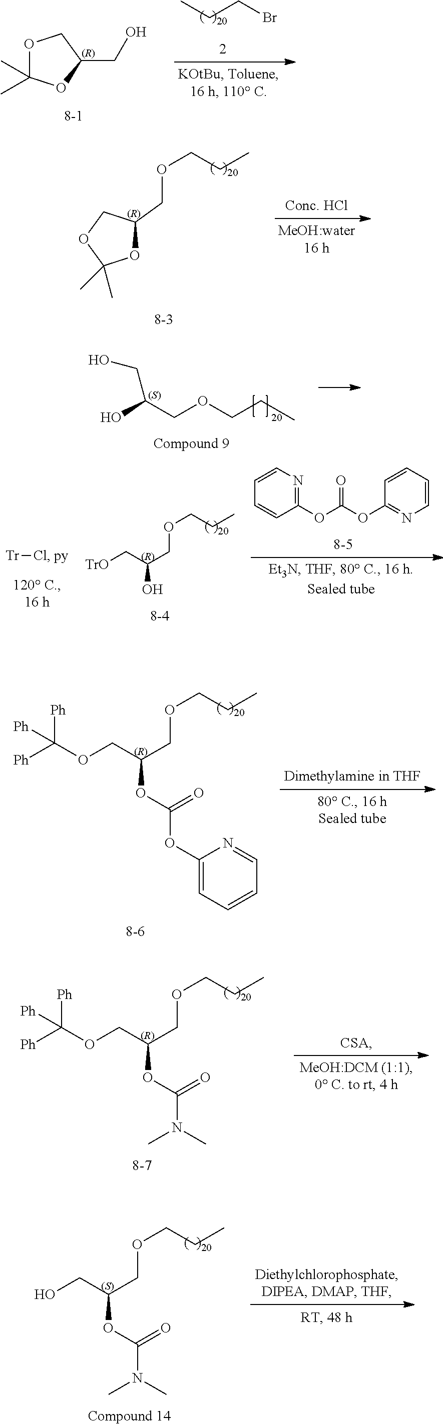

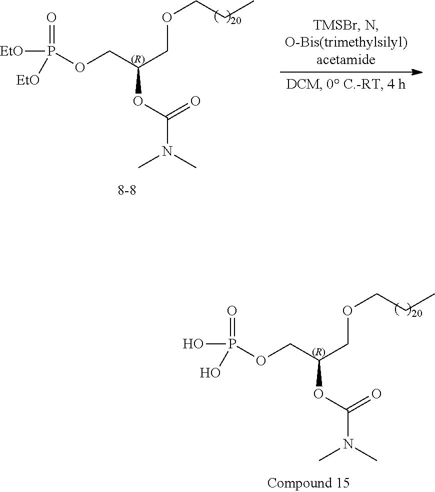

### Synthesis of (R)-4-((docosyloxy)methyl)-2,2-dimethyl-1,3-dioxolane (8-3)

To a stirred solution of 1-bromodocosane (16.96 g, 128.3664 mmol) in Toluene at 0° C., potassium tertiary-butoxide (28.8 g, 256.7328 mmol) and (R)-(2,2-dimethyl-1,3-dioxolan-4-yl)methanol (16.96 g, 128.3664 mmol) was added. The reaction mixture became a thick mass and stirring was stopped, the reaction mixture was warmed to RT and stirred at RT for 1 h, and then the reaction mixture was heated to 110° C. for 16 h. The completion of the reaction was monitored by TLC. After completion of the reaction, ether was added to the reaction mixture and stirred for 10 min. Brine solution was added to the reaction mixture and extracted with ether. The organic layer was dried over anhydrous Na₂SO₄ and concentrated under reduced pressure to get crude product (60 g) as a brown colored solid. Confirmed by 1H NMR: ¹H NMR (CDCl3) 400 MHz δ ppm 5.01-4.91 (m, 1H), 4.29-4.22 (m, 1H), 4.06-4.04 (m, 1H), 3.74-3.72 (m, 1H), 3.53-3.39 (m, 3H), 1.59-1.53 (m, 2H), 1.45-1.18 (brm, 44H), 0.86 (t, J=13.6 Hz, 3H). Crude 1HNMR showed the desired product, along with impurities; 1HNMR values were assigned on the basis of product peaks in following steps.

### Synthesis of Compound 9

To the stirred solution of (R)-4-((docosyloxy)methyl)-2,2-dimethyl-1,3-dioxolane (60 g, 136.1315 mmol) in MeOH (500 mL), conc. HCl (125 mL) was added and heated to 70° C. for 16 h. The completion of the reaction was monitored by TLC. The reaction mixture was diluted with water and filtered, the filtered solid was again stirred with water and filtered to get a solid. The solid was stirred with hexane and filtered to get product. The product contained water, so acetonitrile was added to the product and evaporated three times to remove moisture to afford Compound 9 (30 g, 55% over two steps) as an off white solid. Confirmed by ¹H NMR (CDCl₃) 400 MHz) ¹H NMR (CDCl3) 400 MHz δ ppm 3.86 (bs, 1H), 3.71-3.66 (m, 2H), 3.53-3.44 (m, 4H), 2.59 (bs, 1H), 2.15 (bs, 1H), 1.58-1.54 (m, 2H), 1.38-1.18 (bs, 38H), 0.88 (t, J=6.4 Hz, 3H).

### Synthesis of (R)-1-(docosyloxy)-3-(trityloxy) propan-2-ol (8-4)

To the stirred solution of (S)-3-(docosyloxy)propane-1,2-diol (20.0 g, 49.913 mmol) in pyridine (100.0 mL) at 0° C., trityl chloride (13.91 g, 49.913 mmol) was added at 0° C. and heated to 120° C. for 16 h in sealed tube. (In this step, the starting material, pyridine and trityl chloride should be anhydrous; if the reaction mixture contains any moisture, the reaction will not proceed.) The completion of the reaction was monitored by TLC. After completion of the reaction, the reaction mixture was evaporated under reduced pressure. The crude product was purified by combiflash chromatography using 5% EtOAc in hexane as eluent. After evaporation of fractions, the material was washed with n-pentane (500 mL), stirred for 1 h, filtered and dried to get the white solid as a desired compound with trityl chloride contaminant. The desired product 8-4 (19.4 g, 60%) as white solid was confirmed by 1H NMR. ¹H NMR (400 MHz, CDCl₃): δ=7.25-7.43 (m, 15H), 3.94 (m, 1H), 3.42-3.52 (m, 4H), 3.18 (m, 2H), 1.22-1.48 (m, 38H), 0.87 (m, 3H).

### Synthesis of (R)-1-(docosyloxy)-3-(trityloxy)propan-2-yl pyridin-2-yl carbonate (8-6)

To the stirred solution of (R)-1-(docosyloxy)-3-(trityloxy)propan-2-ol (5 g, 7.7759 mmol) in THF at RT, Et₃N (4.3 mL, 31.1036 mmol) was added followed by the addition of di(pyridin-2-yl) carbonate (3.36 g, 15.5518 mmol) and stirred at 80° C. for 16 h in a sealed tube. The completion of the reaction was monitored by TLC. Due to instability of product reaction mixture, this crude mixture was used in the next step without work-up to afford (R)-1-(docosyloxy)-3-(trityloxy)propan-2-yl pyridin-2-yl carbonate 8-6 (5 g, crude) as a pale brown reaction mixture.

¹H NMR (CDCl3) 400 MHz) δ ppm 7.80-7.76 (m, 1H), 7.47-7.27 (m, 16H), 6.57 (d, J=9.2 Hz, 1H), 6.30-6.27 (m, 1H), 5.14-5.12 (m, 1H), 3.75-3.33 (m, 6H), 1.52-1.49 (m, 2H), 1.4-1.2 (bs, 38H), 0.87 (t, J=6.4 Hz, 3H). 1H NMR of crude compound showed the desired product along with impurities; 1H NMR values were assigned on the basis of product peaks in a following step.

### Synthesis of (R)-1-(docosyloxy)-3-(trityloxy)propan-2-yl dimethylcarbamate (8-7)

To the crude product 8-6 (R)-1-(docosyloxy)-3-(trityloxy)propan-2-yl pyridin-2-yl carbonate in THF 50 ml was added 7% dimethyl amine in THF (40 mL) and heated to 80° C. for 16 h. The completion of the reaction was monitored by TLC. The reaction mixture was evaporated under reduced pressure. The crude product was dissolved in EtOAc (200 mL) and washed with water (2×50 mL), and the organic layer was dried over anhydrous Na₂SO₄ and concentrated under reduced pressure to get product. The crude compound purified by combiflash chromatography. The product was eluted in 5% EtOAc in 1% triethylamine in hexane. The fractions containing product was concentrated and dried completely to afford the title product 7 (6 g, Pure) as off-white solid and characterized by 1HNMR. ¹H NMR (CDCl₃) 400 MHz) δ ppm 7.45-7.43 (m, 5H), 7.30-7.20 (m, 10H), 5.08-5.05 (m, 1H), 3.66-3.63 (m, 2H), 3.41-3.38 (m, 2H), 3.27-3.23 (m, 2H), 2.95-2.94 (bs, 6H), 1.49-1.46 (m, 2H), 1.31-1.23 (brs, 38H), 0.87 (t, J=6.4 Hz, 3H).

### Synthesis of (S)-1-(docosyloxy)-3-hydroxypropan-2-yl dimethylcarbamate (Compound 14)

To the stirred solution (R)-1-(docosyloxy)-3-(trityloxy)propan-2-yl dimethylcarbamate (3.0 g, 4.2011 mmol) in MeOH and DCM 30 mL (1:1) was added camphorsulphonic acid (1.46 g, 6.3017 mmol) at 0° C. The reaction mixture was stirred at RT for 4 h. The completion of the reaction was monitored by TLC. The reaction mixture was concentrated to dryness. The residue was dissolved in ethyl acetate (250 mL), washed with water (50 mL×2), dried over sodium sulphate and concentrated to dryness. The crude compound of a previous batch was mixed with the present batch and purified by combiflash chromatography. The product was eluted in 40% EtOAc in hexanes. The fractions containing product were concentrated and dried completely to afford the title Compound 14 (3.2 g, Pure) as an off-white foam solid, and characterized by 1HNMR. ¹H NMR (CDCl3) 400 MHz) δ ppm 4.87-4.85 (m, 1H), 3.83-3.87 (m, 2H), 3.67-3.59 (m, 2H), 3.47-3.43 (m, 2H), 2.901 (bs, 6H), 2.88-2.87 (m, 1H), 1.57-1.52 (m, 2H), 1.28-1.21 (bs, 38H), 0.88 (t, J=6.4 Hz, 3H). HRMS (M+1)=472.1716.

### Synthesis of (R)-1-((diethoxyphosphoryl)oxy)-3-(docosyloxy)propan-2-yl dimethylcarbamate (8-8)

To a stirred solution of (S)-1-(docosyloxy)-3-hydroxypropan-2-yl dimethylcarbamate Compound 14 (1 g, 2.1196 mmol) in THF (10 mL) at 0° C. DIPEA (1.84 mL, 10.5983 mmol) and DMAP (0.51 g, 4.2392 mmol) was added, followed by the dropwise addition of diethylchlorophosphate (1.52 mL, 10.5983 mmol) and stirred at RT for 24 h. TLC indicated starting material along with product formation. Further DIPEA (0.92 mL, 5.299 mmol), DMAP (0.26 g, 2.1196 mmol) and diethylchlorophosphate (0.76 mL, 5.299 mmol) was added and stirred at RT for another 24 h. The completion of the reaction was monitored by TLC. The reaction mixture was diluted with ethyl acetate (100 mL) and washed with water (2×50 mL), the organic layer was separated and dried over sodium sulphate and concentrated under reduced pressure to get crude product. The crude product was purified by combiflash chromatography. The product was eluted in 30% EtOAc in hexanes. The fractions containing product were concentrated and dried completely to afford the title compound (1 g, pure) as a pale pink waxy solid and characterized by 1H NMR & 31P NMR: ¹H NMR (CDCl3) 400 MHz) δ ppm 5.03-4.89 (m, 1H), 4.23-4.09 (m, 6H), 3.59-3.57 (m, 2H), 2.87 (s, 6H), 1.58-1.50 (m, 2H), 1.33-1.15 (m, 44H), 0.88 (t, J=6.4 Hz, 3H); 31P NMR single peak at −0.437 was observed in CDCl3.

### Synthesis of (R)-1-(docosyloxy)-3-(phosphonooxy)propan-2-yl dimethylcarbamate: Compound 15

To a stirred solution of 8-8 (R)-1-((diethoxyphosphoryl)oxy)-3-(docosyloxy)propan-2-yl dimethylcarbamate (1 g, 1.6451 mmol) in DCM (10 mL) at 0° C., TMSBr (1.3 mL, 9.8708 mmol) was added followed by the addition of N,O-Bis(trimethylsilyl)acetamide (2.4 mL, 9.8708 mmol) and stirred at RT for 4 h. Progress of the reaction is monitored by TLC. The reaction mixture was cooled to 0° C. and 1 mL methanol was added and stirred for 10 min. 1 mL water was added to the reaction mixture and stirred at 0° C. for another 10 min, sat. NaHCO₃ solution (50 ml) was added to the reaction mixture, and washed with EtOAc three times (3×50 mL). The aqueous layer was acidified with 6 N HCl solution slowly at 0° C. and extracted with ether. The organic layer was dried over anhydrous Na₂SO₄ and concentrated under reduced pressure to get crude gummy white solid. The gummy white solid was stirred with acetonitrile and methanol and filtered to afford the title compound (0.35 g, pure) as an off white solid and characterized by 1 HNMR. ¹H NMR (CD3OD) 400 MHz) δ ppm 4.97-4.90 (m, 1H), 4.14-4.08 (m, 2H), 3.60-3.59 (m, 2H), 3.49-3.43 (m, 2H), 3.45-3.43 (m, 2H), 2.92 (d, J=16 Hz, 6H), 1.40-1.20 (m, 38H), 0.89 (t, J=6.4 Hz, 3H). 31PNMR single peak at 1.312 was observed in CD3OD. HRMS: (M+1)=551.044.

### Synthesis of (R)-2-((dimethylcarbamoyl)oxy)-3-ethoxypropyl (2-(trimethylammonio)ethyl) phosphate-icosane (1/1), Compound 16

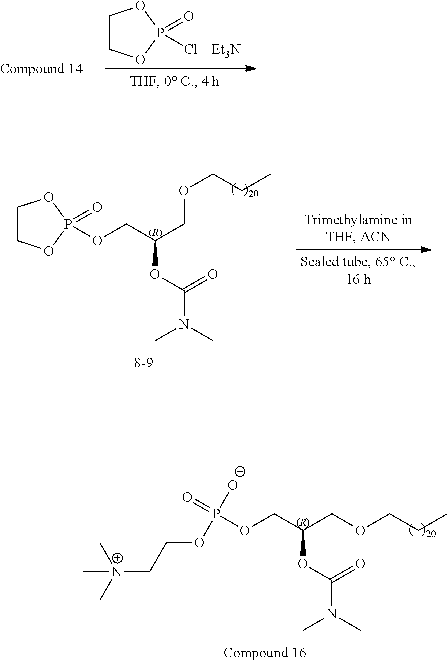

### Synthesis of (R)-1-(docosyloxy)-3-((2-oxido-1,3,2-dioxaphospholan-2-yl)oxy)propan-2-yl dimethylcarbamate (8-9)

To a stirred solution of (S)-1-(docosyloxy)-3-hydroxypropan-2-yl dimethylcarbamate Compound 14 (0.5 g, 1.0598 mmol) in THF at 0° C., triethylamine (0.44 mL, 3.1795 mmol) was added followed by the addition of 2-chloro-1,3,2-dioxaphospholane 2-oxide (0.3 mL, 3.1795 mmol) and stirred at 0° C. for 4 h. The completion of the reaction mixture was monitored by TLC. The reaction mixture was filtered to remove salts and the filtrate was concentrated under reduced pressure to get 0.62 g crude. The crude product was used in the next step based on TLC and crude NMR without any workup and purification due to the unstable nature of the product.

### Synthesis of (R)-2-((dimethylcarbamoyl)oxy)-3-ethoxypropyl (2-(trimethylammonio)ethyl) phosphate-icosane (1/1), Compound 16

To a stirred solution of (R)-1-(docosyloxy)-3-((2-oxido-1,3,2-dioxaphospholan-2-yl)oxy)propan-2-yl dimethylcarbamate (0.62 g, 1.073 mmol) in acetonitrile, trimethylamine (5 mL) was added and heated to 65° C. for 16 h in a sealed tube. Progress of the reaction was monitored by TLC. The reaction mixture was filtered and the solid was dried to get product. The crude product was purified by combiflash chromatography. The product was eluted in 40% MeOH in DCM as eluent. The fractions containing product were concentrated and dried completely to afford impure compound. The compound was stirred with 10% THF in acetonitrile and dried to afford the title compound (0.1 g, pure) as off white solid and characterized by 1HNMR & HRMS. ¹H NMR (CD3OD) 400 MHz) δ ppm 4.982-4.905 (m, 1H), 4.259 (bs, 2H), 4.02-3.98 (m, 2H), 3.61-3.60 (m, 4H), 3.49-3.43 (m, 2H), 3.215 (s, 9H), 2.93-2.89 (m, 6H), 1.54-1.53 (m, 2H), 1.26 (BS, 40H), 0.89 (t, J=6.4 Hz, 3H). 31PNMR single peak at 0.893 was observed in CD3OD. HRMS: (M+1)=636.6127.

### Example P-1: Preparation of Lipid Nanoparticles Comprising an Ether Lipid (ETL) or Ether Phospholipid (ETPL)

This example describes preparation of lipid nanoparticles (LNPs) loaded with a hyperactivating lipid (an ETL or ETPL) in a microfluidic process.

### Materials and Methods

LNPs are synthesized using the NanoAssembir® Ignite™ microfluidic instrument (Precision Nanosystems, Vancouver, BC, Canada). A kit containing GenVoy-ILM™ ionizable lipid mix (Precision Nanosystems, Vancouver, BC, Canada) is used to produce LNPs. The kit without mRNA is used to build empty LNP vehicles, and hyperactivator loaded LNPs are generated by adding the appropriate ETL or ETPL to a molar ratio of 10% of the total LNP content. LNPs are also produced using individual components (without a kit) to determine if ETL or ETPL loading into LNPs can be intentionally varied. Lipids are first dissolved in ethanol and then combined following the molarity percentages shown in Table III. Lipids in ethanol are combined with PBS, pH 7.4 at a 1:3 volumetric ratio. The NanoAssemblr® Ignite™ microfluidic instrument is programmed with a flow rate of 12 mL/min, a start waste of 0.35 mL, and an end waste of 0.05 mL. LNPs are washed in PBS, pH 7.4 to remove residual ethanol, and then are concentrated using Amicon 10K MWCO centrifugal filters by spinning at 2000×g for 30 minutes.

TABLE III
LNP Formulations\hat{ }

| | GenVoy | ETL/ETPL | LNP | ETL/ETPL | LNP | ETL/ETPL | ETL/ETPL |
|---|---|---|---|---|---|---|---|
| | Vehicle | GenVoy | Vehicle | LNP | Vehicle | LNP | LNP |
| Lipid | LNP | LNP | 1 | 1 | 2 | 2 | 3 |
| GenVoy-ILM | 100.0% | 90.0% | — | — | — | — | — |
| MC3 | — | — | 45.0% | 41.0% | — | — | — |
| DSPC | — | — | 15.0% | 10.0% | 50.0% | 25.0% | 10.0% |
| Cholesterol | — | — | 38.5% | 37.5% | 48.5% | 48.5% | 48.5% |
| DMG-PEG2000 | — | — | 1.5% | 1.5% | 1.5% | 1.5% | 1.5% |
| ETL/ETPL | — | 10.0% | — | 10.0% | — | 25.0% | 40.0% |

\hat{ }Percent molarity of components of different LNP formulations. LNP 2 and LNP 3 formulations share the same vehicle (LNP Vehicle 2).

Loading of an ETL or ETPL into LNPs is assessed using HPLC. LNPs in PBS are frozen at −80° C., then lyophilized and stored at −20° C. until they are quantified. LNPs are reconstituted in ethanol, and then mixed with water to dissolve the PBS. A seven point standard curve of ETL or ETPL is prepared in ethanol with water and PBS is added to match sample preparation. Standards and samples are filtered through a 0.45 um filter prior to running on the HPLC. HPLC quantification is performed using an Agilent 1260 Infinity II HPLC equipped with a 1260 Infinity II Evaporative Light Scattering Detector (ELSD). A Luna 5 μm NH2 100 Å, 150×4.6 mm LC Column (Phenomenex, Torrance, CA) with a column temperature of 30° C. is used to detect samples. Two eluents are used: A, 100% water; and B, 100% acetonitrile. An initial mobile phase composed of 5%/95% A/B is used to load the column, with a gradient reaching 24%/76% A/B after 2.5 min. A more shallow gradient is used from 2.5 to 6 min, with A/B slowly reaching 25%/75% during that time frame. A post time of 3 min is used to return the gradient to starting conditions prior to the next sample run. The flow rate is set to 1 mL/min, and the injection volume is 5 μL for samples and standards. The ELSD uses an evaporator temperature of 80° C., a nebulizer temperature of 30° C., and a nitrogen gas flow rate of 0.9 standard liters/min. Agilent CDS 2.6 software is used for HPLC instrument control, data acquisition, and processing.

The size of the LNPs is assessed using dynamic light scattering (DLS) on the NanoBrook Omni particle size and zeta potential analyzer (Brookhaven Instruments Corp., Holtsville, NY). Four measurements are made for each sample for 120 seconds each, with the first measurement made for each sample excluded from downstream analyses as time needed for sample equilibration.

### BIOLOGICAL EXAMPLES

Biological Abbreviations: AUC (area under curve); BAL (bronchoalveolar lavage); cDC (classical DC); DAMP (damage-associated molecular pattern); DC (dendritic cell); DGPC (Compound 1); DGP (Compound); dLN (draining lymph node); DP (drug product); DS (drug substance); GC (germinal center); GMT (geometric mean titer); HA (hemagglutinin); HAI (hemagglutinin inhibition); HD (human donor); IL-1β or IL-1b (interleukin-1beta); IM (intramuscular); IN (intranasal); KP407 or P407 (Kolliphor P407); LDH (lactate dehydrogenase); LPC (lyso phosphatidylcholine); LPS (lipopolysaccharide); MFI (mean fluorescence intensity); moDC (monocyte-derived DC); NP (nucleoprotein); ns (not significant); OVA (ovalbumin); PAMP (pathogen-associated molecular pattern); PBMS (peripheral blood mononuclear cells); PBS (phosphate-buffered saline); PC (phosphatidylcholine); PFU (plaque-forming unit); R848 (resiquimod); RT (RT); SC (subcutaneous); SD (standard deviation); SFC (spot-forming cell); and SFU (spot-forming unit); TCM (T central memory); TEM (T effector memory); TFH (T follicular helper); and WTL (whole tumor lysate).

### INTRODUCTION

Dendritic cell (DC) hyperactivation is a cellular state defined by its ability to secrete IL-1β while remaining viable. IL-1βeta is an important cytokine in the induction of T cell responses. IL-1β is synthesized within a cell in a pro-form that is then cleaved by an activated inflammasome. Dendritic cell (DC) hyperactivation is a cellular state defined by its ability to secrete IL-1β while remaining viable. IL-1β is an important cytokine in the induction of T cell responses. IL-1β is synthesized within a cell in a pro-form that is then cleaved by an activated inflammasome. The mature (active) form of the cytokine is then released via pores formed as a result of inflammasome activation. Previously, inflammasome activation and pore formation was thought to result in pyroptotic cell death. However, hyperactivated DCs remain alive while secreting IL-1β. Additionally, hyperactive DCs have enhanced migratory capacity. As a result, these antigen presenting cells can traffic to lymph nodes where they can signal to other immune cell types and initiate adaptive immune responses. DC hyperactivation leads to enhanced T cell responses with an especially durable memory population (Zhivaki et al., Cell Reports, 33 (7), 2020, 108381). The enhanced T cell responses can be harnessed to treat diseases such as cancer.

Previous work identified oxidized lipids and lyso phosphatidylcholine (PC) lipids as chemical entities that induce DC hyperactivation. Structure-activity relationship data suggests that the acyl chain length of lyso PC lipids is critical to hyperactivity. As the acyl chain length increases, the molecule becomes more potent at inducing IL-1βeta secretion from human DCs. The compound 22:0 lyso PC (1-behenoyl-2-hydroxy-sn-glycero-3-phosphocholine) has been identified as a potent hyperactivating lipid. The inventors sought to develop derivatives of 22:0 lyso PC to increase its stability and enhance its potency.

### Example B-1: Hyperactivation of Human moDCs

### Materials and Methods

Human monocytes were isolated from Leukopaks purchased from Miltenyi using the StraightFrom Leukopak CD14 microbead kit (Miltenyi). Isolations were completed following manufacturer's instructions. Monocytes were then aliquoted and frozen in fetal bovine serum containing 10% dimethyl sulfoxide. For studies with monocyte-derived dendritic cell (moDC) cultures, monocytes were thawed and cultured in RPMI medium containing 10% FBS, 50 units/mL penicillin, 50 mg/mL streptomycin, 2 mM L-glutamine, 1 mM sodium pyruvate, 50 mM beta-mercaptoethanol, 10 mM HEPES, and Gibco MEM non-essential amino acids (R10 media). To differentiate monocytes into moDCs, recombinant human GM-CSF (50 ng/mL) and IL-4 (25 ng/mL) were added to R10 media. Cells were cultured for 6 days with GM-CSF and IL-4, with an additional cell feeding with R10 containing GM-CSF and IL-4 on day 3.

Six days after differentiation, moDC were collected and counted. Cells were plated into 96-well flat-bottom plates at 1×10⁵ cells/well. Cells were treated with or without 1 μg/mL R848 (final) and with or without a hyperactivating lipid (or vehicle control). In some experiments, MCC950, an inhibitor of the NLRP3 inflammasome, was added at a final concentration of 10 uM. In some experiments, nigericin, which causes NLRP3 inflammasome activation and subsequent pyroptosis, was used as a positive control at 20 uM. Cells and stimuli totaled a final volume of 20 uL/well.

Lipid stocks were formulated at 650 μg/mL lipid in 0.5 or 4% Kolliphor P407 (KP407) in PBS. Lipids were prepared from lyophilized stocks by mixing with a cold solution of KP407 at 100 rpm for 1 hour at RT. A 10×PBS solution was then added and the lipids were mixed at RT for an additional 30 min to make the 0.5 or 4% KP407 stock solution isotonic. Lipid stocks were then further diluted in PBS to treat cells.

After an overnight incubation, cells and culture supernatant were used for downstream readouts. One hundred and fifty microliters of cell supernatant were collected. Viability was measured using the CellTiter-Glo assay (Promega) which measures ATP content from cells. Fifty microliters of CellTiter-Glo reagent were added to 50 μL of cells. Luminescence was quantified on a SpectraMax m5e plate reader using an integration time of 500 milliseconds. Viability data were set relative to control conditions where cells were treated with only R848. To measure IL-1β secretion, the human IL-1β Lumit kit (Promega) was used to assay cell culture supernatant. Culture supernatant samples were incubated with enzyme-linked antibodies in a 384-well plate for 1 hour before addition of luminescent substrate. Samples were measured for luminescence with an integration time of 500 milliseconds. IL-1β concentrations of samples was determined by interpolation from a standard curve using 4-parameter logistic regression analysis. Studies were performed on three different human donor samples, and each biological condition was tested in triplicate. Graphed data represent means from each donor.

### Results

Derivatives of 22:0 lyso PC were synthesized containing an ether linkage between the glycerol backbone and acyl chain. DGPC (Compound 1) and DGP (Compound 2) are both ether-linked lipids but differ from each other in that DPGC contains a phosphocholine group whereas the DGP (Compound 2) only contains a phosphate group without the choline attachment. To test these lipids against each other, they were prepared from powder stocks into a solution containing 0.5% KP407. Human monocytes were differentiated into dendritic cells using GM-CSF and IL-4. To hyperactivate cells, the TLR7/8 agonist R848 was added to the cells in combination with one of the lyso lipids. After incubating cells for 24 hours, cell culture supernatant was collected. The remaining cells were measured for viability using CellTiter-Glo, an assay that measures ATP. Cell toxicity was minimal in all of the conditions tested (FIG. 1A). Using the cell-free supernatant, IL-1β was measured (FIG. 1B). When R848 was not added to cultures, IL-13 was not produced. This was an expected result because the TLR agonist initiates the production pro-IL-11p via NF-kB signaling. R848 added by itself also did not produce IL-1β because a second, hyperactivating stimulus is required to cleave and secrete IL-1β. When R848 was added in combination with 22:0 lyso PC, IL-1β was secreted. DGPC (Compound 1), which differs from 22:0 lyso PC in the conversion from an ester to an ether linkage of its acyl chain to the glycerol scaffold, also induced IL-1β secretion. However, at an equimolar incubation, DGPC (Compound 1) induced less IL-1β than 22:0 lyso PC. DGP (Compound 2) has the same chemical bond as DGPC (ether) but lacks the choline group. When DGP (Compound 2) was added to human moDC in conjunction with R848, IL-1β was secreted to a greater extent than 22:0 lyso PC.

These data led to informative conclusions. Firstly, substitution of the ester linkage for an ether bond did not abrogate lipid hyperactivity. The ether bond is preferred due to its increased stability compared to the ester bond. Secondly, the ether substitution did not dramatically change the solubility characteristics of DGPC (Compound 1) from 22:0 lyso PC. Like 22:0 lyso PC, DGPC (Compound 1) can be dissolved in ethanol, methanol, or chloroform to prepare aliquots. Thirdly, the data suggest that the polar head group can be modified, such as the removal of the choline to make DGP (Compound 2). Although the solubility profile of DGP (Compound 2) differs from DGPC (Compound 1) and 22:0 lyso PC, DGP (Compound 2) can still hyperactivate human dendritic cells. DGP (Compound 2) is a more potent hyperactivator compared to DGPC (Compound 1) and 22:0 lyso PC. Its solubility profile might be contributing to its hyperactivation potency.

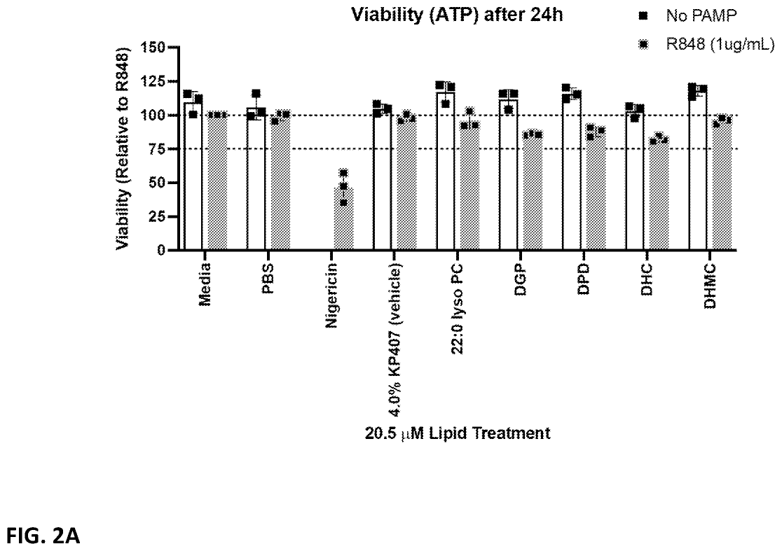

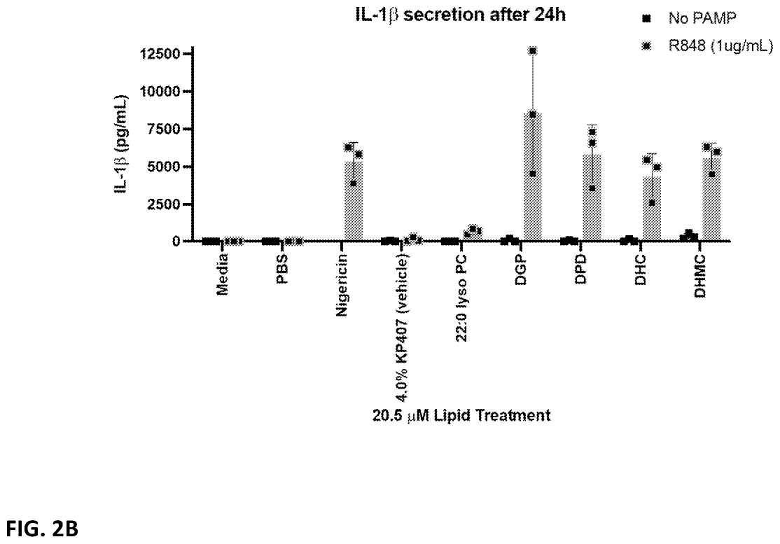

Given the promising data from hyperactivating using DGP (Compound 2) and DGPC (Compound 1), more ether lipid derivatives were tested for their ability to hyperactivate human moDC. DPD (Compound 9) contains neither the phosphate nor choline groups. Testing DPD (Compound 9) would inform us whether the phosphate group is important for the hyperactivation activity observed. DHC (Compound 7) and DHMC (Compound 8) have carbamate moieties attached to the sn-2 position. These sn-2 modifications are potentially advantageous if the chemicals retain their hyperactivity because they would be easier to synthesize. moDC were incubated for 24 hours with or without R848 and with or without a hyperactivating lipid. As control conditions, cells were treated with PBS or PBS containing 4% KP407 as vehicle controls for lipid treatments. Cells were also primed with R848 for 3 hours and then treated with nigericin to induce pyroptosis as a positive control. After the 24 hour treatment, cells were assessed for their viability. Nigericin treatment caused cell death, as expected, leading to approximately 50% decrease in cell viability (FIG. 2A). In all other conditions tested, cell viability was within an expected, acceptable range (FIG. 2A). Cell culture supernatant was collected to measure IL-1β (FIG. 2B). As expected, R848 was required for IL-1β production. Using the pyroptotic stimulus nigericin, IL-1β was produced, albeit at the cost of cell viability. 22:0 lyso PC induced a detectable amount of IL-1β, but in comparison DGP (Compound 2), DPD (Compound 9), DHC (Compound 7), and DHMC (Compound 8) produced significantly more IL-1β. The amount of IL-1β produced is comparable to the levels observed from nigericin but cell viability is maintained in these hyperactivating conditions. These data suggest that the phosphate group is not required for hyperactivation. Additionally, data obtained from testing DHC (Compound 7) and DHMC (Compound 8) further confirm previous observations that relatively small moieties occupying the sn-2 position do not interfere with hyperactivating capacity of lipids.

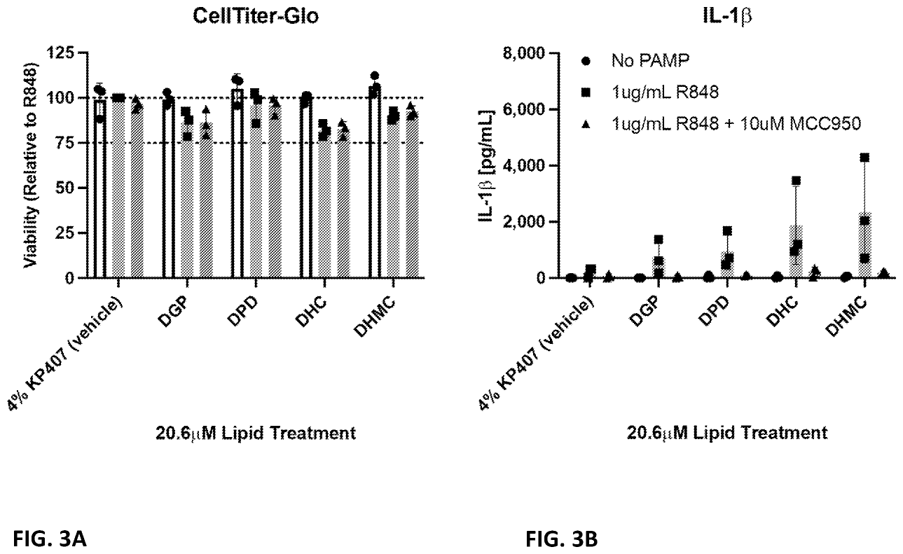

Production of IL-1β while maintaining cell viability are critical features of hyperactivation that allow hyperactive dendritic cells to more advantageously prime adaptive immune responses. Previously identified hyperactivators have been characterized to require the NLRP3 inflammasome for IL-10 release from dendritic cells. To further verify that these ether derivatives mechanistically utilize the same pathway, human moDCs were hyperactivated using DGP (Compound 2), DPD (Compound 9), DHC (Compound 7), or DHMC (Compound 8) in the presence or absence of MCC950, an inhibitor of the NLRP3 inflammasome for one day. Cell viability was within an expected range, and MCC950 treatment did not affect cell viability (FIG. 3A). In comparison to hyperactivations with R848+ hyperactivating lipid, MCC950 treatment diminished the IL-1β output. Thus, DGP (Compound 2), DPD (Compound 9), DHC (Compound 7), and DHMC (Compound 8) depend on the NLRP3 inflammasome for IL-1β secretion, congruent with the mechanism of previously identified hyperactivating lipids.

### Example B-2: Hyperactivation of Murine FLT3L-DCs

### Materials and Methods

Murine bone marrow-derived FLT3L-DCs generation. Leg femur and tibia were removed from mice, cut with scissors, and flushed into sterile tubes. Bone marrow suspension was treated with ACK for 1 minute, then passed through a 40 um cell strainer. Cells were counted and resuspended in media consisting of complete IMDM containing 10% FBS, penicillin and streptomycin, and supplements of L-glutamine and sodium pyruvate (I10). Cells were then plated at 8×10⁶ bone marrow cells per well in a P12 plate. Recombinant mouse FLT3L (Miltenyi) was added to cultures at 200 ng/mL. Differentiated cells were used for subsequent assays on day 8. The efficiency of differentiation was monitored by flow cytometry using BD Symphony A3, and CD11c⁺MHC-II⁺ cells were routinely above 80% of living cells. For each experiment, 5 mice are used to collect BM and to generate DCs.

Lipid preparation. Lipid stocks were formulated at 650 μg/mL lipid in 4% Kolliphor P407 (KP407). Lipids were prepared from lyophilized stocks by mixing with a cold solution of KP407 at 1000 rpm for 1 hour at RT. A 10×PBS solution was then added and the lipids were mixed at RT for an additional 30 min to make the 4% KP407 stock solution isotonic. Lipid stocks were then further diluted in PBS to treat cells.

Murine bone marrow-derived FLT3L-DCs hyperactivation. BMDCs were harvested on day 8 post differentiation, washed with PBS, and re-plated in FLT3L-containing I10 at a concentration of 1×10⁵ cells/mL. Cells were primed with or without 1 μg/mL R848 (final) then treated with or without a hyperactivating lipid (or vehicle control). Forty-eight hours post stimulation, supernatants were collected for cytokine measurement. Viability was measured using the CellTiter-Glo assay (Promega) which measures ATP content from cells. Fifty microliters of CellTiter-Glo reagent were added to 50 μL of cells. Luminescence was quantified on a SpectraMax m5e plate reader using an integration time of 500 milliseconds. Viability data were set relative to control conditions where cells were treated with R848. IL-1β and TNFα cytokine were measured using sandwich ELISAs (Invitrogen).

Murine bone marrow-derived FLT3L-DCs hyperactivation for migration assays. BMDCs were harvested on day 8 post differentiation, washed with PBS, and re-plated in FLT3L-containing I10 at a concentration of 1×10⁶ cells/mL. For DC hyperactivation, 500 μl of R848 was added at a final concentration of 1 μg/mL, and 500 μl of lipids (DGPC (Compound 1), DGP (Compound 2), DHC (Compound 7) or DHMC (Compound 8) or 22:0 lyso PC) prepared in 4% KP407 was diluted to a final concentration of 41 uM. Cells were incubated for 24 hours at 37° C. on a tube rotator. Twenty-four hours post-stimulation, cells were washed with PBS and stained with CFSE (1:1000) for 30 min at 37° C. in the dark. DCs were then counted and 1.10×10⁶ cells were injected subcutaneously (SC) in 100 ul per mouse. 24 hours post-injection, the skin draining lymph nodes (dLN) were dissected. A single cell suspension was prepared, and cells were stained in PBS with Live Dead Fixable dye (ThermoFisher) for 20 min at 4° C. Cells were then washed again and stained for 20 min at 4° C. in MACS buffer (PBS with 1% FCS and 2 mM EDTA) containing the following fluorescently conjugated antibodies: anti-CD11c and anti-I-A/I-E (MHC-II). To determine the absolute number of CD11c MHC-II^{high} among living cells, countBright counting beads (ThermoFisher) were used, following the manufacturer's protocol. Data were acquired on a BD FACS Symphony (Becton-Dickenson). Data were analyzed using FlowJo software (Tree Star). At least five mice were used for each experimental group.

DC migration to the skin dLN following chemical injection. To assess the migration of DCs from the skin to the skin draining lymph node (dLN), mice were subcutaneously injected with 100 ug or 50 ug or 20 ug or 10 ug or 1 μg of R848 in combination with 65 ug or 50 ug or 20 ug of DGP (Compound 2) that was prepared in KP407 at 4.0% final. 24 hours post-injection, the skin draining lymph nodes (dLN) were dissected. A single cell suspension was prepared, and cells were stained in PBS with Live Dead Fixable dye (ThermoFisher) for 20 min at 4° C. Cells were then washed again and stained for 20 min at 4° C. in MACS buffer (PBS with 1% FCS and 2 mM EDTA) containing the following fluorescently conjugated antibodies: anti-CD11c and anti-I-A/I-E (MHC-II). To determine the absolute number of CD11c⁺MHC-II^{high} among living cells, countBright counting beads (ThermoFisher) were used, following the manufacturer's protocol. Data were acquired on a BD FACS Symphony (Becton-Dickenson). Data were analyzed using FlowJo software (Tree Star). Five mice were used for each experimental group.

Ex vivo whole tumor lysate preparation. Syngeneic whole tumor lysates (WTL) were prepared from tumors explants of unimmunized tumor-bearing mice. Briefly, tumors from unimmunized mice bearing a tumor 10-12 mm of size were mechanically disaggregated using gentle MACS dissociator (Miltenyi Biotec) and enzymatically digested using the Tumor Dissociation Kit (Miltenyi Biotec) following the manufacturer's protocol. After digestion, tumor cell suspensions were washed with PBS and passed through 70 μm and then 30 um filters. Tumor cells were then counted and resuspended at 2×10⁷ cells/ml then lysed by 3-4 cycles of freeze-thawing. The lysed cells were further disrupted by repeatedly passing the material through an 18G, then 21G, and finally 250 needles. Lysate was filtered again through 70 μm and 30 um cell strainers and stored in aliquots at −80° C. until use. Protein quantification in the lysates was performed using the BCA assay. WTL were used for immunotherapy at a concentration equivalent to 50 ug per mouse.

Immunotherapy. C57BL/6J mice were injected SC with 50,000 cells on the right upper back. Seven days later, mice were either injected SC with PBS or immunized distally from the tumor injection site with 50 ug of WTL derived from syngeneic tumors in combination with the hyperactivating stimuli (LPS10 ug/mouse+PGPC 65 ug/mouse, R848 100 ug/mouse+22:0 lyso PC 65 ug/mouse, or R848 100 ug/mouse+DGP 65 ug/mouse). Mice received 4 boost injections with same doses of WTL+ hyperactivating stimuli on days 11, 14, 18, and 21 post-tumor inoculation.

### Results

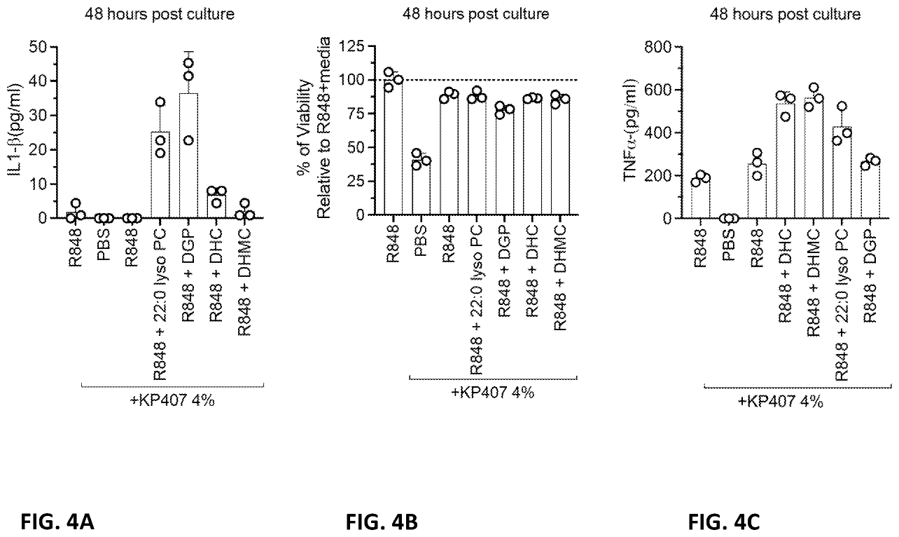

DGP (Compound 2) induces murine DC hyperactivation. FLT3L DCs were primed with 1 μg/mL of R848 for 2-3 hours then treated with 41 uM of lipids (DGP (Compound 2) or 22:0 Lyso PC or DHC (Compound 7) or DHMC (Compound 8)). 48 hours post-stimulation, supernatants were collected to assess cytokine release. As expected, DCs treated with R848 alone or in combination with the vehicle control KP407, or DCs treated with PBS and KP407, did not induce IL-1β secretion. In contrast, DC treated with R848+DGP induced the highest levels of IL-1β secretion from viable cells as compared to DCs treated with R848+22:0 lyso PC, R848+DHC, or R848+DHMC (FIG. 4A). DHC (Compound 7) and DHMC (Compound 8) induced the lowest levels of IL-1β secretion, and viability was not compromised when DCs were treated with either lipid (FIG. 4B). TNFα secretion, which indicates NF-kB activation by R848, was comparable when DCs were treated with R848 alone or R848 in combination with DGP (Compound 2) (FIG. 4C). These data indicate that DGP (Compound 2) can induce a state of DC hyperactivation whereby DCs add to their cytokine secretion repertoire IL-1β while remaining viable.

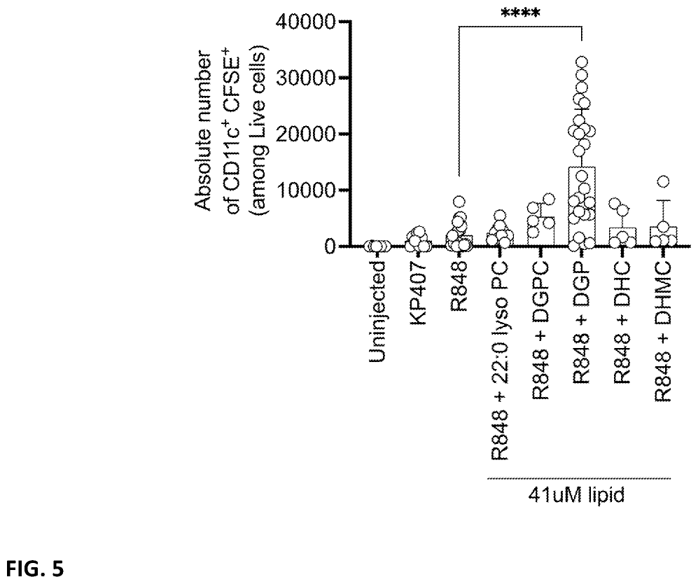

DGP (Compound 2) enhances DC migration to the dLN. Another hallmark of DC hyperactivation is DC hypermigration to dLN. To assess whether DGP induces DC migration, DCs were stimulated with R848 and lipids overnight on a rotator. DCs were then washed with PBS, stained with CFSE, and then injected SC in CD45.1 congenic mice. Twenty-four hours post injection, the draining lymph nodes (dLN) were dissected, and the absolute number of CFSE-labeled DCs that migrated to the dLN was assessed by flow cytometry. Interestingly we found that DGP significantly enhanced DC migration from the skin to the dLN as compared to R848 or KP407 vehicle control (FIG. 5, unpaired t-test, p<0.0001). When compared to other lipids, DGP (Compound 2)-induced migration activity was significantly higher than DGPC (Compound 1). 22:0 lyso PC, DHC (Compound 7), and DHMC (Compound 8). These data highlight that DGP (Compound 2) is a strong hyperactivator in mice that induces hypermigration of DCs to the dLN.

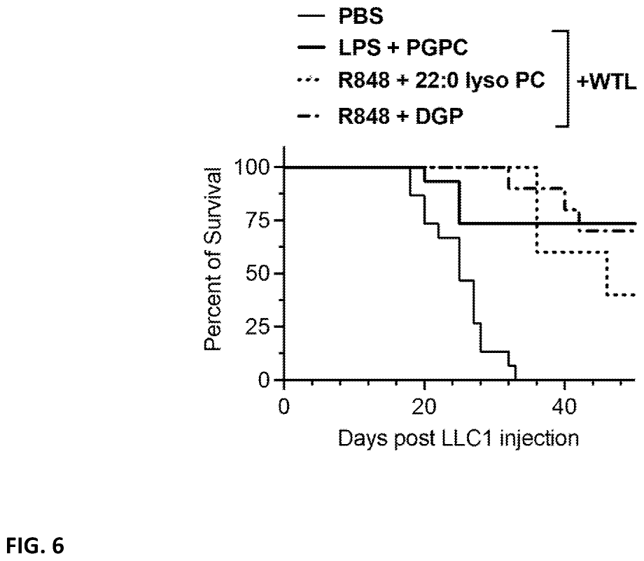

DGP (Compound 2) induces tumor rejection in the LLC1 tumor model. The data above provided the mandate to assess whether DGP induces anti-tumor immunity. We chose to test it in the LLC1 model, which is an “icy” tumor (lacking immune infiltration of the tumor microenvironment) resistant to anti-PD1 and is therefore very difficult to treat. C57BL/6J mice were injected SC with 50,000 cells on the right upper back. Seven days later, mice were either injected SC with PBS or immunized distally from the tumor injection site with WTL derived from syngeneic tumors in combination with the hyperactivating stimuli. LPS+PGPC served as a positive control, and R848+22:0 lyso PC and R848+DGP served as test articles. Mice received 4 boost injections with WTL+ hyperactivating stimuli on days 11, 14, 18, and 21 post-tumor inoculation. Interestingly, we found that similar to LPS+PGPC, R848+DGP controlled tumor growth and strongly enhanced tumor rejection as compared to R848+22:0 lyso PC (FIG. 6). 80% of mice survived for up to 49 days post-injection when they were immunized with LPS+PGPC or R848+DGP. In contrast, more than 50% of mice immunized with R848+22:0 lyso PC succumbed to tumor growth. These data indicate that DGP (Compound 2) induces strong anti-tumor responses in mice.

### Example B-3: Hyperactivation to Induce T Cell Responses In Vivo

### Materials and Methods

Vaccine formulation. DGP (Compound 2) was prepared from powder stock by resuspending in a cold solution of 4% KP407 at 650 μg/mL. The mixture was stirred for 1 hour at RT using a magnetic stir bar at 100 rpm. After 1 hour, 10×PBS was added to make an isotonic solution and stirred at RT for another 30 min. The lipid was then combined with additional components depending on the treatment group. VacciGrade™ clinical grade R848 (InvivoGen) was prepared by dissolving in PBS at a stock of 10 mg/mL for further combination depending on treatment group. For antigen, chicken ovalbumin (OVA) (InvivoGen) and SARS-COV-2 Spike (R&D Systems) protein were dissolved in PBS at 10 mg/mL and 1 mg/mL, respectively, to create stock solutions.

Immunization. Naïve C57BL/6J mice were grouped into four different immunization conditions. As negative controls, mice received PBS injections or antigen only (200 ug OVA and 5 ug spike). A third group received 10 ug of R848 in addition to the antigens. Finally, the fourth group received antigens, 10 ug R848, and 65 ug DGP (Compound 2). Vaccinations were injected subcutaneously in the dorsal flank at 200 uL/mouse. The same immunization treatments were repeated twice at one-week intervals. Seven days after the final immunizations (day 21 from start of study), mice were euthanized and draining lymph nodes were taken for downstream analyses. Organs were kept in MACS tissue storage solution (Miltenyi) at 4° C. until processed. Four mice were allotted to each treatment group, but draining lymph nodes in the PBS treatment group were pooled.

ELISPOT analysis. Spleen dissociation kits (Miltenyi) were used to dissociate draining lymph nodes according to manufacturer's protocol. Cells were counted and plated at 200,000 cells/well. IFNγ ELISPOT kits (R&D Systems) were used according to manufacturer protocol to study IFNγ T cells responses. To restimulate cells, 10 μg/mL of OVA or Spike peptivators were added to cell cultures (Miltenyi). Restimulations were done in triplicate, and responses for each mouse in the study were averaged. Data graphed are the means from each mouse.

### Results

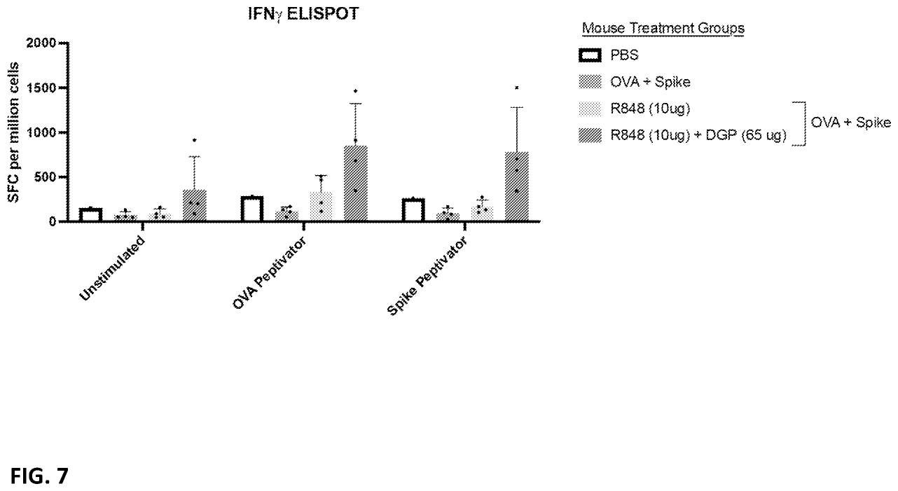

Hyperactivating stimuli act on dendritic cells but ultimately potentiate the T cell response against targeted antigens. To determine if DGP (Compound 2) mechanistically functions like other known hyperactivators, an in vivo murine study was set up. Mice were immunized with various treatment conditions and boosted twice with the same treatments at one-week intervals. As negative controls, mice received either PBS vehicle or OVA+Spike protein antigens. Without any PAMP or DAMP signals, minimal responses would be expected. As treatment groups, mice received the same antigens with the addition of either R848 alone or R848+DGP. One week after the final boost, IFNγ responses in the draining lymph nodes were analyzed via ELISPOT. When left unstimulated, minimal responses were observed with the exception of the hyperactivating immunization containing DGP (Compound 2) (FIG. 7). This observation would suggest that a hyperactivating immunization prolongs and/or strengthens T cell activity compared to other immunization methods like R848 alone. When immune cells were restimulated with peptide libraries derived from OVA or Spike, most treatment groups had minimal increases in responses compared to the unstimulated cells. Notably, immunizing with R848+DGP increased IFNγ spot formation compared to the corresponding unstimulated cells, suggesting that the observed responses are antigen specific (FIG. 7). Additionally, comparison of R848+DGP to other treatment groups within the OVA peptivator or Spike peptivator stimulations demonstrates that immunizations using R848+DGP produce more IFNγ responses (FIG. 7). Interestingly, the depressed responses observed in the antigen only group suggest that the lack of adjuvant induced a tolerogenic response. R848+DGP immunizations elevated responses to both OVA and Spike antigens. Altogether, this in vivo study suggests that immunizations utilizing DGP (Compound 2) increases T cell responses, similar to other hyperactivating lipids.

### Example B-4: Hyperactivation of Human moDCs with Lipid Nanoparticles (LNPs)

This example describes the hyperactivation of human monocyte-derived dendritic cells (moDCs) with a TLR7/8 agonist in combination LNPs loaded with a hyperactivating lipid (ETL or ETPL).

### Materials and Methods

Human monocytes are isolated from Leukopaks purchased from Miltenyi Inc. (San Jose, CA) using the StraightFrom Leukopak CD14 microbead kit according to the manufacturer's instructions. Monocytes are then aliquoted and frozen in fetal bovine serum containing 10% dimethyl sulfoxide. For studies with monocyte-derived dendritic cell (moDC) cultures, monocytes are thawed and cultured in RPMI medium containing 10% FBS, 50 units/mL penicillin, 50 mg/mL streptomycin, 2 mM L-glutamine, 1 mM sodium pyruvate, 50 mM beta-mercaptoethanol, 10 mM HEPES, and Gibco MEM non-essential amino acids (R10 media). To differentiate monocytes into moDCs, recombinant human GM-CSF (50 ng/mL) and IL-4 (25 ng/mL) are added to R10 media. Cells are cultured for 6 days with GM-CSF and IL-4, with an additional cell feeding with R10 media containing GM-CSF and IL-4 on day 3.

Six days after differentiation, moDC are collected and counted. Cells are plated into 96-well flat-bottom plates at 1×10⁵ cells/well. Cells are treated with or without 1 μg/mL R848 (final) and with or without a hyperactivating lipid (or vehicle control). Hyperactivity induced by LNPs is measured using two assays. The CellTiter-Glo assay (Promega) detects ATP as a measure of cell viability. The IL-1β Lumit assay (Promega) measures IL-1β cytokine in the moDC cell culture supernatant. Experimental conditions are tested in triplicate and the mean result from one donor is plotted.

### Example B-5: Hyperactivation of Human moDCs

### Materials and Methods

Human moDC Production. Human monocytes were isolated from Leukopaks purchased from Miltenyi using the StraightFrom Leukopak CD14 microbead kit (Miltenyi) according to the manufacturer's instructions. Monocytes were then aliquoted and frozen in fetal bovine serum containing 10% dimethyl sulfoxide. To differentiate monocyte-derived dendritic cell (moDC) cultures, monocytes were thawed and cultured in RPMI medium containing 10% FBS, 50 units/mL penicillin, 50 mg/mL streptomycin, 2 mM L-glutamine, 1 mM sodium pyruvate, and 50 mM beta-mercaptoethanol (R10 media). To differentiate monocytes into moDCs, recombinant human GM-CSF (50 ng/mL) and IL-4 (25 ng/mL) were added to R10 media. Cells were cultured for 6 days with GM-CSF and IL-4, with an additional cell feeding with R10 containing GM-CSF and IL-4 on day 3.

Hyperactivating Lipid Formulation—Compound 10. Lipid stocks were formulated at 200 μg/mL lipid in 5% Kolliphor P407 (KP407) in PBS. Lipids were prepared from lyophilized stocks by mixing with a cold solution of 5.6% KP407 at 250 rpm for 1.5 hours at 4° C. and then 1 hour at RT on orbital shakers. A 10×PBS solution was then added to make the 5% KP407 stock solution isotonic. Lipid stocks were then further diluted in PBS to treat cells at a final lipid concentration of 41.3 uM.

Hyperactivating Lipid Formulation—Compounds 7 and 8. Lipid stocks were formulated at 650 μg/mL lipid in 4% Kolliphor P407 (KP407) in PBS. Lipids were prepared from lyophilized stocks by mixing with a cold solution of 4.4% KP407 at 1000 rpm for 1 hour at RT on a magnetic stir plate. A 10×PBS solution was then added to reach an isotonic 4% KP407 stock solution. Lipid stocks were then further diluted in PBS to treat cells at a final lipid concentration of 41.3 uM.

Hyperactivating Lipid Formulation—Compounds 11 and 12. Lipid stocks were formulated at 650 μg/mL lipid in 4% Kolliphor P407 (KP407) in PBS. Lipids were prepared from lyophilized stocks by mixing with a cold solution of 4.4% KP407 at 1000 rpm for 1 hour at RT on a magnetic stir plate. A 10×PBS solution was then added to reach an isotonic 4% KP407 stock solution. Lipid stocks were then further diluted in PBS to treat cells at a final lipid concentration of 20.6 uM.

Hyperactivating Lipid Formulation—Compounds 1, 4, 6 and 11-16. Lipid stocks were formulated at 650 μg/mL lipid in 4% Kolliphor P407 (KP407) in PBS. Lipids were prepared from lyophilized stocks by mixing with a cold solution of 4.4% KP407 at 1000 rpm for 1 hour at RT on a magnetic stir plate. A 10×PBS solution was then added to reach an isotonic 4% KP407 stock solution. Lipid stocks were then further diluted in PBS to treat cells at a final lipid concentration of 41.3 uM or 20.6 uM. For vehicle control, 4% KP407 was diluted with PBS to match highest volume (or least diluted) lipid for addition to cells.

Hyperactivation of moDCs. Six days after differentiation, moDCs were collected and counted. Cells were plated into 96-well flat-bottom plates at 1×10⁵ cells/well. Cells were treated with or without 1 μg/mL R848 (final) and with or without a hyperactivating lipid (or vehicle control). Cells and stimuli totaled a final volume of 200 uL/well.

Measurement of Cytokine Secretion and Cell Viability. After an overnight incubation, cells and culture supernatant were used for downstream readouts. One hundred and fifty microliters of cell culture supernatant were collected. Viability was measured using the CellTiter-Glo assay (Promega) which measures ATP content from cells. Fifty microliters of CellTiter-Glo reagent were added to 50 μL of cells. Luminescence was quantified on a SpectraMax m5e plate reader using an integration time of 500 milliseconds. Viability data were set relative to control conditions where cells were treated with only R848. To measure IL-1β secretion, the human IL-1β Lumit kit (Promega) was used. Culture supernatant samples were incubated with enzyme-linked antibodies in a 384-well plate for 1 hour before addition of a luminescent substrate. Samples were measured for luminescence with an integration time of 500 milliseconds. IL-1β concentrations of samples was determined by interpolation from a standard curve using 4-parameter logistic regression analysis. Studies were performed on two different human donor samples, and representative data from one healthy donor (HD) are shown. Each biological condition was tested in triplicate, and replicates are represented in graphs as symbols. The means of replicates are represented by columns, and error bars denote SD. For analysis of results from tests using Compounds 10-12, statistical analysis was done using two-way ANOVA followed by Tukey's multiple comparisons test with a single pooled variance. For analysis of results from tests using Compounds 1, 4, 6 and 11-16, statistical analysis was done using ordinary two-way ANOVA followed by Dunnett's multiple comparisons test with a single pooled variance. In graphs, the number of asterisks represent the following p-values: *p<0.05; **p<0.01; ***p<0.001; ****p<0.0001.

### Results

Compound 10 was tested for its ability to hyperactivate dendritic cells. Human moDCs were treated with or without R848 in combination with Compound 10. As control conditions, the cells were also treated with other lipids that had been previously demonstrated to induce moDC hyperactivation: 22:0 Lyso PC and DPD (Compound 9). As a negative control, cells were treated with a vehicle solution of KP407 in which lipids had been formulated. moDC were treated with 41.25 μM of the indicated lipids.

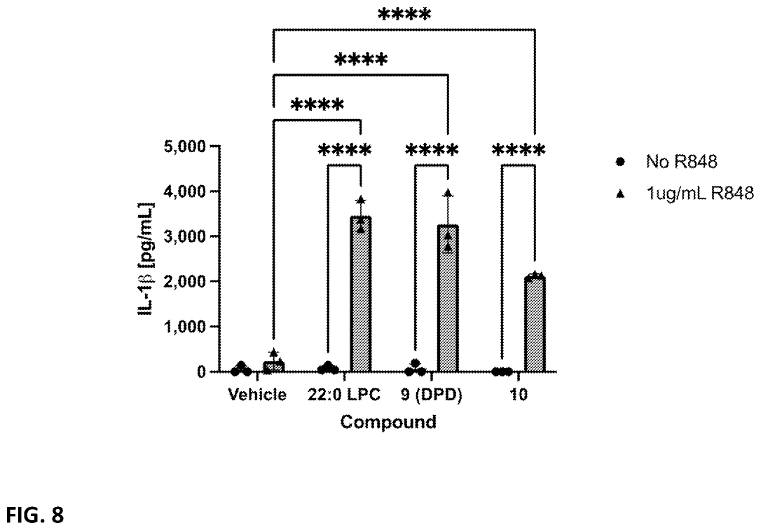

In comparison to the vehicle control, moDC treated with 22:0 Lyso PC or DPD produced significantly more IL-1β when combined with R848, as expected (FIG. 8). When Compound 10 was combined with R848, significantly more IL-1β was secreted from moDC than from moDC treated with the vehicle control. As expected, IL-1β secretion was dependent on the combination of a lipid compound and R848. When R848 was not added to the cell treatments, IL-1β was not secreted by moDCs (FIG. 8).

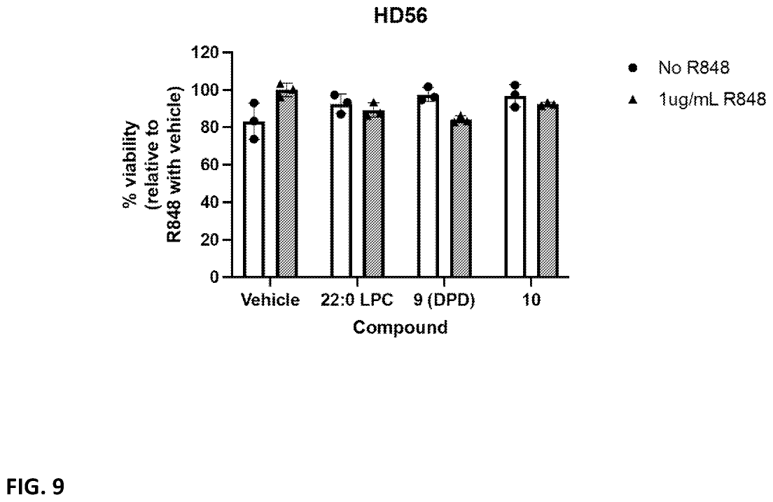

To determine if IL-1β secretion was caused by hyperactivation of moDC or pyroptosis, a cell viability assay was performed. Cells were used in a CellTiter-Glo assay that measures the abundance of adenosine triphosphate as an indicator of cell viability. Across all the conditions tested, the mean cell viability was above 80% (FIG. 9), indicating that treatments did not cause significant cell death. Thus, IL-1β secretion was caused by hyperactivation, as opposed to pyroptosis. Compound 10 maintained high viability, indicative of a hyperactivating lipid that can induce secretion of IL-1β by moDCs without causing substantial cell death.

Compounds 7 and 8 were tested for their ability to hyperactivate dendritic cells in comparison to other lipids that had been established to hyperactivate moDC, namely 22:0 Lyso PC, Compound 9 (DPD), and Compound 2 (DGP). The lipids were formulated in 4% KP407, and cells were treated with 41.3 uM of each compound. As a negative vehicle control, cells were treated with an equivalent amount of KP407. Cells were also stimulated with or without R848, and with and without MCC950 to test the dependence of IL-1β secretion on the NLRP3 inflammasome.

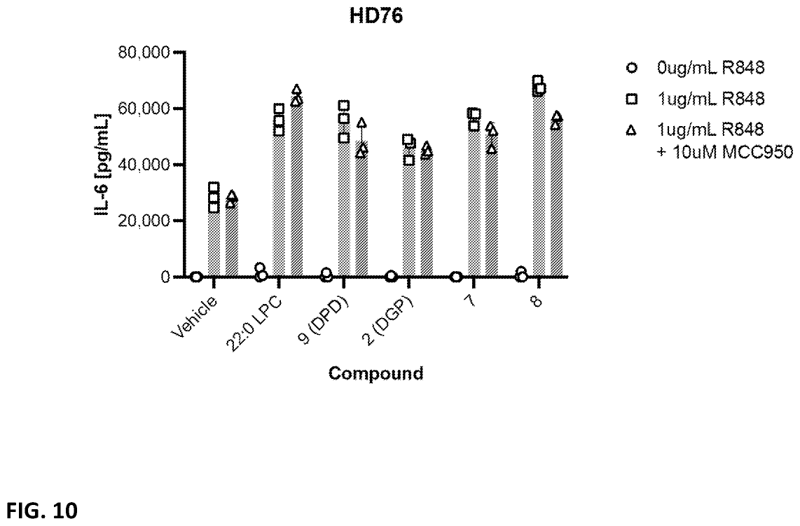

To determine if the cells responded to R848 stimulation as expected, IL-6 secretion was measured. Minimal IL-6 (less than 4000 μg/mL) was detectable in culture supernatants from moDCs when R848 was not added (FIG. 10). In contrast, when cells were stimulated with R848, greater than 20,000 μg/mL IL-6 was detected in the cell culture supernatant (FIG. 10). IL-6 secretion is not dependent upon NLRP3 inflammasome activation, and when the MCC950 inhibitor was added to cultures containing R848, IL-6 was still produced at similar levels. Thus, moDCs responded as expected to R848 stimulation in combination with a hyperactivating lipid.

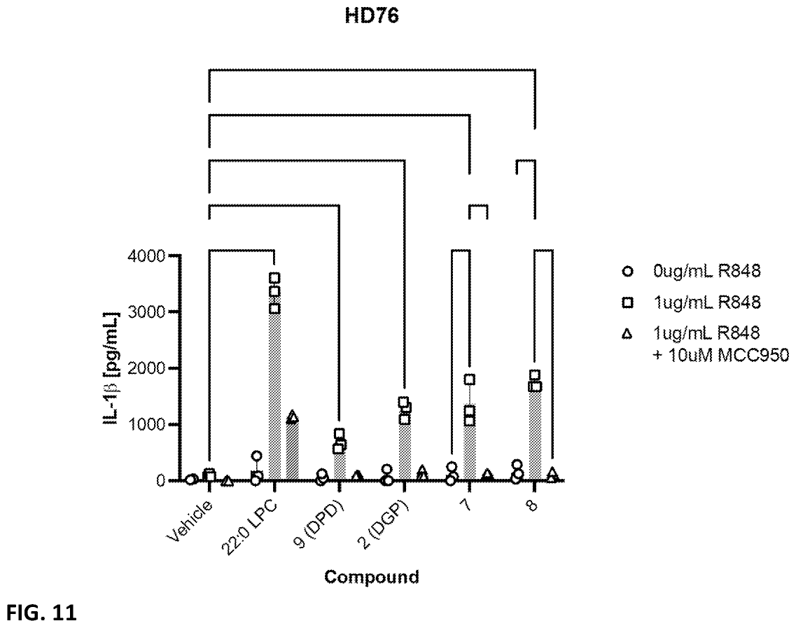

The ability of cells to secrete IL-1β was next measured. When moDC were treated with R848 and the lipid vehicle control, minimal amounts of IL-1β were detected (FIG. 11). Previously identified hyperactivating lipids (22:0 Lyso PC, DPD, and DGP) robustly induced IL-113 secretion when combined with R848 (FIG. 11). Compounds 7 and 8 also induced secretion of IL-1β from moDC when combined with R848 (FIG. 11). The amounts detected were significantly higher than the levels produced by cells contacted with the vehicle control. Similar to 22:0 Lyso PC, DPD, and DGP, moDCs required Compounds 7 and 8 be combined with R848 in order to secrete IL-1β (FIG. 11). When cells were treated with MCC950, IL-1β secretion from Compounds 7 and 8 were significantly reduced (FIG. 11), indicating that IL-1β secretion was dependent on NLRP3 inflammasome activation.

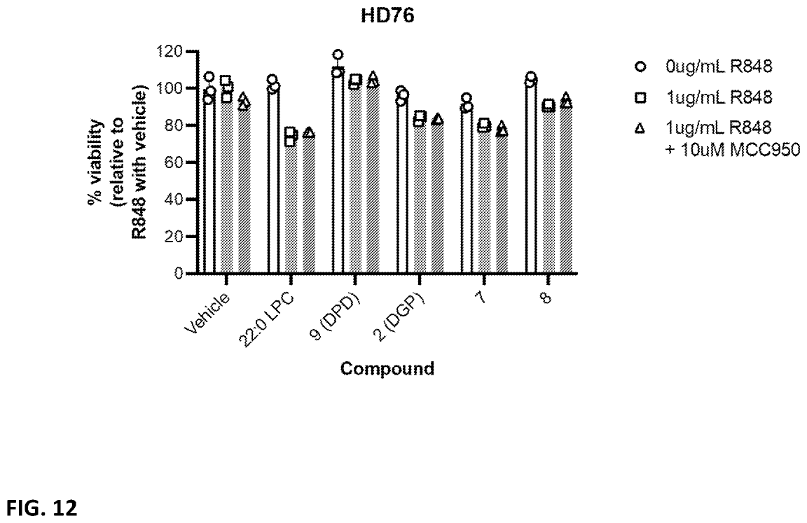

In addition to IL-1β secretion, hyperactivated moDC must also maintain cell viability. Otherwise, the IL-1β secretion would be the result of pyroptosis, which is a distinct mechanism. Cells viability was measured using the CellTiter-Glo Assay. Across all conditions, cells maintained an acceptable level of viability above 75% (FIG. 12). Additionally, cell viability under hyperactivation conditions were comparable between established hyperactivating lipids such as 22:0 Lyso PC, DPD, and DGP, as compared to Compounds 7 and 8 (FIG. 12). The viability data indicate that the IL-1β secreted induced by treatment with Compounds 7 or 8 in combination with R848 was caused by moDC hyperactivation, and not pyroptosis. Thus, Compounds 7 and 8 are hyperactivating lipids.

Compounds 11 and 12 were tested for their ability to hyperactivate human moDC. Cells were treated with or without R848 in combination with the compounds of interest. As a negative control, KP407, which was used to formulate lipids, was added to cultures. To determine the mechanism underlying activity, moDCs were also treated with MCC950 to test the dependence of IL-1β secretion on the activation of the NLRP3 inflammasome.

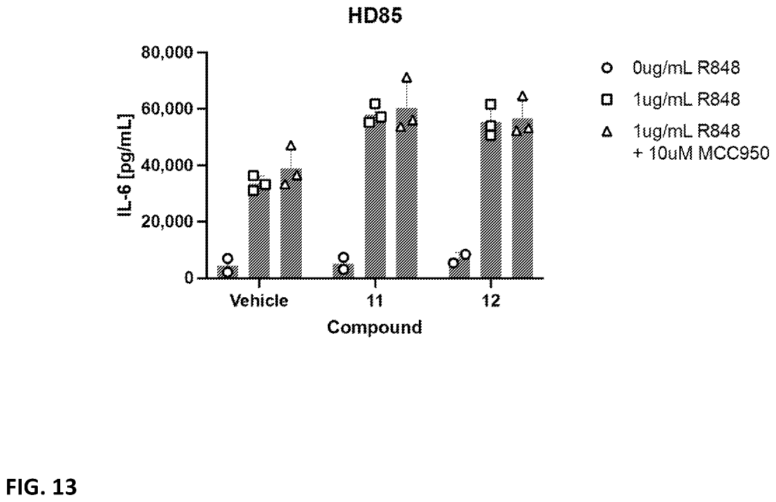

R848 stimulation induces the expression of many inflammatory genes, including IL-6. To confirm that moDC were responsive to R848, IL-6 in cell culture supernatant was measured one day after stimulation. When cells were not treated with R848, minimal IL-6 was produced (FIG. 13). In all cases when R848 was added, significantly higher levels of IL-6 were detected.

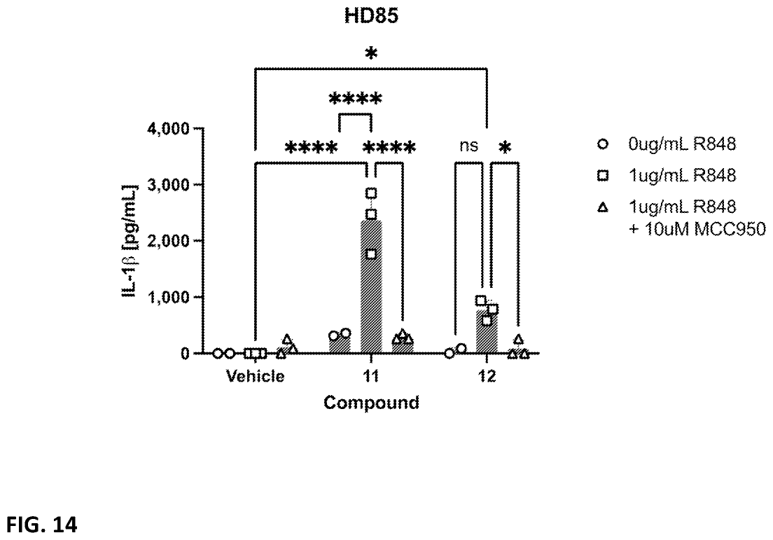

Next, IL-1β secretion was measured. moDC were treated with 20.6 uM of experimental Compounds 11 or 12. When combined with R848, significantly more IL-1β was detected in cell culture supernatant as compared to vehicle treatment (FIG. 14). Compounds 11 and 12 required R848 stimulation to secrete IL-1β, as little to no IL-1β was secreted the absence of R848 (FIG. 14). Additionally, IL-1β secretion was dependent upon NLRP3 inflammasome activation. When cells were additionally treated with 10 uM MCC950, secretion of IL-1β by stimulating with R848 and Compound 11 or 12 was inhibited (FIG. 14).

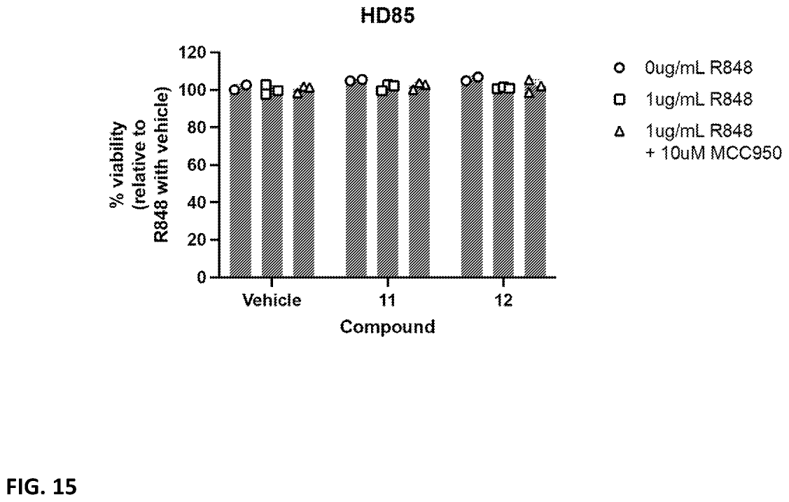

Both Compounds 11 and 12 were able to induce moDC secretion of IL-1β. To determine if IL-1β secretion was caused by hyperactivation or pyroptosis, cell viability was assessed using the CellTiter-Glo assay. Across all conditions, moDCs had comparably high levels of cell viability above 75% (FIG. 15). The high levels of cell viability indicate that IL-1β secretion was due to hyperactivation, not pyroptosis. Thus, Compounds 11 and 12 are hyperactivating lipids.

Compounds 1, 4, 6 and 11-16 were tested in comparison to Compound 2 (DGP) and 22:0 Lyso PC (positive controls) and vehicle (negative control) for their ability to hyperactivate human moDCs. The vehicle consisted of a KP407 solution in which the lipids were formulated. All lipids were tested at an equimolar concentration of 41.25 μM. To hyperactivate moDC, a hyperactivating lipid is combined with an additional signal, in this case R848 was used to stimulate cells via TLR7/8. The hyperactivating lipid then activates the NLRP3 inflammasome, leading to the cleaving of the pro-form of IL-1β and its secretion from cells. As an added control, the NLRP3 inhibitor MCC950 was used in this study to determine the dependence of IL-1β secretion on NLRP3.

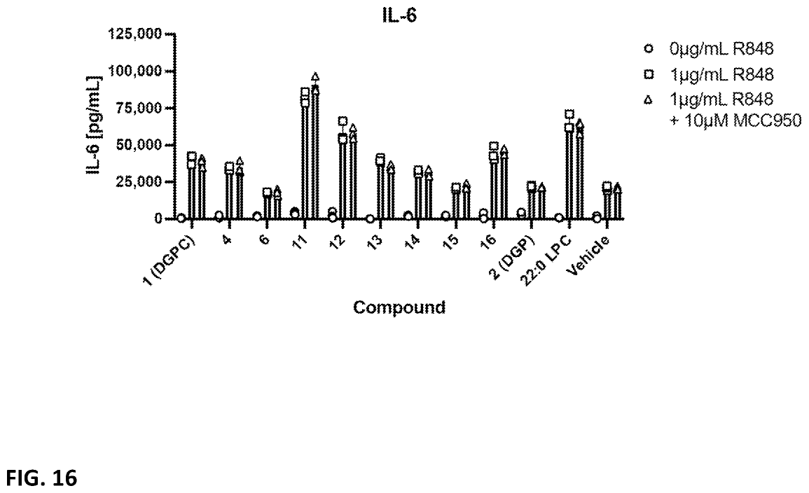

The activity of R848 was verified by measuring the production of the inflammatory cytokine IL-6. When R848 was not added to moDC cultures, minimal IL-6 was detected. In all cases when R848 was added to cell cultures, IL-6 secretion significantly increased compared to test conditions devoid of R848 (FIG. 16, p<0.0001). When MCC950 was added with R848, IL-6 concentrations did not change, indicating that IL-6 secretion was not dependent upon NLRP3 inflammasome activation (FIG. 16). Interestingly, in the negative control (vehicle in the absence of a hyperactivating lipid), IL-6 secretion was measured at approximately 20,000 μg/mL (FIG. 16). When Compounds 2, 6, and 15 were added to cultures, IL-6 was secreted at similar levels. When the other lipids were added in combination with R848, IL-6 secretion increased. These data indicated that IL-6 secretion was controlled by R848 stimulation and was independent of the NLRP3 inflammasome. Addition of lipids could increase IL-6 production but not diminish it.

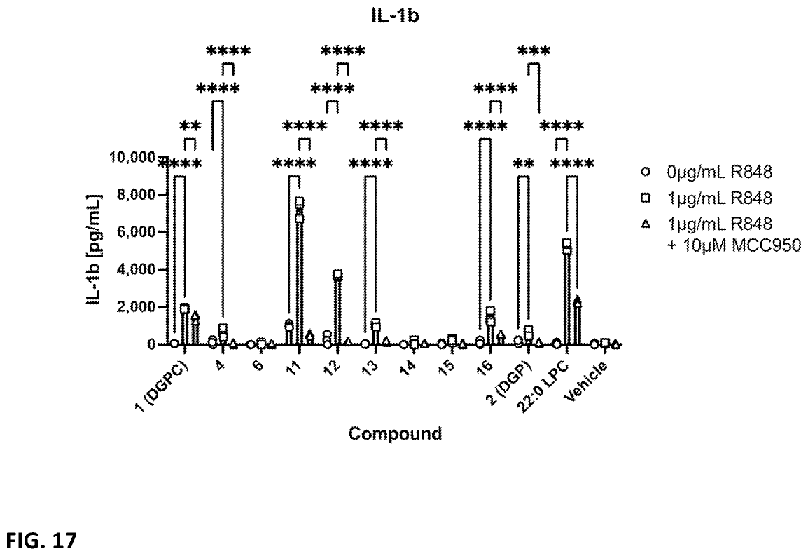

Next, IL-1β in the cell culture supernatant was measured. When R848 was not added to moDC, minimal IL-1β was detectable (FIG. 17). As expected, combining R848 stimulation with Compound 2 or 22:0 LPC resulted in a significant increase in IL-1β compared to the controls devoid of R848 (FIG. 17). Similarly, Compounds 1, 4, 11, 12, 13, and 16 significantly increased IL-1β secretion when combined with R848 (FIG. 17). The IL-1β secretion induced by these compounds was NLRP3-dependent because addition of MCC950 significantly decreased levels of IL-1β secretion (FIG. 17). In contrast to the active lipid compounds, Compounds 6, 14, and 15 induced minimal IL-1β secretion when combined with R848, resembling the vehicle control condition (FIG. 17).

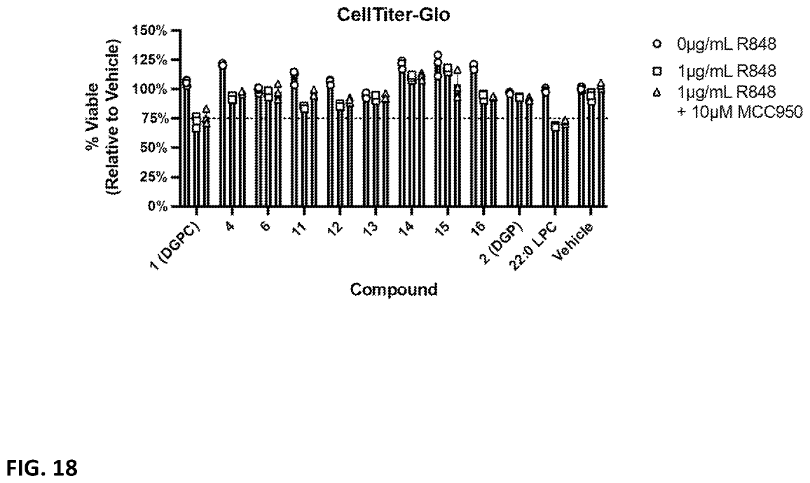

Even when IL-1β secretion is dependent upon NLRP3, chemicals can induce pyroptotic cell death instead of hyperactivation. To determine if moDC were hyperactivated, cell viability was measured after the 24 hour incubation with stimuli. Under most experimental conditions, cell viability was within a reasonable range of +/−25% relative to 100% unstimulated vehicle control (FIG. 18). Thus, Compounds 2, 4, 11, 12, 13, and 16 are hyperactivating lipids.

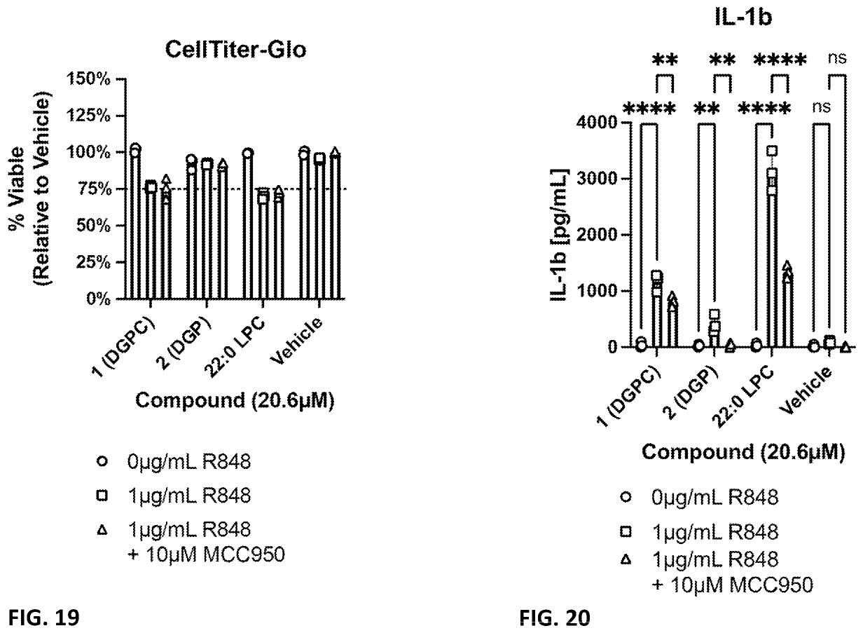

Treatment of moDCs with Compound 1 or 22:0 LPC in combination with R848 resulted in a reduction of cell viability slightly below the 75% viability threshold (FIG. 18). One possibility is that these lipids caused a greater loss in cell viability because they are potent hyperactivating molecules when used at a high concentration can cause cell toxicity. For this reason, these lipids were also tested at a lower concentration of 20.6 μM. Compound 1 had a viability above the 75% viability threshold (FIG. 19), and induced IL-1β secretion that was dependent upon R848 stimulation and the NLRP3 inflammasome (FIG. 20). For moDCs derived from this particular human donor sample, a lower lipid concentration may have been better for screening purposes.

The results of this study were surprising because the compounds differed in their ability to hyperactivate human dendritic cells. Compounds 1, 2, 4, 11, 12, 13, and 16 hyperactivated dendritic cells. In contrast, Compounds 6, 14, and 15 did not. The compounds tested differed in several ways. Lipids could have phosphate and phosphocholine head groups attached to the glycerol backbone of the lipids. The lipid acyl chain was either 18 or 22 carbons in length. The sn-2 alcohol was also sometimes attached to an amide, methyl amide, or dimethyl amide group. None of these modifications eliminated the ability of the compound to hyperactivate cells in all cases. Thus, the modifications introduced in these derivatives are interdependent and consequential to hyperactivity when considered in their totality.

### Example B-6: Hyperactivation of moDCs with R848 and DGP

### Materials and Methods

Monocyte Isolation, Storage, and Differentiation into moDC. Human leukapheresis blood products were freshly obtained from AllCells via same day or overnight shipping at 4° C. Miltenyi StraightFrom Leukopak CD14 MicroBead isolation kits were used according to manufacturer instructions to isolate CD14+ monocytes. Briefly, leukopaks ranging in quantity from 5×10⁹ to 1×10¹⁰ total nucleated cells were evenly aliquoted into 50 mL conical tubes. Cells were incubated with CD14 microbeads and then loaded on the MultiMACS using the program “Possel2”. After washing out the negative population, CD14+ cells were eluted from the columns and counted. An aliquot of monocytes was used to stain for purity of cells to ensure that isolated live cells were 580% CD11c+CD14+ by flow cytometry. Cells were centrifuged at 400×g for 5 min and resuspended at 1.25×10⁷ cells/mL in freezing media (FBS containing 10% DMSO) and aliquoted at 5 mL per cryogenic freezing vial. Vials were placed in CoolCell freezing containers and placed in a −80° C. freezer and stored no longer than 1 week. Frozen cells were transferred for long-term storage to the vapor phase of a liquid nitrogen freezer until use.

To differentiate monocytes into moDC, vials of cells were taken out of the liquid nitrogen freezer and quickly thawed in a 37° C. water bath. Cells were diluted ten-fold in warmed R10++ media containing 50 units/mL benzonase. R10++ media was composed of RPMI 1640 media containing 10% fetal bovine serum, 50 units/mL penicillin, 50 mg/mL streptomycin, 2 mM L-glutamine, 1 mM sodium pyruvate, 50 mM beta-mercaptoethanol, 10 mM HEPES, and 1% Gibco MEM non-essential amino acids. After gentle mixing by inversion of tubes, cells were incubated at 37° C. for 10 min. Cells were pelleted at 400×g for 5 min and supernatant was aspirated. Cells were resuspended in R10++ media and counted on the MoxiGo by diluting an aliquot in PBS containing a final concentration of 2.5 μg/mL propidium iodide. Cells were plated in T75 culture flasks at 3-6×10⁷ cells per flask in a total of 20 mL R10++ media containing a final concentration of 50 ng/mL GM-CSF and 25 ng/mL IL-4. Three days after the start of culture, an additional 10 mL R10++ containing 50 ng/mL GM-CSF and 25 ng/mL IL-4 was added to each flask.

Six days after start of differentiation. R10++ media containing cells were placed into 50 mL conical tubes. Flasks were washed with an additional 10 mL PBS and also collected. Cells were centrifuged at 400×g for 5 min and supernatant was aspirated. Cells were resuspended in R10++ media and counted on the MoxiGo by diluting an aliquot in PBS containing a final concentration of 2.5 μg/mL propidium iodide. Cells were then adjusted to 1×10⁶ cells/mL in R10++ media.

Quality Control Staining of moDC Differentiation. An aliquot of cells was stained to ensure monocyte differentiation into moDC. Cells were plated into a 96-well V-bottom plate at 1×10⁵ cells/well. Cells were also plated for fluorescence minus one (FMO) staining controls. Cells were washed twice with PBS by centrifuging at 400×g for 4 min and removing supernatant. Live/Dead NIR viability dye was diluted 1000-fold in PBS and added to samples at 100 μL/well. For the control FMO sample not receiving live/dead staining, PBS was used to resuspend pelleted cells. Cells were incubated for 10 min at 4° C. Fc block was diluted in FACS buffer 100-fold and added to wells at 100 μL/well. Cells were incubated at 4° C. for another 10 min. Cells were then pelleted at 400×g for 4 min and supernatant was discarded. Cells were then stained with antibody staining cocktail containing CD11c (FITC), CD209 (APC), and SIRPa (PE-Cy7) at 200-fold dilutions. Antibodies were diluted in a buffer mixture composed of FACS buffer and Brilliant Buffer Stain (mixed at 1:1 volume ratio). For FMO controls, staining mixtures were prepared with one target antibody removed to define negative cell populations. Cells were incubated at 4° C. for 15 min in antibody cocktails. After incubation, cells were washed twice with FACS buffer by topping up wells to 200 μL/well, centrifuging at 400×g for 4 min, and discarding supernatant. If cells were analyzed by flow cytometry immediately, they were resuspended in 150 μL/well FACS buffer. If cells were not analyzed immediately, cells were fixed using a solution of 4% PFA in PBS by resuspending cells at 100 μL/well. Cells were incubated in the dark at RT for 20 min. The fixation buffer was diluted with 100 μL/well FACS buffer, and cells were pelleted at 400×g for 4 min. Supernatant was collected and disposed of as hazardous waste. Cells were washed with 200 μL/well FACS buffer and centrifuged at 400×g for 4 min. Supernatant was discarded and cells were resuspended in a final volume of 150 μL/well FACS buffer. Fixed cells were stored at 4° C. protected from light until flow cytometry analysis. From each sample, 75 μL was acquired.

For flow cytometry analysis, fluorescence compensation was set up using compensation beads. One drop of OneComp beads were incubated with 1 μL of a single fluorescent antibody at 4° C. for at least 15 min. For live/dead staining, stock reagent was diluted 1000-fold in PBS. To one drop of ArC positive beads, 100 μL of diluted dye was added. After incubation compensation controls were then washed with at least 1 mL of FACS buffer and pelleted at 400×g for 4 min. Supernatant was discarded and beads were resuspended in 300 μL FACS buffer. To the ArC positive beads, one drop of ArC negative beads was added. Compensation was then set up using these single stain samples on FACS Diva software, which was used to acquire sample data. Unstained OneComp beads were used as an unstained compensation control.

moDC Treatment. Human moDC were plated at 1×10⁵ cells/well in 96-well flat-bottom tissue culture plates in R10++ media by adding 100 μL of cells per well. To each well, a 25 μL solution of R10++ containing 400 ng/mL GM-CSF and 200 ng/mL IL-4 was added. After adding all treatments, cell cultures had a total volume of 200 μL/well resulting in GM-CSF and IL-4 final concentrations of 50 ng/mL and 25 ng/mL, respectively.

Lyophilized R848 stocks were prepared as described above and frozen at −80° C. An aliquot of frozen R848 was thawed and further diluted to 11.4 μM in R10++ (4 μg/mL R848). Cell culture conditions receiving R848 treatment had 50 μL added to wells. For wells not receiving R848, 50 μL R10++ was added to wells. Lyophilized MCC950 was prepared as described above and stored at −20° C. MCC950 was further diluted to 40 uM in a prepared R10++ solution containing 11.4 μM R848. Cells receiving R848 and MCC950 treatment had 50 μL added to wells. Final concentrations of R848 and MCC950 in a total 200 μL volume were respectively 2.85 μM and 10 μM.

A4.44% solution of KP407 dissolved in sterile, distilled water was prepared at a temperature of 4° C. DGP (Compound 2) from powder was prepared by adding KP407 solution to lipid. Using a magnetic stir bar, the solution was agitated at 1000 rpm for 1 hour at RT. Sterile 10×PBS was added to the solution resulting in a 1×PBS concentration. The mixture was then stirred at 1000 rpm for another 30 min at RT. The final stock concentration of DGP was 650 μg/mL in 4% KP407 in 1×PBS. The stock solution was then further diluted in PBS to achieve an 8× concentration of DGP from final target. DGP was then added to cell cultures at 25 μL/well. For wells not receiving DGP, 4% KP407 in 1×PBS was diluted in PBS and added to cells as a vehicle control. Volume of vehicle control used matched the highest concentration of DGP used in the study (82.5 μM).

Plates were sealed with Breathe-Easy Membranes plate sealers and placed in a 37° C., 5% C₀₂ incubator overnight. For study readouts, plates were centrifuged at 400×g for 4 min and cell culture supernatant was collected in 96-well U-bottom plates. Cells were resuspended in PBS and transferred to 96-well V-bottom plates for staining.

IL-1β ELISA. Human IL-1β ELISA kits from Biolegend were used to measure IL-1β in cell culture supernatant. Using kit reagents, ELISA plates were coated overnight at 4° C. with capture antibody diluted 200-fold in 1× coating buffer. All washes done for ELISA plates utilized a plate washer programmed to aspirate liquid and wash wells with 300 μL/well PBST (1×PBS containing 0.1% Tween 20) for 4 cycles per wash. After coating, plates were washed and blocked for 1 hour at RT using Blocking Buffer A from kit reagents. For all incubation steps except the final one of the assay procedure, plates were incubated at RT on an orbital shaker set at 200-300 rpm. During blocking incubation, the IL-1β standard was dissolved in 200 μL of 1× Assay Diluent A. A titration was set up with the highest standard at 1000 μg/mL and subsequent concentrations at two-fold dilutions down to 15.6 μg/mL. A blank sample containing 0 pg/mL IL-113 was also used. Cell culture supernatants were prepared by diluting two-fold in 1× Assay Diluent A. Plates were washed and 50 μL/well of Assay Diluent D was added to all wells. Standards and diluted samples were plated at 50 μL/well and incubated on an orbital shaker for two hours at RT. The detection antibody was prepared by diluting 200-fold in 1× Assay Diluent A. Plates were then washed and 100 μL/well of detection antibody was added. Plates were incubated at RT on the orbital shaker for an hour and then washed. Avidin-HRP reagent was diluted 1000-fold in 1× Assay Diluent A and 100 μL/well was added. Plates were incubated on the orbital shaker for 30 min at RT. Plates underwent two sets of washes before 100 μL/well Substrate Solution F was added. Plates were incubated in the dark for up to 15 min without shaking before 100 μL/well 2N sulfuric acid was added to wells to stop the reaction. Plates were read immediately on the Spectramax M3 plate reader using the absorbance setting for wavelengths 450 and 570 nm.

Background absorbance values at 570 nm were subtracted from 450 nm values. IL-1β concentrations were determined by plotting standards and using 4-Parameter Logistic curve fitting to interpolate sample concentrations. Interpolations were then corrected for 2-fold sample dilutions.

LDH Release Assay. Lactate dehydrogenase (LDH) reagents were stored in −20° C. and allowed to return to RT before starting the assay. About 45 min prior to supernatant collection, 20 μL/well of 10× lysis buffer from LDH release assay kit was added to LDH maximum control wells. Lysis was allowed to occur for 45 min in the 37° C. incubator before plates were spun at 400×g for 4 min. During the incubation, the Substrate Mix was resuspended in 11.4 mL of UltraPure Distilled Water, and 600 μL of Assay Buffer Stock Solution was added to it to make the Reaction Mixture. After vortexing, Reaction Mixture was kept at RT in the dark until use. Freshly collected cell culture was used for the LDH release assay. 25 μL of sample supernatant was added to the wells of a 96-well flat bottom ELISA plate, and an additional 25 μL of kit Reaction Mixture was added to wells. Samples were gently tapped to mix and left to incubate at RT protected from light for 30 min. 25 μL of kit Stop Solution was added to each well and samples were gently tapped to mix samples. Absorbance was read on the Spectramax M3 plate reader at 490 nm and 680 nm.

To analyze data, 680 nm absorbance readings were subtracted from 490 nm values. Values from blank media control samples were used to remove background signal. Samples were divided by the mean of the LDH maximum samples (lysed cells controls) and multiplied by 100 to obtain percent cytotoxicity for a sample. The percent cytotoxicity value was subtracted from 100 to obtain the percent viability.

Staining moDC for Activation Marker Expression. After treatment and supernatant collection, cells were resuspended in 200 μL/well PBS and transferred to 96-well V-bottom plates. To pellet cells and remove supernatant, plates were spun at 400×g for 4 min for this and subsequent steps. Cells were washed again with 200 μL/well PBS and pelleted. Live/dead Near-IR staining dye was diluted 1000-fold in PBS. Cells were resuspended in 100 μL/well live/dead staining dye and incubated at 4° C. for 10 min. Fc block was diluted 100-fold in FACS buffer, and 100 μL/well was added to cells. Cells were incubated with Fc block for 10 min at 4° C. During incubation primary CCR7 antibody (clone 2H4) was diluted 100-fold in FACS buffer. Cells were pelleted and primary CCR7 antibody was added at 100 μL/well. Cells were incubated at 37° C. for 30 min. Biotinylated rat anti-mouse IgM secondary was prepared by diluting 200-fold in FACS buffer. Cells were washed twice with FACS buffer before 100 μL/well secondary antibody was added. Cells were incubated at 4° C. for 20 min. Tertiary staining antibody cocktail was prepared in a mixture of FACS buffer and Brilliant Stain Buffer (1:1 volume ratio of each buffer). Streptavidin-PE (BV421), CD11c (PE-Cy7), CD209 (PE), CD40 (UV563), CD83 (UV737), CD86 (BV711), and HLA-DR (UV395) were all diluted 200-fold. HLA-ABC (BV605) was diluted 100-fold. Cells were incubated at 4° C. for 20 min. Cells were washed twice by adding up to 200 μL/well FACS buffer, pelleting cells, and removing supernatant. Cells were then fixed using 4% paraformaldehyde in PBS. Fixative was left on cells for 20 min at RT protected from the light. The fixation buffer was diluted with 100 μL/well FACS buffer, and cells were pelleted at 400×g for 4 min. Supernatant was collected and disposed of as hazardous waste. Cells were washed with 200 μL/well FACS buffer and centrifuged at 400×g for 4 min. Supernatant was discarded and cells were resuspended in a final volume of 200 μL/well FACS buffer. Fixed cells were stored at 4° C. protected from light until flow cytometry analysis. From each sample, 150 μL was acquired for analysis.

For proper definition of negative and positive gating boundaries, FMO staining controls were used. Cells treated with 2.85 uM R848 were used for FMO staining controls. For these FMO controls, all but one staining antibody was left out of the staining process in order to determine the signal produced by a negative population for that antibody target. In the case of CCR7 staining that required a primary, secondary, and tertiary stain, only the tertiary stain was omitted for the CCR7 FMO control.

For flow cytometry analysis, fluorescence compensation was set up using compensation heads. One drop of OneComp beads were incubated with 1 μL of a single fluorescent antibody at 4° C. for at least 15 min. For CCR7 staining that required a primary, secondary, and tertiary stain, all three reagents were added to the OneComp beads at the same time. For live/dead staining, stock reagent was diluted 1000-fold in PBS. To one drop of ArC positive beads, 100 μL of diluted dye was added. After incubation compensation controls were then washed with at least 1 mL of FACS buffer and pelleted at 400×g for 4 min. Supernatant was discarded and beads were resuspended in 300 μL FACS buffer. To the ArC positive beads, one drop of ArC negative beads was added. Compensation was then set up using these single stain samples on FACS Diva software which was used to acquire sample data. Unstained OneComp beads were used as an unstained compensation control.

Data analysis. Data were analyzed and graphed using Microsoft Excel and GraphPad Prism software. Data are reported in graphs as mean with ranges indicating SD. When data from a single human donor are represented in a graph, data points represent biological replicates. When graphs represent data from multiple donors, each data point represents the mean of three biological replicates from a donor.

For IL-1β and LDH release assay statistical analysis, ordinary two-way ANOVA was conducted, followed by Tukey's multiple comparisons test with a single pooled variance. Statistical testing of staining data utilized one-way ANOVA, followed by Tukey's comparisons with individual variances computed for each comparison. Statistical testing was completed using Prism.

### Results

Human Monocytes Differentiate into moDC with a cDC2 Phenotype. After culturing monocytes in media containing GM-CSF and IL-4 for 6 days, cells were collected for use. Cells were stained with fluorescent antibodies to determine cell surface expression of CD11c and CD209. Both monocytes and moDC express CD11c but only moDC express CD209 (Geijtenbeek et al., Cell, 100(5):575-585, 2000). For the four donors used in this study, 85% or more of the live cell population were CD11c+CD209+, indicating that the monocytes had differentiated into moDC. No more than 2.18% of cells stained CD11c+CD209−, indicating that few cells remained as undifferentiated monocytes. Of the live CD11c+CD209+ cells, greater than 90% of cells stained SIRPa+, a marker of the cDC2 subset. Altogether, staining for CD11c, CD209, and SIRPa indicated that the monocyte cell cultures were successfully differentiated into DCs with a cDC2-like phenotype.

R848+DGP Hyperactivates Human moDC. Human moDC were collected after the differentiation process and plated for in vitro stimulations using various combinations of R848, DGP, and MCC950. Cells were treated with or without 2.85 μM R848 and with or without 10 μM MCC950. DGP was added at multiple concentrations: 0; 10.3; 20.6; 41.3; and 82.5 μM. After an overnight incubation with treatments, cell culture supernatant was collected to determine if cells were hyperactivated.

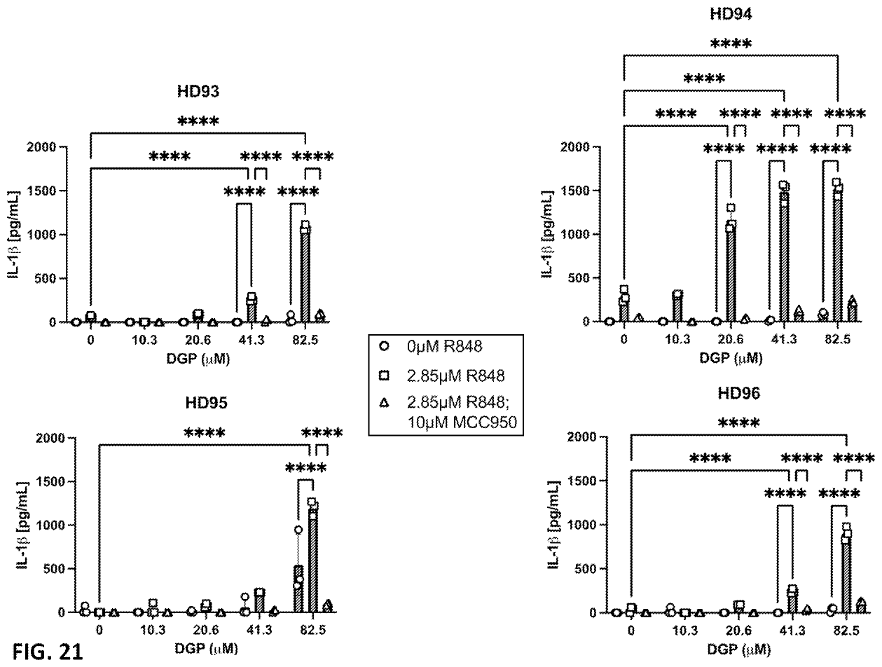

Without addition of R848, IL-1β was undetectable or less than 100 μg/mL (FIG. 21). One exception was donor HD95, which produced detectable levels of IL-1β with treatment of cells with 82.5 μM DGP alone. Treatment of moDC with only R848 also produced minimal IL-1p in the range of 0 to 100 μg/mL (FIG. 21). Another exception was HD94, which produced 288 μg/mL IL-1β. Comparing to R848 treatment alone, treatment of moDC using R848 and DGP at 82.5 μM significantly increased IL-1β secretion in all four donors (FIG. 21, p<0.0001). Depending on the donor, varying concentrations of DGP were sufficient in combination with R848 to significantly increase IL-1β secretion (FIG. 21, all p<0.0001). HD93 and HD96 required at least 41.3 uM DGP, whereas HD94 needed 20.6 μM, and HD95 needed 82.5 μM.

IL-1β release was NLRP3-dependent. When 10 μM MCC950 was added with R848+DGP combinations, IL-1β concentrations in supernatants fell to baseline levels, similar to R848 alone treatment (FIG. 21, p<0.0001). These results indicated that nearly all IL-1β secretion could be attributed to NLRP3 activation. Given the NLRP3-dependence, most if not all detected IL-1β was the mature cleaved form and could not be pro-IL-1β being released by other mechanisms.

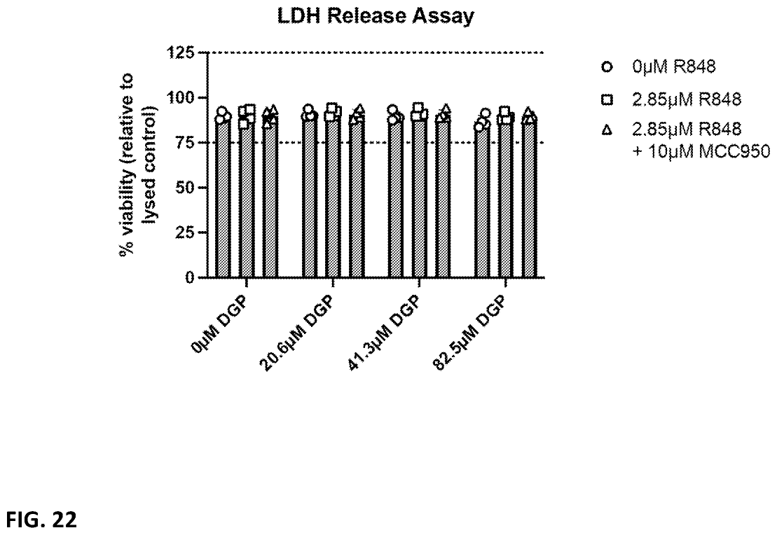

IL-1β release can occur via hyperactivation or pyroptosis. Retention of cell viability is a defining characteristic of hyperactivation that contrasts the cell death that occurs during pyroptosis. To differentiate between hyperactivation and pyroptosis when IL-1β is released, cell viability was assessed. Cell culture supernatant was measured for LDH activity as an indicator of cell cytotoxicity. For all four donors across all conditions indicated, cell viability was within +/−25% of 100% viability, which was set as an acceptable range for cell cultures (FIG. 22). Additionally, LDH activity was nearly the same across all conditions, indicating minimal differences in cell cytotoxicity (FIG. 22).

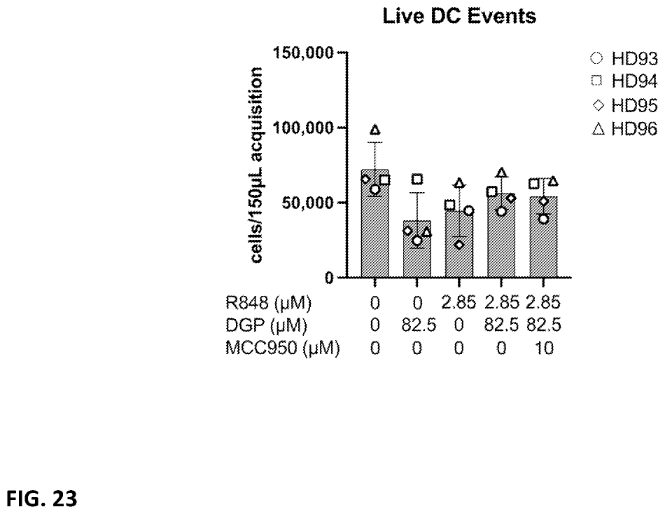

Cells from various treatment conditions were collected for fluorescent staining and flow cytometry. As an additional measure of cell viability, the number of live, CD11c+CD209+ events from each treatment condition were enumerated. No significant differences in live cell numbers were found when comparing any two treatment conditions (FIG. 23). These viability data indicate that in conditions where IL-1β was released, moDC were hyperactivated.

Hyperactivating Treatments Do Not Diminish DC Activation Marker Expression. After cell staining and flow cytometry, expression of various activation markers by moDC was analyzed. Analysis was done by gating on live CD11c+CD209+ events to determine how R848 and DGP affect marker expression individually and in combination. Marker expression was analyzed using two methods, quantifying the percentage positive for staining and quantifying the median fluorescence intensity (MFI) of the population. The percent positive staining indicates how much of the cell population is expressing the activation marker whereas MFI quantifies how much expression of the marker is occurring on individual cells.

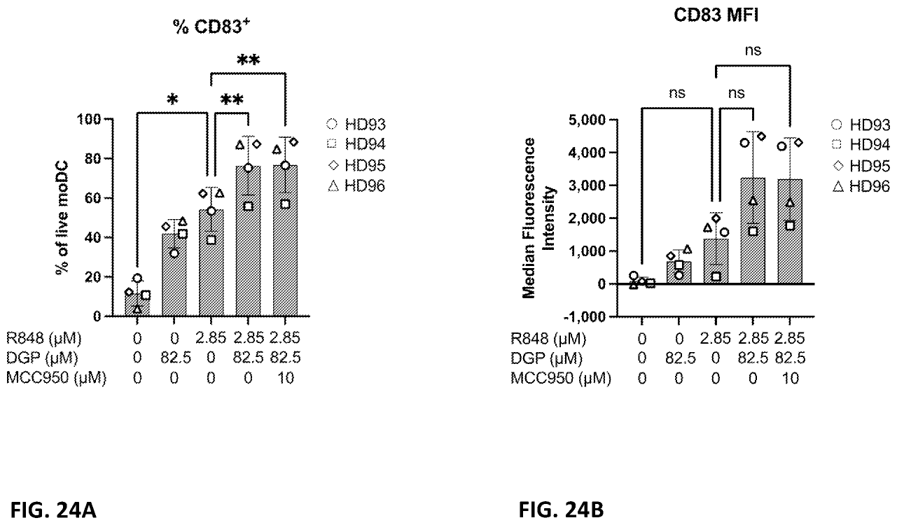

CD83 is often used as a marker of DC activation, and it has been found to regulate the expression of MHC-II molecules (Li et al., Frontiers in Immunology, 10, 1312, 2019). On unstimulated cells a mean of 12% moDC expressed CD83. Treating cells with 82.5 μM DGP increased CD83 expression, but increase was not found to be statistically significant (FIG. 24A). Treatment of cells with R848 resulted in stronger CD83 expression in the live moDC population, resulting in a statistically significant difference in comparison to unstimulated cells (FIG. 24A, p<0.05). By combining R848 with 82.5 μM DGP, a statistically significant increase in percentage of CD83+ cells occurred as compared to R848 treatment alone (p<0.01), and this increase was independent of NLRP3 activation (FIG. 24A). While similar trends were observed with CD83 MFI, differences were not statistically significant (FIG. 24B). These data indicate that R848 induces CD83 expression and that hyperactivation induces more cells to express CD83.

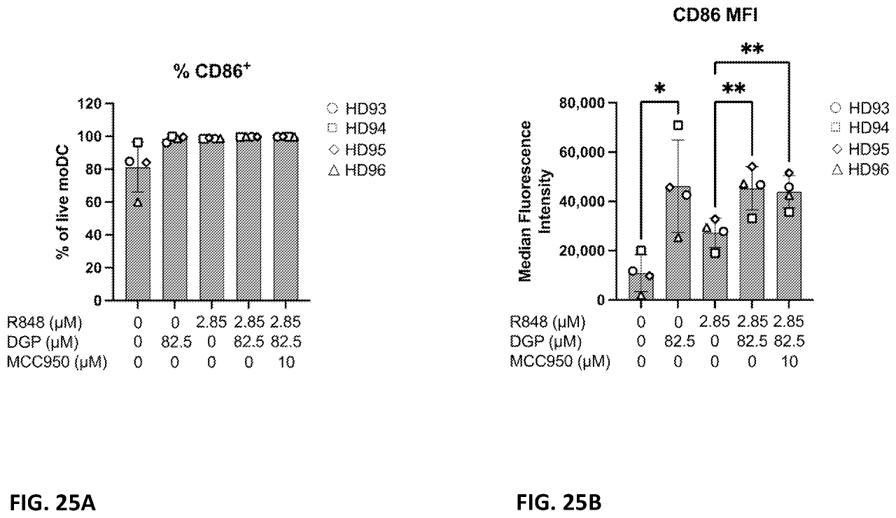

CD86 serves as a co-stimulatory molecule to T cell receptor signaling to fully activate T cells (Lim et al., PLoS One, 7(9), e45185, 2012). On average, 81% of moDC expressed CD86 when left unstimulated, and treatment with R848, DGP, or in combination resulted in >98% cells expressing CD86 (FIG. 25A). MFI analysis indicated that DGP treatment alone resulted in significant increases in CD86 expression as compared to unstimulated cells (FIG. 25B, p<0.05). Compared to R848 treatment alone, hyperactivated cells significantly increased CD86 expression (p<0.01), and inhibition of NLRP3 via MCC950 treatment did not abrogate this upregulation (FIG. 25B, p<0.01). These data indicate that combination of R848 and DGP induces upregulation of CD86 expression independent of NLRP3 inflammasome activation.

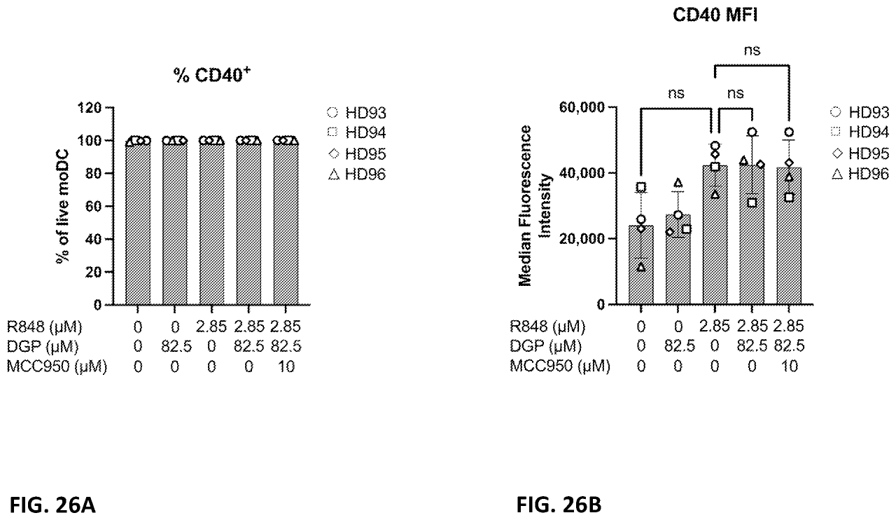

CD40 is a receptor that engages with CD4 T cells during T cell activation to induce IL-12 production from DCs (O'Sullivan and Thomas, Critical Reviews in Immunology, 23(1-2):83-107, 2003). This serves as a positive feedback loop as IL-12 then acts on T cells. Nearly all (>99%) moDC derived from all four donors expressed CD40 (FIG. 26A). On a per cell basis. CD40 expression increased when cells were stimulated with R848 alone or in combination with DGP, but the magnitude of MFI increases varied depending on the donor such that the results were not statistically significant (FIG. 26B).

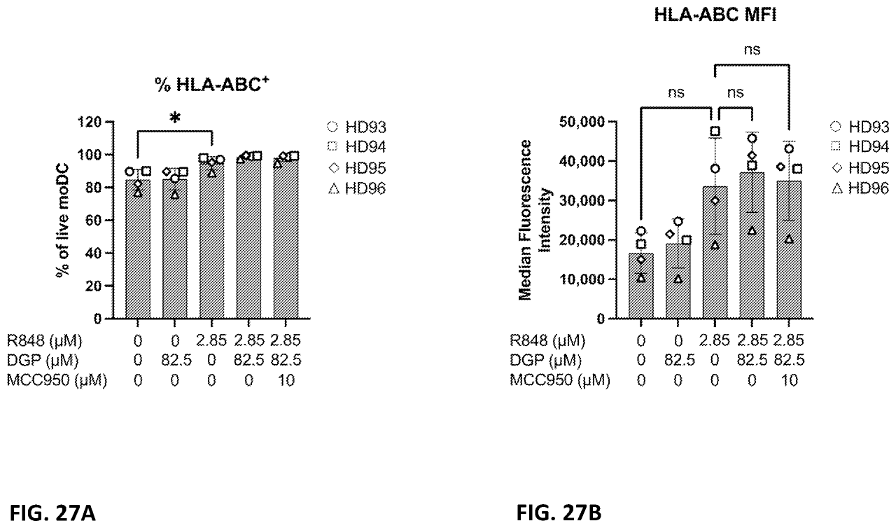

Most moDC (75-99%) stained positive for HLA-ABC expression, which are MHC Class I molecules that are necessary for antigen presentation to CD8+ T cells. R848 alone or in combination with DGP resulted in a small but significant increase in cells staining positive for HLA-ABC compared to untreated cells, indicating that R848 stimulation results in an increase in MHC Class I expression (FIG. 27A, p<0.05). Similar to CD40 staining, MFI signal increased when cells were treated with R848 compared to unstimulated cells. However, the magnitude of increase varied depending on donor leading to a statistically insignificant shift in HLA-ABC MF (FIG. 27B).

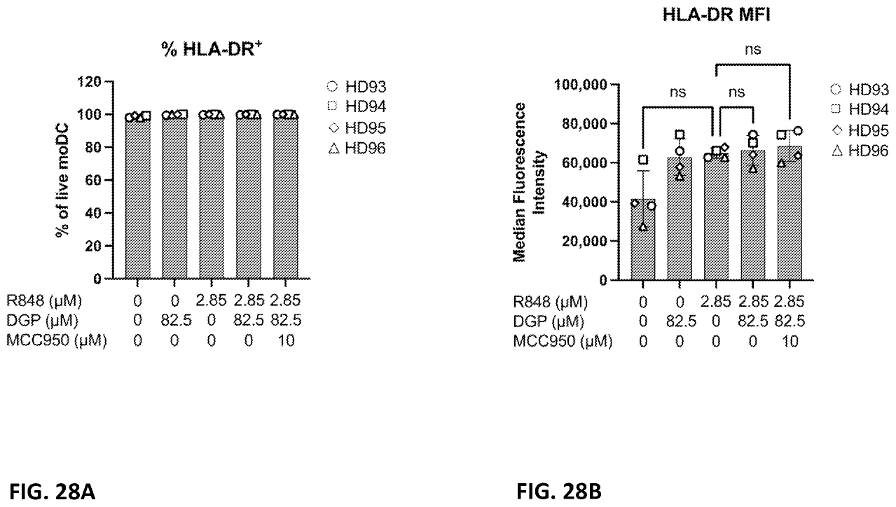

Staining for MHC Class 11 molecules, which are required for antigen presentation to CD4+ T cells was assessed with an anti-HLA-DR antibody. Nearly all cells (>98%) in all conditions tested were HLA-DR+ indicating that MHC Class II molecules are constitutively expressed on moDC (FIG. 28A). The variability observed among donors suggested that treatments with R848, DGP, and MCC950 did not significantly affect the degree of expression on a per cell basis (FIG. 28B).

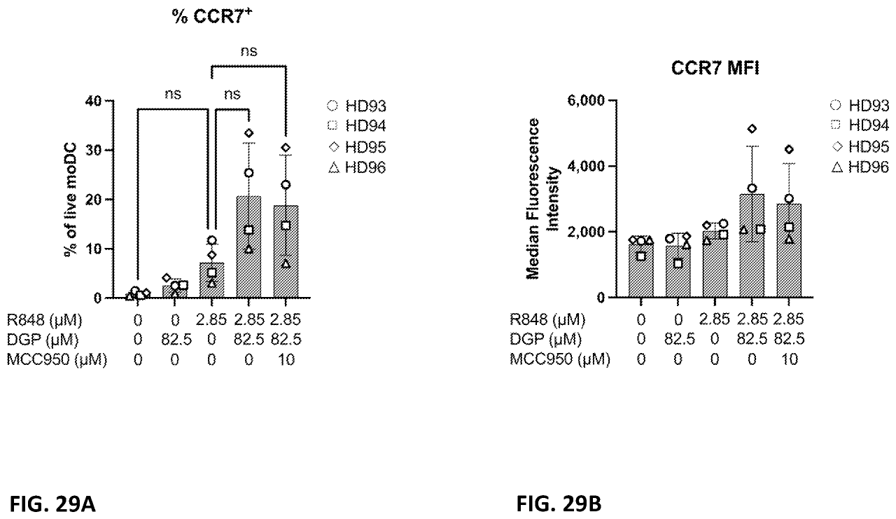

Finally, CCR7 expression was assessed. CCR7 is required for DC homing to lymph nodes, which is an important activity for initiation of T cell responses (Furster et al., Cell, 99(1):23-33, 1999). When left unstimulated, less than 1.5% of cells expressed CCR7 (FIG. 29A). A trend of increased CCR7 expression was observed when cells were treated with R848 or DGP, and further increases in CCR7 expression were observed when R848 was combined with DGP to hyperactivate cells. However, those increases were variable depending upon donor and not statistically significant (FIG. 29A). These trends were similar regardless of whether or not MCC950 was added with the hyperactivating stimuli (FIG. 29A). These data suggest that R848 and DGP may induce CCR7 expression on moDC. At an individual cell basis, MFI measurements suggested that the change in CCR7 expression was minimal and not statistically significant since the MFI observed was similar across the different treatment groups (FIG. 29B).

Altogether, R848 and DGP treatment had variable results on cell expression depending on the activation marker of interest. However, one important theme across all activation markers was that treatment of moDC with R848+DGP did not lead to any significant reductions in activation marker expression compared to unstimulated cells or single agent treatments. Statistically significant increases in expression of CD83 and CD86 observed for hyperactivated moDC was not NLRP3 dependent, suggesting that inflammasomes and IL-1β were not mechanistically involved in the upregulation of CD83 and CD86.

### Example B-7: Cytokine Expression and Migratory Capacity of Hyperactivated moDCs

### Materials and Methods

Monocyte Isolation, Storage, and Differentiation into moDC. Quality Control Staining of moDC Differentiation, moDC Treatment, and LDH Release Assays were performed as described in Example B-6.

Induction of Pyroptosis. Human moDCs were treated with various stimuli for 4 hours in a priming phase, which allowed cells to produce pro-IL-1β. Stock nigericin at 6.7 mM was then added to wells at 3 μL/well to achieve a final concentration of 100 μM.

CellTiter-Glo 2.0 Cell Viability Assay. As an additional method to measure cell viability, CellTiter-Glo 2.0 Cell Viability Assay kit from Promega was used. CellTiter-Glo 2.0 reagent was used according to instructions of the manufacturer. Briefly, frozen reagent was thawed at 4° C. and aliquoted. Aliquots were stored at 4° C. in the dark. After removing 150 μL of the cell culture volume for use in other assays, cells were lysed with 50 μL/well CellTiter-Glo reagent by pipetting up and down. After incubating in the dark at RT for 10 min, samples were transferred to white opaque 96-well plates. Luminescence was measured for 500 ms/well using a Spectramax M3 plate reader. Luminescence values were normalized to the mean values of control samples and multiplied 100-fold to report values as percentage viability.

Lumit Human IL-6 Immunoassay. To determine the concentration of IL-6 secreted into the cell culture supernatant, the Lumit IL-6 Immunoassay was used. Conceptually, two antibodies are used to bind to IL-6 in the sample, and each antibody is tethered to a subunit of an enzyme. Binding of the two antibodies to IL-6 enables the subunits to be in close enough proximity to bind and function. A substrate for the enzyme is then added to create a luminescent signal that indicates the concentration of IL-6 in the supernatant.

The kit standard was prepared in cell culture medium (R10++) starting at 25,000 μg/mL with two-fold dilutions to 24.4 μg/mL. A blank control was also included. Sample supernatants were diluted 5-fold in R10++. To prepare antibodies, 12 μL of each antibody was diluted in 5,976 μL of R10++ (or proportionately modified for the volume required) to reach a 2-fold concentrate. Antibodies were combined with sample supernatant or standards (12.5 μL of each) by pipetting into white opaque 384-well plates. Samples were then incubated at 37° C. for 1 hour. Samples were then allowed to cool to RT in the dark while preparing substrate. For every 3,040 μL of Detection Buffer B used, 175 μL of Detection Substrate B was added. Substrate was mixed by briefly vortexing and was added at 6.25 μL/well. On the Spectramax M3 plate reader, sample luminescence was recorded at 500 ms/well. Prism software was used to construct a standard curve using 4-parameter logistics analysis. Interpolated results were corrected for sample dilution.

LegendPlex Assay. To detect multiple cytokines of interest (IL-1β, IL-10, IL-12p70, IP-10, and IFNa2), the predefined LEGENDplex Human Anti-Virus Immune Response Panel from Biolegend was utilized. Conceptually, each targeted cytokine has antibodies bound to a unique bead population defined by bead size and APC fluorescence signal. When incubated with sample supernatant, cytokines are bound by the antibodies. Biotinylated secondary antibodies are then added to bind the cytokines of interest, and streptavidin-bound PE is then incubated with samples. Using flow cytometry, each bead population specific for a cytokine can be defined based on bead size and APC signal intensity. The PE fluorescence signal on beads then indicates the amount of cytokine present in the sample.

To run the assay, samples were diluted 5-fold in Assay Buffer. Standards were prepared according to manufacturer instructions. Lyophilized standard was reconstituted in 250 μL Assay Buffer. After a 10 minute incubation at RT, this top standard concentration was serially diluted 4-fold to make an 8-point standard curve with the last point set as blank buffer. In each well of a V-bottom plate, 25 μL Assay Buffer was added with 25 μL of sample or standard. Beads were vortexed thoroughly and added at 25 μL/well. Samples were incubated in the dark at RT for 2 hours on an orbital shaker set at 200-300 rpm. The heads were then pelleted at 250×g for 5 min and resuspended in 200 μL/well 1× wash buffer. After pelleting beads again, samples were resuspended in 25 μL/well Detection Antibodies. Samples were incubated for 1 hour at RT in the dark on the orbital shaker. Without washing, 25 μL/well of streptavidin-PE was added to samples and incubated at RT in the dark for 30 min on the orbital shaker. After the incubation, beads were washed again and resuspended in 150 μL/well wash buffer. Samples were analyzed the same day using the Novocyte Quanteon flow cytometer.

Raw data was exported as FCS files and uploaded to the LEGENDplex Data Analysis Software Suite from Qognit for analysis. Five parameter logistics curves were constructed from the standards, and sample results were interpolated using the cloud software. Sample dilutions were incorporated into the analysis, and correct bead population gating was verified on all samples. Results were exported from the cloud software as a Microsoft Excel file.

In Vitro Transwell Migration Assay. To test the capacity of human dendritic cells to migrate, an in vitro migration assay was set up. After differentiation into moDC using GM-CSF and IL-4, cells were plated on 10 cm petri dishes at 4×10⁶ cells/plate in R10++. Cells were treated with various combinations of 2.85 μM R848, 82.5 μM DGP, and 10 μM MCC950 (final concentrations). Additionally, 50 ng/mL GM-CSF and 25 ng/mL IL-4 was added to the plates. Cell cultures were incubated in a total of 8 mL/plate in a 37° C. incubator on an orbital shaker set at 100 rpm overnight.

Cells were collected from plates and any remaining cells on plates were washed off the plates using PBS. After pelleting cells at 400×g for 5 min, cells were resuspended in R10++ and counted. Cell concentrations were adjusted to 5×10⁵ cells/mL and plated in triplicate in the apical chambers of transwells at 1.5×10⁵ cells/well. R10++ media containing 0; 20; or 200 ng/mL CCL19 were prepared and added to the basal chambers at 300 μL/well. For each cell treatment condition, control wells without transwell inserts were plated with an equivalent volume of cells and R10++ media. Cells were allowed to migrate overnight in a 37° C. incubator.

Cells from the basal chamber were collected by resuspending basal chamber media and transferring to collection plates. Wells were additionally incubated for at least 10 min at 4° C. and washed with cold PBS containing 1% EDTA to dislodge any adherent cells. Cells were then stained for flow cytometry analysis. After washing twice with PBS, Live/Dead NIR stain was diluted 1000-fold in PBS and added onto cells at 100 μL/well. Cells were stained at 4° C. for 10 min in the dark while Fc receptor blocking antibody was diluted 100-fold in FACS buffer. Fc block was added to cells at 100 μL/well and incubated for another 10 min at 4° C. Cells were then pelleted at 400×g for 4 min. Surface staining antibodies targeting CD11c and CD209 were diluted 200-fold in a mixture of Brilliant Staining Buffer and FACS buffer (1:1 volume ratio). Cells were resuspended in antibody cocktail at 100 μL/well and incubated at 4° C. for 20 min. FACS buffer was then added to reach a volume of 200 μL/well, and cells were spun at 400×g for 4 min. Cells were washed again with 200 μL/well FACS buffer and pelleted once more. Cells were then fixed using a PBS solution of 4% paraformaldehyde by resuspending cells at 100 μL/well and incubating at RT in the dark for 20 min. The fixation buffer was diluted with 100 μL/well FACS buffer, and cells were pelleted at 400×g for 4 min. Supernatant was collected and disposed of as hazardous waste. Cells were washed with 200 μL/well FACS buffer and centrifuged at 400×g for 4 min. Counting beads were vortexed to resuspend them and then diluted 4-fold in FACS buffer. Supernatant was discarded and cells were resuspended in a final volume of 200 μL/well FACS buffer containing counting beads. Fixed cells were stored at 4° C. protected from light until flow cytometry analysis. From each sample, 150 μL was acquired.

To enumerate migrated DCs, an absolute count was obtained based on the total events acquired of counting beads and live, CD11c+CD209+ single cells. Counts from each cell treatment were normalized to control wells not containing transwells to account for potential plating discrepancies between treatment conditions. Finally, migration was calculated relative to cells treated with only R848 with no CCL19 added.

Data Analysis. Data were analyzed and graphed using Microsoft Excel and GraphPad Prism software. Data are reported in graphs as mean with error bars indicating SDs. Graphs depict data from individual donors, and data points represent biological replicates.

For cytokine and migration data statistical analyses, ordinary two-way ANOVA was conducted, followed by Tukey's multiple comparisons test with a single pooled variance. Statistical testing was completed using Prism software.

### Results

Human Monocytes Differentiate Into moDCs With a cDC2 Phenotype. After culturing monocytes in media containing GM-CSF and IL-4 for 6 days, cells were collected for use. Cells were stained with fluorescent antibodies to determine cell surface expression of CD11c and CD209. Both monocytes and moDC express CD11c but only moDC express CD209 (Geijtenbeek et al., Cell, 100(5):575-585, 2000). For the three donors used in this study, 80% or more of the live cell population were CD11c+CD209+, indicating that the monocytes had differentiated into moDC. No more than 1.6% of cells stained CD11c+CD209− indicating that few cells remained as undifferentiated monocytes. Of the live CD11c+CD209+ cells, greater than 88% of cells stained SIRPa+, a marker of the cDC2 subset. Altogether, staining for CD11c, CD209, and SIRPa indicated that the monocyte cell cultures were successfully differentiated into DCs with a cDC2-like phenotype.

R848+DGP Hyperactivates Human moDCs. Human moDCs were collected after the differentiation process and plated for in vitro stimulations using various combinations of R848, DGP, and MCC950. Cells were treated with or without 2.85 μM R848, with or without 82.5 μM DGP, and with or without 10 μM MCC950. To compare R848+DGP hyperactivation to a pyroptotic condition, cells were treated with R848 combined with 100 μM nigericin (with or without MCC950 treatment). After an overnight incubation with treatments, cell culture supernatant was collected to determine if cells were hyperactivated. For all statistical analyses to compare treatments, ordinary 2-way ANOVA was employed followed by Tukey's multiple comparisons test with a single pooled variance.

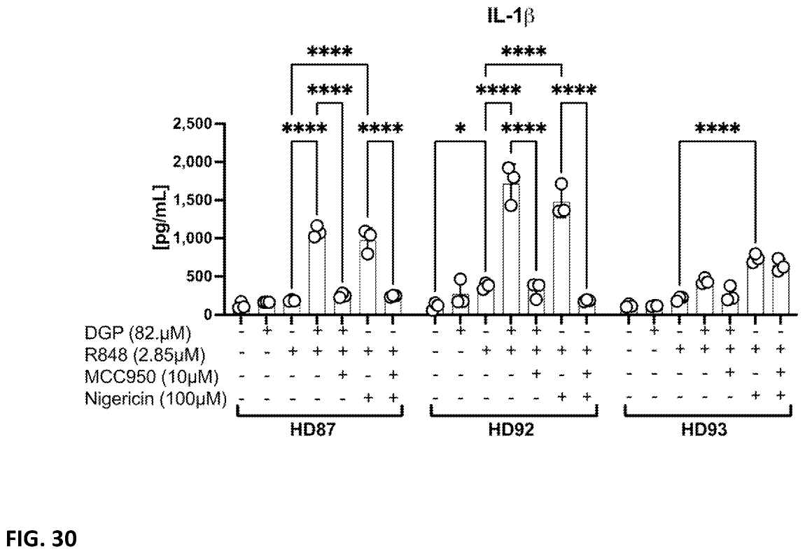

Unstimulated moDCs and cells treated with a single agent (R848 or DGP) yielded low concentrations of IL-1β below 500 μg/mL (FIG. 30). A statistically significant increase of IL-1β was detected when comparing unstimulated cells to R848 treatment only for HD92 (FIG. 30, p<0.05). The combination treatment of R848+DGP induced a statistically significant increase of IL-1β release compared to R848 treatment alone for HD87 and HD92 (FIG. 30, p<0.0001). While HD93 had an increase in IL-1β release when comparing R848 treatment to R848+DGP, the result was not statistically significant (FIG. 30).

IL-1β release was NLRP3-dependent. When 10 μM MCC950 was added with R848+DGP combinations, IL-1β concentrations in supernatants fell to less than 400 μg/mL, similar to R848 alone treatment (FIG. 30, p<0.0001 for HD87 and HD92). These results indicated that the IL-1β secretion from R848+DGP above what was observed with R848 treatment alone could be attributed to NLRP3 activation. Given the NLRP3-dependence, most if not all detected IL-1β was the mature cleaved form and could not be pro-IL-1β being released by other mechanisms.

IL-1β release can also occur via pyroptosis. To stimulate pyroptosis, moDC were treated with a combination of R848+nigericin. R848 primes cells via TLR signaling, and nigericin induces potassium efflux that activates the NLRP3 inflammasome (Muñoz-Planillo et al., Immunity, 38(6):1142-1153, 2013). Compared to R848 treatment alone, all three donors secreted significantly more IL-1β when nigericin was added (FIG. 30, p<0.0001).

When MCC950 was added to the pyroptotic treatment condition, a significant reduction in IL-1β was detected in HD87 and HD92 cultures suggesting that the IL-1β release was NLRP3-dependent (FIG. 30, p<0.0001), in agreement with previous literature suggesting that nigericin induces pyroptosis via the NLRP3 inflammasome (Mariathasan et al., Nature, 440(7081):228-232, 2006).

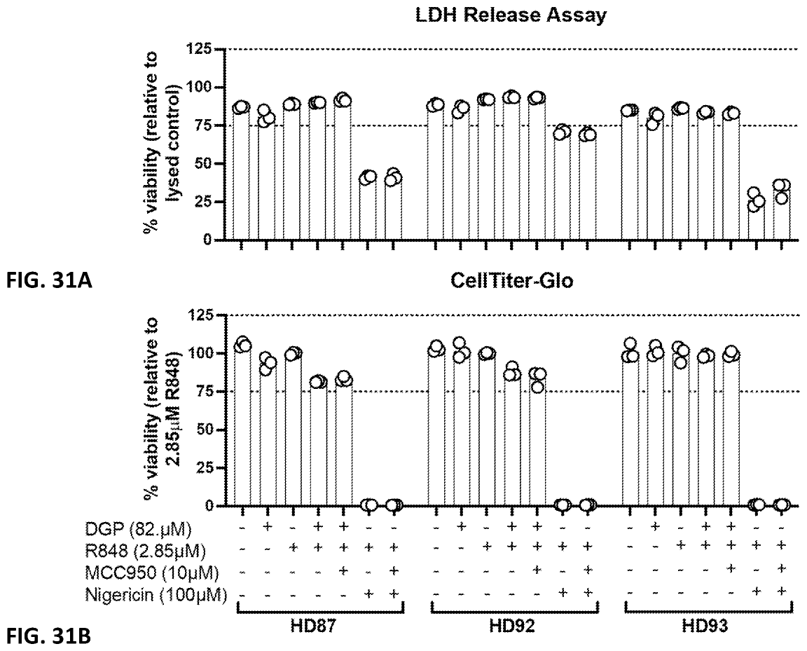

Pyroptosis results in cell death, whereas hyperactivation does not. Using an LDH release assay, the various treatment conditions were measured for cell viability. For all three donors, treatment with nigericin resulted in cell viability falling below 75%, confirming that pyroptosis occurred (FIG. 31A). In contrast, all other treatment conditions remained above 75% viability (FIG. 31A). In particular, these results indicated that R848+DGP induced elevated concentrations of IL-1β that were not accompanied by cell viability loss and are a result of moDC hyperactivation.

To further confirm the viability of cells, a second viability assay (CellTiter-Glo) was employed. The results were more drastic compared to the LDH release assay. Pyroptosis from nigericin treatment resulted in cell viability below 2% (FIG. 31B). In contrast, all other conditions maintained cell viability above 75% (FIG. 31B). The discrepancy in the severity of cytotoxicity was likely due to fundamental differences in the two assays. The LDH assay measures LDH enzymatic activity in the cell culture supernatant whereas the CellTiter-Glo assay measures adenosine triphosphate (ATP) as a proxy for cell number in culture. The two assays likely have differences in the dynamic range in their detection and differences in the half lives of the two molecules being detected. Nonetheless, the two assays demonstrated that treatment with R848+DGP resulted in IL-1β release while viability remained similar to unstimulated cells and cells treated with a single agent. Treatment of cells with R848+nigericin, in contrast, also induced IL-1β release but resulted in pyroptotic cell death. Differences in hyperactivation were apparent when comparing donors. While HD87 and HD92 showed robust hyperactivation as measured by IL-1β secretion, HD93 demonstrated the poorest induction of hyperactivation using R848+DGP.

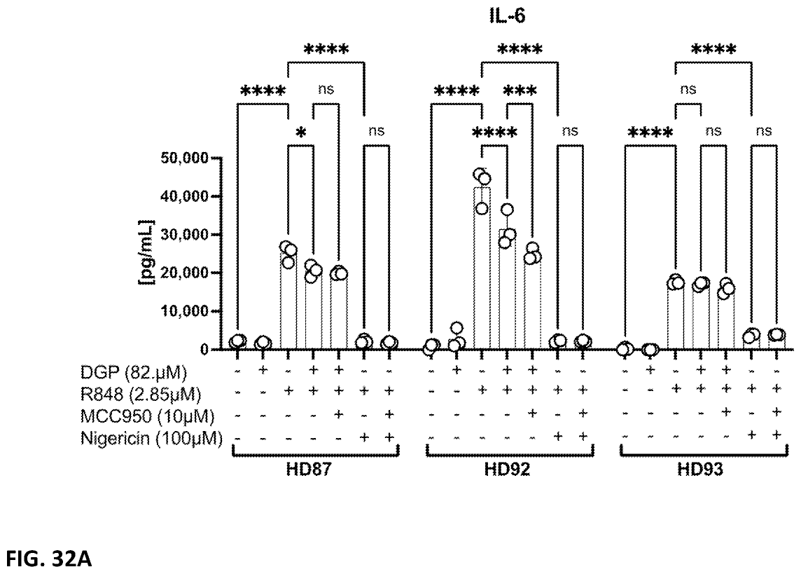

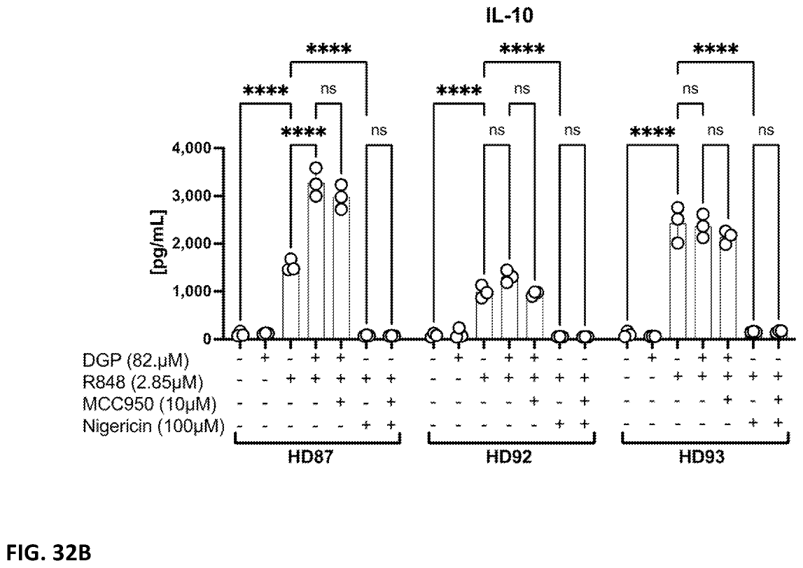

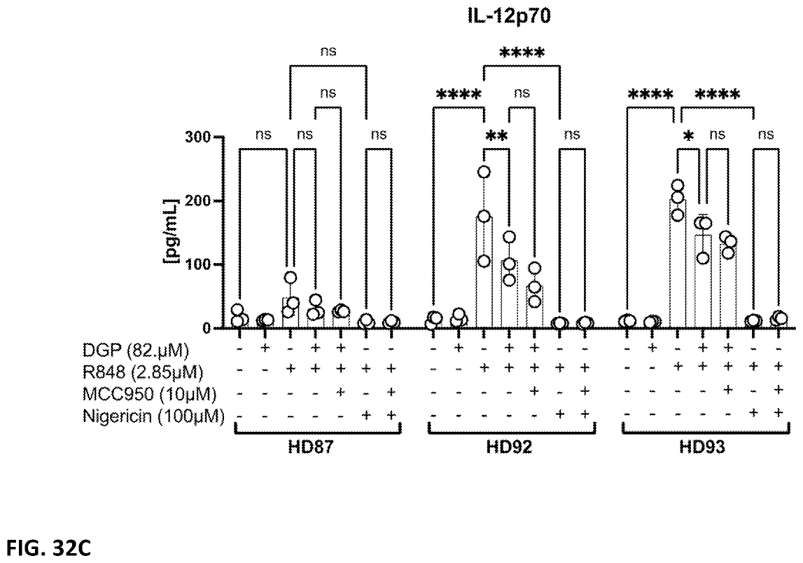

Hyperactivation of moDCs Using R848+DGP Results in R848-Dependent Cytokine Expression. IL-1β is an important inflammatory cytokine, but DCs also release additional inflammatory cytokines to mediate immune responses. Cell culture supernatant was further analyzed for the presence of other cytokines. R848 induces a cell signaling cascade that results in the activation of the transcription factor NF-kB and the expression of inflammatory genes (Jurk et al., Nature Immunology, 3(6), 499, 2002). When measuring for IL-6, minimal signal (<6000 μg/mL) was detected from unstimulated cells, and cells treated with DGP alone showed similar IL-6 levels (FIG. 32A). Stimulating cells with R848 resulted in significant increases in IL-6 for all three donors as expected (FIG. 32A, p<0.0001). Upon hyperactivation, moDC maintained high expression of IL-6, but depending on the donor, IL-6 concentrations were similar or slightly lower than concentrations measured from R848 treatment alone (FIG. 32A). Inhibition of the NLRP3 inflammasome by using MCC950 had minimal impact on expression of IL-6 which was expected since IL-6 is not regulated by the NLRP3 inflammasome (FIG. 32A). When pyroptosis was induced using R848+nigericin treatment, IL-1β was secreted (FIG. 30). In contrast, nigericin in combination with R848 resulted in minimal IL-6 production that was significantly lower than treatment with R848 alone (p<0.0001) and irrespective of MCC950 treatment (FIG. 32A). These results suggested that the pyroptotic stimulus nigericin prevented inflammatory cytokine production mediated by R848, whereas hyperactivation permitted IL-6 expression.

To determine if these observations applied more broadly, other cytokines were measured. IL-1β expression was induced by R848 treatment and not from unstimulated cells or by DGP treatment (FIG. 32B, p<0.0001). Hyperactivation by combining R848 with DGP resulted in equal or higher IL-1β expression compared to R848 treatment alone (FIG. 32B, p<0.0001 for H87, ns for HD92 and HD93). However, pyroptosis via R848+nigericin treatment reduced IL-1β concentrations to background levels observed in the unstimulated cell conditions for all donors and was significantly lower than R848 treatment alone in all donors (FIG. 32B, p<0.0001).

Another measured cytokine, IL-12p70, was also R848-dependent. For donors HD92 and HD93, R848 treatment resulted in mean concentrations of at least 100 μg/mL IL-12p70 (FIG. 32C). These concentrations were significantly increased from those measured from unstimulated cells (FIG. 32C, p<0.0001 for HD92 and HD93). Combining R848 with DGP resulted in a reduction in IL-12p70 (FIG. 32C, p<0.01 for HD92 and p<0.05 for HD93), but it was still measurably increased compared to unstimulated cells. MCC950 addition did not have further effect on IL-12p70 secretion (FIG. 32C). HD87 was not observed to induce IL-12p70 (FIG. 32C). Despite the variability in IL-12p70 induction by R848, none of the three donors induced IL-12p70 when pyroptotic conditions were used. In particular HD92 and HD93 had significantly decreased IL-12p70 when R848 was combined with nigericin, irrespective of MCC950 treatment (FIG. 32C, p<0.0001).

Altogether, the data demonstrated that R848 stimulation induced expression of NF-kB-dependent cytokines IL-6, IL-10, and IL-12p70. In contrast, when R848 was combined with the pyroptotic stimulus nigericin, R848-dependent cytokine expression was ablated. Thus, pyroptosis permitted IL-1β secretion but not NF-kB-dependent cytokines. In contrast, hyperactivation via R848+DGP permitted IL-1β secretion and also NF-kB-dependent cytokines.

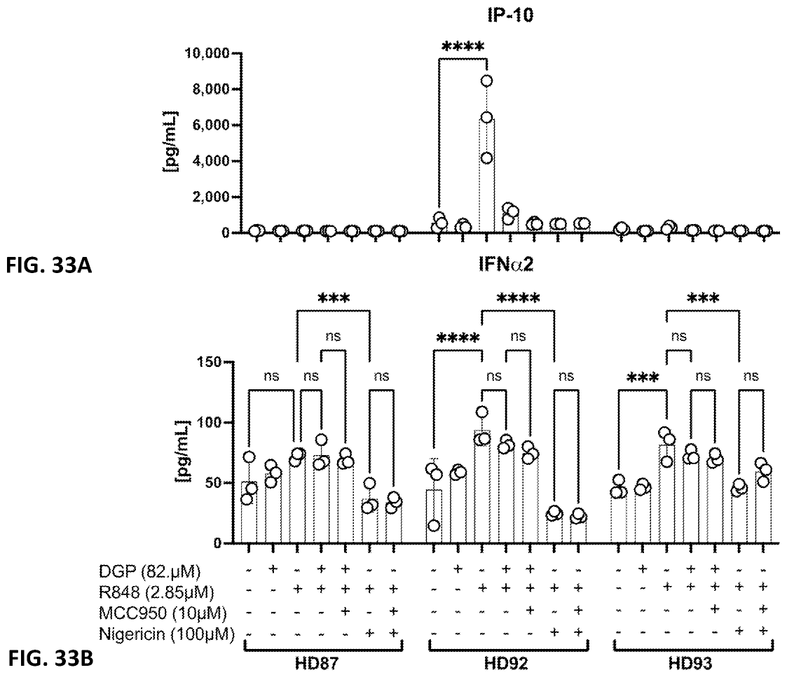

In addition to NF-kB, R848 weakly induces the expression of other inflammatory genes via interferon regulatory transcription factor 3 (IRF3) and IRF7 transcription factors (Alam et al., Frontiers in Immunology, 9, 2982, 2018). When measuring IP-10, a chemoattractant for T cells (Dufour et al., Journal of Immunology, 168(7):3195-3204, 2002), only HD92 produced IP-10 significantly above background when stimulated with R848 (FIG. 33A, p<0.0001). No other moDC stimulation conditions for HD92 produced IP-10 above background (FIG. 33A). Given that only one donor produced IP-10 for one condition, no conclusion can be made regarding IP-10 secretion.

Another IRF-dependent gene, interferon alpha 2 (IFNα2), was upregulated by HD92 and HD93. Unstimulated cells produced approximately 45 μg/mL IFNα2, and HD92 and HD93 respectively produced 94 and 82 μg/mL IFNα2 upon R848 stimulation (FIG. 33B, p<0.0001 for HD92 and p<0.001 for HD93). Hyperactivated cells treated with R848+DGP maintained elevated concentrations of IFNα2 compared to R848 treatment, irrespective of MCC950 addition (FIG. 33B). In contrast, pyroptosis by combining R848 with nigericin resulted in IFNα2 concentrations similar to unstimulated cells, significantly lower than concentrations induced by R848 alone treatment (FIG. 33B, p<0.001 for HD87 and HD93, p<0.0001 for HD92). Similar to the trends seen with NF-kB dependent genes, the IRF-driven gene IFNα2 was inhibited by pyroptosis but hyperactivation allowed for its expression. Thus, the cell death associated with pyroptosis likely renders DCs unable to induce the expression of a broad spectrum of genes that mediate the immune response via NF-kB and IRF3/7 but allows for IL-1β secretion. In contrast, hyperactivation enables IL-1β secretion and many additional inflammatory signals.

Hyperactivation Using R848+DGP Increases CCR7-Dependent Migration of Human moDCs. Given their critical role in instructing T cell activation, another important aspect of DC biology is that DCs migrate from peripheral tissues where they encounter antigen, PAMPs, and DAMPs to lymph nodes where they activate T cells. Migration to lymph nodes is important because it increases the efficiency and probability of DCs to encounter a relevant T cell clone for the antigens being presented. DC migration to lymph nodes requires the chemokine receptor CCR7 (Forster et al., Cell, 99(1):23-33, 1999), and CCL19 is a chemokine ligand for CCR7 (Yoshida et al., J. Bio. Chem., 272(21):13803-13809, 1997). To model the migratory function of DCs, moDCs were treated with various combinations of R848, DGP, and MCC950 and then placed in the apical chamber of a transwell. Cells then had to actively migrate through 5 μm pores, a diameter too small for cells approximately 13 μm in diameter to simply fall through, to reach the basal chamber. As a chemoattractant, CCL19 was added to the basal chamber at 0; 10; or 100 ng/mL. After an overnight incubation, the DCs that successfully migrated to the basal chamber were enumerated. Migration was normalized to the condition where cells were treated with only R848 with no CCL19 chemokine signal.

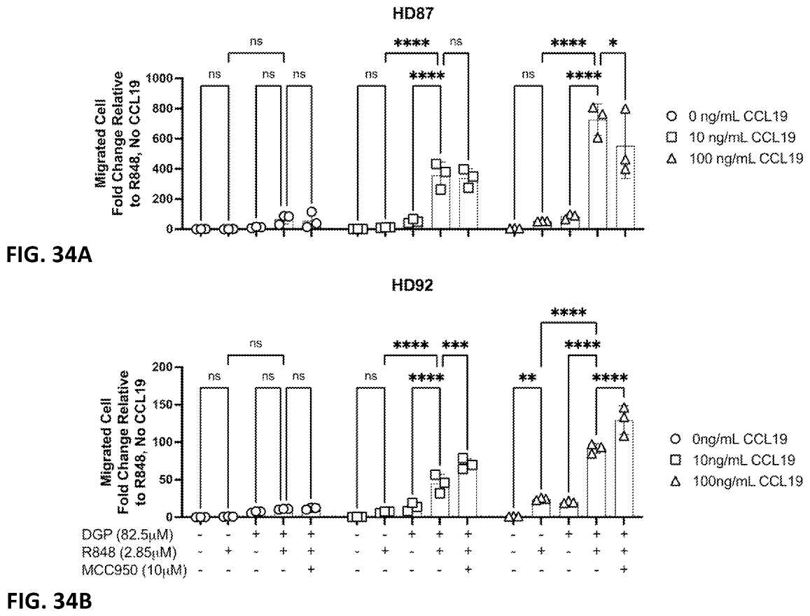

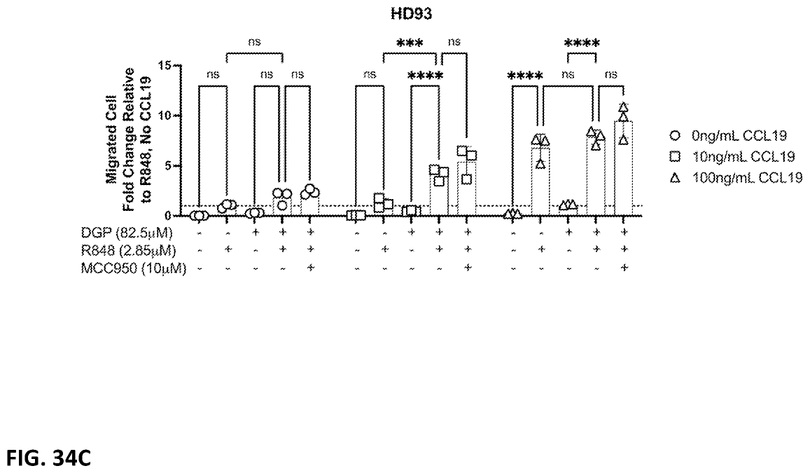

Across all donors, when moDC were not given CCL19, minimal migration to the basal chamber occurred (FIG. 34A-C). Upon addition of CCL19, increased migration was observed in some conditions. HD87 had the most profound results. When CCL19 was added to the basal chamber at 10 ng/mL, R848 treatment did not increase migration of moDC compared to unstimulated cells. In contrast, hyperactivated moDC (R848+DGP) showed statistically increased migration to the basal chamber compared to R848 or DGP treatments alone (FIG. 34A, p<0.0001 for both comparisons). These comparisons indicated that the two components (R848 and DGP) are required in combination to induce this hypermigratory phenotype. Either chemical alone is insufficient to induce migration. Inhibition of NLRP3 via MCC950 treatment did not inhibit the hypermigration observed with R848+DGP (FIG. 34A), suggesting that NLRP3 and its downstream effect of IL-1β secretion are not required for hypermigration. When the concentration of CCL19 was further increased to 100 ng/mL, the stronger chemokine signal was still insufficient to drive increased migration of R848-activated cells compared to unstimulated cells (FIG. 34A). Single treatment with R848 or DGP were not significantly different from unstimulated cells, but R848+DGP hyperactivation increased cell migration significantly compared to treatment with either stimulus individually (FIG. 34A, p<0.0001). At 100 ng/mL CCL19, addition of MCC950 to the hyperactivating stimuli slightly decreased hypermigration as compared to R848+DGP (FIG. 34A, p<0.0001), but this was the only case for all three donors where this was observed. Altogether, the data collected for HD87 demonstrated that hyperactivated moDC migrated to a CCL19 signal in a dose-dependent fashion whereas cells left untreated or treated with R848 or DGP had minimal migratory capacity. The migration observed with hyperactivation did not seem to be NLRP3 dependent.

Compared to HD87, HD92 had similar migration trends although the fold change in migration was not as pronounced at HD87. Again, minimal migration of moDC was observed when CCL19 was not added (FIG. 34B). Addition of 10 ng/mL CCL19 did not increase the migration of R848-treated cells compared to unstimulated cells. In contrast, hyperactivated moDC migrated significantly more than moDC treated with R848 or DGP (FIG. 34B, p<0.0001 for both comparisons). Migration of hyperactivated cells was not inhibited by MCC950 treatment (FIG. 34B). Unlike HD87, when 100 ng/mL CCL19 was applied to HD92, an increase in migration was detected by R848 treated cells compared to unstimulated cells (FIG. 34B, p<0.01). Nonetheless, hyperactivation with R848+DGP led to a further increase in migration compared to treatments of either chemical alone (FIG. 34B, p<0.0001 for both comparisons). Again, at 100 ng/mL CCL19, MCC950 treatment of hyperactivated cells did not deter cells from migrating (FIG. 34B). Quite contrary, MCC950 addition actually increased migration compared to hyperactivation without MCC950 at both 10 and 100 ng/mL CCL19 (FIG. 34B, p<0.001 at 10 ng/mL and p<0.0001 at 100 ng/mL). The reason for this observation was unclear but was observed only for HD92.

Finally, HD93 had the least pronounced migration of the three donors tested. Notably, HD93 also had the poorest induction of IL-1β (FIG. 30) suggesting that these cells were poorly stimulated by R848+DGP. At 0 ng/mL CCL19, minimal migration was observed for any of the cell treatment conditions. At 10 ng/mL CCL19, unstimulated cells and R848-activated cells migrated similarly (FIG. 34C). Hyperactivation induced a significant increase in migration compared to R848 (p<0.001) or DGP (p<0.0001) individually (FIG. 34C). Addition of MCC950 to R848+DGP did not affect the migratory behavior induced by hyperactivation (FIG. 34C). By increasing the CCL19 concentration to 100 ng/mL, the ability of cells to migrate from R848 stimulation alone further increased and was significantly better than unstimulated cells (FIG. 34C, p<0.0001). Interestingly, at this highest concentration of CCL19, no further increase in migration was observed when R848 was combined with DGP compared to R848 alone (FIG. 34C). In contrast, R848+DGP treated moDC migrated better than cells treated with only DGP (FIG. 34C, p<0.0001). Additionally, MCC950 addition to hyperactivating stimuli did not affect migration at 100 ng/mL CCL19 (FIG. 34C). The hypermigratory phenotype of hyperactivated cells was only observed at the lower CCL19 concentration likely because the hyperactivation with R848+DGP was relatively poor for HD93.

The three donors displayed varying degrees of hypermigration when moDCs were hyperactivated. Nonetheless, all three donors displayed enhanced ability to migrate in a CCL19-dependent manner compared to moDC left unstimulated or treated with R848 or DGP as single agents. These data suggest that hyperactivation of DCs using R848 and DGP enables these cells to better migrate to lymph nodes where they may mediate adaptive immune responses.

### Example B-8: Hyperactivated moDCs Improve Th1 and Th17 Cytokine Responses

### Materials and Methods

Monocyte Isolation, Storage, and Differentiation into moDC. Quality Control Staining of moDC Differentiation, and LDH Release Assays were performed as described in Example B-6.

Memory CD4+ T Cell Isolation. The CD14− fraction of the leukapheresis product was collected and used for further isolation of memory CD4+ T cells. First, approximately 5×10⁹ total nucleated cells or less of the blood product were pelleted at 200×g for 10 min. The cells were resuspended in two 50 mL conical tubes each containing 25 mL in volume. Cells were then incubated with microbeads targeting red blood cells and granulocytes at 4° C. for 15 min. Cells were loaded on equilibrated MultiMACS columns using the “Deplete” program. Columns were washed twice with separation buffer and the flow-through of peripheral blood mononuclear cells (PBMCs) were collected and counted. The Memory CD4+ T Cell Isolation Kit from Miltenyi was then used to isolate memory CD4+ T cells from the PBMCs. After following the manufacturer's protocol for antibody and microbead incubations, cells were loaded onto equilibrated MultiMACS columns using the “Deplete” program. After 2 column washes using separation buffer, the negative fraction was collected and counted. An aliquot was stained to confirm the purity of the memory CD4 T cell population. Cells were centrifuged at 400×g for 5 min and resuspended at 1.25×10⁷ cells/mL in freezing media (FBS containing 10% DMSO) and aliquoted at 3 mL per cryogenic freezing vial. Vials were placed in CoolCell freezing containers and placed in a −80° C. freezer for no longer than 1 week. Frozen cells were transferred for long-term storage to the vapor phase of a liquid nitrogen freezer until use.

Treatments of Cells For Analysis of Cytokine Secretion. Human moDCs were plated at 5×10⁴ cells/well in 96-well U-bottom tissue culture plates in R10++ media by adding 50 μL of cells per well. To each well, 50 μL of memory CD4 T cells at 2.5×10⁶ cells/mL was added. For control conditions where only moDC or only CD4 T cells were plated, the missing cells were replaced with an equivalent volume of R10++ media. After adding all treatments, cell cultures had a total volume of 200 μL/well.

Lyophilized R848 stocks were prepared as described above and frozen at −80° C. for storage. An aliquot of frozen R848 was thawed and further diluted to 22.8 μM in R10++ (8 μg/mL R848). A portion of diluted R848 was combined with MCC950 to reach a concentration of 80 μM MCC950. Another portion of diluted R848 was combined with anti-IL-1β blocking antibody to reach a concentration of 80 μg/mL anti-IL-1β. Cell cultures received 25 μL of these mixes depending on desired treatment. For wells not receiving R848 treatment, an equivalent volume of R10++ media was added to wells. For these three chemicals, final concentrations in the wells were 2.85 μM R848, 10 μM MCC950, and 10 μg/mL anti-IL-1β.

A4.44% solution of KP407 dissolved in sterile, distilled water was prepared at a temperature of 4° C. DGP from powder was prepared by adding KP407 solution to lipid. Using a magnetic stir bar, the solution was agitated at 1000 rpm for 1 hour at RT. Sterile 10×PBS was added to the solution resulting in a 1×PBS concentration. The mixture was then stirred at 1000 rpm for another 30 min at RT. The final stock concentration of DGP was 650 μg/mL in 4% KP407 in 1×PBS. The stock solution was then further diluted in PBS to achieve an 8×concentration of DGP from the final target of 41.3 μM. DGP was then added to cell cultures at 25 μL/well. For wells not receiving DGP, 4% KP407 in 1×PBS was diluted in PBS and added to cells as a vehicle control. Volume of vehicle control used matched the volume of DGP used in the study.

To activate memory CD4+ T cells, an anti-CD3 antibody was used to induce T cell receptor signaling. Anti-CD3 antibody was diluted in R10++ media to 0.4 ng/mL and added to wells at 50 μL/well. The final concentration in the wells after addition was 0.1 ng/mL. For control conditions not receiving anti-CD3 antibody, an equivalent volume of R10++ media was added to wells.

Plates were sealed with Breathe-Easy Membranes plate sealers and placed in a 37° C., 5% CO₂ incubator for two days. For study readouts, plates were centrifuged at 400×g for 4 min and cell culture supernatant was collected in 96-well U-bottom plates. Cells were lysed in CellTiter-Glo 2.0 reagent to measure cell viability.

CellTiter-Glo 2.0 Cell Viability Assay. To measure cell viability, CellTiter-Glo 2.0 Cell Viability Assay kit from Promega was used. CellTiter-Glo 2.0 reagent was used according to the manufacturer's instructions. Briefly, frozen reagent was thawed at 4° C. and aliquoted. Aliquots were stored at 4° C. in the dark. After removing 150 μL of the cell culture volume for use in other assays, cells were lysed with 50 μL/well CellTiter-Glo reagent by pipetting up and down. After incubating in the dark at RT for 10 min, samples were transferred to white opaque 96-well plates. Luminescence was measured for 500 ms/well using a Spectramax M3 plate reader. Luminescence values were normalized to the mean values of control samples and multiplied 100-fold to report values as percentage viability.

### Lumit IL-6, IL-1β, IFNγ Immunoassays.

To determine the concentrations of IL-6, IL-1β, and IFNγ secreted into the cell culture supernatant, Lumit Immunoassays were used. Conceptually, two antibodies are used to bind to the target cytokine in the sample, and each antibody is tethered to a subunit of an enzyme. Binding of the two antibodies to the target cytokine enables the subunits to be in close enough proximity to bind and function. A substrate for the enzyme is then added to create a luminescent signal that indicates the cytokine concentration in the supernatant.

The IL-6 and IFNγ kit standards were prepared in cell culture medium (R10++) starting at 25,000 μg/mL with two-fold dilutions to 24.4 μg/mL. The IL-1β standard was prepared by diluting in R10++ medium to 40.000 μg/mL. The top standard was then serially diluted two-fold to 39 μg/mL. Blank controls were included for all standards. Sample supernatants were diluted 5-fold in R10++ for the IL-6 and IFNγ assay. Sample supernatants were used undiluted for the IL-1β immunoassay.

To prepare antibodies for each assay, recommended protocols from the manufacturer were followed to dilute antibodies in R10++ media to reach a 2-fold concentrate. Antibodies were combined with sample supernatant or standards (12.5 μL of each) by pipetting into white opaque 384-well plates. Samples were then incubated at 37° C. for 1 hour. Samples were then allowed to cool to RT in the dark while preparing substrate. For every 3,040 μL of Detection Buffer B used, 175 μL of Detection Substrate B was added. Substrate was mixed by briefly vortexing and was added at 6.25 μL/well. On the Spectramax M3 plate reader, sample luminescence was recorded at 500 ms/well. Prism software was used to construct a standard curve using 4-parameter logistics analysis. Interpolated results were corrected for sample dilution.

LEGENDplex Assay. To detect multiple cytokines of interest (IL-4, IL-5, IL-13, IL-17A, IL-17F, and IL-22), the predefined LEGENDplex Human Th Cytokine Panel from Biolegend was utilized. Conceptually, each targeted cytokine has antibodies bound to a unique bead population defined by bead size and APC fluorescence signal. When incubated with sample supernatant, cytokines are bound by the antibodies. Biotinylated secondary antibodies are then added to bind the cytokines of interest, and streptavidin-bound PE is then incubated with samples. Using flow cytometry, each bead population specific for a cytokine can be defined based on bead size and APC signal intensity. The PE fluorescence signal on beads then indicates the amount of cytokine present in the sample.

To run the assay, samples were diluted 2-fold in Assay Buffer. Standards were prepared according to manufacturer instructions. Lyophilized standard was reconstituted in 250 μL Assay Buffer. After a 10 min incubation at RT, this top standard concentration was serially diluted 4-fold to make an 8-point standard curve with the last point set as blank buffer. In each well of a V-bottom plate, 25 μL Assay Buffer was added with 25 μL of sample or standard. Beads were vortexed thoroughly and added at 25 μL/well. Samples were incubated in the dark at RT for 2 hours on an orbital shaker set at 200-300 rpm. The beads were then pelleted at 250×g for 5 min and resuspended in 200 μL/well 1×wash buffer. After pelleting beads again, samples were resuspended in 25 μL/well Detection Antibodies. Samples were incubated for 1 hour at RT in the dark on the orbital shaker. Without washing, 25 μL/well of streptavidin-PE was added to samples and incubated at RT in the dark for 30 min on the orbital shaker. After the incubation, beads were washed again and resuspended in 150 μL/well wash buffer. Samples were analyzed the same day using the Novocyte Quanteon flow cytometer.

Raw data was exported as FCS files and uploaded to the LEGENDplex Data Analysis Software Suite from Qognit for analysis. Five parameter logistic curves were constructed from the standards, and sample results were interpolated using the cloud software. Sample dilutions were incorporated into the analysis, and correct bead population gating was verified on all samples. Results were exported from the cloud software as a Microsoft Excel file.

Data Analysis. Data were analyzed and graphed using Microsoft Excel and GraphPad Prism software. Data are reported in graphs as mean with ranges indicating SDs. Graphs depict data from individual donors, and data points represent biological replicates.

For statistical analyses, ordinary two-way ANOVA was conducted, followed by Tukey's multiple comparisons test with a single pooled variance. Statistical testing was completed using Prism software.

### Results

Assessment of Starting Cell Materials Used for Coculture. After culturing monocytes in media containing GM-CSF and IL-4 for 6 days, cells were collected for use. Cells were stained with fluorescent antibodies to determine cell surface expression of CD11c and CD209. Both monocytes and moDC express CD11c but only moDC express CD209 (Geijtenbeek et al., Cell, 100(5):575-585, 2000). For the three donors used in this study, 95% or more of the live cell population were CD11c+CD209+, indicating that the monocytes had differentiated into moDC. Of the live CD11c+CD209+ cells, at least 99% of cells stained SIRPa+, a marker of the cDC2 subset. Less than 1% of cells stained CD11c+CD209− indicating that few cells remained as undifferentiated monocytes. Additionally, any undesired cell types such as B and T cells would be CD11c−CD209−. Fewer than 5% of cells were CD11c−CD209−, indicating that the cultures had minimal contaminating cell populations. Altogether, staining for CD11c, CD209, and SIRPa indicated that the monocyte cell cultures were successfully differentiated into a highly pure DC cultures with a cDC2-like phenotype.

Autologous memory CD4+ T cells had been isolated from the same leukapheresis blood products from which the monocytes had been derived. After isolation, a portion of cells were stained to determine the frequency of live, CD3+CD4+ single cells that expressed CD45RO, a memory marker (Merkenschlager et al., European Journal of Immunology, 18(11):1653-1661, 1988). The isolated cell populations from the three donors were greater than 90% pure as indicated by CD19−CD3+CD8−CD4+CD45RO+ staining. Together, the high purity of moDC cultures and memory CD4+ T cell cultures ensured that the predominant cell interactions in a coculture were between moDCs and memory CD4+ T cells.

Hyperactivation of moDCs with R848 and DGP. To understand how DCs affected memory CD4+ T cell reactivation, an in vitro coculture assay was set up. 50,000 moDCs were plated with 250,000 autologous memory CD4+ T cells. moDC were either left unstimulated or treated with R848 and/or DGP. These signals would theoretically induce moDC to express inflammatory signals for T cells, such as cytokines and costimulatory molecules. To stimulate the T cell receptor (TCR) on CD4+ T cells, antibodies binding to CD3 were used. Two days after the start of the coculture, cell culture supernatant was collected to quantify cytokine secretion. To control for possible effects of R848 and DGP directly on T cells, a parallel set of treatments were set up for CD4+ T cells only, where moDCs were not added into the culture. Statistical analysis in this section was performed using two-way ANOVA.

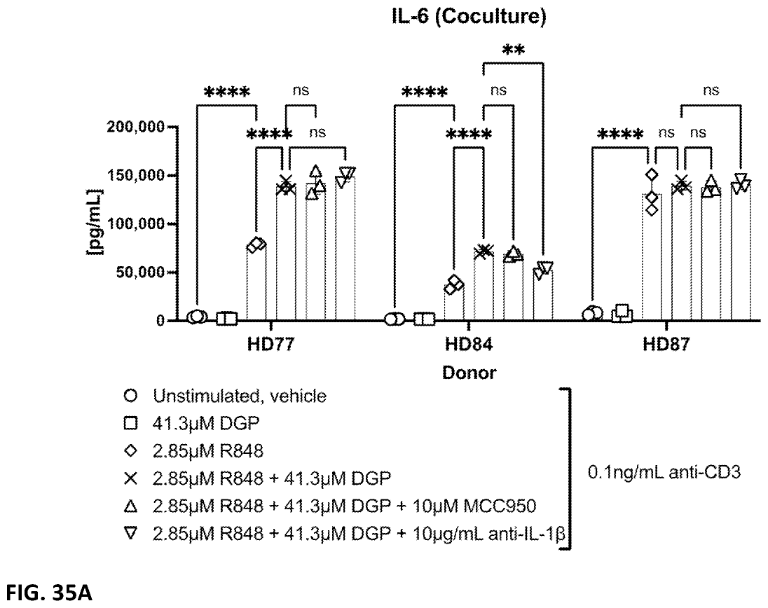

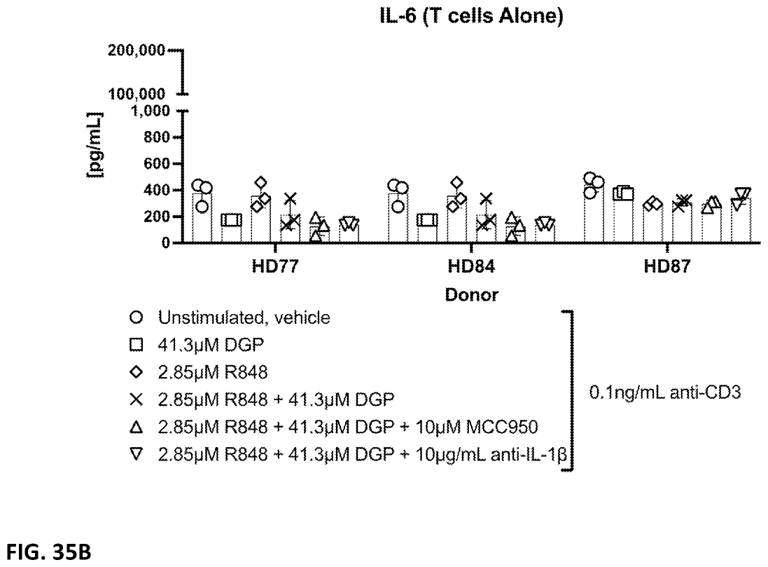

In the coculture supernatant, IL-6 was measured as an indicator of NF-kB activation from R848 stimulation. IL-6 was significantly increased when R848 was added to the coculture compared to unstimulated cells (FIG. 35A, p<0.0001). For two of the three donors, hyperactivation using R848+DGP further increased IL-6 production compared to R848 single agent treatment (FIG. 35A, p<0.0001). Addition of MCC950 to R848+DGP did not significantly change IL-6 secretion, which was an expected result because IL-6 expression is not NLRP3-dependent (FIG. 35A). For 2 of the 3 donors, IL-6 concentrations were also unaffected by the use of anti-IL-1β. Although IL-6 secretion decreased for HD74 when comparing R848+DGP to the equivalent with anti-IL-1β, the cells still produced a comparable amount of IL-6 to R848 single agent treatment (FIG. 35A). Overall, the coculture data demonstrated that R848 stimulated IL-6 production.

When the assay was set up with only T cells in culture, the IL-6 concentrations detected were much lower and was not affected by the different treatments. T cells in the absence of moDCs were plated with all combinations of cell stimuli. In these single-population cultures, no conditions had significantly higher IL-6 concentrations than the unstimulated control (FIG. 35B). The data obtained from coculturing moDCs with T cells and from culturing only T cells indicated that moDCs contributed nearly all of the IL-6 detected in the coculture. Furthermore, IL-6 production was dependent on R848 stimulation of moDC.

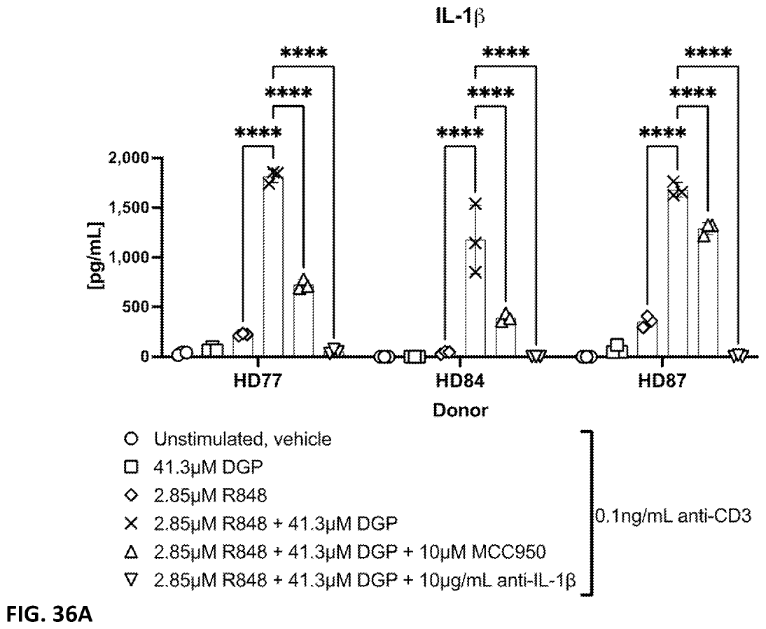

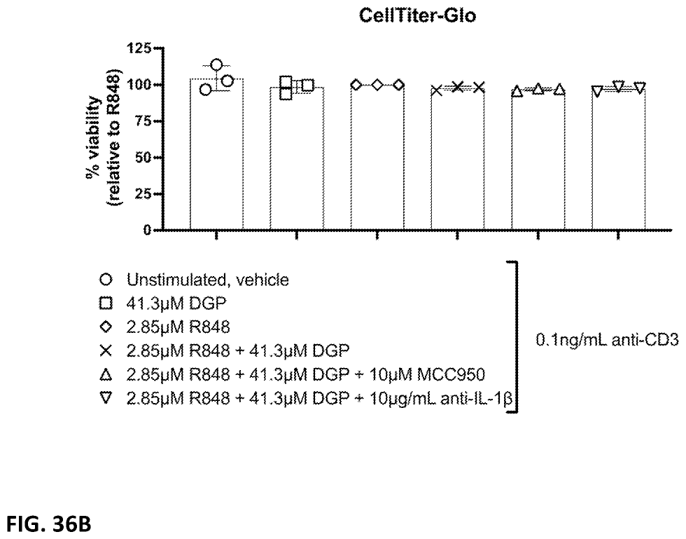

Next, the effect of DGP on the cells in coculture was studied by measuring IL-1β in the coculture supernatant. When moDCs and T cells were treated with R848 or DGP as single agents, minimal IL-1β was detected (FIG. 36A). When R848 was combined with DGP, IL-1β was significantly higher than treatment with R848 alone (FIG. 36A, p<0.0001). By combining MCC950 with R848+DGP, IL-1β was significantly reduced compared to treatment without MCC950 (FIG. 36A, p<0.000 1). The inhibition of IL-1β secretion by MCC950 was incomplete. One possible explanation is that the chemical inhibitor was not as effective over the 2-day culture compared to shorter studies. In the treatment where R848+DGP was combined with the blocking antibody anti-IL-1β, no IL-1β was detectable (FIG. 36A, p<0.0001). The lack of IL-1β detected was likely due to the blocking antibody binding to IL-1β and inhibiting the detection of the cytokine in the detection assay.

When measuring cell viability using CellTiter-Glo, all treatment conditions had similar coculture viabilities within a 25% range of 100% viability (FIG. 36B). Together with the IL-1β data, the viability data demonstrated that the cocultures were hyperactivated when treated with R848+DGP. When measuring IL-1β in T cell cultures where moDC were not added, no IL-1β was detectable (all data values in these conditions were too low to be interpolated). Thus, IL-1β was only produced by moDC upon treatment with hyperactivating stimuli. The lack of IL-13 from T cell cultures indicated that T cells cannot be directly stimulated by R848 and DGP.

R848 and DGP Hyperactivate moDC to Enhance Th1 and Th17 Reactivation Responses. After establishing that R848+DGP stimulation resulted in moDC hyperactivation, the next question was how hyperactivated moDCs affected the reactivation of memory CD4+ T cells in the cocultures. Given the high purity of the isolated cells used in the assay, nearly if not all IFNγ, IL-4, IL-5, IL-13, IL-17A, IL-17F, and IL-22 secretion was attributed to the defined memory CD4 T cells placed in cocultures.

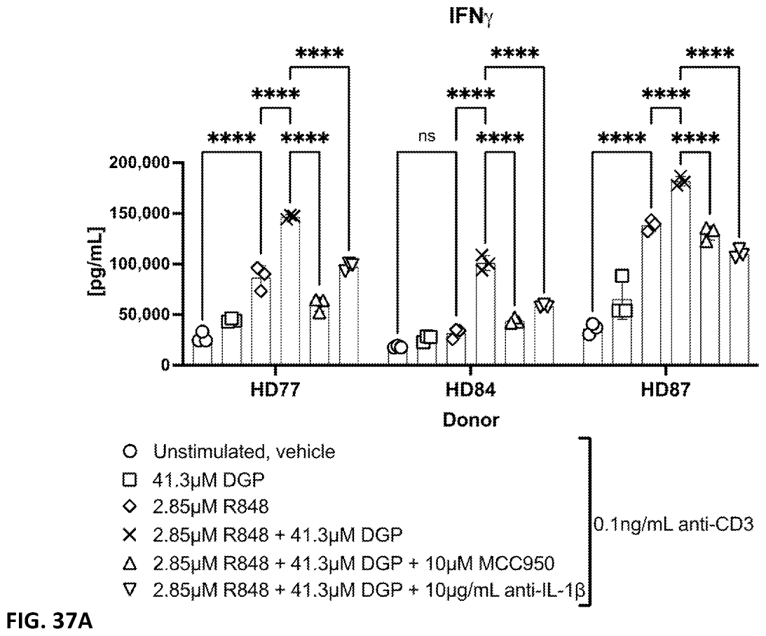

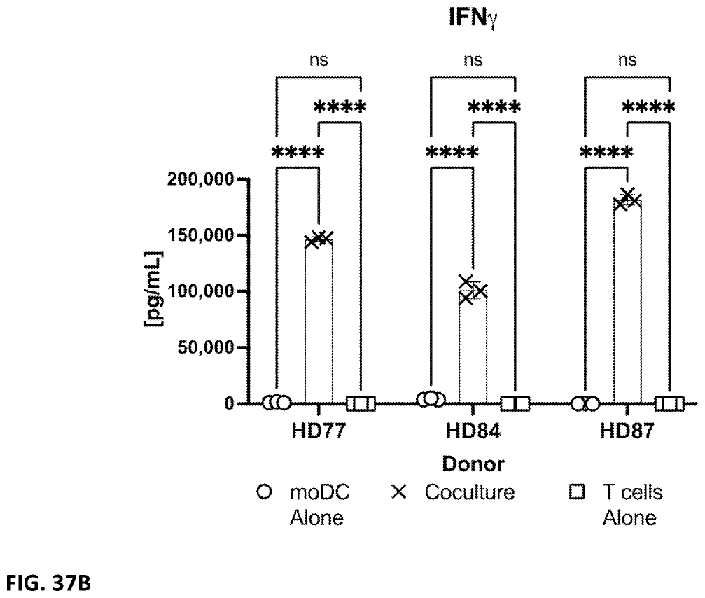

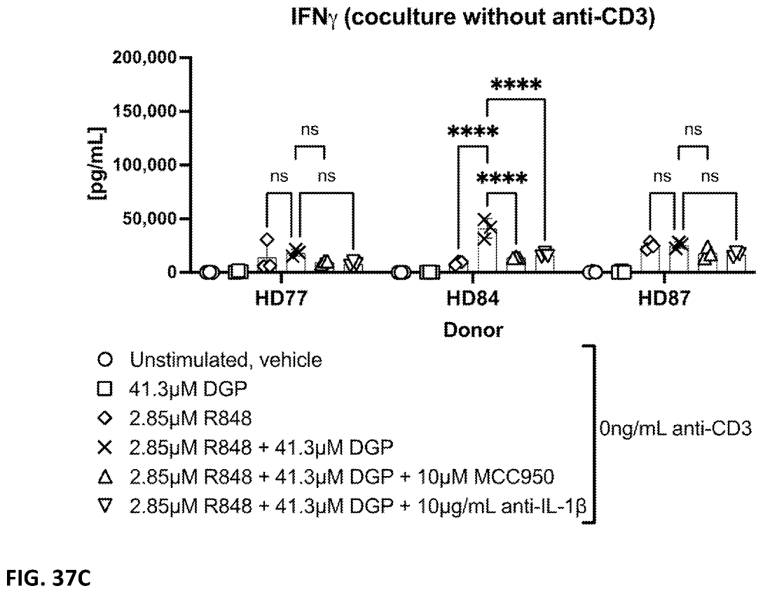

Assessment of Th1 Responses. Coculture supernatants were assayed for the presence of IFNγ, a cytokine produced by Th1 cells. A basal amount of IFNγ was produced by T cells because of anti-CD3 stimulation of the TCR, as indicated by the condition where moDC were left unstimulated (FIG. 37A). Activation of moDC with R848 treatment enhanced the IFNγ response from T cells in two donors (FIG. 37A, p<0.001). When hyperactivated using R848+DGP, a further enhancement of IFNγ production was observed compared to R848 single agent treatment (FIG. 37A, p<0.0001). Thus, hyperactivated moDC induced stronger IFNγ responses from Th1 cells compared to unstimulated moDC or R848-activated moDC.

To evaluate whether the enhancement of IFNγ response was dependent upon NLRP3 inflammasome activation and IL-1β signaling, R848+DGP was combined with MCC950 or anti-IL-1β. When MCC950 or anti-IL-1β was added to the coculture hyperactivations, IFNγ production was significantly reduced (FIG. 37A, p<0.0001). Interestingly, these conditions containing MCC950 and anti-IL-1β had IFNγ levels more similar to the R848 activation condition than the hyperactivation condition, suggesting that the enhancement of the IFNγ response by R848+DGP was entirely mediated by the IL-1β secreted from NLRP3 activation.

As a control to ensure that the IFNγ response was dependent upon interactions between moDCs and T cells, the two cell types were plated in isolation or together in coculture. Cells were treated with R848, DGP, and anti-CD3 antibody. IFNγ was only produced when cells were cocultured (FIG. 37B). In comparison, R848+DGP treatment with anti-CD3 where only moDC or T cells were present resulted in no IFNγ production (FIG. 37B, p<0.0001). These control conditions ensured that IFNγ was not produced by moDC or T cells directly as a result of R848+DGP treatment.

The role of the anti-CD3 antibody in stimulating the IFNγ response was also assessed in a set of control conditions where anti-CD3 was left out of the coculture. Minimal IFNγ was produced from the various moDC/T cell cocultures in the various treatment conditions when anti-CD3 was not present (FIG. 37C). Although one human donor sample, HD84, demonstrated an enhancement of IFNγ production when comparing hyperactivation to activation conditions (p<0.0001), the other two donors showed no significant difference (FIG. 37C). These data demonstrate that while the inflammatory signals from moDC (such as IL-1β, other cytokines, and costimulatory molecules) are important, TCR engagement also has an important role in the reactivation of CD4 T cells.

Altogether, the body of data collected regarding IFNγ production in this experiment demonstrated that the cell state of moDC had important consequences when T cells were reactivated to produce a Th1 response. While engagement of the TCR was needed for a robust IFNγ response, additional signals provided by moDC (as demonstrated by R848 activation of moDC) were also required. R848 and DGP led to hyperactivation of moDC that were superior at inducing memory CD4 T cells to produce IFNγ. The effect was inflammasome and IL-1β dependent. Control conditions demonstrated that R848 and DGP could not act directly upon T cells for IFNγ production, and moDC alone could not produce IFNγ.

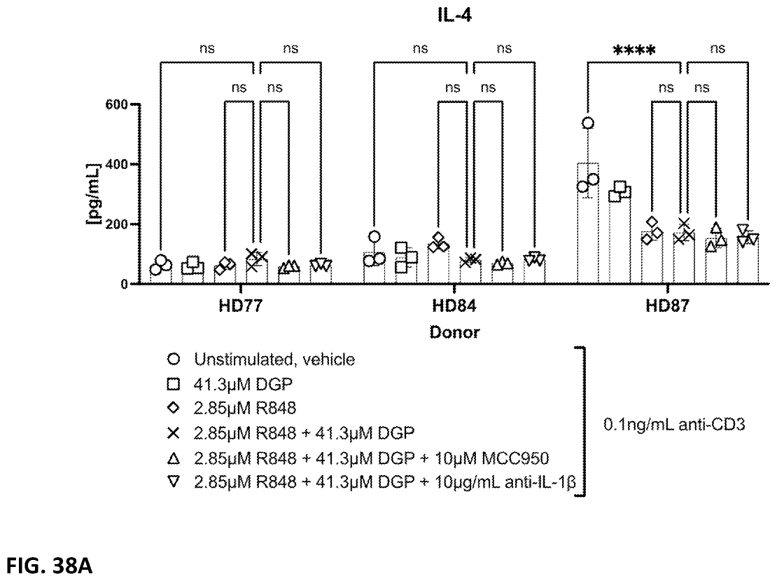

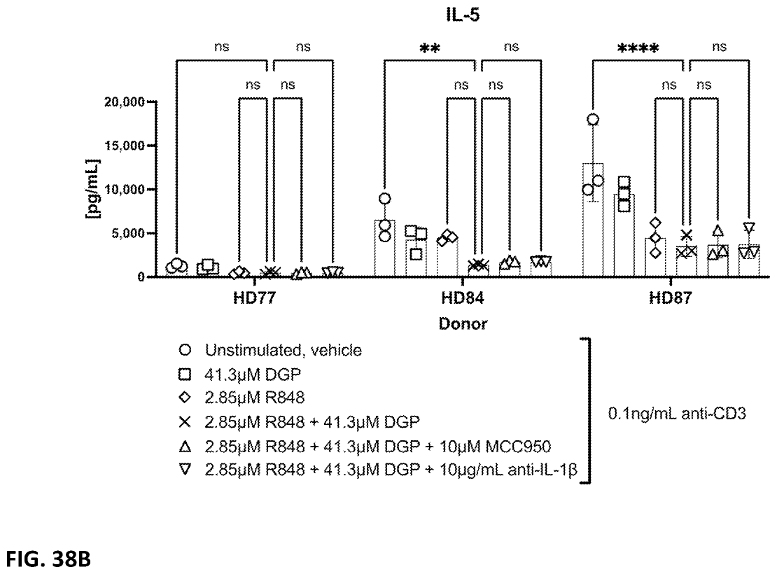

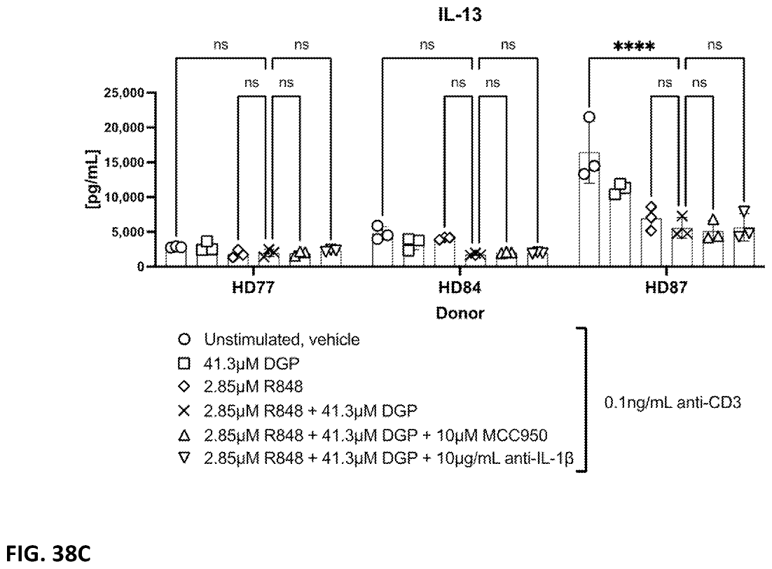

Assessment of Th2 Responses. To assess Th2 responses, IL-4, IL-5, and IL-13 cytokine production was measured. All three cytokines were detectable in coculture supernatants (FIG. 38A-C). When cells were treated with R848, DGP, or in combination, the production of IL-4, IL5, and IL-13 were not elevated compared to unstimulated conditions where only anti-CD3 was provided (FIG. 38A-C). The activities of R848, DGP and the combination of R848 and DGP were confirmed by the production of IL-6 and IL-β (FIG. 35A and FIG. 36A). Therefore, these data indicate that Th2 responses were not enhanced by signals provided by R848+DGP stimulation of moDCs.

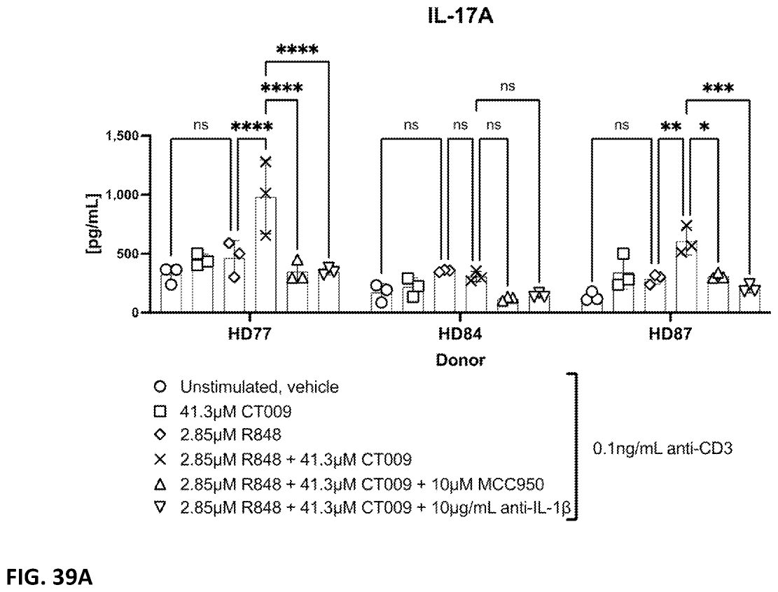

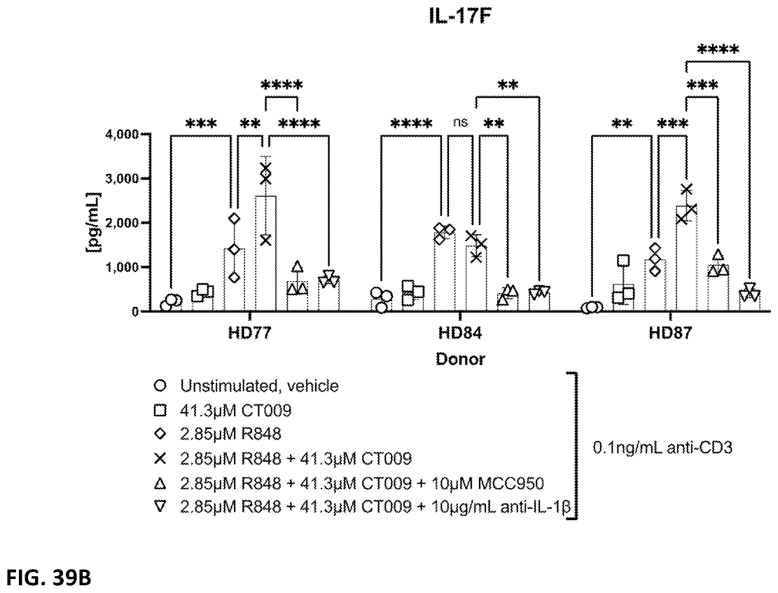

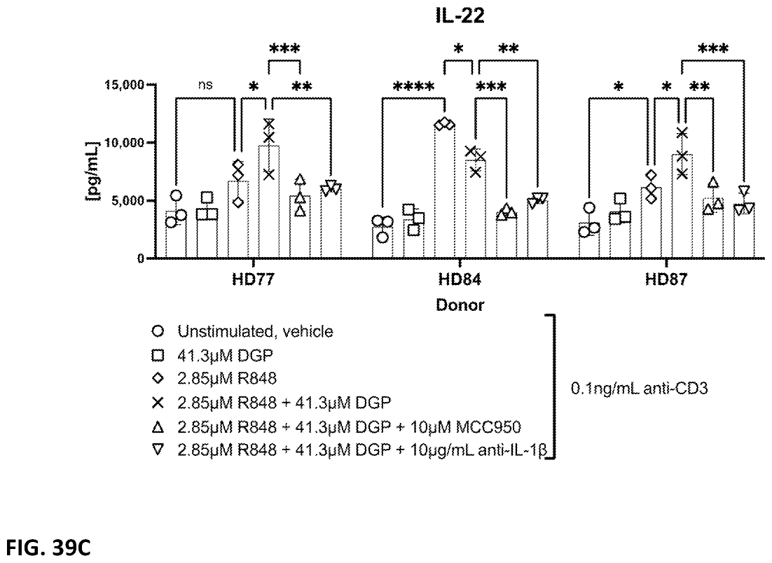

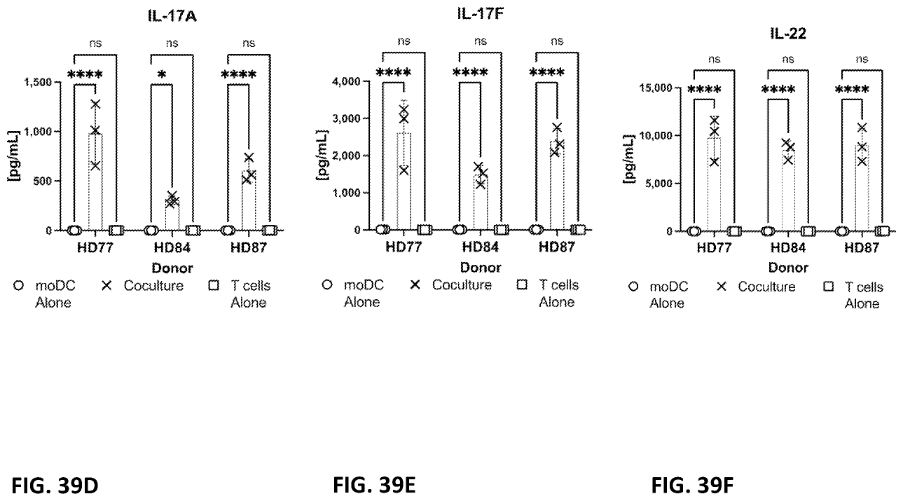

Assessment of Th17 Responses. To assess for Th17 responses, IL-17A, IL-17F, and IL-22 production were measured. IL-17A was produced in the cocultures when moDCs were left unstimulated and only anti-CD3 was provided to T cells. When moDC were activated with R848 stimulation, IL-17A production remained at basal concentrations (FIG. 39A). Hyperactivation of moDC led to donor-dependent results. Comparing R848+DGP treatment to R848 activation, T cells produced similar amounts of IL-17A for HD84 (FIG. 39A). However, HD77 and HD87 had enhanced IL-17A T cell responses compared to R848 activation (FIG. 39A, p<0.0001 and p<0.01, respectively). The enhancements observed were due to the activation of the NLRP3 inflammasome and specifically due to IL-1β signaling as R848+DGP treatments with MCC950 or anti-IL-1β significantly reduced IL-17A to concentrations similarly produced when moDC were activated with R848 (FIG. 39A, p<0.05).

In contrast to IL-17A, production of IL-17F increased when moDC were stimulated with R848 compared to unstimulated cells (FIG. 39B, p<0.01). While HD84 demonstrated an enhancement of IL-17F production when moDC were activated with R848 (p<0.0001), no further increase of IL-17F production was observed when moDC were hyperactivated with R848 and DGP (FIG. 39B). Both HD77 and HD87, unlike HD84, had enhanced IL-17F responses when moDC were hyperactivated compared to activated with R848 alone (FIG. 39B, p<0.01 and p<0.001, respectively). These elevated IL-17F responses from T cells was due to IL-1β signaling because MCC950 and anti-IL-1β treatments significantly reduced IL-17F production (FIG. 39B, p<0.001).

Finally, the Th17 cytokine IL-22 demonstrated similar trends observed with IL-17A and IL-17F. IL-22 production increased when cells were treated with R848 compared to unstimulated moDCs for HD84 and HD87 (FIG. 39C, p<0.05). Like the other cytokines, HD84 did not have increased IL-22 production when hyperactivated (FIG. 39C). For the other two donors, hyperactivation of moDC using R848 and DGP increased the IL-22 produced by T cells (FIG. 39C, p<0.05). Again, inhibition of hyperactivated moDC using MCC950 or anti-IL-1β resulted in a significant reduction in IL-22 (FIG. 39C, p<0.01).

In complementary analyses to what was done with IFNγ, Th17 cytokines were measured in cell cultures containing R848, DGP, and anti-CD3 but missing either moDC or T cells. IL-17A, IL-17F, and IL-22 were produced when cells were cocultured, but when moDC or T cells were cultured alone, R848, DGP, and anti-CD3 signals were insufficient for inducing Th17 cytokine production (FIG. 39D-F). Thus, T cells were not responding directly to R848+DGP to produce Th117 cytokines, and moDC were not producing Th17 cytokines themselves. IL-17A, IL-17F, and IL-22 production depended on the interaction of moDCs with T cells, just like IFNγ.

The data gathered on Th17 cytokines suggested that similar to Th1 cells, Th17 cells were responsive to IL-1β in their production of IL-17A, IL-17F, and IL-22. The results of the Th17 cytokines were not as consistent across all three donors compared to the data gathered on the Th1 IFNγ response. Notably, the concentrations of Th17 cytokines were drastically lower than the IFNγ detected. One possibility for explaining the weaker Th17 data is that the Th17 population is much smaller than the Th1 population among memory CD4 T cells (Liu and Wang, Lupus Science and Medicine, 1(1), e000062, 2014). Nonetheless, the data suggested that Th17 cells likely benefit from being reactivated by moDC hyperactivated with R848+DGP due to the signaling provided by IL-1β. The Th1 and Th17 effects starkly contrasted Th2 reactivation responses where R848+DGP treatment provided no added benefit.

In summary, the experimental data support a model where hyperactivated moDC produce IL-1β, which enhances Th1 and Th17 cytokine responses of memory CD4+ T cells upon their reactivation. In contrast, IL-1β signaling does not regulate Th2 cytokine production.

### Example B-9: Hyperactivated DCs Reactivate Antigen-Specific CD8+ T Cells

Efficient reactivation of memory T cell responses requires T cells to receive multiple signals from DCs for tailored and rapid antigen-specific T cell activation and induction of effector functions (Iwasaki and Medzhitov, Nature Immunology, 16:343-353, 2015). These signal include: 1) MHC-mediated presentation of antigens on the surface of DCs for TCR activation, 2) the secretion of polarization cytokines that induce T cells to secrete effector cytokines such as IFNγ; 3) the upregulation of costimulatory molecules such as CD40, which ensure robust DC interactions with T cells in the lymph node; 4) the production of memory signals such as IL-1β that permit the reactivation of previously primed T cells; and 5) an enhancement of DC migratory activities from the site of immunization to the draining lymph node (dLN).

### Materials and Methods

Murine Bone Marrow-Derived FLT3L-DC Generation. The femur and tibia were removed from 15 mice for each batch of Flt3L-DC generation. The bone marrow was flushed from the bones into sterile tubes by cutting the ends of the bones and centrifuging the bones for 30 s at 10,000×g. Bone marrow cells from 5 mice were pooled into a 50 ml conical tube and pelleted. The volume of reagents reported in this section is adjusted for processing of bone marrow cells from 5 mice. The cells were resuspended in 2 ml of cold ACK solution for 1 minute. The ACK solution was quenched by adding 10 ml of complete I10 media. The cell suspension was centrifuged at 400×g for 5 min to pellet the cells. The cell pellet was resuspended in 15 ml I10 media and passed through a 40 mm cell strainer. Cells were counted using Moxi V cell counter and resuspended in I10 media at a density of 8×10⁶ cells/ml. Cells were then plated at 8×10⁶ bone marrow cells/well in a 12-well tissue culture plate. Recombinant mouse Flt3L (Miltenyi) was added to cultures at a final concentration of 200 ng/ml. Differentiated cells were used for subsequent assays on day 9. The efficiency of differentiation was monitored by flow cytometry using BD Symphony A3, and CD11c+MHC-II+ cells were routinely above 80% of living cells.

Hyperactivation of Murine FLT3L-DCs. For T-cell co-culture, 9 day differentiated Flt3L-DC were resuspended at a density of 5×10⁶ cells/mi, without Flt3L, and 1 ml of the cell suspension was added to 5 ml MacroTubes. The Flt3L-DC were treated under different conditions as listed in Table 9-1, in a final volume of 2 ml. The tubes were incubated at 37° C., 5% CO₂ for 16 hours under static conditions.

TABLE 9-1
Treatment of Flt3L-DCs

| Treatment | OVA | R848 | DGP | Inhibitor |
|---|---|---|---|---|
| Negative control | — | — | — | — |
| R848, no antigen | — | 2.85 μM (1 μg/ml) | — | — |
| DGP, no antigen | — | — | 41 μM | — |
| R848 + DGP, no antigen | — | 2.85 μM (1 μg/ml) | 41 μM | — |
| Negative control + antigen | 100 μg/ml | — | — | — |
| R848 + antigen | 100 μg/ml | 2.85 μM (1 μg/ml) | — | — |
| DGP + antigen | 100 μg/ml | — | 41 μM | — |
| R848 + DGP + antigen | 100 μg/ml | 2.85 μM (1 μg/ml) | 41 μM | — |
| R848 + DGP + antigen + IL1RA | 100 μg/ml | 2.85 μM (1 μg/ml) | 41 μM | IL-1RA = 500 ng/ml |
| R848 + DGP + antigen + MCC950 | 100 μg/ml | 2.85 μM (1 μg/ml) | 41 μM | MCC950 = 50 μM |

Lipid Preparation. Lipid stocks were formulated at 650 μg/mL lipid in 4% Kolliphor P407 (KP407). Lipids were prepared from lyophilized stocks by mixing with a cold solution of KP407 at 1000 rpm for 1 hour at RT. A10×PBS solution was then added and the lipids were mixed at RT for an additional 30 min to make the 4% KP407 stock solution isotonic.

Immunization of mice with OVA. On day 0, 10 mg of OVA was reconstituted in 2 ml of 1×PBS. Using an 18G needle 2 ml of OVA was drawn into a 5 ml syringe and 2 ml of IFA was drawn into a separate 5 ml syringe. The two syringes were attached to the 3-way stopcock. OVA and IFA were mixed by passing through the stopcock 10 times to prepare a cloudy emulsion. The syringe containing the cloudy emulsion was removed from the stopcock and attached a 25G×5/8 needle.

The tubing for the Calibrated Isoflurane machine was connected to the inlet port of the Isoflurane plastic chamber and charcoal filter was connected to the outlet port of the chamber. Isoflurane was checked and appropriate amount was added before transferring mice to the chamber for anesthetization. Each mouse received 500 μg of the OVA/IFA emulsion (200 μl) on day 0. Then, on days 7 and 14, 10 mg OVA was reconstituted in 5 ml of sterile 1×PBS and immunization was repeated at a dose of 200 μg of OVA.

Generation of CD8+ T-cells for Co-Culture Assay. On day 35, spleens and lymph nodes were harvested from three OVA-immunized mice. A70 mm cell strainer was placed on top of each of three 50 ml conical tubes. The cell strainers were wetted with 2 ml PBS. The spleen and lymph nodes from each mouse were transferred onto each strainer and were crushed using a plunger of a 3 ml syringe. The cells were collected in a final volume of 10 ml PBS. The tubes were centrifuged at 400×g for 5 min to pellet the cells, which were subsequently resuspended in 2 ml of ACK lysis buffer for 2 min at RT. Following the ACK incubation, 10 ml of I10 media was added to quench the ACK lysis buffer. The cells were pelleted and resuspended in 5 ml of MACS buffer. The cells were pooled from different mice and the cell density was determined using a Moxi V cell counter. The cells were pelleted and resuspended in MACS buffer at a cell density of 1.1 1×10⁸ cells/ml. 10 ml of mouse CD8a (Ly-2) microbeads were added to the cell suspension for every 1×10⁷ cells and incubated at 4° C. for 10 min. The AutoMACs Pro cell separator was used to select the CD8+ cells. To enrich CD8+ T-cells, the Possels program was selected and CD8+ cells were collected in the positive selection tube. The CD8+ positive cells were pelleted and resuspend in 10 ml of I10 at a cell density of 2×10⁶ cells/ml in I10 media.

Quality Control Staining of Flt3L-DC and CD8⁺ T cells. Cells were washed with PBS and seeded in 22 wells of a 96-well U-bottom plate at a cell density of 2×10⁵ cells/well (10 wells for single stained control, 10 wells for FMO and 2 wells for the antibody cocktail). The plate was centrifuged at 400 g for 5 min to pellet the cells. Live/Dead Fixable Violet stain was prepared by diluting the stock at 1:1000 in PBS. The cells were incubated with 100 μl of the Live/Dead violet staining solution per well for 20 min at 4° C. in the dark. Cells were washed with 200 μml of FACS buffer (PBS+0.5% BSA). A Fc block solution was prepared by diluting Fc Block 1:100 in FACS buffer. 100 μl of Fc Block solution was added to each well and incubated for 10 min at 4° C. Cell surface staining antibody cocktail and single stain antibody solutions were prepared in 250 μl of FACS buffer at a concentration of 1:100; FMO staining cocktails were prepared in 150 μl of FACS buffer. The antibodies used are listed in Table 9-2. Antibody cocktail, single antibody, or FMO were added to individual wells in a volume of 100 μl and incubated at 4° C. for 15 min in the dark. Compensation beads were also stained with single stain antibody solution. Cells were centrifuged at 400×g for 5 min and washed twice with 200 μL FACS buffer. Cells were resuspended in 200 μl of 4% paraformaldehyde and incubated at 4° C. for 20 min. Cells were centrifuged at 400×g for 5 min and washed twice with 200 μL FACS buffer. Data was acquired on BD Symphony A3 and analyzed on FlowJo.

TABLE 9-2
Antibodies Used For Quality Control Staining\hat{ }

| Purpose | mAb | Clone | Fluorophore | Vendor | Catalog No. |
|---|---|---|---|---|---|
| DC | I-A/I-E(MHC-II) | 2G9 | BUV395 | BD Biosciences | 743876 |
| purity check | CD11c | HL3 | PE-Cy7 | BD Biosciences | 558079 |
| | CD45R | RA3-6B2 | AF700 | BD Biosciences | 557957 |
| | CD24 | M1/69 | BV711 | BD Biosciences | 563450 |
| | SIRP1α | P84 | FITC | BD Biosciences | 560316 |
| | CD103 | 2E7 | BB700 | BD Biosciences | 745919 |
| | XCR1 | ZET | PE | BioLegend | 148204 |
| T-cell | CD8α | 53-6.7 | BV605 | BD Biosciences | 563152 |
| purity check | CD3 | 17A2 | APC-Cy7 | Biolegend | 100222 |

\hat{ }Abbreviations: APC = Allophycocyanine, AF = Alexa Fluor, Cy = Cyanine, BUV = BD Horizon Brilliant Ultraviolet, BV = Brilliant Violet, BB = BD Horizin Brilliant Blue, FITC = Fluorescein isothiocyanate, PB = Pacific Blue, PE = Phycoerythrin, PerCP = Peridinin-Chlorophyll-Protein.

T-Cell Co-Culture. Sixteen hours after hyperactivation and antigen uptake, the suspension of Flt3L-DCs were gently layered on top of 2 ml of FBS in a 5 ml MacroTube. The cells were pelleted by centrifuging the tubes at 400×g for 5 min. The cells were washed twice with 2 ml I10 media. After the washes, cells were resuspended in 2 ml of I10, and the cell density was determined. Cells were pelleted and resuspended in I10 media at a cell density of 2×10⁵ cells/ml. 100 μl of each DC cell suspension was added to wells of a round-bottom 96-well plate in triplicate, and 100 μl of CD8+ T-cells from OVA-immunized mice was added to each DC-containing well. An additional 50p of I10 media was added to each well to achieve a final co-culture volume of 250 μl. To the test conditions with IL-1RA, 50 μl of 2.5 mg/μl IL-1RA stock solution in I10 media was added to each well. The co culture plate was incubated at 37° C., 5% CO₂ for 96 hrs. The culture supernatants were then harvested, and IFNγ concentration was measured using a Lumit mouse IFNγ kit from Promega.

### Results

To evaluate the ability of stimulated DC to re-activate antigen-specific CD8+ T cells, a co-culture assay was established. Ovalbumin (OVA) was used as a model antigen for this assay, and CD8+ T-cells derived from OVA-immunized mice were used as a source of antigen-specific CD8+ T-cells. Since cDC1s are uniquely capable of cross-presenting antigens and activating CD8+ T-cells (Gutiérrez-Martinez et al., Frontiers in Immunology, 6:363, 2015), FTL3L-DCs, which mainly harbor the cDC1 subset of DCs, was selected for this co-culture system.

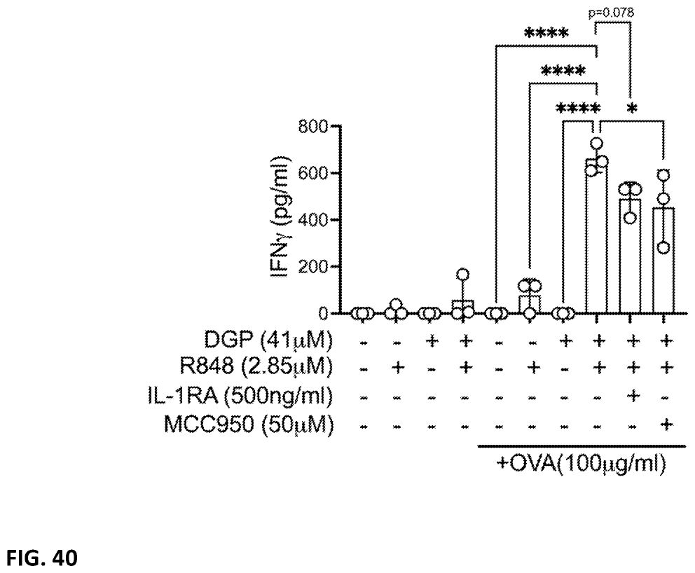

R848+DGP Enhances Antigen-Specific Reactivation of CD8⁺ T Cells. Reactivation was determined by measuring IFNγ concentration of supernatants of CD8+ T-cells co-cultured with Flt3L-DCs. Untreated Flt3L-DCs failed to reactivate CD8+ T cells regardless of whether they were loaded with OVA (FIG. 40). Similarly, DGP-treated Flt3L-DCs did not induce IFNγ secretion in the supernatant regardless of whether they were loaded with OVA (FIG. 40). Furthermore, R848-treated Flt3L-DCs weakly induced CD8+ T cell activation and secretion of IFNγ, even when loaded with OVA (FIG. 40). When Flt3L-DCs were pre-treated with the combination of R848 and DGP in the absence of OVA, the Flt3L-DCs were unable to reactivate T cells. In contrast, robust production of IFNγ by CD8+ T-cells was observed when co-cultured with Flt3l-DCs that were loaded with OVA and treated with R848 in combination with DGP (FIG. 40). Strikingly, the concentration of IFNγ produced by the CD8+ T-cells was 6-fold higher when co-cultured with R848+DGP-treated Flt3L-DCs than with Flt3L-DCs treated with R848 alone or DGP alone (FIG. 40, p=0.0001). These data indicate that R848+DGP-treated Flt3L-DCs are more efficient at reactivating antigen-specific CD8+ T cells as compared to naïve DCs or DCs treated with R848 or DGP alone. Thus, the enhanced antigen-specific CD8+ T cell reactivation is an attribute of the combination of R848 and DGP.

FLT3L-DCs secrete IL-1β following treatment with R848 and DGP via NLRP3 activation. To test whether the underlying mechanism driving CD8+ T cell reactivation by Flt3l-DCs treated with the R848+DGP combination is dependent on IL-1β, CD8+ T cells were co-cultured as described above in presence or absence of an interleukin-1 receptor antagonist (IL-1RA). IL-1RA competitively inhibits binding of IL-1β to its receptor, thus blocking its function. The secretion of IFNγ by CD8+ T cells co-cultured with Flt3L-DCs treated with the R848+DGP combination and loaded with OVA was partially inhibited by IL1-RA (FIG. 40, p=0.078). Therefore, IL-1β signaling alone did not facilitate antigen-specific CD8+ T-cell reactivation. To further assess whether NLRP3 inflammasome activation within hyperactive DCs is important in CD8+ T cell reactivation, NLRP3 activation during DC treatment was inhibited using the small molecule MCC950, which inhibits NLRP3 oligomerization and function. Flt3L-DCs were loaded with OVA in the presence of the R848+DGP combination and MCC950 for 24 hours, before co-culture with CD8+ T-cells derived from OVA-immunized mice. CD8+ T cells co-cultured with Flt3L-DCs treated with the R848+DGP combination and MCC950 had a partially reduced level of reactivation as evidenced by lower levels of IFNγ secretion as compared to CD8+ T cells co-cultured with Flt3L-DCs treated with the R848+DGP combination in the absence of MCC950 (FIG. 40, p=0.0362). Taken together, these data indicate that hyperactive DCs treated with the R848+DGP combination strongly enhance antigen-specific CD8+ T cell reactivation, at least partially via IL-1β and NLRP3 inflammasome activation.

### Example B-10: Incorporation of a Hyperactivating Ether Lipid into Lipid Nanoparticles in the Presence or Absence of mRNA Encoding an Antigen to Improve Vaccine Response

This example describes the preparation and testing of lipid nanoparticles (LNPs) containing an ether lipid with a single alkyl chain, loaded with or without mRNA encoding an antigen. The ether lipid-containing LNPs are suitable for hyperactivating mammalian dendritic cells in combination with a small molecule pathogen-associated molecular pattern (PAMP) (e.g., R848). Hyperactivation of DCs induces pro-inflammatory cytokines and adds to their cytokine repertoire IL-1β, a critical cytokine for memory T cell formation. The aims of these experiments are to: 1) test whether the hyperactivating ether lipid with a single alkyl chain can effectively hyperactivate mammalian dendritic cells when delivered via an LNP, and 2) determine if this DC hyperactivation will enhancing de novo T cell activation and memory T cell reactivation in mice.

### Materials and Methods

LNP Production. LNPs are prepared by combining the following components with or without mRNA: (8-[(2-hydroxyethyl)[6-oxo-6-(undecyloxy)hexyl]amino]-octanoic acid), 1-octylnonyl ester (SM102) or other ionizable lipid, 1,2-distearoyl-sn-glycero-3-phosphocholine (DPSC) or other structural lipid, cholesterol, and 1,2-dimyristoyl-rac-glycero-3-methoxypolyethylene glycol-2000 (DMG-PEG2000) (or other PEG/stealth lipid), in the presence or absence of a hyperactivating ether lipid. Table 6-1 (with mRNA) and Table 6-2 (without mRNA) detail exemplary molar percentages of each component for exemplary LNP formulations.

LNPs are synthesized using the NanoAssemblr Ignite instrument (Precision Nanosystems). Lipids are first dissolved in ethanol and then combined to final concentrations as shown in Table 10-1 and Table 10-2. Lipids in ethanol are combined with sodium citrate buffer (pH 4) with or without mRNA at a 1:3 volumetric ratio, at a flow rate of 12 mL/min. mRNA (e.g., OVA mRNA) is added into sodium citrate buffer for loading into LNPs. After synthesis, LNPs are washed using 10 volumes of PBS, pH 7.4 to remove residual ethanol, and then concentrated using Amicon 10K MWCO centrifugal filters.

TABLE 10-1
Percent Molarity of Lipids in LNP
Formulations Containing mRNA

| | | Ether | Ether | Ether | Ether |
|---|---|---|---|---|---|
| | Vehicle | Lipid | Lipid | Lipid | Lipid |
| Lipid | LNP 0 | LNP 10 | LNP 20 | LNP 30 | LNP 40 |
| SM102 | 35% | 35% | 35% | 35% | 30% |
| DSPC | 33% | 23% | 13% | 3% | 0% |
| Cholesterol | 30.5% | 30.5% | 30.5% | 30.5% | 28.5% |
| DMG-PEG2000 | 1.5% | 1.5% | 1.5% | 1.5% | 1.5% |
| Ether Lipid | 0% | 10% | 20% | 30% | 40% |

TABLE 10-2
Percent Molarity of Lipids in LNP Formulations Lacking mRNA

| | | Ether | Ether | Ether | Ether |
|---|---|---|---|---|---|
| | Vehicle | Lipid | Lipid | Lipid | Lipid |
| Lipid | LNP 0 | LNP 10 | LNP 20 | LNP 30 | LNP 40 |
| DSPC | 50% | 40% | 30% | 20% | 10% |
| Cholesterol | 48.5% | 48.5% | 48.5% | 48.5% | 48.5% |
| DMG-PEG2000 | 1.5% | 1.5% | 1.5% | 1.5% | 1.5% |
| Ether Lipid | 0% | 10% | 20% | 30% | 40% |

LNP Characterization. Loading of the hyperactivating ether lipid into LNPs is assessed using HPLC. LNPs are dissolved in ethanol to release lipids prior to quantification. Standards prepared with known quantities of lipid are filtered through a 0.45 um filter. HPLC quantification is performed using an Agilent 1260 Infinity II HPLC equipped with a 1260 Infinity II Evaporative Light Scattering Detector. Column chemistry, mobile phases, and gradients depend on the structure of the hyperactivating ether lipid that is being detected.

Loading of mRNA into LNPs is quantified using a RiboGreen assay (ThermoFisher) following the manufacturer's protocol. Samples are diluted to fall within the range of the standard curve. LNPs are lysed using Triton X-100 to assess encapsulation of mRNA into LNPs. Both total mRNA and encapsulated mRNA are quantified. The size and charge of the LNPs is assessed using dynamic light scattering (DLS) and zeta potential on the NanoBrook Omni (Brookhaven).

Human moDC Production and Hyperactivation. Human monocyte-derived dendritic cells (moDCs) are produced as described in Example B-5. After differentiation, moDCs are plated into a 96-well flat-bottom plates at 1×10⁵ cells/well. Cells are treated with or without 1 μg/mL R848 (final), with or without LNPs loaded with a hyperactivating ether lipid (or vehicle control) in the presence or absence of mRNA. Hyperactivation induced by LNPs is measured after 24-48 hrs in culture with the LNPs. DC hyperactivation in response to hyperactivating ether lipids in LNPs is assessed as IL-1β secretion by live cells. Cell Viability is assessed using the LDH CyQuant Kit (Invitrogen) following manufacturer's instructions. IL-1β and IL-6 Lumit assays (Promega) are used to measure IL-1β and IL-6 present in moDC cell culture supernatants. In addition to IL-1β secretion, expression of moDC activation markers is quantified using flow cytometry. moDCs collected after 24-48 hrs in culture with LNPs, are stained with Live/Dead stain to identify live cells, followed by staining with antibodies specific for CD11c, CD40, CD86, HLA-DR, and HLA-ABC. Live cells are selected for analysis, and then CD11c⁺ cells are assessed for antigen presentation was assessed using antibodies specific for HLA-DR and HLA-ABC to determine if hyperactivation interferes with antigen presentation. Activation is assessed by staining for CD40 and CD86 to determine if hyperactivation increases expression of activation markers.

Immunization. C57BL/6J mice are immunized subcutaneously with LNPs as detailed in Table 10-3. OVA mRNA-loaded LNPs are used to deliver transcripts for production of OVA antigen in vivo, in combination with LNPs containing either hyperactivating ether lipid and R848 delivered via LNP or exogenously. The OVA mRNA dose is fixed at 1-5 μg/mouse, while the hyperactivating ether lipid is dosed at 50-100 μg/mouse, and the R848 is dosed at 10-50 μg/mouse (Table 10-3). Mice are given a primary immunization on Day 0, with a boost immunization with the same doses on Day 7. For assessment of short-term effector responses, on Day 14, blood and secondary lymphoid organs are collected. For assessment of long-term memory responses, on Day 40, blood and secondary lymphoid organs are collected. Blood is collected for measurement of antibody and T-cell responses. Serum is collected from the blood using serum separation tubes, while blood for cellular analysis is collected using K2EDTA tubes. After blood collection, mice are euthanized, and the draining lymph nodes and spleen are collected and processed into single cell suspensions.

TABLE 10-3
Immunization Groups

| | | Innate |
|---|---|---|
| Group | [mRNA] LNP Type + DC Stimuli | Pathway |
| 1 | LNP Vehicle (LNP 0) | None |
| 2 | R848 | NF-kB |
| 3 | [OVA] LNP Vehicle (LNP 0) | None |
| 4 | [OVA] LNP Vehicle (LNP 0) + R848 | NF-kB |
| 5 | [OVA] Ether Lipid LNP (LNP 10) + R848 | NLRP3 |
| | | NF-kB |
| 6 | [OVA] Ether Lipid LNP (LNP 20) + R848 | NLRP3 |
| | | NF-kB |
| 7 | [OVA] Ether Lipid LNP (LNP 30) + R848 | NF-kB |
| | | NLRP3 |
| 8 | [OVA] Ether Lipid LNP (LNP 40) + R848 | NF-kB |
| | | NLRP3 |

Assessment of Effector and Memory Immune Responses. OVA-specific T cells in the blood and draining lymph node of mice are assessed 14 and 40 days post primary immunization. CD8+ T cells specific for an MHC-I-restricted T cell epitope of ovalbumin (OVA) are quantified in the blood and dLN by SIINFEKL-tetramer staining. Briefly, red blood cells are lysed using a RBC lysis buffer, with lysis completed twice to remove all RBCs in the blood. Cells are washed, then stained for viability (Live/Dead), SIINFEKL-tetramer binding (MBL), and CD3, CD4, and CD8 expression. Cells are fixed with 4% paraformaldehyde, and counting beads are added to permit total cell counts to be determined. Data are collected using a BD FACS Symphony and analyzed using Flowjo (BD).

The frequency of T effector and T memory cells are assessed by flow cytometry, in the blood and draining lymph node of mice receiving OVA LNP immunization. Briefly, cell suspensions are stained with CD3, CD4, CD8, CD62L and CD44 antibodies to measure the frequency of T effector cells (CD44^{Low}CD62L^{neg}) and T memory cells (CD44^{high}CD62L⁺) using a BD FACS Symphony instrument and data is analyzed using Flowjo (BD).

OVA-specific antibodies in serum of mice receiving OVA LNP immunization are assessed 14 and 40 days post primary immunization. OVA-specific total IgG, IgG1, and IgG2b are assessed by ELISA. Briefly, ELISA plates are coated with 10 μg/mL Endofit Ovalbumin (InvivoGen) overnight, then washed and blocked with 2% bovine serum albumin. Plates are washed again, and then serum is added to the plates at a 1:500 dilution, followed by 1:5 dilutions for a total of 7 serum dilutions. Samples are washed, then incubated with detection antibodies specific for IgG, IgG1, or IgG2b, which are conjugated to HRP (Southern Biotech), to detect total, Th2-skewed, and Th1-skewed OVA-specific antibodies, respectively. Plates are washed, then incubated with TMB, and stop solution is added once color development is complete.

OVA-specific T cell responses are determined from assessment of secondary lymphoid organs. Post-immunization, draining lymph nodes and spleens are collected from the mice at early (Day 14) and late (Day 40) time points. Harvested lymph nodes and spleens are dissociated into single cell suspensions, which are used in ELISPOT assays. ELISPOT is used to detect IFNγ and IL-5 secretion by T cells, which are indicative of Th1 and Th2 responses, respectively. Cells from draining lymph nodes and spleens are plated in RPMI medium containing 10% FBS, 100 units/mL penicillin-streptomycin, 2 mM L-glutamine, 1 mM sodium pyruvate, 50 mM beta-mercaptoethanol, 10 mM HEPES, and 1× Gibco MEM non-essential amino acids (R10 media). Cells are plated at 200,000 cells/well in a 96 well ELISPOT plate and restimulated with 10 μg/mL OVA peptivator or 1 μg/mL OVA peptide antigens. As controls, additional plated cells are left unstimulated or stimulated with irrelevant antigens (not used in vaccination). Completion of the assay results in spots that can be visually quantified where cytokines were secreted by T cells, as a method to quantify the number of T cells responding to restimulation.

### Results

Lipid nanoparticles (LNPs) have become an important vaccine delivery tool, especially in the context of mRNA vaccines, which have played a large role in the fight against COVID-19 worldwide. LNPs are particularly useful for delivering mRNA cargo into cells. However, while LNP-based mRNA vaccines are effective at inducing antibody responses to the antigen(s) they encode, mRNA vaccines often elicit limited antigen-specific T cell responses, which negatively impacts their efficacy and longevity. In order to address this problem, the present disclosure describes the addition of a hyperactivating ether lipid to an LNP formulation containing an mRNA encoding an antigen to enhance its immunogenicity.

Hyperactivating Ether Lipid LNP Characterization. Based on the structure and solubilities of many of the ether lipids, these lipids are contemplated to be incorporable into LNPs. It is expected that modulation of various LNP components would affect the physical characteristics, as well as the biological activity of the LNPs loaded with ether lipids. Both loading level and the number of loaded LNPs are contemplated to be key variables affecting the ether lipid payload delivered to cells.

It is expected that LNPs can be prepared with or without ether lipid by combining the following: SM102, DPSC, cholesterol, and DMG-PEG2000. Some possible input molar ratios for these LNP formulations are listed in Table 10-1. As the ether lipids are somewhat structurally similar to DSPC, the ether lipids will likely replace DSPC in the formulation as the amount of ether lipid added to the LNPs increases. To determine if the loading level of ether lipids could be intentionally varied, and to understand how the loading levels would impact the biological activity of the LNPs, several different LNP compositions containing varying levels of ether lipid are prepared. LNPs are prepared either without ether lipid (LNP 0), or loaded with 10% (LNP 10), 20% (LNP 20), 30% (LNP 30), or 40% (LNP 40) molar ratios of ether lipid. LNPs can also be made either without mRNA, or with mRNA encoding antigen (ex. OVA mRNA) at varying loading levels to determine if including ether lipids will impact mRNA loading into LNPs. Exemplary formulations for LNPs that do not encapsulate mRNAs are included in Table 10-2.

All versions of the LNPs are expected to load hyperactivating ether lipid at increasing levels as the molar ratio of hyperactivating ether lipid to other lipid components increases. Increasing the hyperactivating ether lipid concentration is not expected to negatively impact the mRNA loading into LNPs, as the dose levels that are to be achieved allow for low mRNA loading into LNPs. It is expected that the LNPs will be 50-150 nm in size.

Hyperactivation of Human moDCs With Ether Lipid-Loaded LNPs. Human moDCs treated with LNPs loaded with ether lipids at an in-well ether lipid concentration of 50 μM or 100 μM are expected to secrete IL-1β when delivered in combination with R848 at 1-10 μg/mL. Increasing the amount of hyperactivating ether lipid loaded into each LNP is expected to make each LNP more potent, even with the same total dose of ether lipid delivered, and that this will result in increased IL-1β secretion by moDCs when delivered in combination with R848. It is expected that the IL-1β secretion will be dose-dependent. It is also expected that delivering LNPs loaded with ether lipids may increase secretion of other inflammatory cytokines, such as IL-6 and TNFα. It is further expected that delivering LNPs loaded with ether lipids may increase surface expression of activation markers in human moDCs, such as CD40 and CD86.

Ether Lipid-Loaded LNPs Enhance T Cell Responses. Using the assays described above to assess short term (2 weeks post vaccination) and long term (>4 weeks post vaccination) adaptive T cell immunity, effector and memory responses are analyzed. Table 10-3 lists experimental groups to be included in the study. As a negative control, mice of Group 1 receive a sham injection containing no antigen and no adjuvants to serve as a baseline where little to no antigen-specific immune responses are expected to be elicited. Group 2 mice receive mRNA encoding a model antigen (OVA) formulated in LNPs, which represents a standard LNP vaccination protocol. The remaining groups all receive LNP-loaded with mRNA encoding OVA in combination with adjuvants. Depending upon the type of stimuli administered, one of the following is expected: activate cells (induce NF-kB signaling); ii) hyperactivate DCs (induce NF-kB and NLRP3 pathway activation).

Hyperactivating conditions are contemplated to benefit effector responses, with increased OVA-specific T cells 2 weeks after prime immunization. Additionally, hyperactivation-induced IL-1β signaling leads to a durable memory response. By immunizing with a sham treatment (Group 1), little to no effector and memory responses are likely to be observed, while immunizing with antigen mRNA alone or in combination with R848 (Groups 3 and 4) is contemplated to lead to a small effector response and minimal memory formation. Groups 5-8 should allow for DC hyperactivation, which is expected to be an improvement over Group 2 treatment. It is expected that increasing the amount of hyperactivating ether lipid in each LNP will enhance the T cell memory response, which is to be verified with the groups included in this study. Further, the adjuvanting stimuli can be formulated in various ways. For example, hyperactivating ether lipid is encapsulated in an LNP with or without antigen mRNA. R848 is administered as an individual component, within an LNP, or within an LNP containing a hyperactivating ether lipid.

Studying T and B cell responses to vaccination generates in vivo data that is contemplated to demonstrate that combining hyperactivating ether lipid with R848 will result in improvements in antigen-specific immune responses. Assessment of how administration of various DC stimuli affects B cell responses is accomplished by measuring total antigen-specific IgG antibodies, as well as antibody isotypes such as IgG1 (associated with TH2 responses) and IgG2b (associated with TH1 responses) in the serum of immunized mice. Based on previous studies using similarly structured lipids delivered as microparticles, it is expected that mice immunized with LNPs loaded with mRNA encoding an antigen and hyperactivating ether lipid are expected to result in TH1-skewed responses.

Taken together, this example describes the preparation and testing of lipid nanoparticles (LNPs) containing an ether lipid with a single alkyl chain, loaded with or without mRNA encoding an antigen. The ether lipid LNPs are suitable for hyperactivating mammalian dendritic cells in combination with a small molecule PAMP (e.g., R848) that can be delivered via LNP or exogenously. DC hyperactivation induces pro-inflammatory cytokines, and in particular IL-1β, a critical cytokine for memory T cell formation. The hyperactivating ether lipid LNPs are expected to provide more robust effector T cell and memory T cell functions upon injection in vivo.

### Example B-11: Scale Up of DGP (Compound 2) Ether Lipid Drug Product Formulations and Impact of Size on Potency

This example describes the scaled up preparation of DGP (Compound 2) Drug Product (DP) and how it influences size of DGP DP recovered after synthesis. DGP Drug Substance (DS) and DGP DP are modified before and after formulation to maintain the size.

### Materials and Methods

DGP (Compound 2) DP Synthesis. DGP DP was synthesized at a 3 mL scale using the following procedure. A solution of 4.44% Poloxomer 407 (P407) was prepared using sterile, ultrapure water at 4° C., and sterile filtered through a 0.2 μm nylon filter. This 4.44% P407 solution was added to a DGP powder DS to achieve 4.4 mg/mL DGP DP and stirred using a magnetic stir bar at 1000 rpm for 1 hour at RT. Sterile 10×PBS was added to the solution resulting in a 1×PBS concentration and 4 mg/mL DGP DP concentration. The mixture was then stirred at 1000 rpm for another 30 min at RT. For DGP DP prepared at 12 or 18 mL scale, the same procedure was followed, with volumes, vials, and stir bars scaled up to achieve the 4 mg/mL DGP DP preparation.

In cases where DGP DS was micronized prior to use, DGP DS underwent jet milling using an M-50 (Microtech Engineering Company) to reduce DS size to an average of 3-5 μm. In cases where DGP DP was sonicated, as a stand in for other homogenization techniques (high shear and high pressure homogenization), 4 mL aliquots of DGP DP were sonicated using a Qsonica 125 using either 18 mL scale unmodified DGP DS or 100 mL scale DP preparations using micronized DGP DS. For in vitro studies, DGP DP samples were sonicated at 2 W (low energy) or 4 W (medium energy) over two 30 s intervals. For in vivo studies, DGP DP samples were sonicated at 3 W over two 30 s intervals as a mid-energy assessment.

DGP (Compound 2) DP Sizing. DGP DP size was determined using a Malvern Mastersizer 3000 with a Hydro SV attachment. Briefly, DGP DP was diluted to 1 mg/mL in water prior to sizing. The 1 mg/mL DGP DP was added drop-wise to a measurement chamber filled with water under spinning until laser obscuration was 3-5%. Five measurements were taken per sample, with the average of the measurements reported.

Human Monocyte-Derived Dendritic Cell (moDC) Production. Human monocytes were isolated from Leukopaks purchased from Miltenyi Inc. (San Jose, CA) using the StraightFrom® Leukopak® CD14 microbead kit according to the manufacturer's instructions. Monocytes were differentiated into monocyte-derived dendritic cells by culturing in RPMI medium containing 10% FBS, 100 units/mL penicillin-streptomycin, 2 mM L-glutamine, 1 mM sodium pyruvate, 50 mM beta-mercaptoethanol, 10 mM HEPES, and Gibco MEM non-essential amino acids, as well as recombinant human GM-CSF (50 ng/mL) and IL-4 (25 ng/mL) for 6 days, with a feeding with media containing GM-CSF and IL-4 on day 3.

Human moDC Hyperactivation. After differentiation, moDCs were plated in 96-well flat-bottom plates at 1×10⁵ cells/well. Quality of differentiation of moDCs was assessed using flow cytometry as in other examples, with live cells that stained positive for CD11c and CD209 indicating a successful differentiation into moDCs. Cells were treated with or without 10 μg/mL R848 (final), and with or without DGP DP at 20 μg/mL (final). Hyperactivation induced by DGP DP was measured after 24 hrs in culture at 37° C., 5% CO₂. DC hyperactivation in response hyperactivating ether lipid in LNPs was assessed by measuring IL-1β present in moDC cell culture supernatants using a Lumit assay (Promega) following the manufacturer's instructions. Cell Viability was assessed using the LDH CyQuant Kit (Invitrogen) following the manufacturer's instructions.

In vivo DC hypermigration assessment (CCR7 staining). C57BL6J mice were treated subcutaneously as listed in Table 11-1. After 4 hrs, mice were euthanized by CO₂ followed by cervical dislocation, and draining lymph nodes (dLN) were collected and processed to single cell suspension. Single cell suspension from the dLN of treated mice were stained with antibodies against CD69 and CCR7. Single cell suspensions from the draining lymph or spleen were resuspended in 100 μl PBS containing Live/Dead violet (1:1000) and incubated for 20 min at 4° C. Cells were then washed and resuspended in 100 μl FACS buffer containing Fc block (1:100) for 10 min. For myeloid cell staining, cells were then resuspended in 100 μl of antibody cocktail containing CD11b, Ly6c, F4/80, MHC-II, and CD24 (1:100) to identify the cell type and incubated for 20 min at 4° C. on a shaker. Monocytes were identified as CD11b+, Ly6c+/F4/80neg, MHCneg live cells; moDCs were identified as CD11b+, Ly6c+/F4/80neg, MHC+ live cells; macrophages were identified as F4/80+, MHC+/CD24neg live cells; and DCs were identified as CD11c+, MHC-II+/F4/80neg live cells. Anti-mouse CD69 was added to the antibody cocktails at a dilution of (1:100) to assess the mean fluorescence intensity (MFI) of CD69 on monocytes, moDCs, macrophages and DCs. For CCR7 staining, anti-CCR7 was added to the antibody cocktail at a dilution of (1:100) to assess the MFI of CCR7 on DCs. Count bright counting beads were added before the acquisition of samples.

TABLE 11-1
Study Groups

| | | Modification of DGP |
|---|---|---|
| Group | Treatment Dose | Preparation |
| 1 | PBS | Not Applicable |
| 2 | R848 (50 μg) | |
| 3 | DGP (100 μg in 4% KP407) | |
| 4 | R848 (50 μg) + | |
| | DGP (100 μg in 4% KP407) | |
| 5 | R848 (50 μg) + | Micronized DGP Drug |
| | DGP (100 μg in 4% KP407) | Substance |
| 6 | R848 (50 μg) + | Sonicated DGP Drug Product |
| | DGP (100 μg in 4% KP407) | |
| 7 | R848 (50 μg) + | Micronized DGP Drug |
| | DGP (100 μg in 4% KP407) | Substance + |
| | | Sonicated DGP Product |

### Results

Scaling Up DGP (Compound 2) DP Production Increases DGP DP Size. DGP (Compound 2) DP size was determined using a Malvern Mastersizer 3000 with a Hydro SV attachment. At a 3 mL synthesis scale, 50% of DGP particles are less than 13.8 μm in diameter (Table 11-2), with 90% of DGP particles less than 27.5 μm. However, as soon as the scale increases to 12 or even 18 mL, the average size of the DGP increases to 26-27 μm. DGP particles should be large enough to stay at the site of injection (micron scale), they should also be small enough for a dendritic cell to engulf them. As such, an average particle size of less than 15 μm is preferred for DGP activity. Given the increased size after scale up, additional methods to reduce the DGP particle size were added to the synthesis method.

TABLE 11-2
DGP Size Increases as Scale of Preparation Increases

| | Particulate | Particulate | Particulate |
|---|---|---|---|
| | Size (μm) | Size (μm) | Size (μm) |
| Scale | D10 | D50 | D90 |
| 3 mL | 6.83 | 13.8 | 27.5 |
| 12 mL | 12.0 | 25.0 | 51.7 |
| 18 mL | 12.9 | 26.7 | 56.0 |

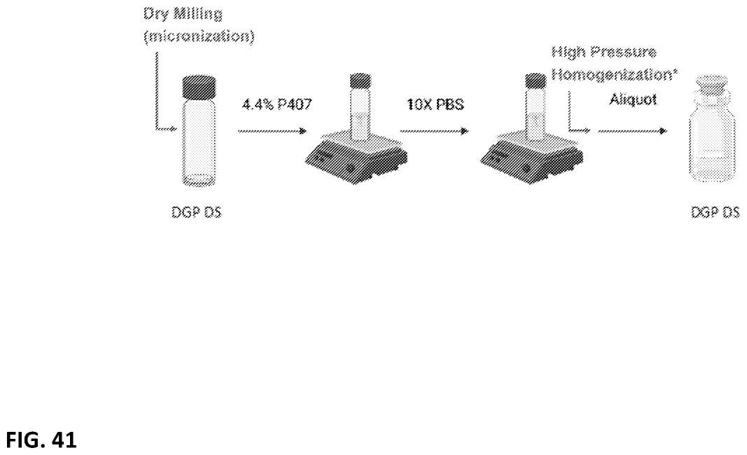

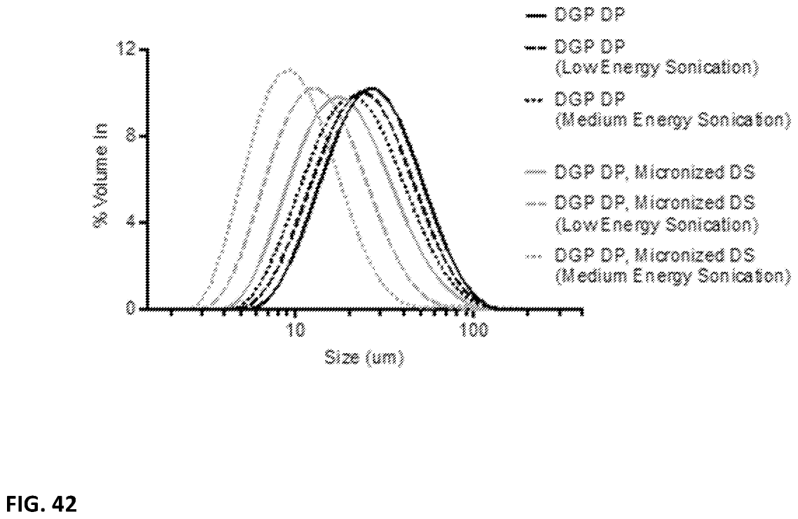

Micronization of DGP (Compound 2) DS and Sonication of DGP DP Reduces the Particle Size as the Preparation Scale Increases. In order to reduce the size of the DGP (Compound 2) DP scale, two additional techniques were added to the DGP synthesis procedure: micronization of DGP DS to an average of 3-5 μm using jet milling and homogenization (high-shear or high pressure homogenization) of the DGP DP to reduce aggregation of the DGP DP during formulation (FIG. 41). For initial studies, sonication was performed in place of homogenization. At both the 18 mL scale and the 100 mL scale, using micronized DGP DS to make the DGP DP reduced the size of the DGP DP (Table 11-3). In addition, sonication of the non-micronized DGP also reduced the particle size of the DGP DP. However, the combination of the two: micronized DGP DS and sonicated DGP DP resulted in the largest drop in DGP size (Table 11-3). Both micronization of DGP DS before DP synthesis and sonication of DGP DP reduced the particle size compared to DGP DP prepared without micronization or sonication (FIG. 42).

TABLE 11-3
DGP DP Particulate Size After DS
Micronization and DP Sonication

| | DGP DS | DGP DP | | | |
|---|---|---|---|---|---|
| | Modifi- | Modifi- | Particulate Size (μm) | | |
| Scale | cations | cations | D10 | D50 | D90 |
| 18 mL | N/A | N/A | 12.9 | 26.7 | 56.0 |
| 18 mL | N/A | Sonication: (4 mL) | 11.3 | 23.9 | 51.3 |
| | | Low energy (~2 W) | | | |
| | | Sonication: (4 mL) | 10.0 | 21.3 | 47.1 |
| | | Medium energy (~4 W) | | | |
| 100 mL | Micron- | N/A | 8.69 | 18.1 | 40.4 |
| | ization | | | | |
| 100 mL | Micron- | Sonication: (4 mL) | 6.49 | 13.2 | 28.2 |
| | ization | Low energy (~2 W) | | | |
| | | Sonication: (4 mL) | 5.04 | 9.62 | 20.0 |
| | | Medium energy (~4W) | | | |

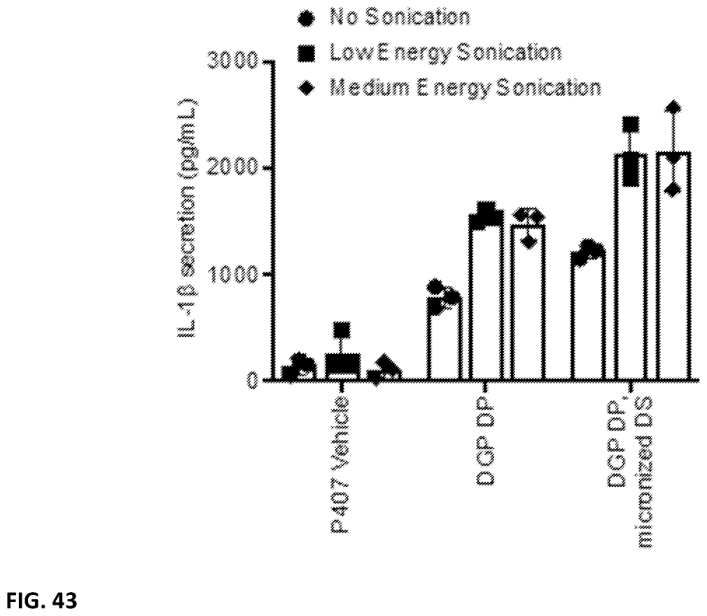

Reducing the Size of DGP (Compound 2) DP Particulates Increases IL-1β Secretion by Human moDCs. One indicator of successful hyperactivation of human moDCs is the secretion of IL-1β from live cells. After differentiation, moDCs were treated with 10 μg/mL R848, and with or without DGP DP at 20 μg/mL. Hyperactivation induced by DGP DP was measured after 24 hrs in culture at 37° C., 5% CO₂. Cell Viability was assessed using the LDH CyQuant Kit, and cell viability seen was >85% compared to cells treated with R848 alone. Treating moDCs with unmodified DGP DP resulted in IL-1β secretion, but that IL-1β secretion was further increased by micronizing the DGP DS, sonicating the DGP DP, or both micronizing the DGP DS and sonicating the DGP DP after synthesis (FIG. 43).

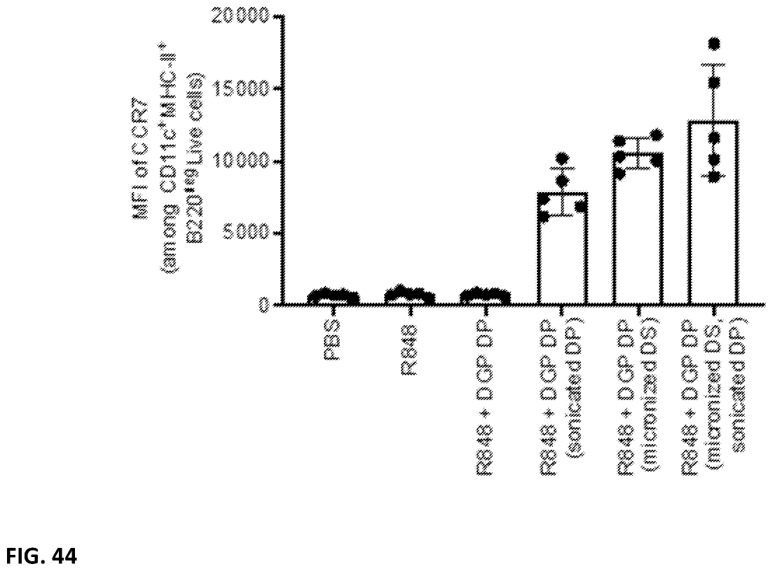

Reducing the Size of DGP (Compound 2) DP Particulates Increases DC Hypermigration. Another hallmark of DC hyperactivation is the ability to hypermigrate from the skin to the draining LN where they can interact with T cells. Homing of DCs (defined as live CD11c⁺MHC-II⁺/B220^{neg} cells in LNs) to the dLNs can be assessed by upregulation of the CCR7. As expected, since it is not a hyperactivating treatment, R848 did not increase CCR7 expression on DCs in LNs compared to PBS. In addition, R848 delivered with DGP DP prepared with no modifications to reduce the size of the DGP DP did not result in increased CCR7 expression by DCs (FIG. 44), even though increased IL-1β was produced by human moDCs in vitro. The hypermigration and IL-1β pathways work independently of one another, though both are important phenotypes of hyperactivated DCs. However, when methods to reduce DGP DP size during synthesis were employed, CCR7 expression by moDCs was significantly increased compared to moDCs of mice treated with R848 alone, or R848+ unmodified DGP DP (FIG. 44), suggesting that the size of the DGP DP can dramatically impact the kinetics of hyperactivation in vivo.

### Example B-12: Comparison of the Effects Induced by Administration of R848+DGP and Hyperactive Dendritic Cells on Antigen-Specific T Cell Responses In Vivo

### Materials and Methods

Mouse Strains. Eight to Twelve weeks old C57BL/6J mice were purchased from Jackson Labs and allowed to acclimate to the Explora BioLabs housing facility for at least one week. In all experiments, mice were randomly assigned to experimental groups. All experimental procedures were approved by the institutional animal care and use committee at Explora BioLabs (Protocol ID: EB17-010-300).

Murine Bone Marrow-Derived FLT3L-DC Generation and Quality Control Staining of Flt3L-DC were performed as described in Example B-9.

Lipid Preparation. Lipid stocks were formulated at 4 mg/mL lipid in 4% Kolliphor P407 (KP407). Lipids were prepared from lyophilized stocks by mixing with a cold solution of KP407 at 1000 rpm using a shaker for 1 hour at RT. A 10×PBS solution was then added and the lipids were mixed at RT for an additional 30 min to make the 4% KP407 stock solution isotonic. Lipids were used for injections or DC treatment within one hour of their preparation using a dose of 100 μg/mouse. For in vitro DC treatments, DGP was prepared as described above at 650 mg/mL lipid, then was further diluted in cell culture media to a final concentration of 41 mM.

Pre-Treatment of Murine Bone Marrow-Derived FLT3L-DCs. Flt3L-DCs were harvested on day 9 post differentiation, washed with PBS, and resuspended in a FLT3L-containing I10 media at concentration of 8×10⁶ cells/ml. 1 mL of cell suspension was added to 5 mL MacroTubes, then cells were subjected to different pre-treatment conditions in a final volume of 2 mL. The tubes were incubated at 37° C., 5% CO₂ for 24 hours on a rotator. After 24 hours, DCs were washed twice with PBS, counted by Propidium iodide staining using the Moxi automated cell counter, then loaded with OVA (500 mg/ml) for 2 hours. DCs were washed twice again with PBS and 1×10⁵ cells were resuspended in 100 μL.

Subcutaneous (SC) Injection. Mice were placed under Isoflurane for a few min for anesthesia. Tubes containing the chemicals (OVA alone or in the presence of one or both of R848 and DGP) were vortexed for 30 seconds, then loaded with a 1 mL Sub-Q syringe. Mice were injected subcutaneously on the upper right back with a 100 μL total volume. Tubes containing pre-treated DC cell suspensions were gently mixed using a pipette, then a 100 μL total volume containing 1×10⁶ DCs were loaded using a 1 mL Sub-Q syringe. Mice were injected subcutaneously on the upper right back. Mice received chemical injections or DC injections with the same treatments and doses at the same site every 7 days for 3 total injections on Day 0, Day 7, and Day 14.

Blood Collection and Processing. Mice were placed under Isoflurane for approximately 10 min for anesthesia. Using a 21G needle, mice were gently poked through the skin to the submandibular space to induce bleeding. Five of six drops were collected in a mini collect K2EDTA blood collection tube. Blood samples were maintained at RT (RT). To process blood, 1 mL of RBC lysis buffer was added to 150 μl of whole blood into each well of a 96 deep-well plate. Samples were then mixed and incubated at RT for 20 min. 600 μl of PBS was then added to all wells and samples are centrifuged at 600×g for 5 min. This step was repeated twice, then pellets were resuspended in 200 μl of PBS and transferred to a 96-well V bottom plate for cell staining.

Draining Lymph Node (dLN) Dissection and Dissociation. Twenty-one days post first SC injection; inguinal, axillary, and brachial draining lymph nodes were collected from the side of injection of each mouse and placed in PBS. The dLNs was then processed using a Miltenyi spleen dissociation kit according to manufacturer's protocol. In brief, the dLN from each mouse was transferred into the gentleMACS C Tube containing the enzyme mix. The dLN was then dissociated using the gentleMACS Program: program 37C_m_SDK_1. Cell suspensions were then collected and filtered through a 30 μm Pre-Separation Filter. Cells were counted using the Moxi automated counter. The dLN samples were then resuspended in complete RPMI (R10) at a density of 2×10⁶ cells/mi.

Enzyme-linked immunosorbent spots assay (ELISPOT). IFNγ ELISPOT plates (R&D Systems) were blocked with R10 media (RPMI-1640 media supplemented with 10% FBS, 100 U/mL Penicillin, 100 mg/ml Streptomycin, 2 mM L-Glutamine, 1 mM Sodium Pyruvate, and 54 mM beta-mercaptoethanol) for 45 min. At the end of blocking, media was discarded and 100 μL of either R10 media alone, or R10 media containing 1 mg/mL of Ovalbumin PepTivator (a pool of peptides consisting of 15-mers with 11 amino acid overlaps covering the complete sequence of OVA). The dLN cell suspension was seeded at 500,000 cells per well in 100 μL and plates were incubated at 37° C. for 18 hours. After incubation, cells were discarded and ELISPOTs were developed according to the manufacturer's instructions. Described briefly, plates were washed four times with 1× wash buffer (R&D Systems) followed by a 2-hour incubation at RT with 100 μl of diluted detection antibody. Plates were washed four times and 100 μl of diluted Streptavidin-Alkaline Phosphatase was added for 2 hours at RT. Plates were washed four times and 100 μl 5-Bromo-4-Chloro-3′ Indolylphosphate p-Toluidine Salt and Nitro Blue Tetrazolium Chloride (BCIP/NBT) substrate was added. Plates were incubated for 1 hour at RT while being hidden from light. Substrate was removed and plates were washed with deionized water. Plates were gently dried with Kimwipes and left to dry overnight at RT. The next day, plates were read on the S6 Universal M2 ELISPOT plate analyzer.

Tetramer Staining. 3×10⁵ cells from dLN of each mouse, or 200 μL of processed blood samples were plated into 96-well V bottom plates. Cells were spun at 400×g for 4 min then washed with PBS at least once before cell staining. Cells were then resuspended in 100 μL PBS containing Live/Dead Aqua (1:1000) and incubated for 20 min at 4° C. Cells were then washed and resuspend in 100 μL of FACS buffer containing Fc block (1:100) for 10 min, then washed again with 100 μL FACS buffer. For tetramer staining, cells were resuspended in 100 μL of FACS buffer containing SIINFEKL-PE tetramer (1:20) and incubated at 37° C. for 2 hours. For surface marker staining, cells were washed with 100 μL FACS buffer and spun for 4 min at 400×g. The cell pellet was stained with anti-CD3, anti-CD4 and anti-CD8a antibodies for 20 min at 4° C. Cells were then washed and resuspended in 100 μL of 4% PFA to fix the cells for 20 min at RT. After fixation, cells are washed twice with FACS buffer and kept at 4° C. overnight in 150 μL FACS buffer. Prior to sample acquisition on the Symphony A3, Countbright counting beads were added to the samples for measurement of absolute cell numbers.

Data Analysis. Graphical data was shown as mean values with error bars indicating the SD of 5 mice per group. Each symbol represents one mouse. All experiments were analyzed using Prism 7 (GraphPad Software).

Statistical differences were calculated by one-way ANOVA with Tukey post-hoc test. Statistical significance for ELISPOT data with more than two groups was tested with two-way ANOVA with Tukey multiple comparison test correction. P values of <0.05 (*), <0.01 (**) or <0.001 (***); <0.0001 (****) indicated significant differences between groups.

### Results

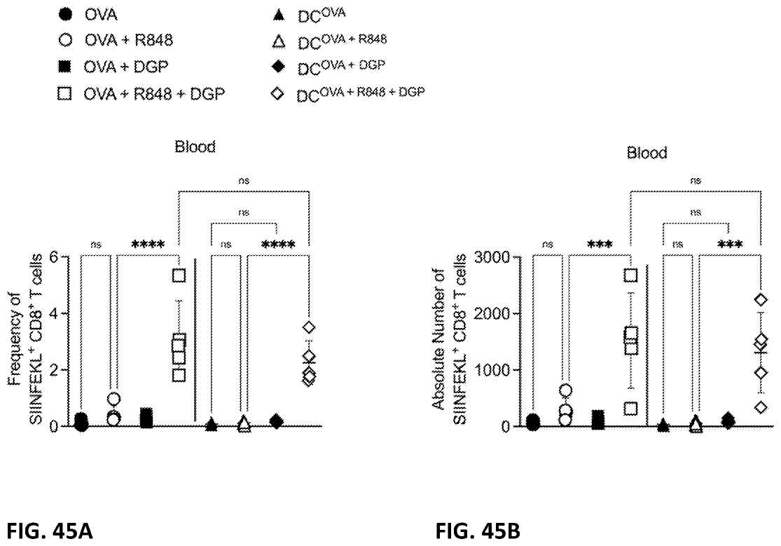

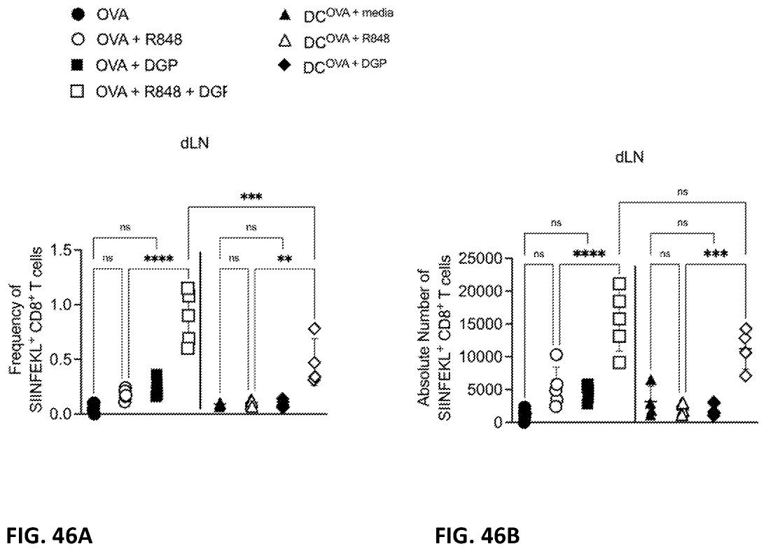

R848+DGP Enhances Antigen-Specific CD8⁺ T Cell Generation In Vivo. To assess if R848+DGP modulates T cell responses, mice were injected SC with OVA antigen alone (OVA+PBS) or OVA antigen in combination with R848 (OVA+R848), DGP (OVA+DGP) or a combination of R848 and DGP (OVA+R848+DGP)]. Mice received one immunization and two boost injections SC at the same site every 7 days. On D21 (after 3 injections) blood and the dLN were collected from the injected mice to measure the abundance of OVA-specific CD8⁺ T cells by flow cytometry using H2 kb-restricted SIINFEKL (OVA 257-264) tetramer. When mice were injected with OVA in combination with R848 alone or DGP alone, the frequency and absolute number of SIINFEKL⁺ CD8⁺ T cells in the blood did not significantly change compared to mice injected with OVA alone (FIG. 45A-B). In contrast, the injection of OVA in combination with R848+DGP induced significantly higher frequency and absolute number of SIINFEKL⁺ CD8⁺ T cells in the blood compared to mice injected with OVA antigen in combination with R848 alone (FIG. 45A-B, p=<0.0001, one-way ANOVA). These data indicate that R848+DGP strongly enhances antigen-specific T cell generation in the blood compared to antigen alone or antigen in combination with R848 alone or DGP alone. Similar trends were observed in the dLN. The injection of OVA in combination with R848 alone or DGP alone did not significantly increase the frequency and absolute number of SIINFEKL⁺ CD8⁺ T cells in the dLN (FIG. 46A-B). In contrast, the injection of OVA with R848+DGP induced significantly higher frequency and absolute number of SIINFEKL⁺ CD8⁺ T cells in the dLN as compared to mice injected with OVA antigen in combination with R848 (FIG. 46A-B, p<0.0001, one-way ANOVA). Overall, these data indicate that R848+DGP significantly enhances antigen-specific CD8+ T cell generation in blood and dLNs as compared to antigen alone or antigen plus R848 or DGP alone.

Hyperactive DCs Enhance Antigen-Specific CD8⁺ T Cell Generation In Vivo. To address whether hyperactive DCs can enhance antigen-specific CD8+ T cell responses similarly to R848+DGP injection, FLT3L-DCs that were differentiated from the bone marrow of mice using Fms-like tyrosine kinase 3 ligand (FLT3L) recombinant cytokine, were used for adoptive cell transfer into recipient mice. FLTL3 cytokine generates conventional DCs (cDCs) that are divided into two major subsets: cDC1s and cDC2s (Kirkling et al., Cell Rep, 23:3658-3672, 2018). Of these subsets, cDC1s are uniquely capable of antigen cross-presentation and can prime naïve CD8⁺ T cells, but also CD4⁺ T cells (Ferris et al., Nature, 584(7822):624-629, 2020). In contrast, cDC2s activate Th2 and Th17 immunity. Three batches of cDCs were generated for 3 subsequent injections on Day 0, 7 and 14. For each batch, FLT3L-DC cultures were stained 9 days post-differentiation to assess the efficiency of cDC generation and measure the frequencies of cDC1 and cDC2 subsets in the culture. cDCs were characterized as CD11c⁺MHC-11CD45R-live cells and accounted for more than 84% of live cells in the three batches generated as measured by flow cytometry (Table 12-1). Among these cDCs, the frequency of cDC1 cells (characterized as CD24⁺SIRP1a) was 62.9%, for injection 1 (Day 0), 58.4% for injection 2 (Day 7) and 64.3% for injection 3 (Day 14). Whereas cDC2 (characterized as CD24⁻ SIRP1a⁺) accounted for 29.3% of cDC for injection 1, 31.8% for injection 2 and 29.0% for injection 3. In addition, the frequency of cross-presenting cells that were defined as CD103⁺XCR1⁺was measured (Theisen et al., Cancer Immunol Res, 7:29039, 2019). CD103⁺XCR1⁺ represented 24% of cDC1 cells for injection 1, 26.6% for injection 2 and 27.2% for injection 3. Both cDC1 and cDC2 subsets can become hyperactivated (Hatscher et al., Sci Signal, 14(680):eabe1757, 2021; and Zhivaki et al., Cell Rep, 33(7):108381, 2020). Therefore, FLT3L-DCs selected to assess the role of hyperactive DCs in antigen-specific CD8⁺ T cell responses.

TABLE 12-1
Efficiency of cDC Differentiation and DC Subset Frequency

| | % cDC | | | % cross- |
|---|---|---|---|---|
| DCs used | (CD11c⁺CD45R⁻ | % | % | presenting DC |
| for | MHC-II⁺Live cells | cDC1 | cDC2 | (XCR1⁺CD103⁺) |
| Injection 1 | 87.9% | 62.9% | 29.3% | 24.0% |
| on Day 0 | | | | |
| Injection 2 | 85.0% | 58.4% | 31.8% | 26.2% |
| on Day 7 | | | | |
| Injection 3 | 84.6% | 64.3% | 29.0% | 27.2% |
| on Day 14 | | | | |

To assess T cell responses following DC injections, recipient mice were injected SC on Day 0, 7 and 14 with FLT3L-DCs that were loaded with OVA and either left untreated (DC^{OVA}) or pre-treated with R848 (DC^{OVA+R848}), pre-treated with DGP (DC^{OVA}+DGP), or pre-treated with R848 and DGP (DC^{OVA+R848+DGP}). Endogenous OVA-specific CD8+ T cells were measured in the blood and dLN on day 21 day using a H2 kb-restricted SIINFEKL (OVA 257-264) tetramer.

Injection of OVA-loaded active DCs that were pre-treated with R848 (DC^{OVA+R848}) or OVA-loaded DCs that were pre-treated with DGP (DC^{OVA+DGP}) did not induce significant increase the frequency or absolute number of OVA-specific T cells compared to naïve DCs that were left untreated and loaded with OVA (DC^{OVA}). In contrast, the injection of hyperactive DCs treated with R848 and DGP and then loaded with OVA (DC^{OVA+R848+DXP}) induced a significant increase in the frequency and the absolute number of SIINFEKL⁺ CD8+ T cells in the blood as compared to the injection of DC^{OVA+R848} (FIG. 45A-B, p<0.0001 for frequency, p=0.0005 for absolute numbers, one-way ANOVA). These data indicated that similarly to R848+DGP treatment, the SC injection of hyperactive DCs enhances antigen-specific CD8+ T cell generation in the blood as compared to naïve DCs, active DCs or DC treated with DGP alone.

Similar trends were observed in the dLN. The injection of OVA-loaded active DCs pre-treated with R848 (DC^{OVA+R848}) or DCs pre-treated with DGP and loaded with OVA (DC^{OVA+DGP}) did not induce significant increase in OVA-specific T cells compared to naïve DCs that were left untreated and loaded with OVA (DC^{OVA}). However, the injection of hyperactive DCs treated with R848 and DGP then loaded with OVA (DC^{OVA+R848+DGP}) significantly enhanced the frequency and absolute number of SIINFEKL⁺ CD8+ T cells in the dLN as compared to the injection of DC^{OVA+R848} (FIG. 46A-B, p=0.0013 for frequency, p=0.0002 for absolute numbers, one-way ANOVA). Overall, these data indicate that hyperactive DCs are superior to naïve DCs, active DCs or DCs treated with DGP alone at inducing antigen-specific T cell responses in the blood and the dLN of recipient mice.

R848+DGP or Hyperactive DCs Strongly Enhances Antigen-Specific IFN-γ Secretion in the dLN. The generation of antigen-specific T cells in the dLN implied that these T cells can induce strong effector functions upon antigen re-encounter. To further assess the specificity and functionality of individual T cells that result from a SC immunization with R848+DGP or hyperactive DCs as described above, the skin dLN from injected mice were isolated twenty-one days post injection, then dLN were enzymatically and mechanically dissociated. Single cell suspensions were then either left unstimulated or re-stimulated with OVA peptivator for an enzyme-linked immunosorbent spot (ELISpot) assay to quantitatively measure antigen-specific T cell functional activities such as (interferon-γ) IFN-γ secretion. As such, the ability of T cells from the dLN to quickly release IFN-γ upon antigen re-encounter is indicative of antigen-specific memory T cell activity. Therefore, this assay was used to measure the magnitude and potency of OVA-specific memory T cells that were generated post-treatment.

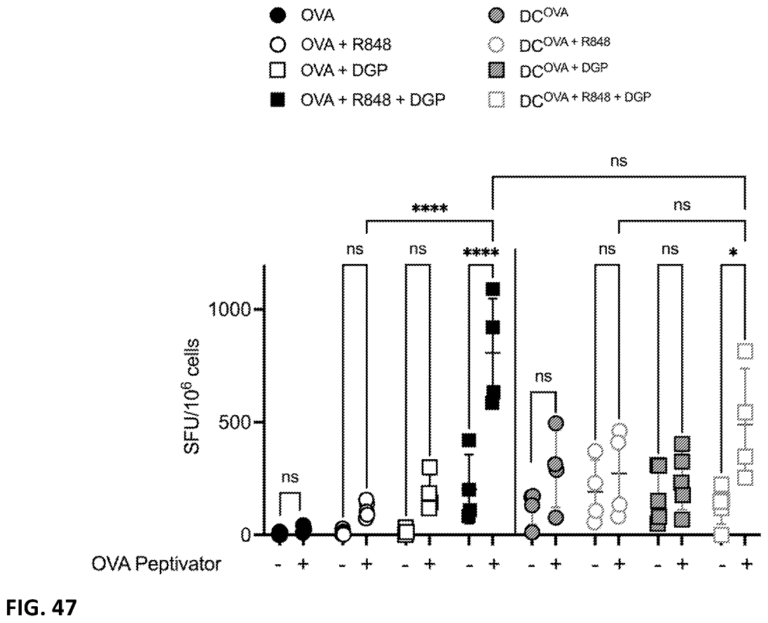

Upon restimulation with OVA peptivator, cells from dLN of mice that were injected with OVA alone, or OVA+R848 or OVA+DGP, did not induce IFN-γ secretion as compared to an unstimulated dLN, indicative of weak or no antigen-specific T cells responses (FIG. 47). In contrast, when mice were injected with OVA in combination with R848+DGP, cells from dLN that were stimulated with OVA peptivator induced strong IFN-γ secretion as compared to unstimulated dLN as revealed by the number of IFN-γ⁺ spots detected (FIG. 47, p<0.0001, 2-way ANOVA). Furthermore, the injection OVA+R848+DGP was superior to OVA+R848 at inducing IFN-γ⁺ T cells upon OVA peptivator stimulation (FIG. 47, p<0.0001, 2-way ANOVA). These data indicate that the SC injection of R848+DGP strongly enhances antigen-specific IFN-γ secretion in the dLN. To further explore whether the adoptive transfer of hyperactive DCs into recipient mice mimics the same effect of R848+DGP injection on IFN-γ activity, single cell suspensions from the dLN of injected mice were either left unstimulated or re-stimulated with OVA peptivator for 18 hours as described above. ELISPOT data revealed that the dLN from mice that were injected with naïve DCs (DC^{OVA}) or active DCs treated with R848 (DC^{OVA+R848}) and loaded with OVA did not induce antigen specific IFN-γ release upon dLN restimulation with OVA peptides. Similarly, the injection of DCs treated with DGP alone then loaded with OVA (DC^{OVA+DGP}) did not result in antigen-specific IFN-γ release. In contrast, hyperactive DCs that were pre-treated with R848+DGP (DC^{OVA+R848+DGP}) induced significantly higher levels of IFN-γ release upon dLN re-stimulation as compared to unstimulated cells (FIG. 47, p=0.0160, 2-way ANOVA). These data corroborated the previous observation (FIG. 46A-B), whereby hyperactive DCs enhanced antigen-specific T cell generation in the dLN. There was no significant difference between the dLN from mice that were injected with active DCs (DC^{OVA+R848}) or hyperactive DCs treated with R848 (DC^{OVA}+R848+^{DGP}). However, when the effect of R848+DGP was compared to the effect of hyperactive DC injection, there was no significant difference in the level of IFN-γ release following dLN restimulation when mice were injected with OVA+R848+DGP compared to mice injected with DC^{OVA+R848+DGP} (FIG. 47).

Overall, these data indicated that R848+DGP injection and hyperactive DC injection can both enhance antigen-specific IFNγ secretion in the dLN and that hyperactive DCs are sufficient to recapitulate the effect seen with R848+DGP injection.

### Example B-13: Assessment of Local and Systemic Inflammation Induced by R848+DGP

The objectives of this study were to determine whether subcutaneous injection of R848+DGP induces local and/or systemic inflammation in mice and modulates expression of activation and hyperactivation markers on myeloid cells in vivo.

### Materials and Methods

Lipid Preparation. Lipid stocks were formulated at 4 mg/mL lipid in 4% Kolliphor P407 (KP407). Lipids were prepared from lyophilized stocks by mixing with a cold solution of KP407 at 1000 rpm using a shaker for 1 hour at RT. A10×PBS solution was then added and the lipids were mixed at RT for an additional 30 min to make the 4% KP407 stock solution isotonic. Lipids were used for chemical injections or DC treatment within one hour of their preparation.

Subcutaneous (SC) Injection. Mice were placed under Isoflurane for a few min for anesthesia. Tubes containing the chemicals were vortexed for 30 seconds, then loaded with a 1 mL Sub-Q syringe. Mice were injected on the upper right back with 100 μL total volume of chemicals as listed in Table 3.

Blood Collection. Mice were placed under Isoflurane for approximately 10 min for anesthesia. Using a 21G needle, mice were gently poked through the skin to the submandibular space to induce bleeding. Five-Six drops were collected in a mini collect K2EDTA blood collection tube. Blood samples were transported back to Corner Therapeutics lab on ice. The blood was then centrifuged at 1500×g for 5 min. After centrifugation, blood was collected into a 96-well round bottom plate and stored at −80° C.

dLN Dissection and Dissociation. 2 hours and 48 hours post injection; inguinal, axillary, and brachial draining lymph nodes were collected from the side of injection of each mouse and placed in PBS. The dLN were then transported on ice to Corner Therapeutics labs. The dLN was then processed using a Miltenyi spleen dissociation kit according to manufacturer's protocol. In brief, the dLN from each mouse was transferred into the gentleMACS C Tube containing the enzyme mix. The dLN were then dissociated using the gentleMACS Program 37C_m_SDK_1. Cell suspensions were then collected and filtered through a 30 μm Pre-Separation Filter. Cells were counted using the Moxi automated counter, then resuspended in 400 ul of FACS buffer for cell staining.

Spleen Dissection and Dissociation. 2 hours and 48 hours post injection; Spleen were collected from each mouse and placed in PBS. The spleen samples were then transported on ice to Corner Therapeutics labs. Spleen samples were then processed a Miltenyi spleen dissociation kit according to manufacturer's protocol. In brief, each spleen was transferred into the gentleMACS C Tube containing the enzyme mix. The spleens were then dissociated using the gentleMACS program 37C_m_SDK_1. ACK lysis was performed for 2 min. Cell suspensions were then collected and filtered through a 30 μm Pre-Separation Filter. Cells were counted using the Moxi automated counter, then resuspended in 400 ul of FACS buffer for cell staining.

Measurement of Serum Chemokines and Cytokines. Secreted cytokines were measured in the supernatant by cytokine bead array using the LEGENDplex™ Mouse Murine Proinflammatory Chemokine, Murine Cytokine Release Syndrome Legendplex and LEGENDplex™ Mouse Cytokine Panel 2 (Biolegend) according to the manufacturer's protocol. Data were collected using a Quanteon Novocyte flow cytometer and analyzed using the cloud-based software provided by Biolegend.

Flow Cytometry. Single cell suspension from the draining lymph or spleen were resuspended in 100 μL PBS containing Live/Dead Violet (1:1000) and incubated for 20 min at 4° C. Cells were then washed and resuspend in 100 μL FACS buffer containing Fc block (1:100) for 10 min. For myeloid cell staining, cells were then resuspended in 100 μL of antibodies detailed in Table 13-1 to identify monocytes as CD11b⁺ Ly6C⁺F4/80^{neg} MHC^{neg}live cells, moDCs as CD11b⁺ Ly6C⁺F4/80^{neg} MHC⁺ live cells, macrophages as F4/80⁺ MHC⁺CD24^{neg} live cells, and DCs as CD11c⁺MHC-II⁺ F4/80^{neg} live cells. Counter Bright counting beads were added before the acquisition of samples.

Data Analysis. In in vivo studies, n refers to the number of animals per condition. Graphical data was shown with each symbol representing one mouse (n—4-5 mice per group). Some data were excluded because of low viability of samples post processing.

Heat maps for the Chemokine and Cytokine panels were generated using the heat map function of Prism. The raw values of the chemokines and cytokines were normalized based on the highest and lowest value for a given cytokine across all conditions. The normalized values were plotted along a three-color gradient scale. Statistical differences were calculated with more than two groups was tested using two-way ANOVA with Tukey multiple comparison test correction. P values of <0.05 (*), <0.01 (**) or <0.001 (***); <0.0001 (****) indicated significant differences between groups. All experiments were analyzed using Prism 7 (GraphPad Software).

TABLE 13-1
Antibodies Used\hat{ }

| Antibody | Clone | Fluorophore | Vendor | Catalog # |
|---|---|---|---|---|
| Live/Dead Violet | N/A | BV421 | Thermofisher | L34955 |
| Fc Block | 2.4G2 | N/A | BD | 553142 |
| Anti-CD11c | HL3 | PE-Cy7 | BD | 558079 |
| Anti-SIRPa | P84 | FITC | BD | 560316 |
| Anti-CD24 | M1/69 | BV711 | BD | 563450 |
| Anti-CD45R | RA3-6B2 | APC | BD | 557957 |
| Anti-Mouse I-A/I-E | 2G9 | BUV395 | BD | 743876 |
| Anti-mouse CD103 | RUO-2E7 | BB700 | BD | 748240 |
| Anti-Mouse CD69 | H1.2F3 | BV786 | BD | 564683 |
| Rat Anti-Mouse CD45 | 30-F11 | BUV563 | BD | 612924 |
| anti-mouse CD69 | H1.2F3 | PE | Biolegend | 104508 |
| anti-mouse Ly-6C | Hk1.4 | APC | Biolegend | 128016 |
| anti-mouse CD14 | Sa14-2 | FITC | Biolegend | 123308 |
| F4/80 Monoclonal Antibody | BM8 | PE | Life Technologies | 12480182 |
| anti-mouse CD69 | H1.2F3 | BV605 | Biolegend | 104530 |
| anti-mouse/human CD11b | M1/70 | APC | Biolegend | 101212 |

\hat{ }Abbreviations: APC = Allophycocyanin, AF = Alexa Fluor, Cy = Cyanine, BUV = BD Horizon Brilliant Ultraviolet, BV = Brilliant Violet, BB = BD Horizon Brilliant Blue, FITC = Fluorescein isothiocyanate, PB = Pacific Blue, PE = Phycoerythrin, PerCP = Peridinin-Chlorophyll-Protein, PE-Cy = Phycoerythrin-Cyanine, N/A = not applicable.

### Results

R848+DGP Induces An Acute Systemic Inflammation That Wanes By 24 Hours Post Subcutaneous Injection. Cytokines and chemokine released from innate immune cells play a key role in the regulation of inflammation and immune responses. Mice were injected SC with either PBS, R848, DGP, or a combination of R848 and DGP to assess the effects of these treatments on systemic inflammatory responses. Serum samples from the blood of injected mice were collected 2 hours, 24 hours and 48 hours post injection. Cytokines and chemokines were then measured by flow cytometry using bead array kits that can detect multiple NFκB dependent cytokines and chemokines as well as type-I and type-III interferon cytokines.

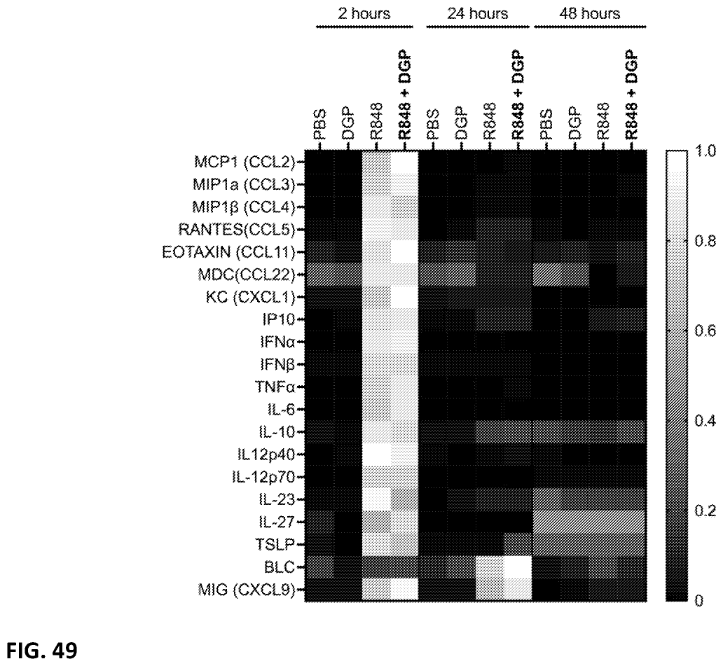

PBS injection did not induce any cytokine release at 2 hours, 24 hours or 48 hours post injection (FIG. 49). Similarly, DGP injection alone was unable to induce any cytokine or chemokine release in the serum at any timepoint. In contrast, as early as 2 hours post injection, R848 alone induced the secretion of a wide range of NF-kB dependent chemokines involved in innate immune recruitment such as CCL2, CCL3, CCL4, as well as NF-kB dependent pro-inflammatory and anti-inflammatory cytokines including IL-6, TNFα, IL-12p40, IL-12p70, IL-10 (FIG. 49). Furthermore, interferon and interferon stimulated cytokines such as IFNα, IFNβ, and IP10, were upregulated by R848 injection (FIG. 49). The levels of these cytokines and chemokines returned to homeostasis (PBS levels) by 24 hours post injection as these cytokines and chemokines were not detected in the serum at 24 hours or 48 hours post injection. Of note, BLC cytokine was not detected 2 hours post injection, but was induced 24 hours post injection and returned to PBS levels 48 hours post injection. CXCL9 was another chemokine detected 24 hours post injection. This chemokine was induced by 2 hours post injection and persisted in the serum 24 hours post injection, but then returned to PBS levels by 48 hours post injection. These data indicated that R848 alone can induce systemic inflammation upon SC. injection. However, this inflammation is transient since the levels of most cytokines and chemokine wane by 24 hours post injection. These data are in accordance with previous observations (Lu et al., Journal of Controlled Release, 306:165-176, 2019).

Notably, R848+DGP behaved similarly to R848 injection alone. As such, R848+DGP induced similar levels of cytokines and chemokines secretion in the blood compared to R848 injection alone (FIG. 49). The levels of these analytes returned to baseline by 24 hours post injection for most detected cytokines except BLC which persisted 24 hours later but waned by 48 hours post injection. Akin to R848 injection, R848+DGP induced CXCL9 secretion 24 hours post injection but waned by 48 hours post injection (FIG. 49).

Overall, these data indicate that: 1) DGP does not unleash an inflammatory response when it is injected on its own; R848 induces an acute and transient inflammation as soon as 2 hours post injection, but the response wanes by 24 hours post injection; and 3) R848+DGP injection (combination of R848 and DGP) induces a transient inflammation that is dependent on R848 and not DGP.

R848+DGP Induce Myeloid Cell Infiltration In The dLN At 4 Hours And 48 Hours Post Subcutaneous Injection. The secretion of immunoregulatory cytokines and chemokines regulate infiltration of peripheral blood leukocytes to sites of inflammation, leukocyte activation, and function (Alvarez et al., Immunity, 29:325-342, 2008). Since R848+DGP induced an acute systemic cytokine and chemokine release, this provided the mandate to further explore the effects of R848+DGP on the local regulation of innate immune cell infiltration in the lymph nodes draining the site of subcutaneous injection. Skin dLN were enzymatically and mechanically dissociated, then single cell suspensions were stained to identify myeloid cell subsets such as monocytes, moDCs, macrophages and conventional DCs. The absolute number of these subsets were measured 4 hours and 48 hours post injection by flow cytometry. To visualize the modulation of the abundance of these subsets, the absolute number of monocytes, moDCs, macrophages and DCs were each normalized to the number of cells detected 4 hours post injection using a heatmap. Three patterns were observed in the dLN: an early cellular infiltration of cells 4 hours post injection that was dependent on R848 activity, an early cellular infiltration of cells 4 hours post injection that was induced by the combination of R848 and DGP, and a later cellular infiltration 48 hours post injection that required the combination of R848 and DGP as detailed below.

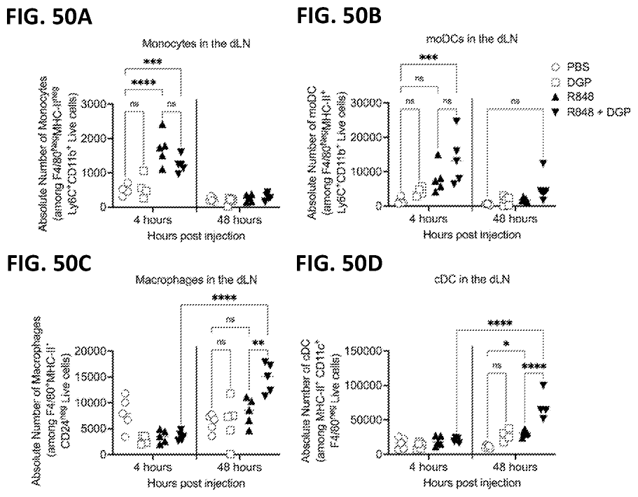

Early Local Cellular Responses Are Dependent On R848 Activity. DGP injection did not induce an increase in the absolute number of cells in the dLN at 4 hours compared to PBS injection. In contrast, the injection of R848 alone or in combination with DGP significantly increased the absolute number of monocytes in the dLN at the 4-hour timepoint compared to PBS injection (FIG. 50A; p<0.0001 for R848, and p=0.0006 for R848+DGP, 2-way ANOVA). No difference was observed between the injection of R848 alone or R848+DGP, indicating that the infiltration of monocytes early after injection is dependent on R848 activity. The absolute number of monocytes returned to baseline levels (PBS levels) by 48 hours post injection, highlighting the transient effect of R848 on monocytes infiltration to the dLN.

Early Local Cellular Responses Induced By The Combination Of R848 And DGP. When monitoring the absolute number of moDCs in the dLN, similar trends were observed as described above (FIG. 50B). DGP injection did not induce an increase in the absolute number of moDCs in the dLN compared to PBS injection (FIG. 50). The injection of R848 alone induced a slight increase in moDC (not statistically significant) compared to PBS injection (FIG. 50C, p=0.1253, 2-way ANOVA). In contrast, the injection of R848 in combination with DGP significantly increased the absolute number of moDCs in the dLN compared to PBS injection at the 4 hours timepoint (FIG. 50B, p=0.0001, 2-way ANOVA). These data indicated that the infiltration of moDCs may be initially controlled by R848 activity, but it is enhanced by the injection of R848 in combination with DGP. Notably, the absolute number of moDCs returned to basal levels by 48 hours post injection, highlighting the transient effect of R848+DGP on infiltration of moDCs to the dLN.

Late Local Cellular Responses Were Induced By The Combination Of R848 And DGP. In contrast to monocytes and moDCs, macrophages and cDCs were not increased at 4 hours post injection regardless of treatment (FIG. 50C and FIG. 50D). 48 hours post injection, macrophages and cDC abundance did not increase upon injection of DGP or R848 alone (FIG. 50C and FIG. 50D). Instead, their abundance was significantly increased when R848 was injected in combination with DGP compared to R848 alone (FIG. 50C, macrophages, p=0.0062, and 2E: cDCs, p=0.0279, 2-way ANOVA), or compared to 4 hours post injection (FIG. 50C and FIG. 50D, p<0.0001, 2-way ANOVA). Thus, the late increase in infiltration of macrophages and cDCs required both R848 and DGP.

Overall, these data indicate that the SC injection of R848+DGP induce local inflammation in vivo by increasing abundance of monocytes and moDC in the dLN at 4 hours post injection, and by increasing abundance of macrophages and cDCs in the dLN at 48 hours post injection.

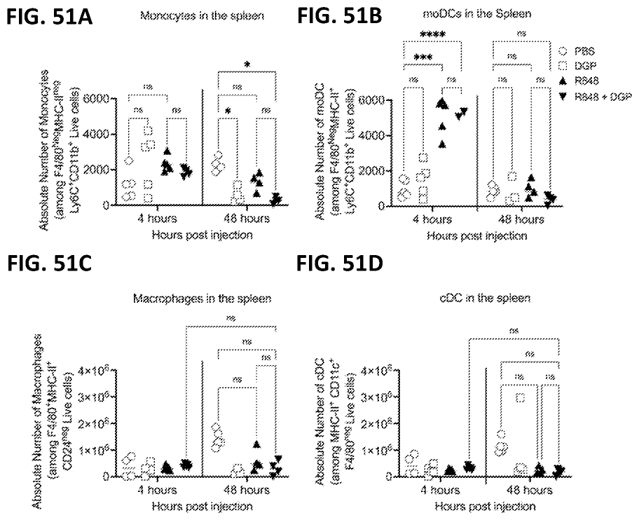

To assess if R848+DGP modulates the abundance of myeloid cells systemically, the absolute number of myeloid cell subsets in the spleen were measured 4 hours and 48 hours post injection by flow cytometry. Similarly, to the dLN analysis, the modulation of the abundance of these subsets were each normalized to the number of cells detected 4 hours post injection using a heatmap. At 4 hours post injection, no significant change in monocytes was observed (FIG. 51A). At 48 hours post injection, while R848 injection did not induced a significant change compared to PBS injected mice, DGP injection alone or in combination with R848 decreased the levels of monocytes in the spleen (FIG. 51A, p=0.0407 for DGP, p=0.0299 for R848+DGP, 2-way ANOVA).

In contrast to monocytes, when mice were injected with R848 alone or in combination with DGP, moDCs abundance increased 4 hours post injection compared to PBS injection (FIG. 51B, p=0.0002 for R848, p<0.0001 for R848+DGP, 2-way ANOVA). No difference was observed between R848 injection alone or in combination with DGP indicating that this increase in moDCs abundance was mediated by R848 activity (FIG. 51B). At the 48 hour timepoint, the absolute number of moDCs returned to basal levels, highlighting the transient effect of R848+DGP on infiltration of moDCs to the spleen.

At 4 hours or 48 hours, no significant changes in macrophages (FIG. 51C) or DCs (FIG. 51D) were observed between PBS and any of the other treatments evaluated.

In summary, R848 enhanced the abundance of moDCs early after injection (4 hours post injection), but not other myeloid cell subsets. At 48 hours post injection, no significant changes were observed between injected mice, except for monocytes which seemed to be reduced when mice were injected with DGP or R848 and DGP.

R848+DGP Induces CD69 Upregulation On Myeloid Cells In The dLN And Spleen Post Subcutaneous Injection. To further monitor local cellular inflammatory responses, CD69 expression was measured on the surface of myeloid cells in the dLN as a hallmark of cell activation by calculating the mean fluorescence intensity (MFI) of CD69 4 hours and 48 hours post injection. To generate a comprehensive heatmap of CD69 expression, the data were normalized to PBS injected mice 4 hours post injection.

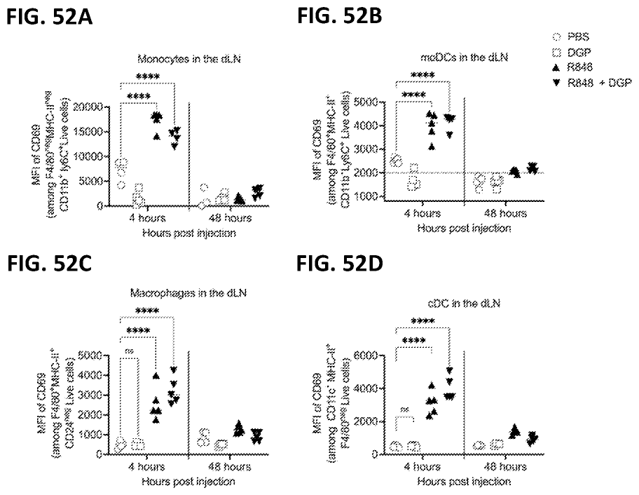

DGP injection alone did not induce CD69 upregulation on myeloid cells in the dLN 4 hours or 48 hours post injection compared to PBS injection (FIG. 52A-D). In contrast, when mice were injected with R848 alone or R848+DGP, CD69 expression was strongly upregulated 4 hours post injection on the surface of myeloid cells in the dLN including monocytes, moDCs, macrophages and cDCs (FIG. 52A-D, p<0.0001 for R848 and R848+DGP, 2-way ANOVA) compared to mice injected with PBS. CD69 expression returned to baseline levels 48 hours post injection, indicating that myeloid cell activation occurs early after injection and is transient (FIG. 52A-D).

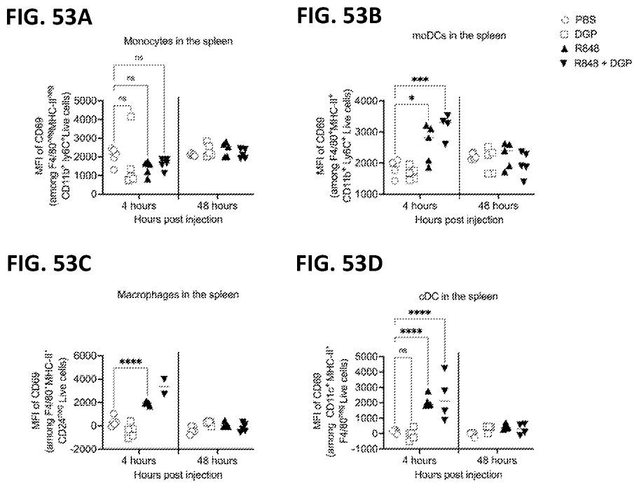

Similar trends were observed in the spleen of injected mice when CD69 MFI was measured 4 hours and 48 hours post injection. While DGP did not induce the upregulation of CD69 on myeloid cells in the spleen, the injection of R848 alone or R848+DGP, strongly upregulated the expression of CD69 on the surface moDCs (FIG. 53B, p=0.0229 for R848, p=0.0001 R848+DGP, 2-way ANOVA), macrophages (FIG. 53C, p<0.0001 for R848, 2-way ANOVA) and cDCs (FIG. 53D, p<0.0001 for R848 and R848+DGP, 2-way ANOVA) compared to mice injected with PBS. Furthermore, CD69 expression returned to baseline levels 48 hours post injection. As for monocytes in the spleen, no significant changes in CD69 expression were observed at 4 hours or 48 hours post injection (FIG. 53A). Notably, no difference was observed between the injection of R848 alone or R848+DGP, suggesting that CD69 upregulation is mediated by R848 while DGP does not contribute to any additional effect (FIG. 53A-D).

Overall, these data indicate that R848+DGP induces myeloid cell activation 4 hours post injection in the dLN and spleen as assessed by CD69 upregulation. Myeloid cell activation is dependent on R848 activity and is indicative of systemic and local transient inflammation.

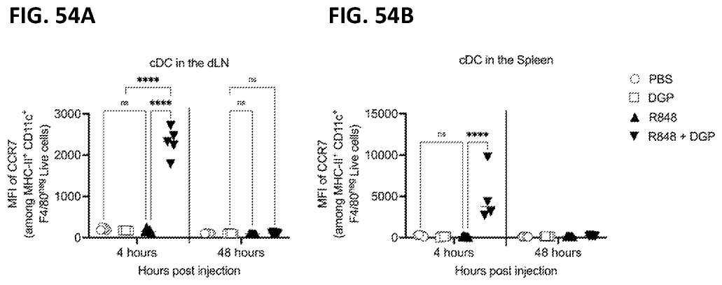

R848+DGP Induces CCR7 Expression on DCs in the dLN and Spleen 4 Hours Post Subcutaneous Injection. Hyperactive DCs are endowed with their ability to hypermigrate from the skin to the dLN via the C-C chemokine receptor type 7 (CCR7) (Zhivaki et al., Cell Rep, 33(7):108381, 2020). To assess if R848+DGP injection modulates CCR7 expression on endogenous DCs, the mean fluorescence intensity (MFI) of CCR7 was measured on the surface of DCs from the dLN and Spleen. While DGP or R848 injection did not induce the upregulation of CCR7 on DCs in the dLN or in the spleen (FIG. 54A-B), the injection of R848+DGP (combination of R848 and DGP) induced a strong upregulation of CCR7 that was observed 4 hours post injection compared to R848 injection alone (FIG. 54A, p<0.0001 and FIG. 54B, p<0.0001 using 2-way ANOVA). The expression of CCR7 returned to its basal levels 48 hours post injection, as no difference was observed between PBS or any other treatment in the dLN and spleen (FIG. 54A-B).

These data indicate that R848+DGP treatment can lead to the upregulation of CCR7 on DCs in the dLN and spleen at an early timepoint. The upregulation of CCR7 is an attribute of R848+DGP and not enhanced by the single agents R848 or DGP alone.

In summary, the SC injection of R848+DGP induced an acute early systemic inflammation, including cytokine and chemokine secretion, myeloid cell activation and myeloid cell infiltration in the spleen that was dependent on R848 activity. Furthermore, the combination of R848 and DGP induced a local inflammation that mobilized innate immune cells recruitment to the dLN, and enhanced expression CCR7 on DCs in the dLN and spleen.

### Example B-14: Immunization of Mice with an Inactivated Influenza Virus Vaccine Combined with R848 and DGP

AFLURIA QUADRIVALENT® (Afluria) is an inactivated, non-adjuvanted, seasonal influenza vaccine (distributed by Seqirus USA Inc., Summit, NJ), which is approved by the USFDA for active immunization against the four A subtype and type B influenza viruses contained in the vaccine. The purpose of this in study was to evaluate the safety and immunogenicity of Afluria when combined with three different doses of R848+DGP and administered via intramuscular (IM) or subcutaneous (SC) injection.

### Materials and Methods

Immunization and Study Design. C57BL/6 mice were used to assess how route of administration influences the immune response to Afluria combined with R848+DGP. Previous examples have shown that 3 injections of antigen combined with R848+DGP each spaced one week apart induced robust antigen-specific T cell responses when assessed on day 21. Stocks of R848 and DGP in KP407 were prepared as described in previous examples. To this end, nine groups of 7 mice were immunized SC or IM on days 0, 7, and 14 (Table 14-1) with Afluria alone or Afluria combined with three different dose levels of R848+DGP:
1) Dose Level 1: 10 ug R848+100 ug DGP;
2) Dose Level 2: 50 ug R848+100 ug DGP; and
3) Dose Level 3: 50 ug R848+200 ug DGP.

Body weights for each mouse were measured on Day 0 (prior to immunization) and twice a week thereafter. On day 21, spleens were harvested, weighed, and processed to single cell suspension for detection of antigen-specific T cells by ELISPOT, ELISA and for flow cytometry. On day 21, serum was collected for detection of antigen-specific antibody titers.

TABLE 14-1
Study Groups

| Group | PAMP | DAMP + Surfactant | Antigen | Route |
|---|---|---|---|---|
| 1 | — | — | — | SC |
| 2 | — | — | Afluria 8 ug | SC |
| 3 | R848 10 ug | DGP 100 ug in KP407 | Afluria 8 ug | SC |
| 4 | R848 50 ug | DGP 100 ug in KP407 | Afluria 8 ug | SC |
| 5 | R848 50 ug | DGP 200 ug in KP407 | Afluria 8 ug | SC |
| 6 | — | — | Afluria 8 ug | IM |
| 7 | R848 10 ug | DGP 100 ug in KP407 | Afluria 8 ug | IM |
| 8 | R848 50 ug | DGP 100 ug in KP407 | Afluria 8 ug | IM |
| 9 | R848 50 ug | DGP 200 ug in KP407 | Afluria 8 ug | IM. |

ELISPOT Assay. IFNγ and IL-5 ELISPOT plates (R&D Systems) were blocked with 200 μL R10 media (RPMI-1640 media supplemented with 10% FBS, 100 U/ml Penicillin, 100 μg/mL Streptomycin, 2 mM L-Glutamine, 1 mM Sodium Pyruvate, and 54 μM β-mercaptoethanol) for 45 min. At the end of blocking, media was discarded and 100 μL of either R10 media alone, R10 media containing 1 μg/mL of SARS-CoV-2 Spike protein PepTivator (irrelevant peptide control), or R10 media containing 20 μg/mL of Afluria were added to respective wells. Splenocytes were seeded at 500,000 cells/well in 100 μL of R10 media and plates were incubated at 37° C. for 20 hours. After incubation, cells were discarded and ELISPOTs were developed according to the manufacturer's instructions. Plates were left to dry overnight at RT. The next day, plates were read on the S6 Universal M2 ELISPOT plate analyzer and spots were quantified using the Smart Count™ function.

ELISA. Splenocytes were seeded at 500,000 cells per well in 100 μL of R10 media in 96-well plates. 100 μL of either R10 media alone, R10 media containing 1 μg/mL of SARS-CoV-2 spike protein PepTivator (irrelevant peptide control), R10 media containing 20 μg/mL of Afluria or R10 media containing 2× Cell Stimulation Cocktail (PMA and ionomycin, positive control) were added to respective wells of 96-well round bottom tissue-culture treated plates, and plates were incubated at 37° C. for 72 hours. Following incubation, plates were spun at 400×g for 5 min and cell culture supernatants were transferred to 96-well non-tissue culture treated plates and stored at −80 until ready for use. Cytokine secretion was measured by IL-5 or IFNγ ELISA kits from Invitrogen according to manufacturer's protocol. Supernatants were thawed at RT and diluted 1:20 (IFNγ) or 1:5 (IL-5) in assay buffer and incubated at 4 C overnight on a rocking platform. The following morning, plates were developed according to manufacturer's protocol.

Hemagglutination Inhibition (HAI) Assay. All steps in the HA and HAI assay, including washing of the RBCs, were performed in accordance with the World Health Organization guidelines (World Health Organization, 2011).

Washing of guinea pig RBCs. 5 ml of Guinea Pig RBCs in Alsevers solution were transferred to a 50 mL conical and centrifuged at 300×g for 10 min at RT. Supernatant was discarded and 45 mL of PBS were added and the tube was lightly inverted 3 times to mix. RBCs were centrifuged at 300×g for 10 min at RT. This step was repeated one more time. After centrifugation, the RBC pellet was resuspended in 12 mL PBS and transferred to a 15 mL conical and centrifuged one more time at 300×g for 10 min. Supernatant was discarded and 9 ml of PBS were added to obtain a 10% guinea pig RBC solution which was kept on ice and further diluted in PBS to 0.75% prior to use.

HA assay to determine 4 HA units. 100 μl of undiluted Afluria was added to the first well of a non-sterile 96 well round bottom plate. Two-fold serial dilutions were performed by transferring 50 μl Afluria mixture to 50 μl of PBS for a total of 23 dilutions. 50 μl of 0.75% Guinea Pig RBCs were added to all wells and samples were mixed by pipetting. Plates were incubated for 1 hour at RT. Results were obtained by taking a picture of the plate by using an iPhone 11 that was mounted to a VIVI MAO gooseneck cell phone holder.

HAI assay setup. Prior to assay, 24 ul mouse sera was treated with 72 μl receptor destroying enzyme (RDE) in sterile 96-well round bottom tissue culture-treated plates. Plates were sealed with plate sealer and samples were incubated for 20 hours at 37° C. Samples were then incubated at 56° C. for 1 hour to inactivate RDE and 144 ul of 0.9% sterile NaCl were added to each sample.

To test sera for non-specific hemagglutination prior to HAI assay, 50 μl of RDE-treated serum was added to 50 μl of PBS in 96-well round bottom plates. Two-fold serial dilutions were performed by transferring 50 μl of serum mixture into 50 μl PBS for a total of 2 additional dilutions. 50 μl of 0.75% Guinea Pig RBCs were added to wells and samples were mixed by pipetting. Plates were incubated for 1 hour at RT. Results was obtained by taking pictures of each plate with an iPhone 11 mounted to a VIVI MAO gooseneck cell phone holder with each plate being 32 cm away from the phone.

To ensure that 4HA units of Afluria are used for HAI assay, a back-titration control was set up as follows: 100 μL of 28.8 ng/ml Afluria (diluted in PBS) was added to the first well in a 96 well round bottom plate. Two-fold serial dilutions in PBS were performed by transferring 50 μl of Afluria mixture into 50 μl of PBS for a total of 11 additional dilutions. 50 μL of 0.75% Guinea Pig RBCs were added to wells and plates were incubated for 1 hour at RT. Results was obtained by taking pictures of each plate with an iPhone 11 mounted to a VIVI MAO gooseneck cell phone holder with each plate being 32 cm away from the phone.

HAI assay. 50 μL of RDE-treated serum was added to 96 well round bottom plates in duplicate. Two-fold serial dilutions were performed by transferring 25 μl of serum into 25 μl of PBS for a total of 11 additional dilutions. 25 ul of Afluria (28.8 ng/mL in PBS) was then added to all wells and samples were mixed by pipetting. Plates were incubated for 30 min at RT. Following incubation, 50 μL of 0.75% guinea pig RBCs were added to all wells and mixed. The plates were then incubated for 1 hour at RT. Results was obtained by taking pictures of each plate with an iPhone 11 mounted to a VIVI MAO gooseneck cell phone holder with each plate being 32 cm away from the phone.

Afluria-specific Antibody ELISA. Nunc MaxiSorp ELISA plates were coated with 100 μL PBS containing 0.5 μg/mL Afluria and incubated overnight at 4° C. The following morning, plates were washed with wash buffer (PBS containing 0.05% Tween 20) and blocked with 300 μL PBS containing 2% BSA for 1 hour at RT on a rocking platform. Mouse serum was thawed at RT and diluted 1 to 800 in PBS containing 1% BSA then serially diluted 1:2 six more times. After blocking, plates were washed, 100 μl of diluted serum was added, and plates were incubated overnight at 4° C. on a rocking platform. The following day, plates were washed and 100 μL of Immunoglobulin G (IgG)-Horseradish Peroxidase (HRP), IgG1-HRP, or IgG2b-HRP (all diluted 1:5,000 in PBS containing 1% BSA) was added and incubated for two hours at RT with no rocking. Plates were washed and 100 μL of 3,3′,5,5′-Tetramethylbenzidine (TMB) was added to all wells. Plates were incubated for one minute at RT while hidden from light and the reaction was stopped by the addition of 50 μL of 2N sulfuric acid. Plates were read on a SpectraMax M5 spectrophotometer at 450 and 570 nm wavelengths. OD values were determined by subtracting 570 nm absorbance from 450 nm absorbance value for each data point.

IgG Affinity Assay. Nunc MaxiSorp ELISA plates were coated with 100 μL PBS containing 0.5 μg/mL Afluria and incubated overnight at 4° C. The following morning, plates were washed with wash buffer (PBS containing 0.05% Tween 20) and blocked with 300 μL PBS containing 2% BSA for 1 hour at RT on a rocking platform. Mouse serum was thawed at RT and diluted 1 to 800 in PBS containing 1% BSA then serially diluted 1:2 six more times. After blocking, plates were washed, 100 μl of diluted serum was added, and plates were incubated overnight at 4° C. on a rocking platform. The following day, plates were washed and 100 μL of 5.3M Urea or PBS containing 1% BSA was added to wells and incubated for 10 min at RT. Plates were washed and 100 μl of goat anti-mouse IgG-HRP (1:5000 in PBS containing 1% BSA) was added to wells. Plates were incubated for two hours at RT with no rocking. Plates were washed and 100 μL TMB was added to all wells. Plates were incubated for one minute at RT while hidden from light and the reaction was stopped by the addition of 50 μL of 2N sulfuric acid. Plates were read on a SpectraMax M5 spectrophotometer at 450 and 570 nm wavelengths. OD values were determined by subtracting 570 nm absorbance from 450 nm absorbance value for each data point.

Flow Cytometry. One million splenocytes from each mouse were plated into a 96-well Clear Round Bottom plate and centrifuged at 400×g for 4 min. Supernatant was discarded and cells were resuspended in 100 μl PBS containing Live/Dead Aqua at a 1:1000 dilution and incubated for 20 min at 4° C. Following incubation, cells were washed with 100 uL of FACS buffer (0.5% Bovine serum albumin in PBS), centrifuged at 400×g for 4 min and supernatant was discarded. Cells were resuspended in 100 μl FACS buffer containing mouse Fc Block at a 1:100 dilution and incubated for 10 min at 4° C. Following incubation, cells were washed with 100 μL of FACS buffer, centrifuged at 400×g for 4 min and supernatant was discarded.

T cell/NK cell panel. Cells were resuspended in 100 μl FACS buffer containing anti-mouse CXCR5 biotin at a 1:50 dilution and incubated for 30 min at 4° C. followed by incubation at 37° C. for 30 min. Following incubation, cells were washed with 100 uL of FACS buffer, centrifuged at 400×g for 4 min and supernatant was discarded. Cells were resuspended in 100 uL surface antibody cocktail prepared with a 1:1 ratio of FACS buffer and Brilliant Stain Buffer (BD Biosciences) containing antibodies for the following markers: CD3, CD4, CD8, ICOS, CD44, CD62L, CD19, NK1.1. The cells were incubated for 30 min at 4° C. Following the incubation, the cells were washed with 100 uL of FACS buffer, centrifuged at 400×g for 4 min and supernatant was discarded. Intracellular staining was performed with the Foxp3 Transcription Staining Buffer Set according to manufacturer's protocol. Following fixation, cells were resuspended in 1:100 dilution of anti-Foxp3 (MF-14) in permeabilization buffer (1×) and incubated overnight at 4° C. Following overnight incubation, the cells were washed with 100 uL of FACS buffer, centrifuged at 400×g for 4 min and supernatant was discarded. Cells were resuspended in FACS buffer containing Liquid Counting Beads at a 1:5 dilution. Samples were acquired on a BD FACSymphony A3.

B cell/DC panel. Cells were resuspended in 100 uL surface antibody cocktail prepared with a 1:1 ratio of FACS buffer and Brilliant Stain Buffer (BD Biosciences) containing antibodies for the following markers: CD3, CD19, GL7, Fas, CD138, CD11c, IA/IE, IgG1, IgM, B220, CD38, CD73, IgG2a. The cells were incubated for 30 min at 4° C. Following the incubation, the cells were washed with 100 μL of FACS buffer, centrifuged at 400×g for 4 min and supernatant was discarded. Cells were resuspended in 100 μL of BD Cytofix/Cytoperm (554722) and incubated for 20 min at 4° C. Following incubation, the cells were washed with BD Perm/Wash (554723) prepared at 1:10 dilution with UltraPure Distilled Water (10977-015), centrifuged at 400×g for 4 min, and supernatant was discarded. Cell pellets were resuspended in 100 μL of BD Perm/Wash buffer containing antibodies for IgG1 and IgM, and incubated overnight at 4° C. Following overnight incubation, the cells were washed with 100 μL of FACS buffer, centrifuged at 400×g for 4 min, and supernatant was discarded. The cells were resuspended in FACS buffer containing Liquid Counting Beads (335925) at a 1:5 dilution

Statistical Analysis. Data were analyzed and graphed using Microsoft Excel and GraphPad Prism software. Data are reported in bar graphs as means with SD, geometric means with SD (Hemagglutination inhibition), or in tables as means and associated p-values. On bar graphs, each data point represents an individual mouse. Statistical significance of all dose levels within each injection route was determined using One-way ANOVA with multiple comparisons to Afluria alone. For comparison of each dose level when injected SC versus IM, Student's t-test was used to determine statistical significance.

The HAI titer was determined by taking the reciprocal value of the last serum dilution that shows inhibition of RBC hemagglutination by Afluria (World Health Organization, 2011).

For calculation of endpoint titer, a positive cutoff optical density (OD) value was determined by measuring the mean OD and SD of undiluted serum from a naïve C57BL/6 mouse and setting the cut-off at two SDs above this mean (Fox et al., iScience, 23(11), 101735, 2020). Endpoint titer was determined using Prism. OD values for serially diluted serum were plotted in an XY table in Prism and this positive cut-off value was entered in the first empty row of each sample. Data was transformed as X=log X and this was analyzed using non-linear regression (4PL), X is log(concentration). Unknowns were interpolated from standard curve. Nonlinear fit data (Interpolated X mean values) were transformed as X=10^{X} to obtain plotted endpoint titer values.

For calculation of affinity index, serially diluted serum was incubated with Afluria coated plates and either treated or not with Urea. Area under the curve was calculated for each sample, and affinity index is reported as AUC with Urea/AUC without Urea*100.

### Results

Safety Measurements Demonstrate That Afluria Combined With R848+DGP was Well-Tolerated in Mice at Three Different Dose Levels. To determine how Afluria alone or combined with R848+DGP at different dose levels affected mouse growth, body weights of each mouse were recorded at the time of immunization (Day 0) and twice a week thereafter. On day 21, final body weights were recorded prior to sacrifice. The average body weight of each immunization group did not drop by more than 2% at any time point measured and was not significantly different than PBS at any time point measured (Two-Way ANOVA). This indicated that Afluria alone or combined with R848+DGP did not affect expected weight gain when administered SC or IM.

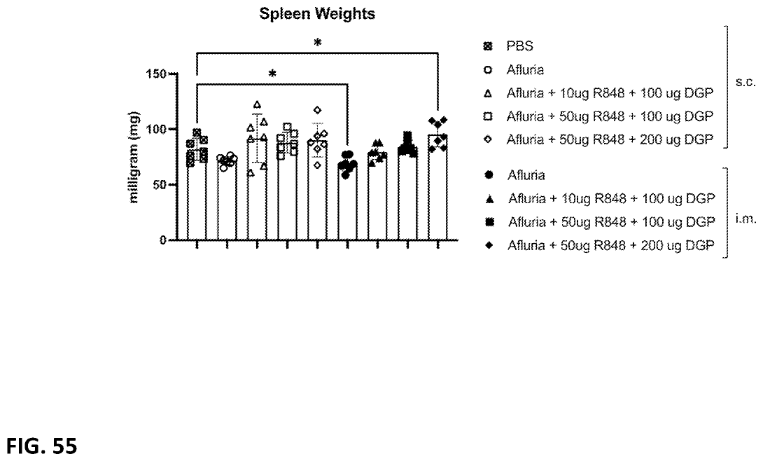

Spleen weights were also recorded at the time of harvest in order to assess whether Afluria alone or combined with R848+DGP induced splenomegaly. When injected SC, Afluria alone or combined with R848+DGP at each dose level evaluated did not induce a significant change in spleen weight compared to PBS (FIG. 55). When injected IM, Afluria alone induced slightly lower spleen weights than PBS (p=0.02, One-Way ANOVA) while Afluria+R848+DGP at Dose Level 3 induced slightly higher spleen weights compared to PBS, suggesting an elevated immune response to this dose level (p=0.02, One-Way ANOVA) (FIG. 55). Taken together, these data demonstrate that combining Afluria with R848+DGP did not induce weight loss, and increased spleen weights only at dose level 3 when given IM but not for any other condition.

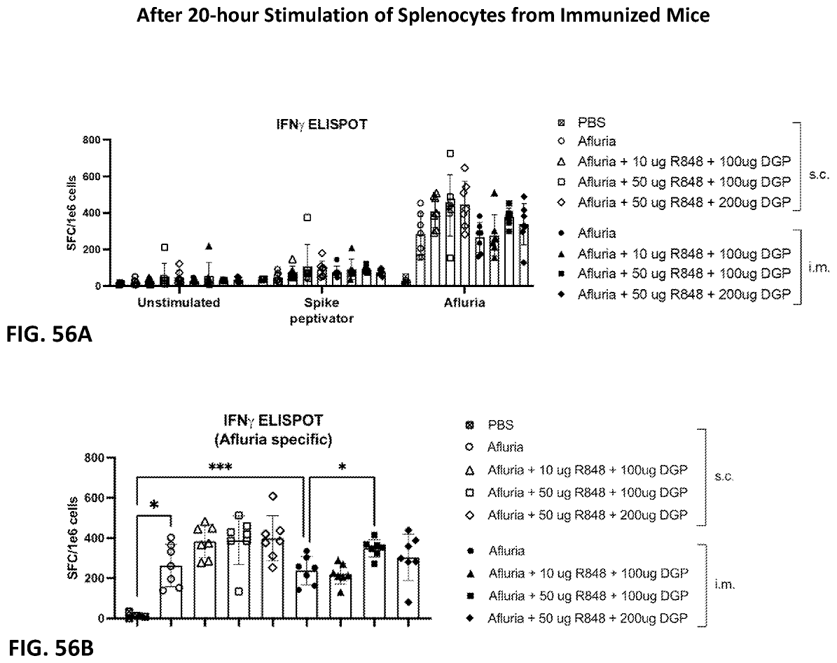

Afluria Combined with R848 and DGP Induced a Stronger Antigen-specific IFNγT cell Response Compared to Afluria Alone when Administered By SC Injection. To assess how route of administration influences the generation of antigen-specific T cells, Afluria alone or Afluria combined with three different dose levels of R848+DGP was administered to mice either SC or IM on days 0, 7, 14. Spleens were harvested from mice on day 21. Total splenocytes were plated for IFNγ ELISPOT assay and cultured for 20 hours with media alone (unstimulated), SARS-CoV-2 Spike protein PepTivator (negative peptide control), or Afluria vaccine. Background levels of IFNγ+ spot forming cells (SFCs) were low as demonstrated by a mean of 57 SFCs/1×10⁶ cells when splenocytes were left unstimulated or stimulated with irrelevant peptide control compared to a mean of 333 SFCs/1×10⁶ cells when stimulated with Afluria (FIG. 56A). In order to correct for any background IFNγ secretion, the number of SFCs produced in unstimulated conditions was subtracted from the number of SFCs produced when cells were stimulated with Afluria, yielding Afluria-specific IFNγ+ SFCs. When injected SC, Afluria alone induced significantly more IFNγ+ SFCs than PBS (p=0.002, One-Way ANOVA) (FIG. 56B). This was not elevated by the addition of R848+DGP at any of the dose levels evaluated when injected SC, however Afluria combined with R848+DGP at Dose Level 2 and Dose Level 3 trended towards more IFNγ compared to Afluria alone (p=0.08, p=0.05) (FIG. 56B). When injected IM, Afluria alone also induced significantly more IFNγ+ SFCs than PBS (p=0.0001, One-Way ANOVA) (FIG. 56B), and this was further increased by the addition of R848+DGP but only at Dose Level 3 (p=0.03, One-Way ANOVA) (FIG. 56B).

When comparing the same dose levels between injection routes. Afluria combined with R848+DGP at Dose Level 1 induced significantly more IFNγ+SFCs when injected SC compared to IM (p=0.0007, Student's t-test) while all other immunization conditions induced a comparable response regardless of injection route (Table 14-2).

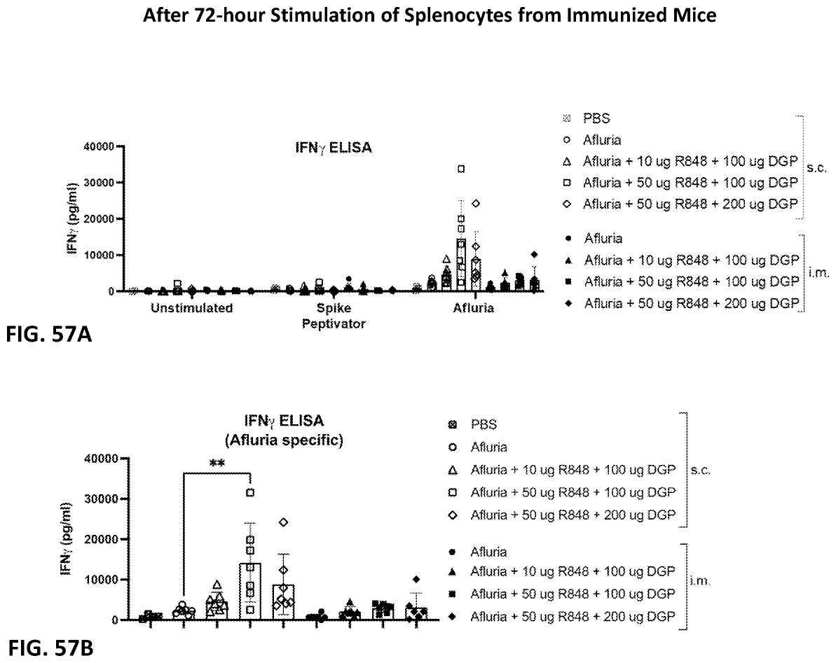

As an additional readout, and to assess the capacity for prolonged IFNγ secretion from antigen-specific T cells, splenocytes were also plated with the stimuli listed above and cultured for 72 hours. IFNγ in cell culture supernatants was quantified by ELISA. Similar to ELISPOT results, background levels of IFNγ secretion were low as demonstrated by an average of 385 μg/mL when cells were left unstimulated or stimulated with irrelevant peptide compared to an average of 4827 μg/mL when stimulated with Afluria (FIG. 57A). In order to correct for any background IFNγ secretion, IFNγ concentrations in unstimulated conditions were subtracted from IFNγ concentrations when cells were stimulated with Afluria, yielding Afluria-specific IFNγ+ secretion. When injected SC, Afluria alone did not lead to increased IFNγ secretion compared to PBS, however the addition of R848+DGP at dose Level 2 induced significantly higher IFNγ secretion compared to Afluria alone (p=0.003, One-Way ANOVA) (FIG. 57B). When injected IM, Afluria alone or combined with R848+DGP at each of the dose levels evaluated did not induce significantly more IFNγ over 72 hours compared to PBS (FIG. 57B).

When comparing the same dose levels between injection routes, Afluria alone as well as Afluria combined with Dose Level 1 and Dose Level 2 induced significantly more IFNγ secretion when injected SC compared to IM (p=0.002, p=0.03, p=0.01, respectively, Student's t-test) (Table 14-2).

Taken together these data demonstrate that when injected SC or IM, Afluria alone is able to induce antigen-specific IFNγ+ cells, and this is further elevated by the addition R848+DGP Dose Level 3 when injected IM (FIG. 56B). Interestingly, the ELISA data show that this IFNγ secretion induced by IM injection of Afluria alone or combined with R848+DGP IM is transient, as IFNγ concentrations in cell culture supernatants are not higher than those induced by PBS after 72 hours (FIG. 57B). Importantly, while Afluria+R848+DGP Dose Level 2 administered SC induced similar frequencies of antigen-specific cells compared to Afluria alone (FIG. 56B), these T cells produced significantly more IFNγ (FIG. 57B), demonstrating that the T cell response induced by Afluria+R848+DGP administered SC is more robust than that induced by Afluria alone.

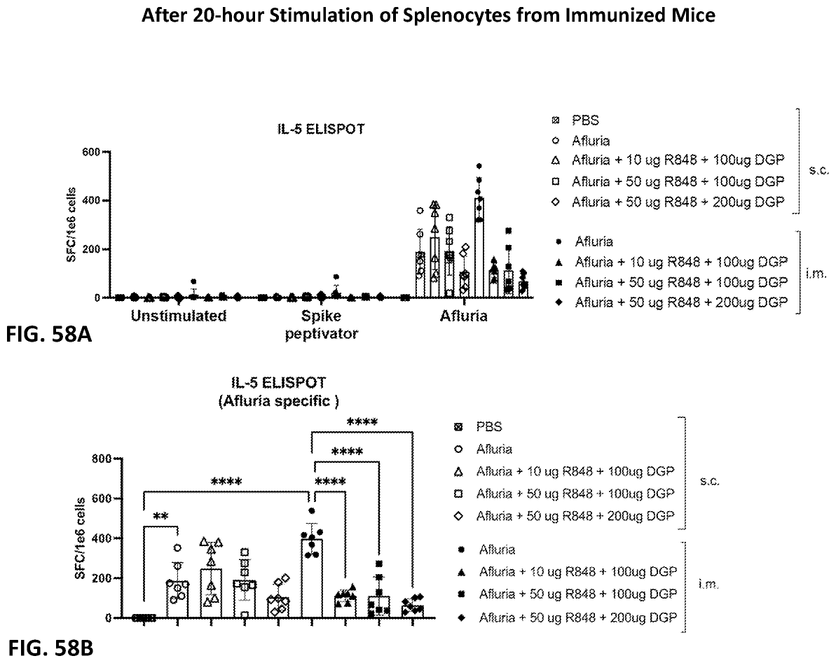

Afluria Combined with R848+DGP Induced Less IL-5 Secretion Compared to Afluria Alone when Administered By IM Injection. CD4⁺ helper T cells can be divided into different subsets based on cytokine secretion and function. T helper type 1 (Th1) cells promote pro-inflammatory type 1 immunity characterized by high levels of IFNγ and are mainly responsible for clearing intracellular pathogens, while T helper type 2 (Th2) cells secrete IL-4, IL-5 and IL-13 and drive the production of antibodies as well as the elimination of extracellular pathogens. In parallel to IFNγ ELISPOT described above, total splenocytes were also plated for IL-5 ELISPOT assay and cultured for 20 hours with the same stimuli. Background levels of IL-5+ SFCs were low as demonstrated by a mean of 4 SFCs/1×10⁶ cells when splenocytes were left unstimulated or stimulated with irrelevant peptide control compared to a mean of 180 SFCs/1×10⁶ cells when stimulated with Afluria (FIG. 58A).

When injected SC, Afluria alone induced significantly more IL-5+ SFC compared to PBS (p=0.007, One-Way ANOVA) and this was not decreased by the addition of R848+DGP at any of the dose levels evaluated (FIG. 58B). When injected IM, Afluria alone also had significantly more IL-5+ SFCs compared to PBS (p<0.0001, One-Way ANOVA), and this was decreased by the addition of R848+DGP at each dose level evaluated (p<0.0001, p<0.0001, p<0.0001, respectively, One-Way ANOVA) (FIG. 58B). When comparing the same dose levels between injection routes, Afluria alone and Afluria combined with R848+DGP at Dose Level 1 induced significantly fewer IL-5+ SFCs when injected IM than when injected SC (p=0.0005, p=0.02, respectively, Student's t-test) (Table 14-2).

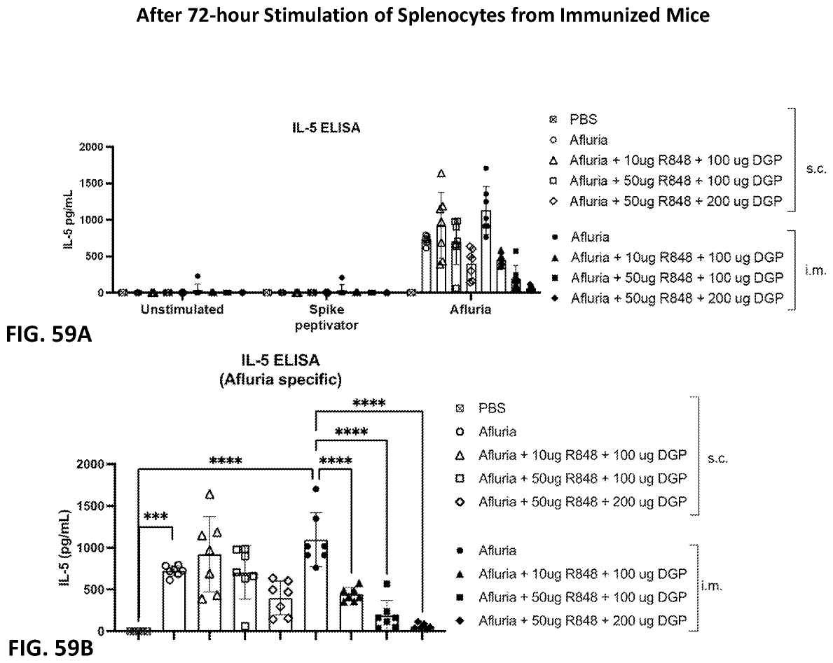

As an additional readout, and to assess the capacity for prolonged IL-5 secretion from antigen-specific T cells, splenocytes were also plated with the same stimuli listed above and cultured for 72 hours. IL-5 in cell culture supernatants was quantified by ELISA. Similar to ELISPOT results, background levels of IL-5 secretion were low as demonstrated by a mean of 5.6 μg/mL when splenocytes were left unstimulated or stimulated with irrelevant peptide control compared to a mean of 582 μg/mL when stimulated with Afluria (FIG. 59A). When injected SC, Afluria alone induced more IL-5 secretion compared to PBS (p=0.0004, One-Way ANOVA) and this was not changed by the addition of R848+DGP at any of the dose levels evaluated (FIG. 59B). When injected IM, Afluria alone also induced significantly more IL-5 secretion than PBS (p<0.0001) and this was decreased by the addition of R848+DGP at all Dose Levels evaluated (p<0.0001, for all dose levels, One-Way ANOVA) (FIG. 59B). When comparing the same dose levels between injection routes, Afluria alone and Afluria combined with R848+DGP at all dose levels evaluated injected IM induced significantly less IL-5 than when injected SC (p=0.01, p=0.02, p=0.003, p=0.002, respectively, Student's t-test) (Table 14-2).

Taken together, these data demonstrate that IM, but not SC, injection of Afluria combined with R848+DGP induced fewer Th2 cells and less IL-5 secretion compared to Afluria alone.

TABLE 14-2
Cytokine Secretion

| Group | Mean IFNγ SFC SC | Mean IFNγ SFC IM | p-value |
|---|---|---|---|
| Afluria | 263.6 | 238.1 | 0.6 |
| Dose Level 1 | 383.4 | 221.3 | 0.0007 |
| Dose Level 2 | 389.1 | 348.2 | 0.4 |
| Dose Level 3 | 398.7 | 303.7 | 0.1 |
| | Mean IFNγ pg/mL SC | Mean IFNγ pg/mL IM | p-value |
| Afluria | 2305 | 793.0 | 0.002 |
| Dose Level 1 | 4598 | 2135 | 0.03 |
| Dose Level 2 | 14190 | 2956 | 0.01 |
| Dose Level 3 | 8808 | 3104 | 0.1 |
| | Mean IL-5 SFC SC | Mean IL-5 SFC IM | p-value |
| Afluria | 187 | 398.6 | 0.0005 |
| Dose Level 1 | 248.7 | 103.6 | 0.02 |
| Dose Level 2 | 191.1 | 110.3 | 0.2 |
| Dose Level 3 | 104.1 | 65.0 | 0.2 |
| | Mean IL-5 pg/mL SC | Mean IL-5 pg/mL IM | p-value |
| Afluria | 722.4 | 1096 | 0.01 |
| Dose Level 1 | 923 | 444 | 0.02 |
| Dose Level 2 | 703.1 | 189.1 | 0.003 |
| Dose Level 3 | 399.5 | 59.6 | 0.002 |

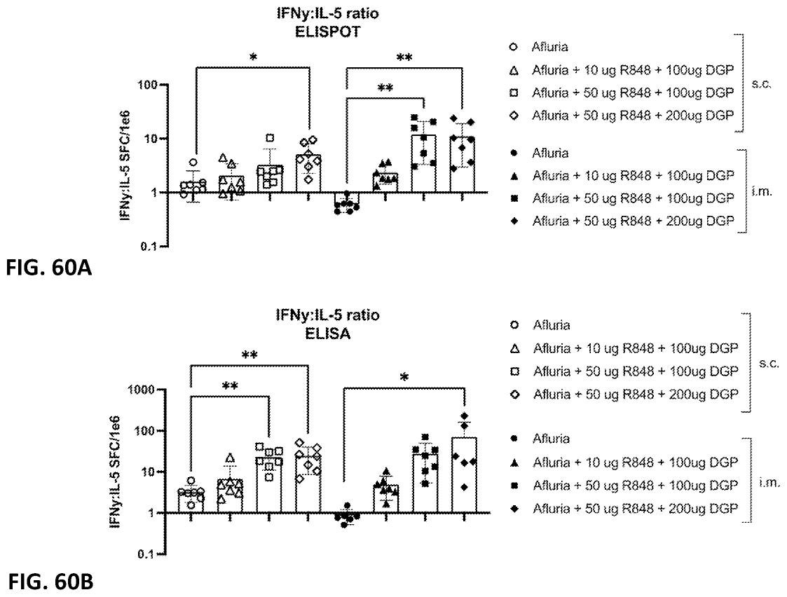

Afluria Combined with R848+DGP Induced a Th1-skewed Immune Response When Administered By SC or IM Injection. Vaccine adjuvants are increasingly used not only to amplify the magnitude of the immune response, but also to try and promote a specific immune response that is appropriate to the pathogenic threat (Howard et al., Viruses, 14(7), 1493, 2022). In the context of viral infection, a type 1 immune response driven by Th1 cells is particularly important for viral clearance and subsequent protection (L'Huillier et al., Scientific Reports, 10(1), 10104, 2020; and Fernandez-Sesma et al., J Virol, 80(13):6295-6304, 2006). To this end, the ability of R848+DGP, combined with Afluria, to induce a Th1-skewed immune response was assessed by quantifying the Th2 cytokine IL-5 and comparing the ratio of IFNγ to IL-5 production for each mouse. When injected SC, Afluria+R848+DGP induced a higher ratio of IFNγ:IL-5 SFCs compared to Afluria alone but only for Dose Level 3 (p=0.02, One-Way ANOVA) (FIG. 60A). When injected IM, Afluria+R848+DGP induced a higher ratio of IFNγ:IL-5 SFCs compared to Afluria alone at dose Level 2 and 3 (p=0.004, p=0.009, respectively, One-Way ANOVA) (FIG. 60A).

Similarly, when evaluating prolonged IFNγ:IL-5 secretion, SC administration of Afluria+R848+DGP induced a higher ratio of prolonged IFNγ:IL-5 secretion compared to Afluria alone for Dose Level 3 (p=0.005, p=0.003, respectively, One-Way ANOVA) (FIG. 60B). When injected IM, Afluria+R848+DGP induced a higher ratio of prolonged IFNγ:IL-5 secretion compared to Afluria alone at Dose Level 3 (p=0.04) (FIG. 60B). Collectively, these data demonstrate that Afluria+R848+DGP at Dose level 2 and 3 induced a Th1-skewed response compared to Afluria alone when administered by either the SC or IM route.

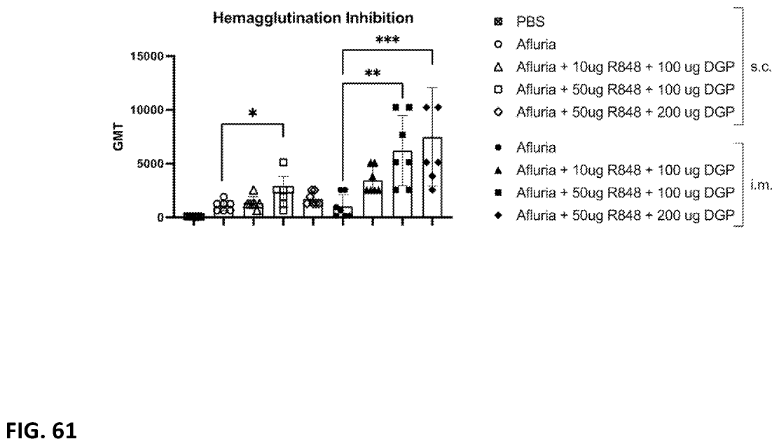

Afluria Combined with R848+DGP Induced a Stronger Antigen-specific Antibody Response Compared to Afluria Alone when Administered By SC or IM Injection. Serum was collected from each mouse 21 days after the first immunization and used for three different assays to measure antigen-specific antibody responses. First, a hemagglutination inhibition (HAI) assay was performed using Afluria as the viral antigen. When injected SC, Afluria alone did not induce significantly higher titers of antibody compared to PBS, however Afluria combined with R848+DGP at Dose Level 2 administered SC induced higher antibody titers compared to Afluria alone (p=0.01, One-Way ANOVA) (FIG. 61). When injected IM, Afluria alone did not induce higher titers of antibody compared to PBS, however Afluria combined with R848+DGP at Dose Level 2 and 3 administered IM induced higher antibody titers compared to Afluria alone (p=0.003, p=0.0003, respectively, One-Way ANOVA) (FIG. 61). When comparing the same dose levels between injection routes, Afluria combined with R848+DGP at all dose levels evaluated induced significantly higher antibody titers when injected IM compared to SC (p=0.001, p=0.01, p=0.006 respectively, Student's t-test) (Table 14-3).

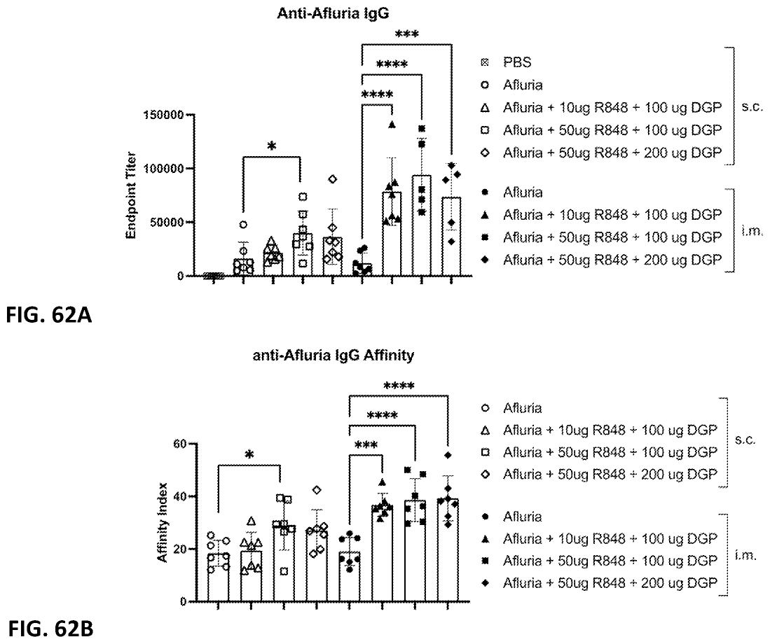

As an additional readout, antigen-specific IgG endpoint titers were assessed by ELISA using Afluria as coating antigen. Similar to HAI results, when injected SC, Afluria alone did not induce higher titers of antigen-specific IgG compared to PBS, however Afluria combined with R848+DGP at Dose Level 2 administered SC induced significantly higher IgG titers compared to Afluria alone (p=0.04, One-Way ANOVA) (FIG. 62A). When injected IM, Afluria alone also did not induce higher titers of antibody compared to PBS, however Afluria combined with R848+DGP at each dose level evaluated when administered IM induced significantly higher antibody titers compared to Afluria alone (p<0.0001, p<0.0001, p=0.0006, respectively, One-Way ANOVA) (FIG. 62A). When comparing the same dose levels between injection routes, Afluria combined with R848+DGP at all dose levels evaluated induced significantly higher antibody titers when administered IM compared to SC (p=0.0001, p=0.006, p=0.05, respectively, Student's t-test) (Table 14-3).

Lastly, the affinity of antigen-specific IgG was measured by quantifying the amount of Afluria-specific IgG via ELISA as above and then calculating the area under the curve (AUC) for each sample with and without urea treatment. The affinity index is reported as: (AUC with urea treatment/AUC without urea treatment)*100. Mirroring antibody titer data above, when injected SC, Afluria combined with R848+DGP at Dose Level 2 induced significantly higher affinity IgG compared to Afluria alone (p=0.04, One-Way ANOVA) (FIG. 62B). When injected IM, Afluria combined with R848+DGP at each dose level evaluated induced higher affinity IgG compared to Afluria alone (p=0.0002, p<0.0001, p<0.0001, respectively, One-way ANOVA) (FIG. 62B). When comparing the same dose levels between injection routes, Afluria combined with R848+DGP at all dose levels evaluated induced higher affinity antigen-specific IgG when administered IM compared to SC (p=0.0001, p=0.006, p=0.05, respectively, Student's t-test) (Table 14-3).

Taken together, these data demonstrate that Afluria combined with R848+DGP induced a stronger antigen-specific antibody response compared to Afluria alone when injected SC or IM, and that the antibody response was higher overall when Afluria+R848+DGP was administered IM.

Afluria Combined with R848+DGP Enhances Splenic DC, Memory T and Germinal Center Cell Populations. To assess how Afluria combined with R848+DGP affected splenic leukocyte frequencies, flow cytometry was performed on spleen samples from each mouse.

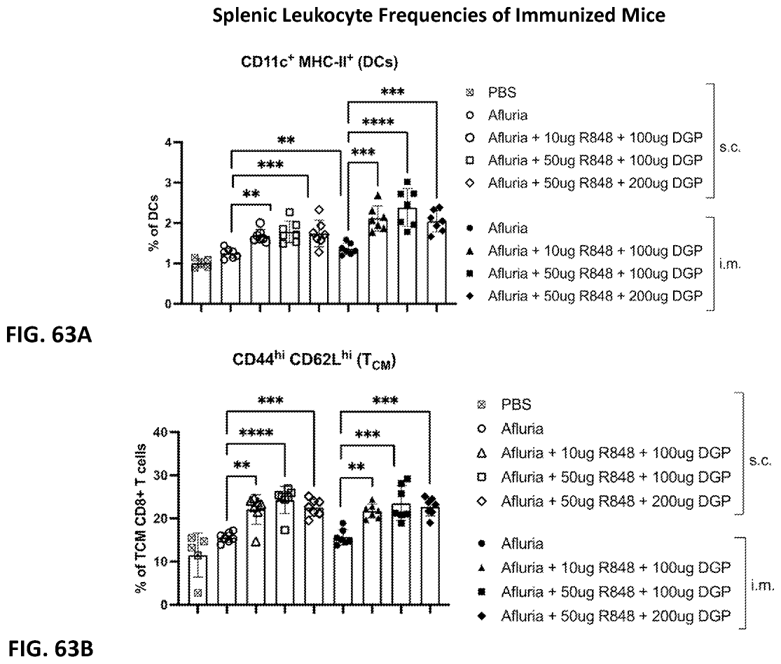

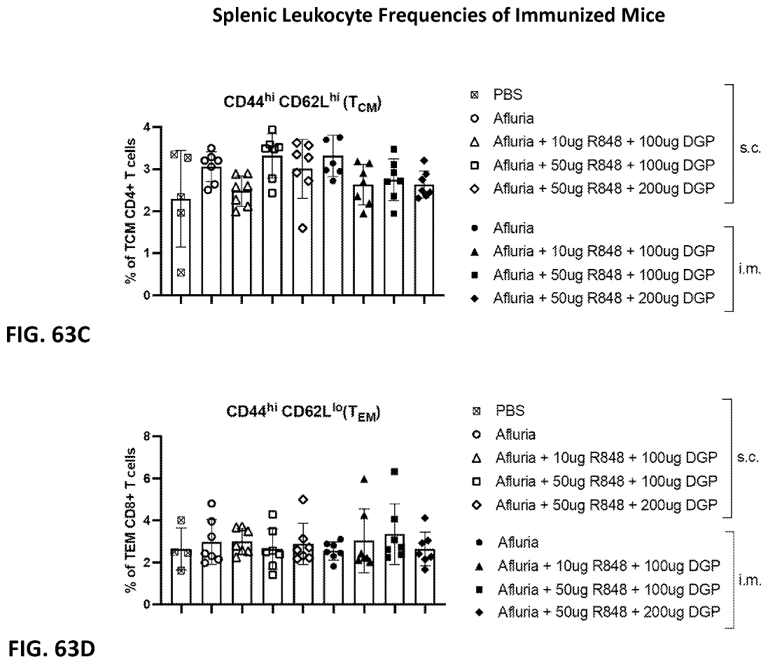

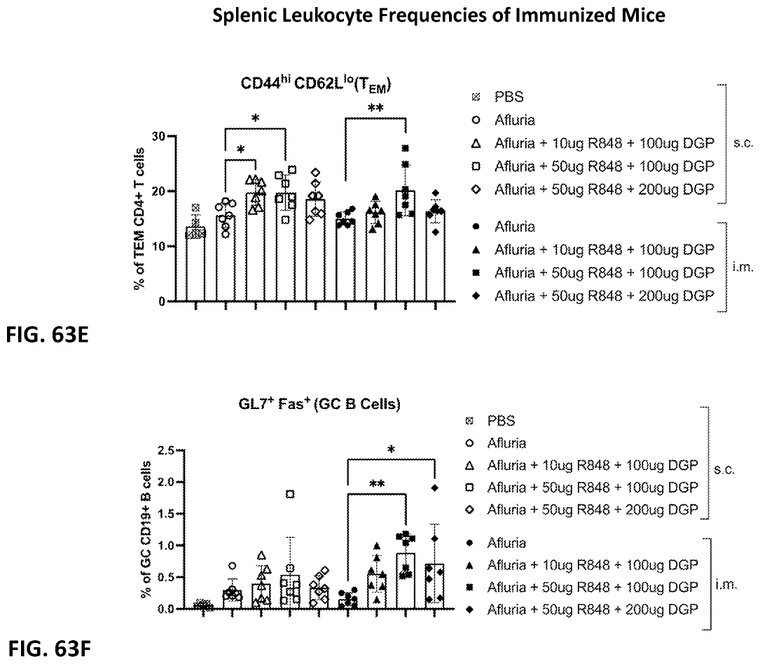

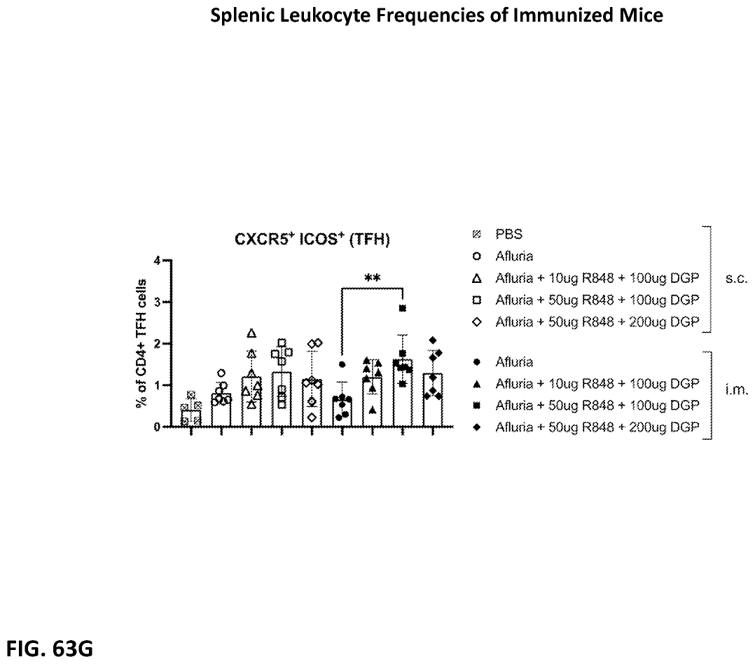

Frequency of DCs. When injected SC, Afluria alone did not lead to increased frequencies of splenic CD11c+ MCH-11+ DCs compared to PBS, however Afluria combined with R848+DGP administered SC at each dose level evaluated led to significantly more splenic DCs compared to Afluria alone (p=0.004, p=0.0004, p=0.001, respectively, One-Way ANOVA) (FIG. 63A). Similarly, when injected IM, Afluria alone did not lead to increased frequencies of splenic DCs compared to PBS, however Afluria combined with R848+DGP administered IM at each dose level evaluated led to significantly more splenic DCs compared to Afluria alone (p=0.0002, p<0.0001, p=0.0007, respectively, One-Way ANOVA) (FIG. 63A). This indicated that Afluria combined with R848+DGP leads to enhanced splenic infiltration of DCs whether administered SC or IM, and this effect can be observed up to 7 days post injection.

Frequency of Circulating Memory CD4+ and CD8+ T Cells. Next, the frequencies of circulating memory T cell populations were assessed within CD4⁺ and CD8⁺ T cell compartments. Circulating memory T cells can be divided into two groups: T central memory (TCM) and T effector memory (TEM), though the relative contributions of these populations in recall responses are not well understood. TCM are found in lymphoid tissues and blood, are highly proliferative upon recall, and become increasingly dominant in the recall response over time (Jameson and Masopust, Immunity, 48(2):214-226, 2018). Importantly, circulating CD8⁺ TCMs were shown to be critical for replenishing tissue-resident memory CD8+ T cells in the lung following influenza infection in mice (Slitter et al., Science Immunology, 2(7), eaag2031, 2017). TEM are a diverse subset of memory T cells found in blood which can traffic to peripheral tissues upon infection (Jameson and Masopust, supra, 2018).

CD8+ TCM. When injected SC, Afluria alone did not lead to increased frequencies of CD8+ TCM cells compared to PBS, however Afluria combined with R848+DGP administered SC at each dose level evaluated led to significantly more CD8+ TCMs compared to Afluria alone (p=0.002, p<0.0001, p=0.0009, respectively, One-Way ANOVA) (FIG. 63B). Similarly, when injected IM, Afluria alone did not lead to increased frequencies of splenic CD8+ TCMs compared to PBS, however Afluria combined with R848+DGP administered IM at each dose level evaluated led to significantly more CD8+ TCMs compared to Afluria alone (p=0.004, p=0.0002, p=0.0008, respectively, One-Way ANOVA) (FIG. 63B).

CD4+ TCM. TCMs within the CD4+ T cell compartment were similar across all injection groups (FIG. 63C).

CD8+ TEM. The frequency of TEMs within the CD8+ T cell compartment were also similar across all injection groups (FIG. 63D).

CD4+ TEM. When injected SC, Afluria alone did not induce higher frequencies of CD4+ TEMs compared to PBS, however Afluria combined with R848+DGP administered SC at Dose Level 2 and 3 led to significantly more CD4+ TEMs compared to Afluria alone (p=0.02, p=0.03, respectively, One-Way ANOVA). (FIG. 63E). Similarly, when injected IM, Afluria alone did not lead to increased frequencies of CD4 TEMs compared to PBS, however Afluria combined with R848+DGP administered IM at Dose Level 2 led to significantly more CD4+ TEMs compared to Afluria alone (p=0.005, One-Way ANOVA) (FIG. 63E). These data indicate that Afluria combined with R848+DGP enhance CD8+ TCM and CD4+ TEM compared to Afluria alone when administered via SC or IM injection routes.

Frequency of Germinal Center B Cells and T Follicular Helper Cells. Lastly, frequencies of cells involved in high affinity antibody production were evaluated. When injected SC, Afluria alone did not enhance frequencies of germinal center (GC) B cells (FIG. 63F) or T follicular helper (TFH) cells (FIG. 63G) compared to PBS. Furthermore, SC administration of Afluria combined with R848+DGP at each dose level evaluated did not enhance GC B or TFH cell frequencies compared to Afluria alone (FIG. 63F-G). When injected IM, Afluria alone did not enhance frequencies of GC B cells or TFH cells compared to PBS, however frequencies of GC B cells were increased when Afluria combined with Dose Levels 2 and 3 were administered IM (p=0.002, p=0.02, respectively, One-Way ANOVA) (FIG. 63F) and TFH cells were enhanced when Afluria combined with Dose Level 2 was administered IM (p=0.002, One-Way ANOVA) (FIG. 63G).

TABLE 14-3
Antibody Production

| Group | GMT SC | GMT IM | p-value |
|---|---|---|---|
| Afluria | 1097 | 1029 | 0.9 |
| Dose Level 1 | 1371 | 3474 | 0.001 |
| Dose Level 2 | 2377 | 6217 | 0.01 |
| Dose Level 3 | 1737 | 7491 | 0.006 |
| | Endpoint Titer SC | Endpoint Titer IM | p-value |
| Afluria | 16170 | 11880 | 0.5 |
| Dose Level 1 | 21519 | 78555 | 0.0005 |
| Dose Level 2 | 28.9 | 38.6 | 0.06 |
| Dose Level 3 | 27.01 | 39.3 | 0.02 |
| | Mean Affinity | Mean Affinity | |
| | Index SC | Index IM | p-value |
| Afluria | 18.4 | 19 | 0.8 |
| Dose Level 1 | 19.4 | 36.8 | 0.0001 |
| Dose Level 2 | 39985 | 94191 | 0.006 |
| Dose Level 3 | 36448 | 73706 | 0.05 |

These data demonstrate that IM administration of Afluria+R848+DGP leads to enhanced frequencies of cells involved in antibody production, consistent with the overall enhanced antibody response seen with IM route of administration (FIG. 61 and FIG. 62A-B).

### Example B-15: Activation of Canine PBMCs with R848+DGP

### Materials and Methods

Canine PBMC Isolation. PBMCs were isolated from whole blood using density gradient centrifugation. Whole blood was diluted 1:2 in PBS containing 2.5 mM EDTA and layered onto Ficoll-Paque Plus (density=1.077 g/mL). Blood was centrifuged at 800×g for 30 min with the brake off. Following centrifugation, the buffy coat containing PBMCs was collected, washed with 3× volume wash buffer (1×PBS, 2.5 mM EDTA, 1% FBS) and centrifuged at 800×g for 10 min. Cells were washed again with 30 mL of wash buffer and centrifuged at 800×g for 5 min. Red blood cells were lysed using Ack lysis buffer and the reaction was quenched with 1×PBS. Cells were centrifuged at 400×g for 5 min and resuspended in 5 mL of R10 media (RPMI-1640 media supplemented with 10% FBS, 100 U/mL Penicillin, 100 μg/mL Streptomycin, 2 mM L-Glutamine, and 1 mM Sodium Pyruvate) for counting.

Canine PBMC Hyperactivation. PBMCs were plated at 500,000 cells/well in 96-well round bottom tissue-culture treated plates and cultured with media alone, R848 alone (1 ug/mL), DGP alone (41.3 μM), or R848+DGP. Cells were also treated with the NLRP-3 inflammasome inhibitor MCC950 (10 μM) in order to assess inflammasome-dependent cytokine secretion. Following 48 hours incubation, cells were assessed for viability using Promega Cell Titer Glo (Catalog No. G9681) and secretion of IL-1β, IL-6 and IFNγ was measured by ELISA (R&D DY3747, DY609, DY718).

Statistical Analysis. PBMCs were isolated from 4 different beagle donors. Each activation condition was tested in triplicate, and reported data points are the mean of each triplicate per sample. Statistical significance was determined by One-Way ANOVA followed by Dunnett's multiple comparisons test.

### Results

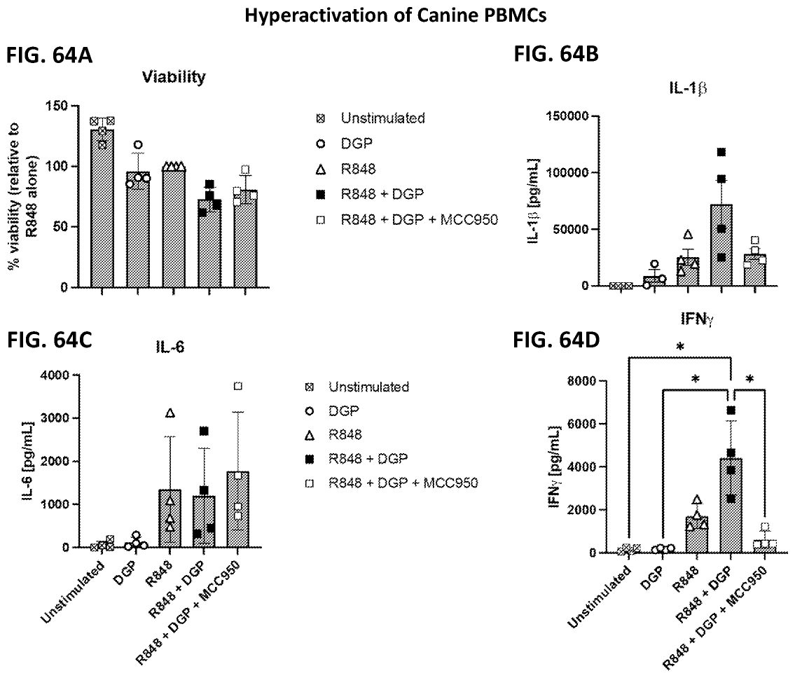

Canine PBMCs Are Hyperactivated by R848+DGP in an NLRP-3 Inflammasome-Dependent Manner. To assess whether canine PBMCs are hyperactivated by R848+DGP, PBMCs were isolated from fresh whole blood and treated with media alone, R848 alone, DGP alone, or R848+DGP. After 48 hours, cells remained viable across all conditions tested (FIG. 64A). Although not statistically significant, canine PBMCs treated with R848+DGP secreted more IL-1β compared to cells treated with R848 alone or DGP alone (FIG. 64B), indicating that canine PBMCs can be hyperactivated by R848+DGP. Additionally, when treated with MCC950, levels of IL-1β induced by R848+DGP decreased (FIG. 64B), indicating that these hyperactivating stimuli work through the NLRP3 inflammasome in canine PBMCs, similar to what has been observed in human and murine cells. As expected, IL-6 was induced in any condition that included R848, and this was not affected when cells were treated with MCC950 (FIG. 64C).

Similar to human and nonhuman primate PBMCs, canine PBMCs secreted high levels of IFNγ when treated with R848+DGP compared to unstimulated cells or cells treated with DGP alone or R848 alone (p<0.0001, p<0.001, P=0.001, respectively). As phagocytic cells are believed to be the main targets of hyperactivating stimuli and are unlikely to produce IFNγ, this cytokine is likely being produced by neighboring cells such as T cells or NK cells. Furthermore, IFNγ levels were decreased when MCC950 was added (p<0.0001) (FIG. 64D), indicating that IFNγ secretion is dependent on NLRP3 inflammasome activity. Since IL-1β can act on T cells to induce IFNγ secretion, this is likely the result of decreased IL-1β observed with MCC950 treatment (FIG. 64B).

### Example B-16: Enhanced Antigen-Specific Immune Responses in Dogs when Immunized with Protein Antigen in Combination with R848+DGP

### Materials and Methods

Immunization and Study Design. This study consisted of 3 groups of naïve research beagles, each containing one female and one male. Beagles in group 1 were immunized with 50 ug recombinant SARS-CoV-2 Spike protein and 50 ug recombinant Influenza A (HINI) HA protein, beagles in group 2 were immunized with Spike and HA combined with R848 (0.01 mg/kg) and 100 ug DGP, beagles in group 3 were immunized with Spike and HA combined with R848 (0.01 mg/kg) and 200 ug GDP. All immunizations were given intradermally, and dogs were boosted similarly on days 14 and day 28. Whole blood was received on day −3 (baseline), day 21 and day 42 for isolation of PBMCs for T cell restimulation assays. Whole blood was also used on d-3 and d42 for complete blood count analysis. Serum was collected on day −3 (baseline), day 28 and day 42 and used to assess antigen-specific antibody titers. Beagles were observed daily for overall wellbeing and weekly for clinical observations of distress or injection site reactions for a total of 42 days.

Canine PBMC Isolation. PBMCs were isolated from fresh whole blood using density gradient centrifugation. Whole blood was diluted 1:2 in PBS containing 2.5 mM EDTA and layered onto Ficoll-Paque Plus (density=1.077 g/mL). Blood was centrifuged at 800×g for 30 min with the brake off. Following centrifugation, the buffy coat containing PBMCs was collected, washed with 3× volume wash buffer (1×PBS, 2.5 mM EDTA, 1% FBS) and centrifuged at 800×g for 10 min. Cells were washed again with 30 mL of wash buffer and centrifuged at 800×g for 5 min. Red blood cells were lysed using Ack lysis buffer and the reaction was quenched with 1×PBS. Cells were centrifuged at 400×g for 5 min and resuspended in 5 mL of R10 media (RPMI-1640 media supplemented with 10% FBS, 100 U/mL Penicillin, 100 μg/mL Streptomycin, 2 mM L-Glutamine, and 1 mM Sodium Pyruvate) for counting.

PBMC Restimulation and IFNγ Enzyme Linked Immunosorbent Assay (ELISA). 100 μl of R10 media alone, R10 media containing 1 μg/mL SARS-CoV-2 Prot_S Spike PepTivator, R10 media containing 1 μg/mL Influenza A (H1N1) HA PepTivator, or R10 media containing 2× Cell Stimulation Cocktail (Thermofisher 004970-93) was added to tissue-culture treated 96-well round bottom plates. PBMCs were seeded at 500,000 cells per well in 100 μL and plates were incubated at 37° C. for 72 hours. Following incubation, plates were centrifuged at 300×g for 5 min and supernatants were collected and stored at −80° C. until ready for use. ELISAs were performed using R&D Systems canine IFNγ DuoSet ELISA kit (Cat #DY781B) according to manufacturer's instructions.

Antibody Endpoint Titer ELISA. Nunc MaxiSorp ELISA plates were coated with 100 μL PBS containing 2.5 μg/mL of recombinant SARS-CoV-2 spike protein or 2.5 μg/mL of Influenza A virus (H1N1) HA protein and incubated overnight at 4° C. The following morning, plates were washed with wash buffer (PBS containing 0.05% Tween 20) and blocked with 300 μL PBS containing 2% BSA for 1 hour at RT on a rocking platform. Canine serum was thawed at RT and diluted 1 to 640 in PBS containing 1% BSA, followed by 6 additional 3-fold serial dilutions. After blocking, plates were washed, serially diluted serum was added, and plates were incubated overnight at 4° C. on a rocking platform. The following day, plates were washed and 100 μL of Immunoglobulin G (IgG)-Horseradish Peroxidase (HRP) (1:5,000 in PBS containing 1% BSA) was added for two hours at RT with no rocking. Plates were washed and 100 μL of 3,3′,5,5′-Tetramethylbenzidine (TMB) was added to all wells. Plates were incubated for two min at RT while hidden from light and the reaction was stopped by the addition of 50 μL of 2N sulfuric acid. Plates were read on a SpectraMax M5 spectrophotometer at 450 and 570 nm wavelengths, and the 570 nm absorbance value was subtracted from the 450 nm absorbance value.

For calculation of endpoint titer, a positive cutoff optical density (OD) value was determined by measuring the mean OD and SD of undiluted serum from a naïve C57BL/6 mouse and setting the cut-off at two SDs above this mean (Fox et al., 2020). Endpoint titer was determined using Prism as described previously (Yang et al., 2022): OD values for serially diluted serum were plotted in an XY table in Prism and this positive cut-off value was entered in the first empty row of each sample. Data was transformed as X=log X and this was analyzed using non-linear regression (4PL), X is log(concentration). Unknowns were interpolated from standard curve. Nonlinear fit data (Interpolated X mean values) were transformed as X=10^{X} to obtain plotted endpoint titer values.

CBC and Serum Cytokines. Fresh whole blood was sent to IDEXX Laboratories for complete blood count analysis on day −3 and day 42. Serum cytokines were assessed by ELISA for detection of IL-6 (R&D CA6000) and TNFα (R&D CATA00) using undiluted serum samples.

Statistical Analysis. PBMCs and serum samples were received from 6 different animals. T and B cell assays were performed in duplicate and reported data are the mean of each duplicate for each canine sample.

### Results

Protein Antigen Combined with R848+DGP Was Well-tolerated in Research Beagles. In order to evaluate the preliminary safety of antigen combined with R848+DGP in dogs, six research beagles were immunized intradermally on day 0 with recombinant Influenza A HA (HA) and SARS-CoV-2 Spike (Spike) proteins alone or in combination with R848 (0.01 mg/kg) and two dose levels of DGP (100 ug/dog or 200 ug/dog). Dogs received additional boost injections on days 14 and 28 and were monitored for a total of 42 days. Weekly physical exams noted regular weight gain, temperature, and heartrate for all dogs. When assessing injection sites post-dosing, it was noted that all 6 dogs had some form of injection site swelling and redness after injection which resolved within 1 week. Notably, the female beagle in group 2 had injection site swelling and redness after immunization that developed into an open wound after the second injection. This wound was healing until after the third and final injection, when it again became swollen and purulent. By day 42 this wound was completely healed, and no other dogs showed any signs of injection site reactions or distress.

Complete blood counts (CBC) showed that frequencies of leukocytes and HGB-related measurements were within the normal range for research beagles at baseline and on day 42 (Table 16-1), indicating that treatment with antigen alone or combined with R848 and DGP did not lead to deviations from expected circulating blood cell counts. Taken together these data demonstrate that Antigen combined with R848 and GDP was well-tolerated at the doses used in this study.

TABLE 16-1
Complete Blood Count (CBC) Results

| | Gender | Day −3 | Day 42 | Cells |
|---|---|---|---|---|
| Group 1 | Female | 7.7 | DNR | Neutrophils (K/μL) |
| Spike and HA | Male | 4.2 | 6.1 | Range: 3.0-10.5 |
| Group 2 | Female | 5.2 | 4.5 | |
| Spike and HA | Male | 8.4 | 6.1 | |
| R848 + DGP 100 ug | | | | |
| Group 3 | Female | 7.9 | 5.8 | |
| Spike and HA | Male | 4.4 | 4.8 | |
| R848 + DGP 200 ug | | | | |
| Group 1 | Female | 11.6 | DNR | WBC (K/μL) |
| Spike and HA | Male | 6.7 | 9.5 | Range: 6.0-13.0 |
| Group 2 | Female | 8.4 | 6.9 | |
| Spike and HA | Male | 12.3 | 9.5 | |
| R848 + DGP 100 ug | | | | |
| Group 3 | Female | 11.4 | 8.7 | |
| Spike and HA | Male | 7.7 | 8.3 | |
| R848 + DGP 200 ug | | | | |
| Group 1 | Female | 3.2 | DNR | Lymphocytes (K/μL) |
| Spike and HA | Male | 1.8 | 2.5 | Range: 1.0-4.0 |
| Group 2 | Female | 2.5 | 2.1 | |
| Spike and HA | Male | 1.7 | 2.5 | |
| R848 + DGP 100 ug | | | | |
| Group 3 | Female | 2.4 | 2.3 | |
| Spike and HA | Male | 2.8 | 2.9 | |
| R848 + DGP 200 ug | | | | |
| Group 1 | Female | 348 | DNR | Monocytes (per μL) |
| Spike and HA | Male | 288 | 580 | Range: 150-1200 |
| Group 2 | Female | 218 | 159 | |
| Spike and HA | Male | 615 | 219 | |
| R848 + DGP 100 ug | | | | |
| Group 3 | Female | 274 | 165 | |
| Spike and HA | Male | 200 | 241 | |
| R848 + DGP 200 ug | | | | |
| Group 1 | Female | 0.3 | DNR | Eosinophils (per μL) |
| Spike and HA | Male | 0.3 | 0.2 | Range: 0-1.5 |
| Group 2 | Female | 0.4 | 0.1 | |
| Spike and HA | Male | 1.5 | 0.7 | |
| R848 + DGP 100 ug | | | | |
| Group 3 | Female | 0.8 | 0.5 | |
| Spike and HA | Male | 0.3 | 0.3 | |
| R848 + DGP 200 ug | | | | |
| Group 1 | Female | 23 | DNR | Basophils (per μL) |
| Spike and HA | Male | 20 | 29 | Range: 0-50 |
| Group 2 | Female | 17 | 21 | |
| Spike and HA | Male | 25 | 10 | |
| R848 + DGP 100 ug | | | | |
| Group 3 | Female | 11 | 17 | |
| Spike and HA | Male | 31 | 33 | |
| R848 + DGP 200 ug | | | | |
| Group 1 | Female | 7.20 | DNR | RBC (M/μL) |
| Spike and HA | Male | 8.66 | 6.11 | Range: 5.6-8.0 |
| Group 2 | Female | 6.83 | 5.30 | |
| Spike and HA | Male | 6.78 | 5.17 | |
| R848 + DGP 100 ug | | | | |
| Group 3 | Female | 6.32 | 5.24 | |
| Spike and HA | Male | 6.63 | 6.01 | |
| R848 + DGP 200 ug | | | | |
| Group 1 | Female | 17.1 | DNR | HGB (g/dL) |
| Spike and HA | Male | 19.6 | 14.9 | Range: 14-19 |
| Group 2 | Female | 15.8 | 12.4 | |
| Spike and HA | Male | 15.7 | 12.0 | |
| R848 + DGP 100 ug | | | | |
| Group 3 | Female | 14.7 | 12.2 | |
| Spike and HA | Male | 15.9 | 14.6 | |
| R848 + DGP 200 ug | | | | |

Abbreviations: DNR: Did not respond due to degradation of sample.
Reference ranges published by UC Davis School of Veterinary Medicine.

Beagles Immunized with Antigen Combined with R848+DGP Developed Stronger Antigen-specific T cell Responses Compared to Beagles Immunized with Antigen Alone. Previous studies in mice have shown that antigen combined with R848 and DGP induced a stronger antigen-specific T cell response compared to antigen alone. Furthermore, when two different protein antigens are used, R848+DGP induced a stronger antigen specific T cell response to both immunizing antigens. In order to assess how R848+DGP influences the generation of antigen-specific T cells in dogs, PBMCs were collected prior to immunization (day −3) and one week after each boost (days 21 and 35). Total PBMCs were left unstimulated or restimulated for 72 hours with HA PepTivator mix (Miltenyi 130-099-803) or Spike PepTivator mix (Miltenyi 130-126-701) and IFNγ secretion was measured by ELISA in cell culture supernatants. In order to assess antigen-specific IFNγ secretion above background, IFNγ concentrations from unstimulated cells were subtracted from stimulated cells for each condition.

Prior to immunization (d-3), IFNγ secretion was at or below the level of detection in PBMCs from all 6 dogs after restimulation with HA peptides (Table 16-2) and in 5 out of 6 dogs after restimulation with Spike peptides (Table 16-3). Dogs that received antigen alone did not exhibit an increase in IFNγ secretion compared to baseline (d-3) when restimulated with HA or Spike peptides at either time point post immunization (Table 16-2, Table 16-3, respectively). In contrast, all four dogs that received antigen combined with R848+DGP had high levels of IFNγ secretion compared to baseline at d21 and d35 when restimulated with Spike peptides (Table 16-2). Additionally, all four dogs receiving antigen combined with R848 and DGP had high levels of IFNγ secretion compared to baseline when restimulated with HA peptides at d21, and this was also observed for three out of four dogs by day 35 (Table 16-3). Together these data demonstrate that intradermal administration of Spike and HA proteins alone does not induce a detectable antigen-specific IFNγ T cell response to either antigen, however the addition of R848 and DGP at two different dose levels significantly increases the generation of antigen-specific T cell responses to both antigens.

TABLE 16-2
Concentrations of HA-specific IFNγ
in PBMC Restimulation Supernatants

| | | IFNγ pg/mL | IFNγ pg/mL | IFNγ pg/mL |
|---|---|---|---|---|
| | | d −3 baseline | d 21 | d 35 |
| Group 1 | Female | 35.1 | 0.0 | 0.0 |
| Spike and HA | Male | 0.0 | 0.0 | 0.0 |
| Group 2 | Female | 0.0 | 762.1 | 1070.6 |
| Spike and HA | Male | 0.0 | 2213.3 | 3481.13 |
| R848 + DGP 100 ug | | | | |
| Group 3 | Female | 0.0 | 2280.1 | 1535.6 |
| Spike and HA | Male | 0.0 | 856.2 | 351.8 |
| R848 + DGP 200 ug | | | | |

TABLE 16-3
Concentrations of Spike-specific IFNγ
in PBMC Restimulation Supernatants

| | | IFNγ pg/mL | IFNγ pg/mL | IFNγ pg/mL |
|---|---|---|---|---|
| | | d −3 | d 21 | d 35 |
| Group 1 | Female | 216.0 | 0.0 | 0.0 |
| Spike and HA | Male | 0.0 | 25.3 | 0.0 |
| Group 2 | Female | 0.0 | 482.0 | 94.6 |
| Spike and HA | Male | 0.0 | 1284.0 | 843.2 |
| R848 + DGP 100 ug | | | | |
| Group 3 | Female | 0.0 | 444.5 | 0 |
| Spike and HA | Male | 0.0 | 380.8 | 221.3 |
| R848 + DGP 200 ug | | | | |

Beagles Immunized with Antigen Combined with R848+DGP Developed Higher Titers of Antigen-specific Antibodies Compared to Beagles Immunized with Antigen Alone. In order to assess how R848+DGP influences the production of antigen-specific antibodies in dogs, titers of HA-specific and Spike-specific IgG antibodies were determined from serum that was collected prior to immunization (day −3) and on days 28 and 42. Titers of HA-specific IgG were similar across all groups at baseline and were increased in all six dogs when assessed at day 28 and day 42 (Table 16-4). Furthermore, while dogs immunized with antigen alone exhibited increased titers of HA-specific IgG from baseline at days 28 and 42, the fold change increase from baseline was much higher in dogs that received antigen combined with R848 and DGP (Table 16-5).

Similarly, titers of Spike-specific IgG were similar across all groups at baseline and were increased in all six dogs at day 28 and day 42 (Table 16-6). Additionally, while dogs immunized with antigen alone exhibited increased titers of Spike-specific IgG from baseline at days 28 and 42, the fold change increase from baseline was higher for dogs that received antigen combined with R848 and DGP (Table 16-7). Together these data demonstrate that intradermal administration of Spike and HA proteins leads to production of antigen-specific antibodies, which is further increased by the addition of R848 and DGP at two different dose levels.

TABLE 16-4
Titers of HA-specific IgG Antibodies in Serum of Immunized Dogs

| | | Endpoint | Endpoint | Endpoint |
|---|---|---|---|---|
| | | titer | titer | titer |
| | | d −3 | d 28 | d 42 |
| Group 1 | Female | 544 | 37,653 | 84,113 |
| Spike and HA | Male | 634 | 29,330 | 27,760 |
| Group 2 | Female | 554 | 283,755 | 160,586 |
| Spike and HA | Male | 593 | 165,841 | 204,154 |
| R848 + DGP 100 ug | | | | |
| Group 3 | Female | 550 | 172,965 | 192,198 |
| Spike and HA | Male | 562 | 97,822 | 161,916 |
| R848 + DGP 200 ug | | | | |

TABLE 16-5
Fold change of HA-specific IgG Antibodies from Baseline

| | | Fold | | |
|---|---|---|---|---|
| | | change | Fold | Fold |
| Fold Change from | | d −3 | Change | Change |
| Baseline | | (baseline) | d 28 | d 42 |
| Group 1 | Female | 1 | 69 | 155 |
| Spike and HA | Male | 1 | 46 | 44 |
| Group 2 | Female | 1 | 512 | 290 |
| Spike and HA | Male | 1 | 280 | 344 |
| R848 + DGP 100 ug | | | | |
| Group 3 | Female | 1 | 315 | 350 |
| Spike and HA | Male | 1 | 174 | 288 |
| R848 + DGP 200 ug | | | | |

TABLE 16-6
Titers of Spike-specific IgG Antibodies
in Serum of Immunized Dogs

| | | Endpoint | | |
|---|---|---|---|---|
| | | titer | Endpoint | Endpoint |
| | | d −3 | titer | titer |
| | | (baseline) | d 28 | d 42 |
| Group 1 | Female | 626 | 30,569 | 46,810 |
| Spike and HA | Male | 609 | 26,633 | 20,276 |
| Group 2 | Female | 572 | 176,149 | 89,645 |
| Spike and HA | Male | 579 | 45,215 | 36,694 |
| R848 + DGP 100 ug | | | | |
| Group 3 | Female | 618 | 62,946 | 63,507 |
| Spike and HA | Male | 626 | 78,191 | 61,780 |
| R848 + DGP 200 ug | | | | |

TABLE 16-7
Fold change of Spike-specific IgG Antibodies from Baseline

| | | Fold change | | |
|---|---|---|---|---|
| Fold Change from | | d −3 | Fold Change | Fold Change |
| Baseline | | (baseline) | d 28 | d 42 |
| Group 1 | Female | 1 | 49 | 75 |
| Spike and HA | Male | 1 | 44 | 33 |
| Group 2 | Female | 1 | 308 | 157 |
| Spike and HA | Male | 1 | 78 | 63 |
| R848 + DGP 100 ug | | | | |
| Group 3 | Female | 1 | 102 | 103 |
| Spike and HA | Male | 1 | 125 | 99 |
| R848 + DGP 200 ug | | | | |

### Example B-17: R848+DGP Induces Protection Against Live Influenza a Virus Challenge

PR8 is a live virus that is propagated in specific pathogen-free chicken embryonated eggs. This strain was isolated in 1934 from a human patient in Puerto Rico and was deposited by the Centers for Disease Control and Prevention. The PR8 influenza virus strain is an attenuated virus, and is unable to replicate in humans, as a result of over 100 passages in each of mice, ferrets and embryonated chicken eggs. According to the ATCC, there have been no reports of laboratory-acquired infections with strain A/PR/8/34.

### Material and Methods

PR8 Virus. Influenza A virus (H1N1), also called PR8 (ATCC VR-95PQ) is a high-titer, purified, live virus suspended in 1×PBS+0.1% sodium azide. This product was prepared from ATCC VR-95 via purification through sucrose gradient centrifugation and is devoid of cellular debris and contaminants. Influenza PR8 strain has been used for the past 30 years to produce inactivated influenza vaccines. Virus inactivation was done by heating the live PR8 virus at 56 degrees for 45 min. Inactivated virus was titrated for HA content and used for immunization.

Immunization. Ten BALB/c mice per group were immunized subcutaneously on D-14 with either PBS or inactivated virus alone or inactivated virus in combination with R848+DGP, or inactivated virus plus AddaVax™ squalene-oil-in-water adjuvant (InvivoGen), which has similar formulation to the MF59® adjuvant of Novartis. Mice were then challenged on day 0 with 1000 PFU of live PR8 virus (ATCC) injected intranasal (25 uL in one nostril) on D0. Details for each group are listed in Table 17-1.

TABLE 17-1
Immunization Groups

| | | | Other | | |
|---|---|---|---|---|---|
| Group | R848 | DGP | Adjuvant | Antigen | Schedule |
| PBS | x | x | x | x | Immunization: |
| Inactivated PR8 | x | | x | Inactivated virus, | SC 100 ul on D −14. |
| virus | | | | 8 ng/mouse of HA | Challenge: |
| R848 + DGP + | 50 ug | 100 ug | x | Inactivated virus, | IN with 1:100,000 |
| Inactivated PR8 | | | | 8 ng/mouse of HA | (1000PFU) on D 0 |
| virus | | | | | |
| AddaVax ™ + | x | x | AddaVax ™ | Inactivated virus, | |
| Inactivated PR8 | | | (1:1) | 8 ng/mouse of HA | |
| virus | | | | | |

Clinical Score Assessment. Out of the 10 mice per group: 5 mice per group were assessed for body weight changes throughout the study, as well as mortality and morbidity using a murine scoring system as shown in Table 17-2. Four areas of focus for clinical score were established: appearance, mobility, breathing and eyes. Each mouse was assigned a clinical score that would be the mean of the scores in all four areas. Body weight and clinical scores were assessed daily starting on D0 and until D10.

TABLE 17-2
Murine Behavioral and Clinical Score

| | Score | | | |
|---|---|---|---|---|
| Area | 0 | 1 | 2 | 3 |
| Appearance | Smooth | Slightly ruffled fur | Majority of fur on | Piloerection, puffy |
| | coat | | back is ruffed. | appearance |
| Mobility | Active | Active, avoids | Active only when | Non-responsive, |
| | | standing upright | provoked | even when provoked |
| Breathing | Normal | Slightly labored | Labored breathing | Heavily labored |
| | | | | breathing |
| Eyes | Open | Not fully open, | Half closed, | Mostly or |
| | | potentially | potential secretions | completely closed |
| | | secretions | | |

Blood Collection. Blood was collected from all mice on D-1 (one day before challenge). Briefly, BALB/c mice were placed under Isoflurane for approximately 10 min for anesthesia. Using a 21G needle, mice were gently poked through the skin to the submandibular space to induce bleeding. Five-Six drops were collected in a mini collect K2EDTA blood collection tube. Blood samples were transported back to Corner Therapeutics lab at RT and were allowed to warm up to RT for at least 15 min. To process the blood, 1 mL of RBC lysis buffer was added to 150 μl of whole blood into each well of 96 deep-well plate. Samples were then mixed with multi-channel and incubated at RT for 20 min. 600 μl of PBS is then added to all wells and samples are centrifuged at 600×g for 5 min. This step is repeated twice, then pellets are resuspended in 200 μl of PBS and transferred to 96-well V bottom plate for tetramer staining.

Tetramer Staining. 200 μL of processed blood samples (as described above) were plated into 96-well V bottom plates. Cells were spun at 400×g for 4 min then washed with PBS at least once before cell staining. Cells were then resuspended in 100 ul PBS containing Live/Dead Aqua (1:1000) and incubated for 20 min at 4° C. Cells were then washed and resuspend in 100 ul of FACS buffer containing Fc block (1:100) for 10 min., then washed again with 100 ul FACS buffer. For tetramer staining, cells were resuspended in 100 μl of FACS buffer containing H-2Kd Influenza NP Tetramer-TYQRTRALV-PE and H-2Kd Influenza NP Tetramer-TYQRTRALV-APC (1:20) and incubated at 37° C. for 2 hours. For surface markers staining, cells were washed with 100 μl FACS buffer and spun for 4 min at 400×g. The cell pellet was stained with anti-mouse CD3, anti-mouse CD4 and anti-mouse CD8□ antibodies as indicated in Table 6 for 20 min at 4° C. Cells were then washed and resuspended in 100 μl of 4% PFA to fix the cells for 20 min at RT. After fixation, cells are washed twice with FACS buffer and kept in 4° C. overnight in 150p FACS buffer. Prior to sample acquisition on the Symphony A3, Countbright counting beads are added to the samples to allow the measurement of absolute number of cells.

Antibody Responses. HA-specific antibodies and NP-specific antibodies in the serum of mice receiving immunization were assessed on D-1 (one day before challenge with live PR8 virus). NP and HA-specific total IgG were assessed using ELISA. Briefly, ELISA plates were coated with 1 ug/mL HA or NP recombinant proteins overnight, then washed and blocked with 2% bovine serum albumin. Plates were washed again, and then serum was added to the plates at a 1:500 dilution, followed by 1:5 dilutions completed for a total of 7 serum dilutions tested. Samples were washed, then incubated with detection antibody specific for IgG conjugated to HRP (Southern Biotech). Plates were washed, then incubated with TMB, and stop solution was added once color development was completed.

Plaque Assay. The viral load in the BAL was measured by plaque assay. For the plaque assay, 3.5×10⁵ MDCK cells were seeded into two 6 well plate per BAL. The cells were cultured in MDCK media (EMEM+10% FBS+Pen-Strep) for 16 hrs. The wells were washed once with 3 ml 1×PBS and once with 3 ml of infection media (IMDM+Pen-Strep+0.2% BSA+1 mg/ml of TPCK-trypsin). Ten-fold dilutions of BAL (up to 10⁻⁶ dilution) in infection media were prepared and MDCK cells were infected with 500 ml of each virus dilutions in duplicates. MDCK cells were infected for 1 hr at 37° C. After the infection, inoculum was removed and 3 ml of 0.3% agarose prepared in infection media was layered on top of the cells. The cells were incubated at 37° C. for 72 hrs, followed by addition of 4% paraformaldehyde to fix the cells. One hour after fixation, agarose layer was removed gently without disrupting the monolayer. Add 2 ml of 2% crystal violet to stain the live cells for 10 min at RT. The plates were washed under the running tap water to remove the crystal violet stain. Plates were allowed to dry overnight. The plaques were counted and based dilution of the virus the plaque forming units per ml was calculated.

HA ELISA. For quantitative determination of Influenza Hemagglutinin (HA) levels in the BAL, Influenza A H1N1 (A/Puerto Rico/8/1934) Hemagglutinin/HA ELISA Pair Set (Sino Biologics) was used for a sandwich ELISA.

BAL Collection. The bronchoalveolar lavage fluid (BAL) was collected from the lungs for viral load testing by inserting a catheter into the trachea of mice. During the procedure, 3 washes with PBS is performed by injecting 1 mL of PBS each time to wash the airways and retrieve the fluid sample. The collected BAL from each mouse is centrifuged for 5 min at 4 degrees before freezing it at −80 degrees.

Data Analysis. In in vivo studies, n refers to the number of animals per condition. Graphical data was shown as mean values with error bars indicating the SD of 5 mice/group. Each symbol represents one mouse. All experiments were analyzed using Prism 7 (GraphPad Software).

Statistical differences were calculated by one-way ANOVA with Tukey post-hoc test. P values of <0.05 (*), <0.01 (**) or <0.001 (***); <0.0001 (****) indicated significant differences between groups.

### Results

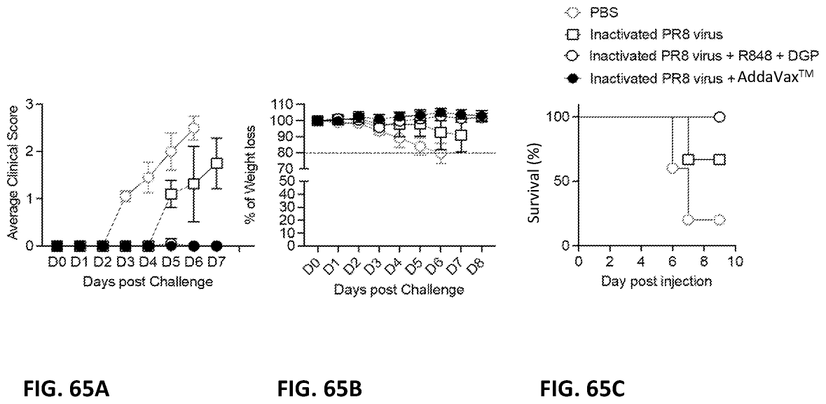

Immunization with inactivated PR8 virus in combination with R848+DGP or AddaVax™ induce BALB/c mice protection against live PR8 virus challenge. R848+DGP is expected to act as an adjuvant that increases the immunogenicity of vaccines by enhancing antigen-specific T cell responses. To test this, mice were immunized with PBS, or inactivated virus alone, or inactivated virus in combination with R848+DGP, or inactivated virus with the AddaVax™ squalene-oil-in-water adjuvant (InvivoGen), which has a similar formulation to the MF590 adjuvant (Novartis). 14 days post immunization, mice were challenged intranasally (IN) with 1000 PFU of live PR8 virus. Mice were then monitored daily for weight and survival measurement, as well as clinical score assessment. Mice were euthanized if they lost more than 20% of their body weight. When mice were immunized with PBS then challenged with a live PR8 virus, all mice developed clinical symptoms starting day 2 post challenge and all mice lost 20% of their body weight by Day 6 post challenge. As a result, 90% of the PBS immunized mice succumbed to the challenge by day 8 post PR8 challenge (FIG. 65A). When mice were immunized with the inactivated virus alone then challenged with PR8 virus, clinical symptoms were slightly delayed (FIG. 65) compared to mice immunized with PBS. In addition, immunization with inactivated virus delayed the loss of weight and protected 60% of the challenged mice. These data are consistent with previous studies showing that the inactivated virus by itself provides protection against virus re-encounter. Interestingly, the immunization of mice in the presence of R848+DGP or AddaVax™ adjuvants completely protected mice against PR8 challenge (FIG. 65A-C), as observed by the absence of clinical symptoms, absence of weight loss and survival of 100% of immunized mice. These data demonstrate that R848+DGP acts similarly to the AddaVax™ adjuvant by inducing complete protection against virus challenge.

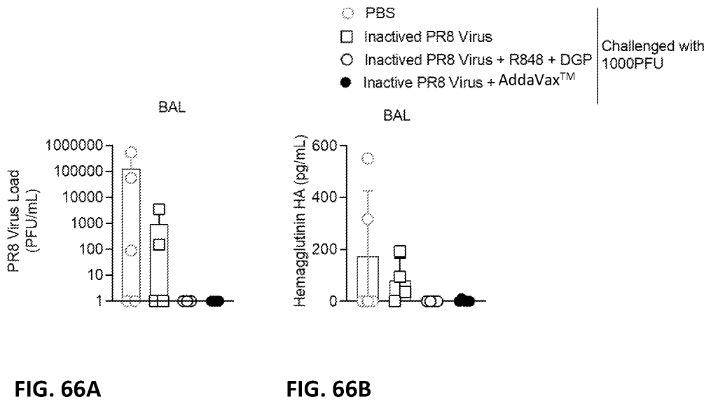

Mice immunized with inactivated PR8 virus in combination with R848+DGP or AddaVax™ exhibit low viral load in the lungs. To assess whether the protection against PR8 virus correlates with reduced viral presence in the lung, the viral load of PR8 in the bronchoalveolar lavage (BAL) fluid was measured by plaque assay and HA ELISA on day 5 post-challenge. When mice were immunized with PBS then challenged with PR8, a high viral load was detected in at least 3 out of 5 mice as determined by measuring PFU/mL in the BAL fluid (FIG. 66A). While the viral load was lower in the BAL of mice that were immunization with inactivated virus, immunization with inactivated virus in combination with R848+DGP or AddaVax™ completely abrogated the presence of virus in the BAL fluid (FIG. 66A). These data indicated that R848+DGP and AddaVax™ strongly inhibited virus replication in the lung compartment, and were confirmed by HA ELISA, which detects the levels of hemagglutinin in the BAL fluid (FIG. 66B). While mice immunized with PBS or inactivated virus alone showed detectable levels of HA in BAL fluid, no HA was detected in the BAL fluid of any mouse following the immunization with the inactivated virus in combination with R848+DGP or AddaVax™ (FIG. 66B). In summary these data indicated that R848+DGP and AddaVax™ induce higher levels of protection against live PR8 virus as compared to immunization with the inactivated virus alone. Moreover, the absence of virus detected in the BAL fluid correlated with the low clinical score, low weight loss and survival of all the mice that were immunized with inactivated virus in combination with R848+DGP or AddaVax™.

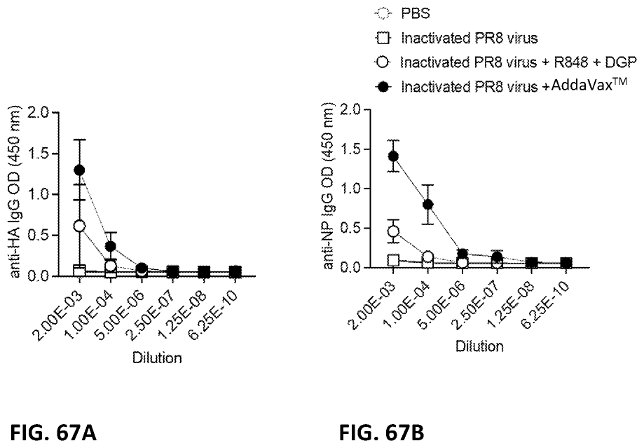

AddaVax™ induces strong antibody responses against viral proteins. To assess the mechanism by which immunized mice were protected against PR8 challenge, systemic B cells responses were measured in the serum of immunized mice. One day prior to virus challenge, serum samples from the immunized mice were collected, and antibodies against the viral hemagglutinin (HA) and nucleoprotein (NP) were measured by ELISA by serial dilution of serum. While mice immunized with PBS or inactivated virus alone did not induce any anti-HA and anti-NP IgG in the serum, immunization with inactivated virus in combination with R848+DGP enhanced anti-HA and anti-NP responses compared to immunization with inactivated virus alone (FIG. 67A-B). Notably, AddaVax™ outperformed R848+DGP by inducing significantly higher levels of anti-HA and anti-NP in the serum. These data suggest that AddaVax™ provides protection against PR8 via antibody responses. R848+DGP also induced antibody responses, albeit to a lesser extent than AddaVax™. But since the threshold antibody level needed to provide protection against PR8 challenge is not known, it is unclear whether the difference in antibody titers elicited by the two vaccines is clinically meaningful.

R848+DGP induces strong T cell responses against the conserved viral nucleoprotein. To further assess the mechanism by which mice immunized with R848+DGP induced protection, systemic antigen-specific T cells responses were measured in blood of immunized mice. One day prior to the virus challenge, whole blood samples from the immunized mice were collected, and antigen-specific T cell responses were determined by tetramer staining. Anti-NP T cells were detected by using two H-2Kd tetramer conjugated to two fluorochromes: H-2Kd Influenza NP Tetramer-TYQRTRALV-PE and H-2Kd Influenza NP Tetramer-TYQRTRALV-APC.

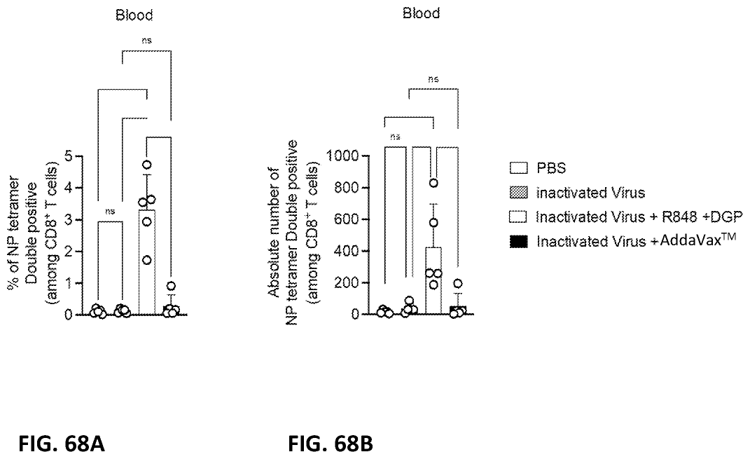

While immunization with PBS or inactivated virus alone did not induce any anti-NP specific T cells, immunization with inactivated virus in combination with R848+DGP strongly induced anti-NP T cells in the blood as detected by the absolute number and percentage of tetramer double positive T cells compared to immunization with inactivated virus alone (FIG. 68A-B). Notably, R848+DGP outperformed AddaVax™ by inducing significantly stronger levels of anti-NP T cells in the blood. These observations suggest that while AddaVax™ provides protection against PR8 via antibody responses, R848+DGP induces protection by generating strong antigen specific T cell responses. Since NP protein is a highly conserved protein across many influenza strains, these data provide the mandate to assess whether R848+DGP can induce cross-protection against influenza B or other stains of influenza A that differ in HA proteins but share the same NP sequence.

Although the present disclosure has been described in some detail by way of illustration and example for purposes of clarity and understanding, it will be apparent to those skilled in the art that certain changes and modifications may be practiced. Therefore, the examples herein should not be construed as limiting the scope of the present disclosure.

We claim:

1. A compound of Formula (IV-F): 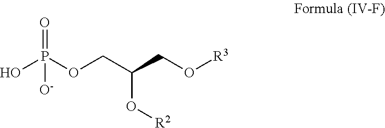
wherein R² is H, —(C═O)—NH₂, —(C═O)—NH(R⁵), —(C═O)—N(R⁵)₂, or —CH₂—C₆H₅;
R³ is C₂₁-C₂₄ n-alkyl; and
each R⁵ is independently C₁-C₄ alkyl;
or a protonated or deprotonated form thereof; or a salt thereof.

2. The compound of claim 1, wherein R² is H.

3. The compound of claim 1, wherein R² is —(C═O)—NH₂.

4. The compound of claim 1, wherein R² is —(C═O)—NH(R⁵).

5. The compound of claim 1, wherein R² is —(C═O)—N(R⁵)₂.

6. The compound of any one of claims 1-5, wherein R³ is C₂₁ n-alkyl.

7. The compound of any one of claims 1-6, wherein R³ is unsubstituted.

8. The compound of any one of claims 1-2 or 4-7, wherein R⁵ is —CH₃.

9. A compound of the formula: 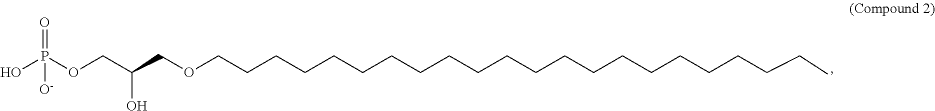 [SMILES: CCCCCCCCCCCCCCCCCCCCCCOC[C@@H](O)COP(=O)([O-])O]
or a protonated or deprotonated form thereof, or a salt thereof.

10. A compound of the formula: 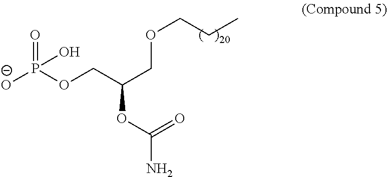
or a protonated or deprotonated form thereof, or a salt thereof.

11. A compound of the formula: 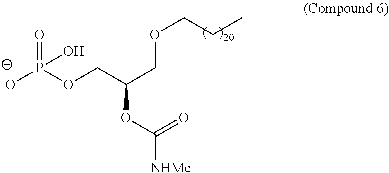
or a protonated or deprotonated form thereof, or a salt thereof.

12. A compound of the formula: 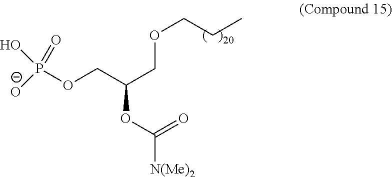
or a protonated or deprotonated form thereof, or a salt thereof.

13. The compound of any one of claims 1-12, wherein the compound is isolated.

14. A composition comprising a compound of any one of claims 1-13 and a TLR agonist.

15. The composition of claim 14, wherein the TLR agonist comprises a TLR7/8 agonist.

16. The composition of any one of claims 1-15, further comprising an antigen.

17. The composition of any one of claims 1-16, further comprising dendritic cells.

18. The composition of any one of claims 14-17, wherein the TLR agonist is a small molecule with a molecule weight of 900 daltons or less.

19. The composition of any one of claims 15-18, wherein the TLR7/8 agonist comprises an imidazoquinoline compound.

20. The composition of any one of claims 15-19, wherein the TLR7/8 agonist comprises resiquimod (R848).

21. A composition comprising an isolated ether lipid (ETL) of Formula (I): 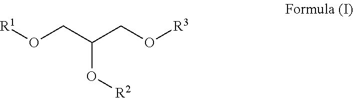
wherein:
R¹ is H or 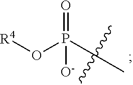
R² is H, C₁-C₄ alkyl, —(C═O)—NH₂, —(C═O)—NH(R⁵)₂, —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; and
a TLR agonist.

22. The composition of claim 21, wherein the TLR agonist comprises a TLR7/8 agonist.

23. The composition of claim 21 or claim 22, wherein R³ is C₁₈-C₂₂ n-alkyl or C₂₁-C₂₄ n-alkyl.

24. The composition of any one of claims 21-23, wherein R³ is C₁₆-C₂₀ n-alkyl.

25. The composition of any one of claims 21-24, further comprising an antigen.

26. The composition of any one of claims 21-25, further comprising dendritic cells.

27. A composition comprising an isolated ether lipid (ETL) of Formula (I): 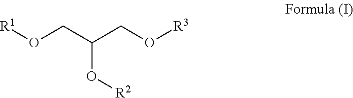
wherein:
R¹ is H or 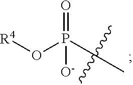
R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R is H or (CH₃)₃N⁺—(CH₂)₂—; and
each R⁵ is independently C₁-C₄ alkyl;
or a protonated or deprotonated form thereof; or a pharmaceutically acceptable salt thereof; and
an antigen.

28. The composition of claim 27, further comprising dendritic cells.

29. The composition of claim 27 or claim 28, further comprising a TLR agonist.

30. The composition of claim 29, wherein the TLR agonist comprises a TLR7/8 agonist.

31. A composition comprising an isolated ether lipid (ETL) of Formula (I): 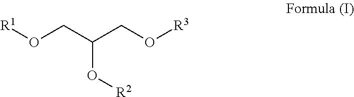
wherein:
R¹ is H or 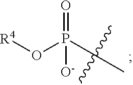
R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁻—(CH₂)₂—; and
each R⁵ is independently C₁-C₄ alkyl;
or a protonated or deprotonated form thereof; or a pharmaceutically acceptable salt thereof; and
dendritic cells.

32. The composition of claim 31, further comprising a TLR agonist.

33. The composition of claim 32, wherein the TLR agonist comprises a TLR7/8 agonist.

34. The composition of any one of claims 31-33, further comprising an antigen.

35. A composition of any one of claims 21-34, wherein R³ is C₂₂ n-alkyl.

36. The composition of any one of claims 21-35, wherein the ETL is an ether phospholipid (ETPL) which comprises 1-docosyl-sn-glycerol-3-phosphocholine (DGPC), or a pharmaceutically acceptable salt thereof.

37. The composition of any one of claims 21-35, wherein the ETL is an ETPL which comprises 1-docosyl-sn-glycerol-3-phosphate (DGP), or a pharmaceutically acceptable salt thereof.

38. The composition of any one of claims 21-37, wherein the TLR agonist is a small molecule with a molecule weight of 900 daltons or less.

39. The composition of any one of claims 21-38, wherein the TLR agonist comprises a TLR7/8 agonist.

40. The composition of claim 39, wherein the TLR7/8 agonist comprises an imidazoquinoline compound.

41. The composition of claim 39, wherein the TLR7/8 agonist comprises resiquimod (R848).

42. The composition of any one of claims 14-41, wherein the TLR7/8 agonist does not inhibit NLR family pyrin domain containing 3 (NLRP3).

43. The composition of any one of claims 21-34, wherein the ETPL comprises one or both of DGPC and DGP, and the TLR7/8 agonist comprises resiquimod (R848).

44. The composition of any one of claims 14-43, wherein the antigen is present in a biological sample obtained from an individual.

45. The composition of claim 44, wherein the biological sample comprises biopsy tissue.

46. The composition of claim 44, wherein the biological sample comprises cells.

47. The composition of claim 44, wherein the biological sample does not comprise cells.

48. The composition of claim 44, wherein the biological sample comprises pus from an abscess.

49. The composition of any one of claims 16-48, wherein the antigen comprises a proteinaceous antigen.

50. The composition of claim 49, wherein the antigen comprises a tumor antigen.

51. The composition of claim 50, wherein the tumor antigen comprises a synthetic or recombinant neoantigen.

52. The composition of claim 50, wherein the tumor antigen comprises a tumor cell lysate.

53. The composition of claim 49, wherein the antigen comprises a microbial antigen and the microbial antigen comprises one or more of a viral antigen, a bacterial antigen, a protozoan antigen, and a fungal antigen.

54. The composition of claim 53, wherein the microbial antigen comprises a purified or recombinant surface protein.

55. The composition of claim 53, wherein the microbial antigen comprises an inactivated, whole virus.

56. The composition of any one of claims 14-55, wherein the composition does not comprise liposomes.

57. The composition of any one of claims 14-56, wherein the composition does not comprise LPS or MPLA.

58. The composition of any one of claims 14-57, wherein the composition does not comprise oxPAPC or a species of oxPAPC, optionally wherein the composition does not comprise HOdiA-PC, KOdiA-PC, HOOA-PC, KOOA-PC, and/or PGPC.

59. The composition of any one of claims 14-58, wherein the composition does not comprise lysophosphatidylcholine (LPC), optionally wherein the composition does not comprise 1-behenoyl-2-hydroxy-sn-glycero-3-phosphocholine [LPC(22:0)].

60. The composition of any one of claims 14-59, further comprising an adjuvant, wherein the adjuvant comprises an aluminum salt adjuvant, a squalene-in-water emulsion, a saponin, or combinations thereof.

61. The composition of any one of claims 14-60, wherein the n-alkyl group is unsubstituted.

62. A pharmaceutical formulation comprising the composition of any one of claims 14-61 and a pharmaceutically acceptable excipient.

63. A method for production of hyperactivated dendritic cells, the method comprising contacting the dendritic cells with a composition comprising effective amounts of an isolated ether lipid (ETL) of
i) Formula (I): 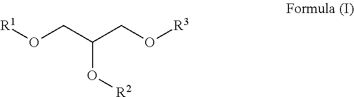
wherein:
R¹ is H or 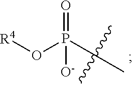
R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof;
or
ii) Formula (IV-F): 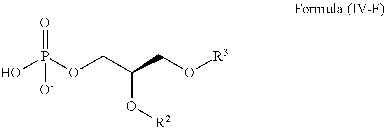
wherein R² is H, —(C═O)—NH₂, —(C═O)—NH(R⁵), —(C═O)—N(R⁵)₂, or —CH₂—C₆H₅;
R³ is C₂₁-C₂₄ n-alkyl; and
each R⁵ is independently C₁-C₄ alkyl;
or a protonated or deprotonated form thereof; or a salt thereof;
and
a TLR7/8 agonist for production of hyperactivated dendritic cells, wherein the hyperactivated dendritic cells secrete IL-1beta without undergoing pyroptosis.

64. The method of claim 63, wherein the dendritic cells are contacted ex vivo with the composition of any one of claims 14-61 or the formulation of claim 62.

65. The method of claim 63, wherein the dendritic cells are contacted in vivo with the formulation of claim 62.

66. A pharmaceutical formulation comprising at least 10³, 10⁴, 10⁵ or 10⁶ of the hyperactivated dendritic cells produced by the method of claim 64, and a pharmaceutically acceptable excipient.

67. A method of stimulating an immune response against an antigen, comprising administering an effective amount of the formulation of claim 62 to an individual in need thereof to stimulate the immune response against the antigen.

68. A method of treating cancer, comprising administering an effective amount of the formulation of claim 62 to an individual in need thereof to treat the cancer.

69. A method of inhibiting abnormal cell proliferation, comprising administering an effective amount of the formulation of claim 62 to an individual in need thereof to inhibit abnormal cell proliferation.

70. A method of treating an infectious disease, comprising administering an effective amount of the formulation of claim 62 to an individual in need thereof to treat the infectious disease.

71. Use of the formulation of claim 62 for inducing an immune response against the antigen in an individual in need thereof.

72. Use of the formulation of claim 62 for inducing an anti-tumor immune response in an individual in need thereof, wherein the individual is or was tumor-bearing.

73. Use of the formulation of claim 62 for inducing an anti-microbe immune response in an individual in need thereof, wherein the individual is infected with the microbe or has not been exposed to the microbe.

74. The composition, formulation, method or use of any one of claims 44-73, wherein the individual is a mammalian subject.

75. The composition, formulation, method or use of any one of claims 44-73, wherein the individual is a human subject.

76. A method of preparing an immunogenic composition, the method comprising:
a) depleting leukocytes from a suspension of cells prepared from a tumor to obtain a tumor cell-enriched suspension;
b) lysing cells from the tumor cell-enriched suspension to obtain a tumor cell lysate; and
c) contacting the tumor cell lysate with an isolated ether lipid (ETL) of
i) Formula (I): 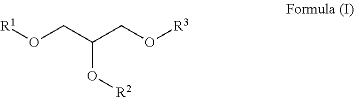
wherein:
R¹ is H or 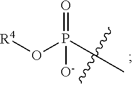
R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof; or
ii) Formula (IV-F): 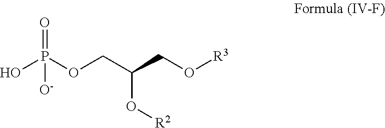
wherein R² is H, —(C═O)—NH₂, —(C═O)—NH(R⁵), —(C═O)—N(R⁵)₂, or —CH₂—C₆H₅;
R³ is C₂₁-C₂₄ n-alkyl; and
each R⁵ is independently C₁-C₄ alkyl;
or a protonated or deprotonated form thereof; or a salt thereof;
and
a toll-like receptor (TLR) agonist to obtain the immunogenic composition.

77. The method of claim 76, wherein the TLR agonist comprises a TLR7/8 agonist.

78. The method of claim 76 or claim 77, wherein the leukocytes are depleted in step a) by negative selection using an anti-CD45 antibody.

79. The method of any one of claims 76-78, wherein the cells are lysed in step b) by one or more freeze-thaw cycles.

80. The method of any one of claims 76-79, wherein R³ in Formula (I) is C₁₈-C₂₂ alkyl or C₁₈-C₂₄ alkyl.

81. The method of any one of claims 76-79, wherein R³ in Formula (I) is C₁₆-C₂₀ alkyl.

82. The method of any one of claims 76-79, wherein R³ is C₂₁-C₂₄ alkyl.

83. The method of any one of claims 76-79, wherein the ETL comprises one or both of DGPC and DGP, or a pharmaceutically acceptable salt thereof.

84. The method of any one of claims 76-83, wherein the TLR7/8 agonist is a small molecule with a molecule weight of 900 daltons or less.

85. The method of claim 84, wherein the TLR7/8 agonist comprises an imidazoquinoline compound.

86. The method of claim 85, wherein the TLR7/8 agonist comprises resiquimod (R848).

87. The method of any one of claims 84-86, wherein the TLR7/8 agonist does not inhibit NLR family pyrin domain containing 3 (NLRP3).

88. The method of claim 83, wherein the ETL comprises one or both of DGPC and DGP or a pharmaceutically acceptable salt thereof, and the TLR7/8 agonist comprises resiquimod (R848).

89. The method of any one of claims 76-88, further comprising before step a) obtaining a sample from the tumor from a mammalian subject with cancer and preparing the suspension of cells from the sample.

90. An immunogenic composition prepared by the method of any one of claims 76-89.

91. A method of eliciting an anti-cancer immune response, the method comprising: administering to a mammalian subject with cancer an effective amount of the immunogenic composition of claim 90.

92. The method of claim 92, wherein the anti-cancer immune response comprises cellular immune response.

93. The method of claim 91, wherein the anti-cancer immune response comprises cancer antigen-induced IL-1beta secretion and/or activation of CD8+ T lymphocytes.

94. The method of any one of claims 91-93, wherein the cancer is a non-hematologic cancer.

95. The method of claim 94, wherein the non-hematologic cancer is a carcinoma, a sarcoma, or a melanoma.

96. The method of any one of claims 91-95, wherein the cancer is a lymphoma.

97. A method of treating cancer, the method comprising:
a) preparing an immunogenic composition comprising a tumor cell lysate, an isolated ether lipid (ETL) of
i) Formula (I): 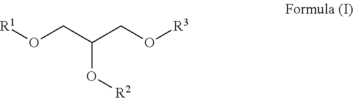
wherein:
R¹ is H or 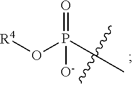
R² is H, C₁-C₄ alkyl, —(C═O)—NH₂, —(C═O)—NH(R⁵), —(C═O)—N(R⁵)₂, or —CH₂—C(H₅);
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof:
or
ii) Formula (IV-F): 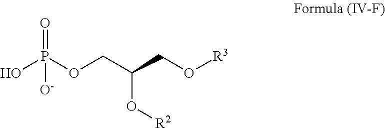
wherein
R² is H, —(C═O)—NH₂, —(C═O)—NH(R⁵), —(C═O)—N(R⁵)₂, or —CH₂—C₆H₅;
R³ is C₂₁-C₂₄ n-alkyl; and
each R⁵ is independently C₁-C₄ alkyl;
or a protonated or deprotonated form thereof; or a salt thereof;
and
a toll-like receptor (TLR) agonist, wherein the tumor cell lysate is or has been prepared from a sample of a tumor obtained from the mammalian subject with cancer; and
b) administering to the subject an effective amount of the immunogenic composition.

98. The method of claim 97, wherein the TLR agonist comprises a TLR7/8 agonist.

99. The method of claim 97 or claim 98, wherein R³ in Formula (I) is a C₁₈-C₂₂ alkyl chain or a C₁₈-C₂₄ alkyl chain.

100. The method of claim 97 or claim 98, wherein R³ in Formula (I) is C₁₆-C₂₀ alkyl.

101. The method of claim 97 or claim 98, wherein R³ is C₂₁-C₂₄ alkyl.

102. The method of claim 97 or claim 98, wherein the ETL comprises one or both of DGPC and DGP, or a pharmaceutically acceptable salt thereof.

103. The method of any one of claims 97-102, wherein the TLR7/8 agonist is a small molecule with a molecule weight of 900 daltons or less.

104. The method of claim 103, wherein the TLR7/8 agonist comprises an imidazoquinoline compound.

105. The method of claim 104, wherein the TLR7/8 agonist comprises resiquimod (R848).

106. The method of any one of claims 97-105, wherein the ETL comprises DGPC or a pharmaceutically acceptable salt thereof, and the TLR7/8 agonist comprises resiquimod (R848).

107. The method of any one of claims 97-105, wherein the ETL comprises DGP or a pharmaceutically acceptable salt thereof, and the TLR7/8 agonist comprises resiquimod (R848).

108. The method of any one of claims 97-107, further comprising administering to the subject an effective amount of an additional therapeutic agent.

109. The method of claim 108, wherein the additional therapeutic agent comprises one or more of the group consisting of an immune checkpoint inhibitor, an antineoplastic agent, and radiation therapy.

110. A composition comprising an isolated ether lipid (ETL) of:
i) Formula (I): 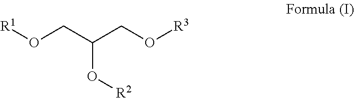
wherein:
R¹ is H or 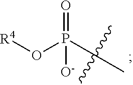
R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof;
or
ii) Formula (IV-F): 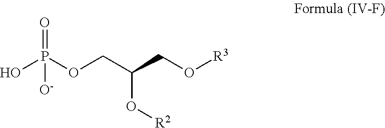
wherein R² is H, —(C═O)—NH₂, —(C═O)—NH(R⁵), —(C═O)—N(R⁵)₂, or —CH₂—C₆H₅;
R³ is C₂₁-C₂₄ n-alkyl; and
each R⁵ is independently C₁-C₄ alkyl;
or a protonated or deprotonated form thereof; or a salt thereof;
and
a pathogen recognition receptor (PRR) agonist.

111. The composition of claim 110, wherein the PRR agonist is an agonist of a toll-like receptor (TLR), a NOD-like receptor (NLR), a RIG-I-like receptor (RLR), or a C-type lectin receptor (CLR).

112. The composition of claim 110, wherein the PRR agonist is an agonist of a cytosolic DNA sensor (CDS) or a stimulator of IFN genes (STING).

113. The composition of claim 110, wherein the PRR agonist comprises one or more of R848, TL8-506, LPS, Pam2CSK4, and ODN 2336.

114. The composition of any one of claims 110-113, further comprising an antigen.

115. The composition of any one of claims 110-114, further comprising dendritic cells.

116. A pharmaceutical formulation comprising the composition of any one of claims 110-115 and a pharmaceutically acceptable excipient.

117. A pharmaceutical formulation comprising an isolated ether lipid (ETL) of
i) Formula (I): 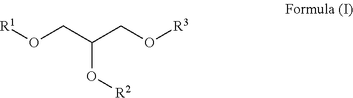
wherein:
R¹ is H or 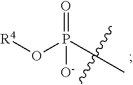
R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁻—(CH₂)₂—; and
each R⁵ is independently C₁-C₄ alkyl;
or a protonated or deprotonated form thereof; or a pharmaceutically acceptable salt thereof;
or
ii) Formula (IV-F): 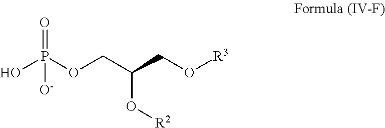
wherein R² is H, —(C═O)—NH₂, —(C═O)—NH(R⁵), —(C═O)—N(R⁵)₂, or —CH₂—C₆H₅;
R³ is C₂₁-C₂₄ n-alkyl; and
each R⁵ is independently C₁-C₄ alkyl;
or a protonated or deprotonated form thereof; or a salt thereof;
and
a pharmaceutically acceptable excipient.

118. The pharmaceutical formulation of claim 117, wherein R³ is C₂₂ n-alkyl.

119. The pharmaceutical formulation of claim 118, wherein the ETL comprises one or both of DGPC and DGP, or a pharmaceutically acceptable salt thereof.

120. A composition for hyperactivation of human dendritic cells, comprising an isolated ether lipid (ETL) of
i) Formula (I): 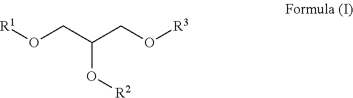
wherein:
R¹ is H or 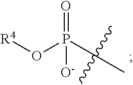
R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
where R⁴ is H or (CH₃)₃N⁺—(CH₂)₂—; and
each R⁵ is independently C₁-C₄ alkyl;
or a protonated or deprotonated form thereof; or a pharmaceutically acceptable salt thereof;
or
ii) Formula (IV-F): 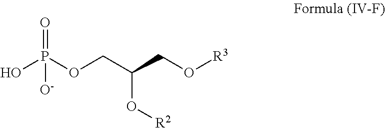
wherein R² is H, —(C═O)—NH₂, —(C═O)—NH(R⁵), —(C═O)—N(R⁵)₂, or —CH₂—C₆H₅;
R³ is C₂₁-C₂₄ n-alkyl; and
each R⁵ is independently C₁-C₄ alkyl;
or a protonated or deprotonated form thereof; or a salt thereof;
and
a pathogen recognition receptor (PRR) agonist, wherein the alkyl chain is a C₂₂ n-alkyl chain, and wherein the composition is effective for achieving a higher level of dendritic cell hyperactivation than a comparator composition comprising PGPC in place of the ETL.

121. The composition of claim 120, wherein R³ is C₂₂ n-alkyl.

122. The composition of claim 120 or claim 121, wherein the higher level of dendritic cell hyperactivation comprises induction of IL-1beta secretion from the human dendritic cells in vitro at a level that is at least 2, 3 or 4 fold higher when contacted with the composition comprising the ETL and the PRR agonist than when contacted with the comparator composition comprising the PGPC and the PRR agonist, wherein the PRR agonist is LPS.

123. The composition of claim 122, wherein the concentration of the ETL and the concentration of the PGPC are the same concentration in a range of from about 10 μM to about 80 μM, and the LPS is present at a concentration of 1 μg/ml in both the composition and the comparator composition.

124. The composition of claim 122 or claim 123, wherein the higher level of dendritic cell hyperactivation comprises a lipid activity index for IL-1beta secretion from the human dendritic cells for the composition comprising the ETL and the PRR agonist that is at least 4, 5 or 6 fold higher in activity units than that of the comparator composition comprising the PGPC and the PRR agonist.

125. The composition, formulation, method or use of any one of claims 44-73, wherein the individual is a human subject.

126. The composition, formulation, method or use of any one of claims 44-73, wherein the individual is a canine subject.

127. The composition, formulation, method or use of any one of claims 89-124, wherein the mammalian subject is a human patient.

128. The composition, formulation, method or use of any one of claims 89-124, wherein the mammalian subject is a non-human patient.

129. The composition, formulation, method or use of any one of claims 89-124, wherein the mammalian subject is a canine patient.

130. The composition, formulation, method or use of any one of claim 14-125 or 127, wherein the dendritic cells are human dendritic cells.

131. The composition, formulation, method or use of any one of claim 14-74, 76-119 or 129, wherein the dendritic cells are canine dendritic cells.

132. The composition, method or use of claim 130 or claim 131, wherein the dendritic cells are present in a composition comprising peripheral blood mononuclear cells (PBMCs).

133. The composition, method or use of any one of claims 42-54 or claims 109-110, wherein the hyperactivated dendritic cells secrete one or both of IFNγ and TNFα.

134. The composition, formulation, method or use of any one of claims 14-133, further comprising a surfactant.

135. The composition, formulation, method or use of claim 134, wherein the surfactant comprises a non-ionic surfactant.

136. The composition, formulation, method or use of claim 135, wherein the non-ionic surfactant comprises an ethylene oxide-propylene oxide copolymer (a poloxamer).

137. The composition, formulation, method or use of claim 135, wherein the non-ionic surfactant comprises one or more of Poloxamer 407, Poloxamer 188, and P123.

138. The composition, formulation, method or use of claim 135, wherein the non-ionic surfactant comprises Poloxamer 407.

139. The composition, formulation, method or use of any one of claims 135-138, wherein i) the ETL is dissolved in an alcohol to form an ETL alcohol solution; ii) the ETL alcohol solution is mixed with the non-ionic surfactant to form a mixture; and iii) the alcohol is evaporated from the mixture to form particles comprising the ETL and the non-ionic surfactant.

140. The composition, formulation, method or use of any one of claims 135-139, wherein the non-ionic surfactant is present in an amount of about 2.5% to 25% (w/w), optionally about 5% to 20% (w/w), optionally about 15% (w/w).

141. The composition, formulation, method or use of any one of claims 135-140, wherein the ETL and non-ionic surfactant are present in particles with a diameter of about 1000 to 15,000 nanometers, optionally with a diameter of about 5000 nanometers.

142. An isolated ether lipid (ETL) of Formula (I): 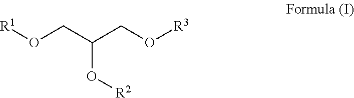
wherein R¹ is H or 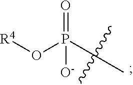
R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
wherein R⁴ is H or (CH₃)₃N⁺—(CH₂)₂—; and
each R⁵ is independently C₁-C₄ alkyl;
or a protonated or deprotonated form thereof; or a salt thereof.

143. The isolated ether lipid of claim 142, wherein the isolated ether lipid is a compound of Formula (II): 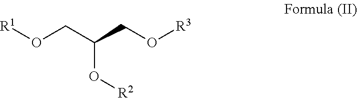
wherein R¹ is H or 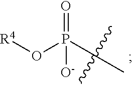
R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
wherein R⁴ is H or (CH₃)₃N⁺—(CH₂)₂—; and
each R⁵ is independently C₁-C₄ alkyl;
or a protonated or deprotonated form thereof; or a salt thereof.

144. The isolated ether lipid of claim 142, wherein the isolated ether lipid is a compound of Formula (III): 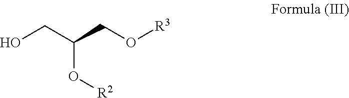
wherein R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl; and
each R⁵ is independently C₁-C₄ alkyl;
or a salt thereof.

145. The isolated ether lipid of claim 142, wherein the isolated ether lipid is an isolated ether phospholipid (ETPL) compound of Formula (IV): 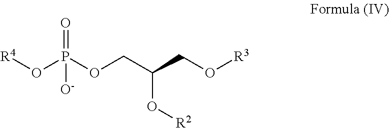
wherein R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl;
R⁴ is H or (CH₃)₃N⁺—(CH₂)₂—; and
each R⁵ is independently C₁-C₄ alkyl;
or a protonated or deprotonated form thereof; or a salt thereof.

146. The isolated ether lipid of claim 142, wherein the isolated ether lipid is an isolated ether phospholipid (ETPL) compound of Formula (IV-A): 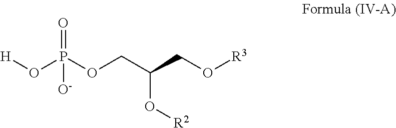
wherein R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl; and
each R⁵ is independently C₁-C₄ alkyl;
or a protonated or deprotonated form thereof; or a salt thereof.

147. The isolated ether lipid of claim 142, wherein the isolated ether lipid is an isolated ether phospholipid (ETPL) compound of Formula (IV-B): 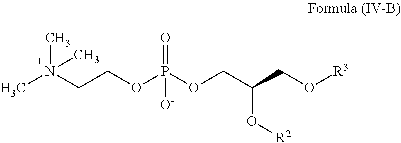
wherein R² is H, C₁-C₄ alkyl, —(C═O)—NH₂, —(C═O)—NH(R⁵), —(C═O)—N(R⁵)₂, or —CH₂—C₆H₅;
R³ is C₁₃-C₂₄ n-alkyl; and
each R⁵ is independently C₁-C₄ alkyl;
or a protonated form thereof; or a salt thereof.

148. The isolated ether lipid of claim 142, wherein the isolated ether lipid is an isolated ether phospholipid (ETPL) compound of Formula (IV-C): 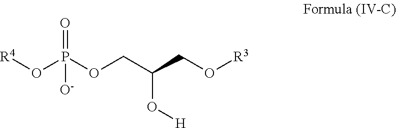
wherein R³ is C₁₃-C₂₄ n-alkyl; and
R⁴ is H or (CH₃)₃N⁺—(CH₂)₂—:
or a protonated or deprotonated form thereof; or a salt thereof.

149. A compound of formula 2: 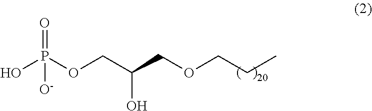
or a protonated form thereof; or a pharmaceutically acceptable salt thereof.

150. The compound of claim 149, wherein said compound is isolated.

151. An isolated compound 1 of formula 1: 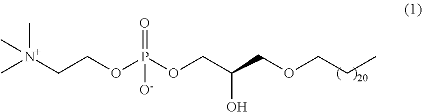
or a protonated form thereof; or a pharmaceutically acceptable salt thereof.

152. A compound of Formula (III-A-1): 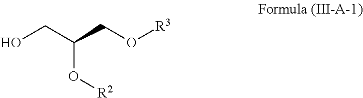
wherein:
R² is —(C═O)—NH₂, —(C═O)—NH(R⁵), or —(C═O)—N(R⁵)₂;
R³ is C₂₁-C₂₄ n-alkyl; and
each R⁵ is independently C₁-C₄ alkyl;
or a pharmaceutically acceptable salt thereof.

153. The compound of claim 152, wherein R² is —(C═O)—NH₂.

154. The compound of claim 152, wherein R² is —(C═O)—NH—CH₃.

155. The compound of claim 152, wherein R² is —(C═O)—N(CH₃)₂.

156. The compound of any one of claims 152-155, wherein R³ is C₂₂ n-alkyl.

157. A compound 7 of formula 7: 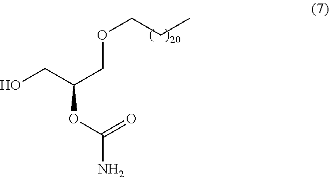
or a pharmaceutically acceptable salt thereof.

158. The compound of claim 157, wherein said compound is isolated.

159. A compound 8 of formula 8: 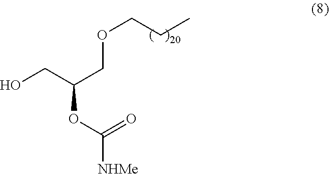
or a pharmaceutically acceptable salt thereof.

160. The compound of claim 159, wherein said compound is isolated.

161. A compound of the formula: 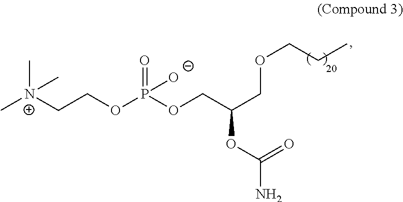 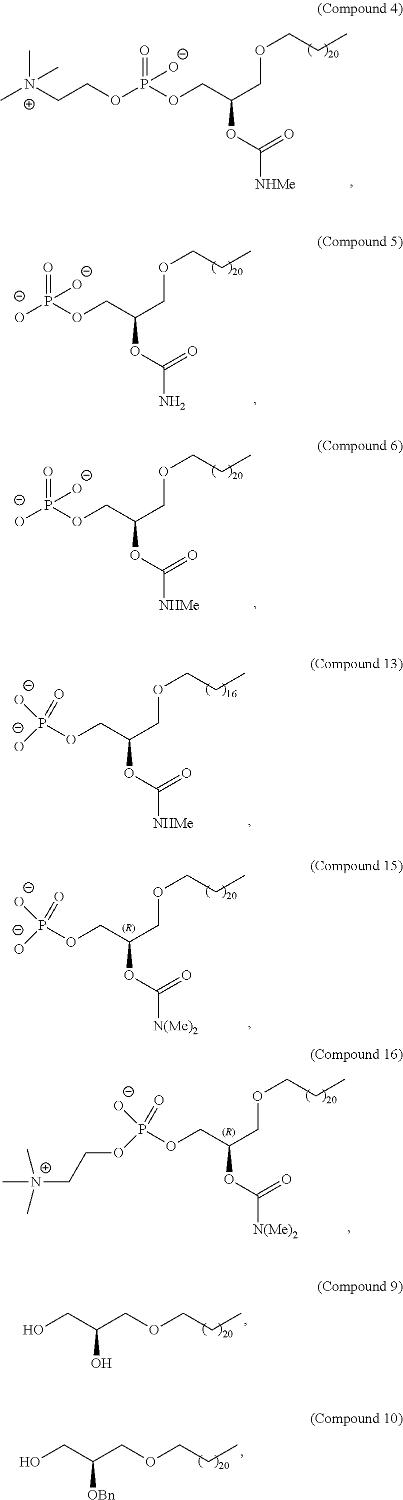 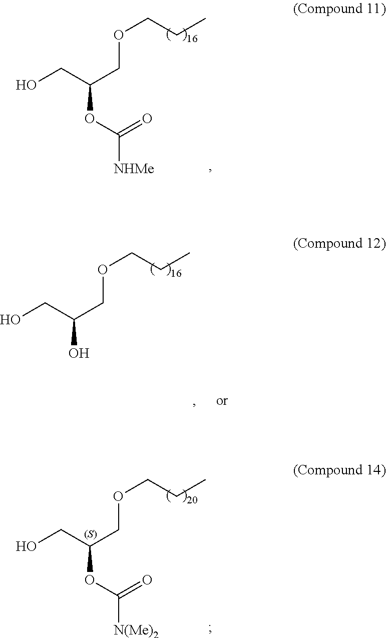
or a protonated form thereof; or a salt thereof.

162. A compound of claim 161, wherein said compound is isolated.

163. A composition comprising the compound of any one of claims 142-162 and a pharmaceutically acceptable excipient.

164. The composition of claim 163, further comprising a surfactant.

165. The composition of claim 164, wherein the surfactant is selected from the group consisting of a non-ionic surfactant, a wetting agent, P407, P188, polysorbate 80, a thickening agent, and carboxymethyl cellulose.

166. The composition of any one of claims 163-165, comprising particles having a diameter less than between about 5 microns and about 20 microns (D₅₀<5 microns to 20 microns), where the particles comprise an ether lipid and a non-ionic surfactant.

167. The composition of any one of claims 163-166, wherein the pharmaceutically acceptable excipient comprises phosphate-buffered saline.

168. The composition of any one of claims 163-167, wherein the pharmaceutically acceptable excipient comprises an aqueous solution of an ethylene oxide-propylene oxide copolymer (a poloxamer), or further comprises an ethylene oxide-propylene oxide copolymer.

169. The composition of any one of claims 163-165, wherein the pharmaceutically acceptable excipient comprises phosphate-buffered saline and at least one of Poloxamer 407, Poloxamer 188, and P123.

170. The composition of any one of claims 163-169, wherein said composition is sterile.

171. An article of manufacture comprising a container enclosing a liquid formulation of the compound of any one of claims 142-170 and a pharmaceutically acceptable excipient.

172. The article of manufacture of claim 171, wherein the container is a syringe.

173. The article of manufacture of claim 172, wherein the syringe is further contained within an injection device.

174. The article of manufacture of claim 173, wherein the injection device is an auto-injector.

175. A composition comprising an isolated ether lipid (ETL) or ether phospholipid (ETPL) compound of Formula (I), Formula (II), Formula (III), Formula (III-A), Formula (III-A-1), Formula (III-A-2), Formula (III-B), Formula (III-B-1), Formula (III-B-2), Formula (IV), Formula (IV-A), Formula (IV-A-1), Formula (IV-A-2), Formula (IV-B), Formula (IV-B-1), Formula (IV-B-2), Formula (IV-C), Formula (IV-D), Formula (IV-E), Formula (IV-F), Formula (A), Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16 as disclosed herein; or a protonated or deprotonated form thereof where possible, or a pharmaceutically acceptable salt thereof; and at least one further lipid, wherein the at least one further lipid is selected from the group consisting of an ionizable lipid, a cationic lipid, a further phospholipid, a pegylated lipid, a structural lipid, and mixtures thereof.

176. The composition of claim 175, wherein the ETL or ETPL and the at least one further lipid are part of a lipid nanoparticle (LNP).

177. The composition of claim 175 or claim 176, further comprising an antigen.

178. The composition of any one of claims 175-177, further comprising dendritic cells.

179. The composition of any one of claims 175-178, further comprising a TLR agonist.

180. The composition of any one of claims 175-178, further comprising a TLR7/8 agonist.

181. The composition, method, or use of any one of claims 16, 25, 27, 34, 67, 71, 114, or 177, wherein the antigen comprises one or more viral antigens.

182. The composition, method, or use of claim 181, wherein the one or more viral antigens comprise one or both of influenza A and influenza B antigens.

183. The composition, method, or use of claim 182, wherein the one or both of influenza A and influenza B antigens comprise one or both of hemagglutinin and nucleoprotein.

184. The composition, method, or use of any one of claims 181-183, wherein the viral antigens comprise inactivated virions, optionally wherein the inactivated virions comprise inactivated, split virions.

185. The composition, method, or use of any one of claim 182-184, comprising both influenza A and influenza B antigens of an HINI influenza A virus, an H3N2 influenza A virus, a Victoria lineage influenza B virus, and a Yamagata lineage influenza B virus.
